SEQUENCE LISTING (14 sequences)

SEQ ID NO: 1 - protein, 1128 aa, Human gammaherpesvirus 4
MQGAQTSEDNLGSQSQPGPCGYIYFYPLATYPLREVATLGTGYAGHRCLTVPLLCGITVEPGFSINVKALHRRPDPNCGLLRATSYHRDIYVFHNAHMVPPIFEGPGLEALCGETREVFGYDAYSALPRESSKPGDFFPEGLDPSAYLGAVAITEAFKERLYSGNLVAIPSLKQEVAVGQSASVRVPLYDKEVFPEGVPQLRQFYNSDLSRCMHEALYTGLAQALRVRRVGKLVELLEKQSLQDQAKVAKVAPLKEFPASTISHPDSGALMIVDSAACELAVSYAPAMLEASHETPASLNYDSWPLFADCEGPEARVAALHRYNASLAPHVSTQIFATNSVLYVSGVSKSTGQGKESLFNSFYMTHGLGTLQEGTWDPCRRPCFSGWGGPDVTGTNGPGNYAVEHLVYAASFSPNLLARYAYYLQFCQGQKSSLTPVPETGSYVAGAAASPMCSLCEGRAPAVCLNTLFFRLRDRFPPVMSTQRRDPYVISGASGSYNETDFLGNFLNFIDKEDDGQRPDDEPRYTYWQLNQNLLERLSRLGIDAEGKLEKEPHGPRDFVKMFKDVDAAVDAEVVQFMNSMAKNNITYKDLVKSCYHVMQYSCNPFAQPACPIFTQLFYRSLLTILQDISLPICMCYENDNPGLGQSPPEWLKGHYQTLCTNFRSLAIDKGVLTAKEAKVVHGEPTCDLPDLDAALQGRVYGRRLPVRMSKVLMLCPRNIKIKNRVVFTGENAALQNSFIKSTTRRENYIINGPYMKFLNTYHKTLFPDTKLSSLYLWHNFSRRRSVPVPSGASAEEYSDLALFVDGGSRAHEESNVIDVVPGNLVTYAKQRLNNAILKACGQTQFYISLIQGLVPRTQSVPARDYPHVLGTRAVESAAAYAEATSSLTATTVVCAATDCLSQVCKARPVVTLPVTINKYTGVNGNNQIFQAGNLGYFMGRGVDRNLLQAPGAGLRKQAGGSSMRKKFVFATPTLGLTVKRRTQAATTYEIENIRAGLEAIISQKQEEDCVFDVVCNLVDAMGEACASLTRDDAEYLLGRFSVLADSVLETLATIASSGIEWTAEAARDFLEGVWGGPGAAQDNFISVAEPVSTASQASAGLLLGGGGQGSGGRRKRRLATVLPGLEV

SEQ ID NO: 2 - DNA, 171823 bp, Human gammaherpesvirus 4
  source 1..171823: mol_type = genomic DNA
agaattcgtcttgctctattcacccttacttttcttcttgcccgttctctttcttagtatgaatccagtatgcctgcctgtaattgttgcgccctacctcttttggctggcggctattgccgcctcgtgtttcacggcctcagttagtaccgttgtgaccgccaccggcttggccctctcacttctactcttggcagcagtggccagctcatatgccgctgcacaaaggaaactgctgacaccggtgacagtgcttactgcggttgtcacttgtgagtacacacgcaccatttacaatgcatgatgttcgtgagattgatctgtctctaacagttcacttcctctgcttttctcctcagtctttgcaatttgcctaacatggaggattgaggacccaccttttaattctcttctgtttgcattgctggccgcagctggcggactacaaggcatttacggttagtgtgcctctgttatgaaatgcaggtttgacttcatatgtatgccttggcatgacgtcaactttacttttatttcagttctggtgatgcttgtgctcctgatactagcgtacagaaggagatggcgccgtttgactgtttgtggcggcatcatgtttttggcatgtgtacttgtcctcatcgtcgacgctgttttgcagctgagtcccctccttggagctgtaactgtggtttccatgacgctgctgctactggctttcgtcctctggctctcttcgccagggggcctaggtactcttggtgcagcccttttaacattggcagcaggtaagccacacgtgtgacattgcttgcctttttgccacatgttttctggacacaggactaaccatgccatctctgattatagctctggcactgctagcgtcactgattttgggcacacttaacttgactacaatgttccttctcatgctcctatggacacttggtaagttttcccttcctttaactcattacttgttcttttgtaatcgcagctctaacttggcatctcttttacagtggttctcctgatttgctcttcgtgctcttcatgtccactgagcaagatccttctggcacgactgttcctatatgctctcgcactcttgttgctagcctccgcgctaatcgctggtggcagtattttgcaaacaaacttcaagagtttaagcagcactgaatttatacccagtgagtatctatttgttactcctgtttagttgaagaaaacaagctattggattgtaacacacattttacgctttgttccttagatttgttctgcatgttattactgattgtcgctggcatactcttcattcttgctatcctgaccgaatggggcagtggaaatagaacatacggtccagtttttatgtgcctcggtggcctgctcaccatggtagccggcgctgtgtggctgacggtgatgtctaacacgcttttgtctgcctggattcttacagcaggattcctgattttcctcattggtaagtgtgacaccaacaggtgttgccttgttatgtcaccgttctgacacatgacttacatgggtttggcttttgtaggctttgccctctttggggtcattagatgctgccgctactgctgctactactgccttacactggaaagtgaggagcgcccaccgaccccatatcgcaacactgtataaaggtaagtattattaaattttagagacactatcacgtgtaacttgacgtgcaaggatggaagagaggggcagggaaacgcaaatgccggttgcccggtatgggggcccgtttattatggtaaggctcttcgggcaagatggagaggcaaacatacaggaggaaaggctatatgagctactctctgacccacgctccgcgctcggcctagacccggggcccctgattgctgagaacctgctgctagtggcgctgcgtggcaccaacaacgatcccaggcctcagcgtcaggagagggccagagaactggccctcgttggcattctactaggaaacggcgagcagggtgaacacttgggcacggagagtgccctggaggcctcaggcaacaactatgtgtatgcctacggaccagactggatggcaaggccttccacatggtccgcggaaatccagcaattcctgcgactcctgggcgccacgtacgtgcttcgcgtggagatgggcaggcagtttggcttcgaggtgcatagaagccggccctccttccgtcagttccaggccatcaatcaccttgtcctgtttgacaacgcccttcgcaagtacgattccggccaggtggcggcgggcttccagagggcccttctggtggccgggccagagaccgctgacacgaggccggacctccgcaagctgaatgagtgggtgtttggtggcagggctgctggtggcagacagctggccgacgagctaaagatcgtgtccgcgctgcgagacacttactcgggccacttggtccttcagcccacggagacccttgacacatggaaggtgttgagcagggacacacgaaccgctcatagtttggagcacggattcattcatgccgcggggaccatccaggccaactgcccacagctgtttatgagacgccagcaccccggcctctttcccttcgttaatgcaatagcatcatcgctgggctggtactaccagaccgccaccggccccggagcagatgccagggcggcggcccggcgccaacaggcctttcagaccagggcggcggctgaatgccatgccaaaagcggggtgccggtcgtggccggcttctacaggaccatcaacgccacgctcaagggaggagagggcctacagcccactatgtttaacggggagctgggggccatcaagcaccaggcacttgacactgtgaggtatgactacggccactatctcataatgttggggccattccagccatggagcggactgacggcccctccgtgcccctacgccgaaagttcatgggcacaggcggccgtgcagacggccctcgagctgttctcggccctgtacccggccccgtgcatctcgggctacgcgcgccccccgggccccagtgctgtgatcgagcatctggggtccctagttccaaaggggggtctgctgttgtttctgtctcacctaccggatgatgttaaggacgggctcggagaaatggggccggccagggccacgggacctggaatgcagcagtttgtcagcagctacttcctcaaccccgcctgttccaacgtcttcattacagtgaggcagcgaggggagaagatcaacggccgtaccgtcctccaagcgctcggacgcgcatgcgatatggcaggctgccagcactatgtgctgggctccacggttcccctcggtggactcaactttgtcaacgacctggcgtccccggtttccaccgccgagatgatggatgatttctctcccttcttcaccgtggagtttcccccgattcaagaggagggcgcaagttctccggtacccttagatgtggacgagagcatggacatctctccgtcttacgagttgccctggctctcgctggagtcatgcctcacaagcatcctgtcacaccccaccgtgggaagcaaggagcacttggtcaggcacacggacagggtcagcggaggacgcgtggcacagcagcccggggtaggtcccctggacctgccgctggcggactacgccttcgttgcccacagtcaggtctggaccaggcccggtggggctcctcccttgccctatcgtacctgggatcgaatgacagagaagctgcttgtctccgcaaaacccggcggagagaacgttaaggtttcaggtaccgtgattacattgggagaacaggggtacaaagtgtcgttggatctgagggagggaaccaggctggcaatggctgaggcgctgctgaacgcagcatgtgccccaatcttggatccggaagacgtcttgctcaccctgcatctacacctggatccgcgccgggcagacaactcggccgtgatggaggctatgacggcggcgagtgactacgcgcgtggcctgggcgtgaagctgacctttggctcggcctcctgccccgagaccggctcgtccgcctccaacttcatgaccgtggtggcctctgtctccgccccaggggaattctcgggtcctctgatcacgccagtgcttcagaagacgggcagtctcctgattgcggtgcgttgcggggatggcaagatccagggagggtcgctgtttgagcagctctttagcgacgtggccacgaccccacgggcacccgaggcgttgtctctgaagaatctcttccgggcagtccagcagctggtcaagagcggcatcgtgctgtcagggcatgacatcagcgacgggggcctggtgacctgcctggtggagatggccctggccgggcagcggggagtgaccatcactatgccggtggcctccgactacctcccggagatgtttgcagagcaccccggcctggtgtttgaggtggaggagcgcagcgtgggtgaggtgctgcagaccctgcgctccatgaacatgtacccggcagtcctcggtcgagtgggcgagcaaggtccagatcaaatgtttgaggtgcagcacggcccagagacggtgttgcgccagtcgctgcgcctgctgctgggaacctggtcatcctttgccagcgagcagtacgagtgcctgcgaccagatcggattaaccggtccatgcacgtgtccgactacggctataacgaagcactggcagtctccccgttgacaggaaagaatctcagcccacgccggttggtgacagagcctgacccacgatgtcaggtggccgtgctatgcgccccgggcaccaggggccatgaaagcctcctggcggccttcacgaatgccggatgcctgtgccgacgggtgttctttcgcgaggttagggacaacacgttcctcgacaagtacgtgggtctggccatcggaggagttcatggggccagggactctgccctggcaggccgtgccaccgtggcgctgattaatcgtttccccgccctgcgtgacgctattctaaagttcctcaacaggccagatacgttctcggtggccttgggggagctgggggtgcaagttttggctggcctgggggccgtggggtcaacagataatccacccgcccctggcgtggaagttaatgtccagagatcacctctgattctggcccccaacgcctctggcatgtttgagtcccgctggctgaacattagcatcccggcgaccaccagctctgtcatgctgcgtggcctccggggctgcgtcctgccttgttgggtgcaaggctcgtgcctgggcctgcaatttactaacctcgggatgccatatgttttgcagaatgcccaccagatcgcctgccacttccacagcaatggcacggatgcctggcgctttgctatgaattatccaagaaaccccacggagcagggcaacattgcagggctctgttcacgcgatggtcgtcatctggctctcctgtgtgacccctcactttgtacagacttttggcaatgggagcacattccccccgcctttgggcaccccacggggtgctccccctggacacttatgtttcaagcagctcacctatggtcactcaggcacggtcgcccctccgagtgaccagtcaccttccagactatgcatacactgaatttagcctgatattgtccccctagccccgggcccagccctcctcagaaaactctgcatggagaagctggacgtgaacctcccccccagacctgtgtgctgtatttacaaacactacaataaacccaatgtgcaaatgtggtttgtatggctactttgtgttcctaaaaaatgcaacaatagaagtggaaaccctcagtcacgggacattaacctcaaccacaaaatgggggttggagaaagtaaccacatatactggagatgattcatgggctgggggttcccggacaatacacccatctggagttcaacctaattacatggtagataaattaagagtccctcctcaccactcgaaactatggcagacattctataagataacgaggagagatgaggtgagggcagaggacattgggcaggtgtgggccacggggcagctggccatatcccccgcactacagaagtgtaagcaaagtgaagggctcggaaggcaggcggggcctagcaatgtcacagctaaatgcccaccagggcacacactcaagcggggtctcggagctcctaggtcagaccacgaaaggtcagcctgcaaggtggatggcgtgttttctgaggttatccccgctacgtgcagtgctgggtgatagagaccctagaatgtgtcgaaatgaccaagcgtccccgcagcggggctcccaacacgggttcccagagagggtaaaagagggggccataaagcccagggtgtaaaacaccgaccgcgccaccagatggcacacgtgggggaaatgagggttagcataggcaacccccgcctacacaccaactatagcaaaccccgccccgtcacggtgacgtagtctgtcttgaggagatgtagacttgtagacactgcaaaacctcaggacctacgctgccctagaggttttgctagggaggagacgtgtgtggctgtagccacccgtcccgggtacaagtcccgggtggtgaggacggtgtctgtggttgtcttcccagactctgctttctgccgtcttcggtcaagtaccagctggtggtccgcatgttttgatccaaacttttgttttaggatttatgcatccattatcccgcagttccacctaaacggggcttaacgttgcatcccagaagatgcacgcttaaccccgcctacaaccgtgacgtagctgtttaccagcatgtatagagttacggttcgctacatcaaacaggacagccgttgccctagtggtttcggacacaccgccaacgctcagtgcggtgctaccgacccgaggtcaagtcccgggggaggagaagagaggcttcccgcctagagcatttgcaagtcaggattctctaatccctctgggagaagggtattcggcttgtccgctatttttttgtggctagttttgcacccacaacatgtaagggcccgctacccctacaacacaaaacaaactatctcccctaaccatccttttgccaatcaattctgtgacagggtttcctggacacccagtcttagttcaggtagacacccagttatgcagtgccaccaattccaaccatttttaaacctcctggaattctatcattaaacggcatgcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaaggcaatgttgtgttgcagtccacagactgcaaagtctgctccaggatgaaagccactcagtgttggcaaatgtgcacatccatttataaggatgtcaactacagtcagagaacccctttgtgtttggtccccccccgtgtcacatgtggaacagggcccagttggcaagttgtaccaaccaactgaagggattacatgcactgccccgcgggaaatacgtcctacccaggaacccgaaacagtgtttcccagaagctgtaaaaatagaacgccctggaactgccccactgtgcaatgcagcttttagccatgccatgctctataaatcacttccctatctcaggtaggcctgcacaccttaggtatggagcgaaggttagtggtcactctgcagtgcctggtgctgctttacctggcacctgagtgtggaggtacagaccaatgtgacaattttccccaaatgttgagggacctaagagatgccttcagtcgtgttaaaacctttttccagacaaaggacgaggtagataaccttttgctcaaggagtctctgctagaggactttaagggctaccttggatgccaggccctgtcagaaatgatccaattctacctggaggaagtcatgccacaggctgaaaaccaggaccctgaagccaaagaccatgtcaattctttgggtgaaaatctaaagaccctacggctccgcctgcgcaggtgccacaggttcctgccgtgtgagaacaagagtaaagctgtggaacagataaaaaatgcctttaacaagctgcaggaaaaaggaatttacaaagccatgagtgaatttgacatttttattaactacatagaagcatacatgacaattaaagccaggtgataattccataccctggaagcaggagatgggtgcatttcaccccaaccccccctttcgactgtcatttacaataaaatgaaaccttttattcttgattgcctcttgtgttcttgccgcccaggtaccttcctgtgttctccccacgggaaaaagaatagcttctgcagaaggccattgacgcaagttttgcccgtggggattacccgacccagccacttacagcacattttgttctaggtccatcttaggagcccgggccagcattctatcagcttaacgggaagagaagtggggagggcactcgcccactaaccttaacacctgcagcctacaaaagtacactagctgtttgctctattcgccactagagaccgccaagatgcgaaactacaggcccgggcccaggccttgcagggcagacggttaggctgacaaggggacaagtgtggcaggtgggcgggaaggggcacaagaatgccggcgaaactggaccacggtccaccccgccctcaagcgtccgggagccgggcggctcggctaaggagggcggccttgcgaacaattattagtagctaccaacaagggcccccagatgccccccaccagtcacccggccgtgtccactcacatattccactcttatttttaaattaatgtgtcccaattagaaacccaagcgcagaaattagttgagaggctagtgttttaaacatgcaccctaggccagccagagataatgtcacaagattatcaagttggtgtaaacacgccgtgggaaaaaatttatggttcagtgcgtcgagtgctatctttggaacagtagaaaattgaaccttgttggcgggagaaggaataacgccttatctgggaggagcgacggattatagccaataagagagctcaagacgcagggctcgcaaagtatagtggccccgtgggaccttagaggtggagcaacgtctaaagtggtaataacaccaggcggggctgggcaaaggggtcctacgggcgggattaattacgccttgcttacgcaagctcagttaattcgcccacgacttgaaaaatgtagcccttaaccaattggcggcccctaagggggggactaaggtcccactacaaaaactctgtgttctgctgcaaattttagatcagatggcatagagacaaggacaccgaagacccccagagccctcatcgcagggttcttaccatgcggccatgtaggcccacttaacactacaagacctacgcctctccattcatcatgtaacccacaaatcatctaaaccgtaagtctaagggcctcctgaggttttctcaggaggccctaatgtataattaatcatgcatttgattttaaaaaagtaggttacactcattttaggccagactttatttgcagattaataatttatgtgattctccttccctctaggactgaagaaacagcctcctgcacgtgagcatgtatctgaaataattattatgtcataagtgtaatgattagaaagtcataaacccacttccctttacatgaatctgggcactgaattttggggtacttctaaagactaacgtgttcgatttcggggtcacttccccttttataagtgtgtgaacagtgatttcagtaaaacctaagagatatttggtgtcacttccgcattttaagtttcagaaaattttaaaattaaaattgaaatttctctcaaaataattccaatgaaaacttcaaagaatcttatgtatgtaattcttttgccccaaactgggcttcagatgccttctattgcactctcacaaaaacattctggacacatgtgccagacgcctgggcctctaaggccctcgggtccccctggaccccggcctcagcaaccctgctgctcccctcctgccaccccagcctccccccctccccgtcccccttcgctcctgttcctcccccggtccccagtagggccgcctgcccccctgcacccagtacctgcccctcttggccacgcaccccgggccaggccaccttagacccggccaagccccatccctgaagacccagcggccattctctctggtaacgagcagagaagaagtagaggcccgcggccattgggcccagattgagagaccagtccaggggcccgaggttggagccagcgggcacccgaggtcccagcacccggtccctccggggggcagagacaggcagggccccccggcagctggccccgaggaggcgcccggagtggggccggtcggctgggctggccgagcccgggtctgggaggtctggggtggcgagcctgctgtctcaggaggggcctggctccgccgggtggccctggggtaagtctgggaggcagagggtcggcctaggcccggggaagtggagggggatcgcccgggtctctgttggcagagtccgggcgatcctctgagaccctccgggcccggacggtcgccctcagccccccagacagaccccagggtctccaggcagggtccggcatcttcaggggcagcaggctcaccaccacaggccccccagacccgggtctcggccagccgagccgaccggccccgcgcctggcgcctcctcggggccagccgccggggttggttctgcccctctctctgtccttcagaggaaccagggacctcgggcaccccagagcccctcgggcccgcctccaggcgccctcctggtctccgctcccctctgagccccgttaaacccaaagaatgtctgaggggagccaccctcggggcccaggccccagagtccagaggtcaggggcacctcagggtgcctccccgggtcccaggccagccggagggaccccggcagcccgggcggccccagaggccggttcctcgccccttccccgggcttcagagcccaggatgtcccccagaagggaccctaggcgtcccctctcctcccctccaggcccgagcctctccctcgcggagaggggcctctttgggccctcaagtccagccccaccgagacccgagtggcccggatccccccaccggcccttctctctgtccccctgctcctctccaaccttcgctccaccctagaccccagcttctggcctccccgggtccaccaggccagccggagggaccccggcagcccgggcgagtcgccttccctctcccctggcctctccttcccgcctcccacccgagccccctcagcttgcctccccaccgggtccatcaggccggccggagggaccccggcggcccggtgtcagtcccccctgcagccgcccagtctctgcctccaggcaagggcgccagcttttctccccccagcctgaggcccagtctcctgtgcactgtctgtaaagtccagcctcccacgcccgtccacggctcccgggcccagcctcgtccacccctccccacggtggacaggccctctgtccacccgggccatccccgcccccctgtgtccaccccagtcccgtccaggggggactttatgtgacccttgggcctggctccccatagactcccatgtaagcctgcctcgagtaggtgcctccagagccccttttgcccccctggcggcccagcccgacccccgggcgcccccaaactttgtccagatgtccaggggtccccgagggtgaggcccagccccctcccgcccctgtccactgccccggtccccccagaagcccccaaaagtagaggctcaggccatgcgcgccctgtcaccaggcctgccaaagagccagatctaaggccgggagaggcagccccaaagcgggtgcagtaacaggtaatctctggtagtgatttggacccgaaatctgacactttagagctctggaggactttaaaactctaaaaatcaaaactttagaggcgaatgggcgccattttgtccccacgcgcgcataatggcggacctaggcctaaaacccccaggaagcgggtctatggttggctgcgctgctgctatctttagaggggaaaagaggaataagcccccagacaggggagtgggcttgtttgtgacttcaccaaaggtcagggcccaagggggttcgcgttgctaggccaccttctcagtccagcgcgtttacgtaagccagacagcagccaattgtcagttctagggagggggaccactgcccctggtataaagtggtcctgcagctatttctggtcgcatcagagcgccaggagtccacacaaatgtaagagggggtcttctacctctccctagccctccgccccctccaaggactcgggcccagtttctaacttttccccttccctccctcgtcttgccctgcgcccggggccaccttcatcaccgtcgctgactccgccatccaagcctaggggagaccgaagtgaaggccctggaccaacccggcccgggccccccggtatcgggccagaggtaagtggactttaattttttctgctaagcccaacactccaccacacccaggcacacactacacacacccacccgtctcagggtcccctcggacagctcctaagaaggcaccggtcgcccagtcctaccagagggggccaagaacccagacgagtccgtagaagggtcctcgtccagcaagaagaggaggtggtaagcggttcaccttcaggggtaagtaacctgacctctccagggctcacataaagggaggcttagtatacatgcttcttgcttttcacaggaacctgggggctagtctgggtgggattaggctgcctcaagttgcatcagccagggcttcatgccctcctcagttccctagtccccgggcttcaggccccctccgtccccgtcctccagagacccgggcttcaggccctgcctctcctgttacccttttagaaccacagcctggacacatgtgccagacgccttggcctctaaggccctcgggtccccctggaccccggcctcagcaaccctgctgctcccctcctgccaccccagcctccccccctccccgtcccccttcgctcctgatcctcccccggtccccagtagggccgcctgcccccctgcacccagtacctgcccctcttggccacgcaccccgggccaggccaccttagacccggccaagccccatccctgaagacccagcggccattctctctggtaacgagcagagaagaagtagaggcccgcggccattgggcccagattgagagaccagtccaggggcccgaggttggagccagcgggcacccgaggtcccagcacccggtccctccggggggcagagacaggcagggccccccggcagctggccccgaggaggcgcccggagtggggccggtcggctgggctggccgagcccgggtctgggaggtctggggtggcgagcctgctgtctcaggaggggcctggctccgccgggtggccctggggtaagtctgggaggcagagggtcggcctaggcccggggaagtggagggggatcgcccgggtctctgttggcagagtccgggcgatcctctgagaccctccgggcccggacggtcgccctcagccccccagacagaccccagggtctccaggcagggtccggcatcttcaggggcagcaggctcaccaccacaggccccccagacccgggtctcggccagccgagccgaccggccccgcgcctggcgcctcctcggggccagccgccggggttggttctgcccctctctctgtccttcagaggaaccagggacctcgggcaccccagagcccctcgggcccgcctccaggcgccctcctggtctccgctcccctctgagccccgttaaacccaaagaatgtctgaggggagccaccctcggggcccaggccccagagtccagaggtcaggggcacctcagggtgcctccccgggtcccaggccagccggagggaccccggcagcccgggcggccccagaggccggttcctcgccccttccccgggcttcagagcccaggatgtcccccagaagggaccctaggcgtcccctctcctcccctccaggcccgagcctctccctcgcggagaggggcctctttgggccctcaagtccagccccaccgagacccgagtggcccggatccccccaccggcccttctctctgtccccctgctcctctccaaccttcgctccaccctagaccccagcttctggcctccccgggtccaccaggccagccggagggaccccggcagcccgggcgagtcgccttccctctcccctggcctctccttcccgcctcccacccgagccccctcagcttgcctccccaccgggtccatcaggccggccggagggaccccggcggcccggtgtcagtcccccctgcagccgcccagtctctgcctccaggcaagggcgccagcttttctccccccagcctgaggcccagtctcctgtgcactgtctgtaaagtccagcctcccacgcccgtccacggctcccgggcccagcctcgtccacccctccccacggtggacaggccctctgtccacccgggccatccccgcccccctgtgtccaccccagtcccgtccaggggggactttatgtgacccttgggcctggctccccatagactcccatgtaagcctgcctcgagtaggtgcctccagagccccttttgcccccctggcggcccagcccgacccccgggcgcccccaaactttgtccagatgtccaggggtccccgagggtgaggcccagccccctcccgcccctgtccactgccccggtccccccagaagcccccaaaagtagaggctcaggccatgcgcgccctgtcaccaggcctgccaaagagccagatctaaggccgggagaggcagccccaaagcgggtgcagtaacaggtaatctctggtagtgatttggacccgaaatctgacactttagagctctggaggactttaaaactctaaaaatcaaaactttagaggcgaatgggcgccattttgtccccacgcgcgcataatggcggacctaggcctaaaacccccaggaagcgggtctatggttggctgcgctgctgctatctttagaggggaaaagaggaataagcccccagacaggggagtgggcttgtttgtgacttcaccaaaggtcagggcccaagggggttcgcgttgctaggccaccttctcagtccagcgcgtttacgtaagccagacagcagccaattgtcagttctagggagggggaccactgcccctggtataaagtggtcctgcagctatttctggtcgcatcagagcgccaggagtccacacaaatgtaagagggggtcttctacctctccctagccctccgccccctccaaggactcgggcccagtttctaacttttccccttccctccctcgtcttgccctgcgcccggggccaccttcatcaccgtcgctgactccgccatccaagcctaggggagaccgaagtgaaggccctggaccaacccggcccgggccccccggtatcgggccagaggtaagtggactttaattttttctgctaagcccaacactccaccacacccaggcacacactacacacacccacccgtctcagggtcccctcggacagctcctaagaaggcaccggtcgcccagtcctaccagagggggccaagaacccagacgagtccgtagaagggtcctcgtccagcaagaagaggaggtggtaagcggttcaccttcaggggtaagtaacctgacctctccagggctcacataaagggaggcttagtatacatgcttcttgcttttcacaggaacctgggggctagtctgggtgggattaggctgcctcaagttgcatcagccagggcttcatgccctcctcagttccctagtccccgggcttcaggccccctccgtccccgtcctccagagacccgggcttcaggccctgcctctcctgttacccttttagaaccacagcctggacacatgtgccagacgccttggcctctaaggccctcgggtccccctggaccccggcctcagcaaccctgctgctcccctcctgccaccccagcctccccccctccccgtcccccttcgctcctgatcctcccccggtccccagtagggccgcctgcccccctgcacccagtacctgcccctcttggccacgcaccccgggccaggccaccttagacccggccaagccccatccctgaagacccagcggccattctctctggtaacgagcagagaagaagtagaggcccgcggccattgggcccagattgagagaccagtccaggggcccgaggttggagccagcgggcacccgaggtcccagcacccggtccctccggggggcagagacaggcagggccccccggcagctggccccgaggaggcgcccggagtggggccggtcggctgggctggccgagcccgggtctgggaggtctggggtggcgagcctgctgtctcaggaggggcctggctccgccgggtggccctggggtaagtctgggaggcagagggtcggcctaggcccggggaagtggagggggatcgcccgggtctctgttggcagagtccgggcgatcctctgagaccctccgggcccggacggtcgccctcagccccccagacagaccccagggtctccaggcagggtccggcatcttcaggggcagcaggctcaccaccacaggccccccagacccgggtctcggccagccgagccgaccggccccgcgcctggcgcctcctcggggccagccgccggggttggttctgcccctctctctgtccttcagaggaaccagggacctcgggcaccccagagcccctcgggcccgcctccaggcgccctcctggtctccgctcccctctgagccccgttaaacccaaagaatgtctgaggggagccaccctcggggcccaggccccagagtccagaggtcaggggcacctcagggtgcctccccgggtcccaggccagccggagggaccccggcagcccgggcggccccagaggccggttcctcgccccttccccgggcttcagagcccaggatgtcccccagaagggaccctaggcgtcccctctcctcccctccaggcccgagcctctccctcgcggagaggggcctctttgggccctcaagtccagccccaccgagacccgagtggcccggatccccccaccggcccttctctctgtccccctgctcctctccaaccttcgctccaccctagaccccagcttctggcctccccgggtccaccaggccagccggagggaccccggcagcccgggcgagtcgccttccctctcccctggcctctccttcccgcctcccacccgagccccctcagcttgcctccccaccgggtccatcaggccggccggagggaccccggcggcccggtgtcagtcccccctgcagccgcccagtctctgcctccaggcaagggcgccagcttttctccccccagcctgaggcccagtctcctgtgcactgtctgtaaagtccagcctcccacgcccgtccacggctcccgggcccagcctcgtccacccctccccacggtggacaggccctctgtccacccgggccatccccgcccccctgtgtccaccccagtcccgtccaggggggactttatgtgacccttgggcctggctccccatagactcccatgtaagcctgcctcgagtaggtgcctccagagccccttttgcccccctggcggcccagcccgacccccgggcgcccccaaactttgtccagatgtccaggggtccccgagggtgaggcccagccccctcccgcccctgtccactgccccggtccccccagaagcccccaaaagtagaggctcaggccatgcgcgccctgtcaccaggcctgccaaagagccagatctaaggccgggagaggcagccccaaagcgggtgcagtaacaggtaatctctggtagtgatttggacccgaaatctgacactttagagctctggaggactttaaaactctaaaaatcaaaactttagaggcgaatgggcgccattttgtccccacgcgcgcataatggcggacctaggcctaaaacccccaggaagcgggtctatggttggctgcgctgctgctatctttagaggggaaaagaggaataagcccccagacaggggagtgggcttgtttgtgacttcaccaaaggtcagggcccaagggggttcgcgttgctaggccaccttctcagtccagcgcgtttacgtaagccagacagcagccaattgtcagttctagggagggggaccactgcccctggtataaagtggtcctgcagctatttctggtcgcatcagagcgccaggagtccacacaaatgtaagagggggtcttctacctctccctagccctccgccccctccaaggactcgggcccagtttctaacttttccccttccctccctcgtcttgccctgcgcccggggccaccttcatcaccgtcgctgactccgccatccaagcctaggggagaccgaagtgaaggccctggaccaacccggcccgggccccccggtatcgggccagaggtaagtggactttaattttttctgctaagcccaacactccaccacacccaggcacacactacacacacccacccgtctcagggtcccctcggacagctcctaagaaggcaccggtcgcccagtcctaccagagggggccaagaacccagacgagtccgtagaagggtcctcgtccagcaagaagaggaggtggtaagcggttcaccttcaggggtaagtaacctgacctctccagggctcacataaagggaggcttagtatacatgcttcttgcttttcacaggaacctgggggctagtctgggtgggattaggctgcctcaagttgcatcagccagggcttcatgccctcctcagttccctagtccccgggcttcaggccccctccgtccccgtcctccagagacccgggcttcaggccctgcctctcctgttacccttttagaaccacagcctggacacatgtgccagacgccttggcctctaaggccctcgggtccccctggaccccggcctcagcaaccctgctgctcccctcctgccaccccagcctccccccctccccgtcccccttcgctcctgatcctcccccggtccccagtagggccgcctgcccccctgcacccagtacctgcccctcttggccacgcaccccgggccaggccaccttagacccggccaagccccatccctgaagacccagcggccattctctctggtaacgagcagagaagaagtagaggcccgcggccattgggcccagattgagagaccagtccaggggcccgaggttggagccagcgggcacccgaggtcccagcacccggtccctccggggggcagagacaggcagggccccccggcagctggccccgaggaggcgcccggagtggggccggtcggctgggctggccgagcccgggtctgggaggtctggggtggcgagcctgctgtctcaggaggggcctggctccgccgggtggccctggggtaagtctgggaggcagagggtcggcctaggcccggggaagtggagggggatcgcccgggtctctgttggcagagtccgggcgatcctctgagaccctccgggcccggacggtcgccctcagccccccagacagaccccagggtctccaggcagggtccggcatcttcaggggcagcaggctcaccaccacaggccccccagacccgggtctcggccagccgagccgaccggccccgcgcctggcgcctcctcggggccagccgccggggttggttctgcccctctctctgtccttcagaggaaccagggacctcgggcaccccagagcccctcgggcccgcctccaggcgccctcctggtctccgctcccctctgagccccgttaaacccaaagaatgtctgaggggagccaccctcggggcccaggccccagagtccagaggtcaggggcacctcagggtgcctccccgggtcccaggccagccggagggaccccggcagcccgggcggccccagaggccggttcctcgccccttccccgggcttcagagcccaggatgtcccccagaagggaccctaggcgtcccctctcctcccctccaggcccgagcctctccctcgcggagaggggcctctttgggccctcaagtccagccccaccgagacccgagtggcccggatccccccaccggcccttctctctgtccccctgctcctctccaaccttcgctccaccctagaccccagcttctggcctccccgggtccaccaggccagccggagggaccccggcagcccgggcgagtcgccttccctctcccctggcctctccttcccgcctcccacccgagccccctcagcttgcctccccaccgggtccatcaggccggccggagggaccccggcggcccggtgtcagtcccccctgcagccgcccagtctctgcctccaggcaagggcgccagcttttctccccccagcctgaggcccagtctcctgtgcactgtctgtaaagtccagcctcccacgcccgtccacggctcccgggcccagcctcgtccacccctccccacggtggacaggccctctgtccacccgggccatccccgcccccctgtgtccaccccagtcccgtccaggggggactttatgtgacccttgggcctggctccccatagactcccatgtaagcctgcctcgagtaggtgcctccagagccccttttgcccccctggcggcccagcccgacccccgggcgcccccaaactttgtccagatgtccaggggtccccgagggtgaggcccagccccctcccgcccctgtccactgccccggtccccccagaagcccccaaaagtagaggctcaggccatgcgcgccctgtcaccaggcctgccaaagagccagatctaaggccgggagaggcagccccaaagcgggtgcagtaacaggtaatctctggtagtgatttggacccgaaatctgacactttagagctctggaggactttaaaactctaaaaatcaaaactttagaggcgaatgggcgccattttgtccccacgcgcgcataatggcggacctaggcctaaaacccccaggaagcgggtctatggttggctgcgctgctgctatctttagaggggaaaagaggaataagcccccagacaggggagtgggcttgtttgtgacttcaccaaaggtcagggcccaagggggttcgcgttgctaggccaccttctcagtccagcgcgtttacgtaagccagacagcagccaattgtcagttctagggagggggaccactgcccctggtataaagtggtcctgcagctatttctggtcgcatcagagcgccaggagtccacacaaatgtaagagggggtcttctacctctccctagccctccgccccctccaaggactcgggcccagtttctaacttttccccttccctccctcgtcttgccctgcgcccggggccaccttcatcaccgtcgctgactccgccatccaagcctaggggagaccgaagtgaaggccctggaccaacccggcccgggccccccggtatcgggccagaggtaagtggactttaattttttctgctaagcccaacactccaccacacccaggcacacactacacacacccacccgtctcagggtcccctcggacagctcctaagaaggcaccggtcgcccagtcctaccagagggggccaagaacccagacgagtccgtagaagggtcctcgtccagcaagaagaggaggtggtaagcggttcaccttcaggggtaagtaacctgacctctccagggctcacataaagggaggcttagtatacatgcttcttgcttttcacaggaacctgggggctagtctgggtgggattaggctgcctcaagttgcatcagccagggcttcatgccctcctcagttccctagtccccgggcttcaggccccctccgtccccgtcctccagagacccgggcttcaggccctgcctctcctgttacccttttagaaccacagcctggacacatgtgccagacgccttggcctctaaggccctcgggtccccctggaccccggcctcagcaaccctgctgctcccctcctgccaccccagcctccccccctccccgtcccccttcgctcctgatcctcccccggtccccagtagggccgcctgcccccctgcacccagtacctgcccctcttggccacgcaccccgggccaggccaccttagacccggccaagccccatccctgaagacccagcggccattctctctggtaacgagcagagaagaagtagaggcccgcggccattgggcccagattgagagaccagtccaggggcccgaggttggagccagcgggcacccgaggtcccagcacccggtccctccggggggcagagacaggcagggccccccggcagctggccccgaggaggcgcccggagtggggccggtcggctgggctggccgagcccgggtctgggaggtctggggtggcgagcctgctgtctcaggaggggcctggctccgccgggtggccctggggtaagtctgggaggcagagggtcggcctaggcccggggaagtggagggggatcgcccgggtctctgttggcagagtccgggcgatcctctgagaccctccgggcccggacggtcgccctcagccccccagacagaccccagggtctccaggcagggtccggcatcttcaggggcagcaggctcaccaccacaggccccccagacccgggtctcggccagccgagccgaccggccccgcgcctggcgcctcctcggggccagccgccggggttggttctgcccctctctctgtccttcagaggaaccagggacctcgggcaccccagagcccctcgggcccgcctccaggcgccctcctggtctccgctcccctctgagccccgttaaacccaaagaatgtctgaggggagccaccctcggggcccaggccccagagtccagaggtcaggggcacctcagggtgcctccccgggtcccaggccagccggagggaccccggcagcccgggcggccccagaggccggttcctcgccccttccccgggcttcagagcccaggatgtcccccagaagggaccctaggcgtcccctctcctcccctccaggcccgagcctctccctcgcggagaggggcctctttgggccctcaagtccagccccaccgagacccgagtggcccggatccccccaccggcccttctctctgtccccctgctcctctccaaccttcgctccaccctagaccccagcttctggcctccccgggtccaccaggccagccggagggaccccggcagcccgggcgagtcgccttccctctcccctggcctctccttcccgcctcccacccgagccccctcagcttgcctccccaccgggtccatcaggccggccggagggaccccggcggcccggtgtcagtcccccctgcagccgcccagtctctgcctccaggcaagggcgccagcttttctccccccagcctgaggcccagtctcctgtgcactgtctgtaaagtccagcctcccacgcccgtccacggctcccgggcccagcctcgtccacccctccccacggtggacaggccctctgtccacccgggccatccccgcccccctgtgtccaccccagtcccgtccaggggggactttatgtgacccttgggcctggctccccatagactcccatgtaagcctgcctcgagtaggtgcctccagagccccttttgcccccctggcggcccagcccgacccccgggcgcccccaaactttgtccagatgtccaggggtccccgagggtgaggcccagccccctcccgcccctgtccactgccccggtccccccagaagcccccaaaagtagaggctcaggccatgcgcgccctgtcaccaggcctgccaaagagccagatctaaggccgggagaggcagccccaaagcgggtgcagtaacaggtaatctctggtagtgatttggacccgaaatctgacactttagagctctggaggactttaaaactctaaaaatcaaaactttagaggcgaatgggcgccattttgtccccacgcgcgcataatggcggacctaggcctaaaacccccaggaagcgggtctatggttggctgcgctgctgctatctttagaggggaaaagaggaataagcccccagacaggggagtgggcttgtttgtgacttcaccaaaggtcagggcccaagggggttcgcgttgctaggccaccttctcagtccagcgcgtttacgtaagccagacagcagccaattgtcagttctagggagggggaccactgcccctggtataaagtggtcctgcagctatttctggtcgcatcagagcgccaggagtccacacaaatgtaagagggggtcttctacctctccctagccctccgccccctccaaggactcgggcccagtttctaacttttccccttccctccctcgtcttgccctgcgcccggggccaccttcatcaccgtcgctgactccgccatccaagcctaggggagaccgaagtgaaggccctggaccaacccggcccgggccccccggtatcgggccagaggtaagtggactttaattttttctgctaagcccaacactccaccacacccaggcacacactacacacacccacccgtctcagggtcccctcggacagctcctaagaaggcaccggtcgcccagtcctaccagagggggccaagaacccagacgagtccgtagaagggtcctcgtccagcaagaagaggaggtggtaagcggttcaccttcaggggtaagtaacctgacctctccagggctcacataaagggaggcttagtatacatgcttcttgcttttcacaggaacctgggggctagtctgggtgggattaggctgcctcaagttgcatcagccagggcttcatgccctcctcagttccctagtccccgggcttcaggccccctccgtccccgtcctccagagacccgggcttcaggccctgcctctcctgttacccttttagaaccacagcctggacacatgtgccagacgccttggcctctaaggccctcgggtccccctggaccccggcctcagcaaccctgctgctcccctcctgccaccccagcctccccccctccccgtcccccttcgctcctgatcctcccccggtccccagtagggccgcctgcccccctgcacccagtacctgcccctcttggccacgcaccccgggccaggccaccttagacccggccaagccccatccctgaagacccagcggccattctctctggtaacgagcagagaagaagtagaggcccgcggccattgggcccagattgagagaccagtccaggggcccgaggttggagccagcgggcacccgaggtcccagcacccggtccctccggggggcagagacaggcagggccccccggcagctggccccgaggaggcgcccggagtggggccggtcggctgggctggccgagcccgggtctgggaggtctggggtggcgagcctgctgtctcaggaggggcctggctccgccgggtggccctggggtaagtctgggaggcagagggtcggcctaggcccggggaagtggagggggatcgcccgggtctctgttggcagagtccgggcgatcctctgagaccctccgggcccggacggtcgccctcagccccccagacagaccccagggtctccaggcagggtccggcatcttcaggggcagcaggctcaccaccacaggccccccagacccgggtctcggccagccgagccgaccggccccgcgcctggcgcctcctcggggccagccgccggggttggttctgcccctctctctgtccttcagaggaaccagggacctcgggcaccccagagcccctcgggcccgcctccaggcgccctcctggtctccgctcccctctgagccccgttaaacccaaagaatgtctgaggggagccaccctcggggcccaggccccagagtccagaggtcaggggcacctcagggtgcctccccgggtcccaggccagccggagggaccccggcagcccgggcggccccagaggccggttcctcgccccttccccgggcttcagagcccaggatgtcccccagaagggaccctaggcgtcccctctcctcccctccaggcccgagcctctccctcgcggagaggggcctctttgggccctcaagtccagccccaccgagacccgagtggcccggatccccccaccggcccttctctctgtccccctgctcctctccaaccttcgctccaccctagaccccagcttctggcctccccgggtccaccaggccagccggagggaccccggcagcccgggcgagtcgccttccctctcccctggcctctccttcccgcctcccacccgagccccctcagcttgcctccccaccgggtccatcaggccggccggagggaccccggcggcccggtgtcagtcccccctgcagccgcccagtctctgcctccaggcaagggcgccagcttttctccccccagcctgaggcccagtctcctgtgcactgtctgtaaagtccagcctcccacgcccgtccacggctcccgggcccagcctcgtccacccctccccacggtggacaggccctctgtccacccgggccatccccgcccccctgtgtccaccccagtcccgtccaggggggactttatgtgacccttgggcctggctccccatagactcccatgtaagcctgcctcgagtaggtgcctccagagccccttttgcccccctggcggcccagcccgacccccgggcgcccccaaactttgtccagatgtccaggggtccccgagggtgaggcccagccccctcccgcccctgtccactgccccggtccccccagaagcccccaaaagtagaggctcaggccatgcgcgccctgtcaccaggcctgccaaagagccagatctaaggccgggagaggcagccccaaagcgggtgcagtaacaggtaatctctggtagtgatttggacccgaaatctgacactttagagctctggaggactttaaaactctaaaaatcaaaactttagaggcgaatgggcgccattttgtccccacgcgcgcataatggcggacctaggcctaaaacccccaggaagcgggtctatggttggctgcgctgctgctatctttagaggggaaaagaggaataagcccccagacaggggagtgggcttgtttgtgacttcaccaaaggtcagggcccaagggggttcgcgttgctaggccaccttctcagtccagcgcgtttacgtaagccagacagcagccaattgtcagttctagggagggggaccactgcccctggtataaagtggtcctgcagctatttctggtcgcatcagagcgccaggagtccacacaaatgtaagagggggtcttctacctctccctagccctccgccccctccaaggactcgggcccagtttctaacttttccccttccctccctcgtcttgccctgcgcccggggccaccttcatcaccgtcgctgactccgccatccaagcctaggggagaccgaagtgaaggccctggaccaacccggcccgggccccccggtatcgggccagaggtaagtggactttaattttttctgctaagcccaacactccaccacacccaggcacacactacacacacccacccgtctcagggtcccctcggacagctcctaagaaggcaccggtcgcccagtcctaccagagggggccaagaacccagacgagtccgtagaagggtcctcgtccagcaagaagaggaggtggtaagcggttcaccttcaggggtaagtaacctgacctctccagggctcacataaagggaggcttagtatacatgcttcttgcttttcacaggaacctgggggctagtctgggtgggattaggctgcctcaagttgcatcagccagggcttcatgccctcctcagttccctagtccccgggcttcaggccccctccgtccccgtcctccagagacccgggcttcaggccctgcctctcctgttacccttttagaaccacagcctggacacatgtgccagacgccttggcctctaaggccctcgggtccccctggaccccggcctcagcaaccctgctgctcccctcctgccaccccagcctccccccctccccgtcccccttcgctcctgatcctcccccggtccccagtagggccgcctgcccccctgcacccagtacctgcccctcttggccacgcaccccgggccaggccaccttagacccggccaagccccatccctgaagacccagcggccattctctctggtaacgagcagagaagaagtagaggcccgcggccattgggcccagattgagagaccagtccaggggcccgaggttggagccagcgggcacccgaggtcccagcacccggtccctccggggggcagagacaggcagggccccccggcagctggccccgaggaggcgcccggagtggggccggtcggctgggctggccgagcccgggtctgggaggtctggggtggcgagcctgctgtctcaggaggggcctggctccgccgggtggccctggggtaagtctgggaggcagagggtcggcctaggcccggggaagtggagggggatcgcccgggtctctgttggcagagtccgggcgatcctctgagaccctccgggcccggacggtcgccctcagccccccagacagaccccagggtctccaggcagggtccggcatcttcaggggcagcaggctcaccaccacaggccccccagacccgggtctcggccagccgagccgaccggccccgcgcctggcgcctcctcggggccagccgccggggttggttctgcccctctctctgtccttcagaggaaccagggacctcgggcaccccagagcccctcgggcccgcctccaggcgccctcctggtctccgctcccctctgagccccgttaaacccaaagaatgtctgaggggagccaccctcggggcccaggccccagagtccagaggtcaggggcacctcagggtgcctccccgggtcccaggccagccggagggaccccggcagcccgggcggccccagaggccggttcctcgccccttccccgggcttcagagcccaggatgtcccccagaagggaccctaggcgtcccctctcctcccctccaggcccgagcctctccctcgcggagaggggcctctttgggccctcaagtccagccccaccgagacccgagtggcccggatccccccaccggcccttctctctgtccccctgctcctctccaaccttcgctccaccctagaccccagcttctggcctccccgggtccaccaggccagccggagggaccccggcagcccgggcgagtcgccttccctctcccctggcctctccttcccgcctcccacccgagccccctcagcttgcctccccaccgggtccatcaggccggccggagggaccccggcggcccggtgtcagtcccccctgcagccgcccagtctctgcctccaggcaagggcgccagcttttctccccccagcctgaggcccagtctcctgtgcactgtctgtaaagtccagcctcccacgcccgtccacggctcccgggcccagcctcgtccacccctccccacggtggacaggccctctgtccacccgggccatccccgcccccctgtgtccaccccagtcccgtccaggggggactttatgtgacccttgggcctggctccccatagactcccatgtaagcctgcctcgagtaggtgcctccagagccccttttgcccccctggcggcccagcccgacccccgggcgcccccaaactttgtccagatgtccaggggtccccgagggtgaggcccagccccctcccgcccctgtccactgccccggtccccccagaagcccccaaaagtagaggctcaggccatgcgcgccctgtcaccaggcctgccaaagagccagatctaaggccgggagaggcagccccaaagcgggtgcagtaacaggtaatctctggtagtgatttggacccgaaatctgacactttagagctctggaggactttaaaactctaaaaatcaaaactttagaggcgaatgggcgccattttgtccccacgcgcgcataatggcggacctaggcctaaaacccccaggaagcgggtctatggttggctgcgctgctgctatctttagaggggaaaagaggaataagcccccagacaggggagtgggcttgtttgtgacttcaccaaaggtcagggcccaagggggttcgcgttgctaggccaccttctcagtccagcgcgtttacgtaagccagacagcagccaattgtcagttctagggagggggaccactgcccctggtataaagtggtcctgcagctatttctggtcgcatcagagcgccaggagtccacacaaatgtaagagggggtcttctacctctccctagccctccgccccctccaaggactcgggcccagtttctaacttttccccttccctccctcgtcttgccctgcgcccggggccaccttcatcaccgtcgctgactccgccatccaagcctaggggagaccgaagtgaaggccctggaccaacccggcccgggccccccggtatcgggccagaggtaagtggactttaattttttctgctaagcccaacactccaccacacccaggcacacactacacacacccacccgtctcagggtcccctcggacagctcctaagaaggcaccggtcgcccagtcctaccagagggggccaagaacccagacgagtccgtagaagggtcctcgtccagcaagaagaggaggtggtaagcggttcaccttcaggggtaagtaacctgacctctccagggctcacataaagggaggcttagtatacatgcttcttgcttttcacaggaacctgggggctagtctgggtgggattaggctgcctcaagttgcatcagccagggcttcatgccctcctcagttccctagtccccgggcttcaggccccctccgtccccgtcctccagagacccgggcttcaggccctgcctctcctgttacccttttagaaccacagcctggacacatgtgccagacgccttggcctctaaggccctcgggtccccctggaccccggcctcagcaaccctgctgctcccctcctgccaccccagcctccccccctccccgtcccccttcgctcctgatcctcccccggtccccagtagggccgcctgcccccctgcacccagtacctgcccctcttggccacgcaccccgggccaggccaccttagacccggccaagccccatccctgaagacccagcggccattctctctggtaacgagcagagaagaagtagaggcccgcggccattgggcccagattgagagaccagtccaggggcccgaggttggagccagcgggcacccgaggtcccagcacccggtccctccggggggcagagacaggcagggccccccggcagctggccccgaggaggcgcccggagtggggccggtcggctgggctggccgagcccgggtctgggaggtctggggtggcgagcctgctgtctcaggaggggcctggctccgccgggtggccctggggtaagtctgggaggcagagggtcggcctaggcccggggaagtggagggggatcgcccgggtctctgttggcagagtccgggcgatcctctgagaccctccgggcccggacggtcgccctcagccccccagacagaccccagggtctccaggcagggtccggcatcttcaggggcagcaggctcaccaccacaggccccccagacccgggtctcggccagccgagccgaccggccccgcgcctggcgcctcctcggggccagccgccggggttggttctgcccctctctctgtccttcagaggaaccagggacctcgggcaccccagagcccctcgggcccgcctccaggcgccctcctggtctccgctcccctctgagccccgttaaacccaaagaatgtctgaggggagccaccctcggggcccaggccccagagtccagaggtcaggggcacctcagggtgcctccccgggtcccaggccagccggagggaccccggcagcccgggcggccccagaggccggttcctcgccccttccccgggcttcagagcccaggatgtcccccagaagggaccctaggcgtcccctctcctcccctccaggcccgagcctctccctcgcggagaggggcctctttgggccctcaagtccagccccaccgagacccgagtggcccggatccccccaccggcccttctctctgtccccctgctcctctccaaccttcgctccaccctagaccccagcttctggcctccccgggtccaccaggccagccggagggaccccggcagcccgggcgagtcgccttccctctcccctggcctctccttcccgcctcccacccgagccccctcagcttgcctccccaccgggtccatcaggccggccggagggaccccggcggcccggtgtcagtcccccctgcagccgcccagtctctgcctccaggcaagggcgccagcttttctccccccagcctgaggcccagtctcctgtgcactgtctgtaaagtccagcctcccacgcccgtccacggctcccgggcccagcctcgtccacccctccccacggtggacaggccctctgtccacccgggccatccccgcccccctgtgtccaccccagtcccgtccaggggggactttatgtgacccttgggcctggctccccatagactcccatgtaagcctgcctcgagtaggtgcctccagagccccttttgcccccctggcggcccagcccgacccccgggcgcccccaaactttgtccagatgtccaggggtccccgagggtgaggcccagccccctctcgcccaagctgctttgattcctgggatatttttgggaatggtgttaactttctccccttgtatttgctattcaatcaacctgattccccctgctcatacctccacttacaaccaagccactacggccacgtccccggcctcccgctcgggtaagtgctttttcatttttagccccagcccctcctctataagttctaggcaaacctccaatcaccagccaccttccaatgtagtctcttagagagtggctgctacgcattagagaccactttgagccacccacagtaaccacccagcgccaatctgtctacatagaagaagaagaggatgaagactaagtcacaggcttagccaggtgatttgtgaatttcagtttatttactttcttccaatcaagctttcccagcctccgcttgttaggtcctagttatgggttttccatgggggacttagtatccgttctattagattaacgtgcaagacgctaaacttaaccaaggtcagccaagggacgcgtgttatcccaggctgcccaccctgaggatttccccccaaaatcctcctaccctctctttatgccatgtgtgttgttggcttgtgttagtgctatgtaatgcgttgccgccaggtggcagcctgtttatagatgtgcagtaccccttaatgttaggtctgctttagggctgccaggtggcgcaatctaggattaattcacctgtatccctttccctccacccgcagtaacccagcactggcgtgtgacgtggtgtaaagttttgcctgaacctgtggttgggcaggtacatgccaacaaccttctaagcacccgcgcttgtgttttgctttatctgccgccatcatgcctacattctatcttgcgttacatgggggacaaacatatcatctaattgttgacacggatagtcttggaaacccgtcactctcagtaattccctcgaatccctaccaggaacaactgtcagacactccattaattccactaacaatctttgttggggaaaacacgggggtgcccccaccactcccaccaccccccccaccaccacccccaccacccccaccacccccaccacccccaccacccccaccacctccaccaccttcaccaccacccccgcccccaccacccccaccacctcagcgcagggatgcctggacacaagagccatcacctcttgatagggatccgctaggatatgacgtcgggcatggacctctagcatctgctatgcgaatgctttggatggctaattatattgtaagacaatcacggggtgaccggggccttattttgccacaaggcccacaaacagcccctcaggccaggttggtccagccacatgtcccccctctacgcccgacagcacccaccattttgtcacctctgtcacaaccgaggcttacccctccacaaccactcatgatgccaccaaggcctacccctcctacccctctgccacctgcaacactaacggtgccaccaaggcctacccgtcctaccactctgccacccacaccactactcacggtactacaaaggcctaccgaacttcaacccacaccatcaccaccacgcatgcatctccctgtcttgcatgtgccagaccaatcaatgcaccctcttactcatcaaagcaccccaaatgatccagatagtccagaaccacggtccccgactgtattttataacattccacctatgccattacccccctcacaattgccaccaccagcagcaccagcacagccacctccaggggtcatcaacgaccaacaattacatcatctaccctcggggccaccatggtggccacccatctgcgaccccccgcaaccctctaagactcaaggccagagccggggacagagcagggggaggggcaggggcaggggcaggggcaggggcaagggcaagtccagggacaagcaacgcaagcccggtggaccttggagaccagagccaaacacctccagtcctagcatgcctgaactaagtccagtcctcggtcttcatcagggacaaggggctggggactcaccaactcctggcccatccaatgccgcccccgtttgtagaaattcacacacggcaacccctaacgtttcaccaatacatgaaccggagtcccataatagcccagaggctcccattctcttccccgatgattggtatcctccatctatagaccccgcagacttagacgaaagttgggattacatttttgagacaacagaatctcctagctcagatgaagattatgtggagggacccagtaaaagacctcgcccctccatccagtaaaaacccttgccctctccagcaaccaatgtatcccaaataaatgttacttcttttgctcttaaccattgacacgcctgtcattctatcaattaaacaagggaaaaaggtttagctattccaccaacacgaccccaaggaaggcttgccaaaattggtgccttgctctcagcactttgccagcaacttatagcatggtaggcagctcaactcggcccgtcttactgcccagcctactctccactcccagtccatgttcgcactcctatgcatttcctgccctcccacttttaccccagtcccaacccaaaaccacacacaacacatagaattgttagtttaaacagtttattgataggtggctgcttttagcctaattgtgtattgctctcgttgccaaaacctgttgtaagggccggcacccgcaacatggggaaaacataaccgccgccatcccatggggagggtagaggcggttgacatgtaggtgagtagtgtaagaagcatggcgaagtagacaggttacttttagagtgtagtgtacagggccgggcgcaacagtgccaccaacccggggtctgagcattccatgggcagcagggacactgcactaccgccaggtcctggggcagccggggttcctggcgctccgggggcagccgggcggccgccggtgggtccgctgggccgctgccccgctccgggtggggggtggccccgctgggcaccgctgcgccgccgccaggtcctggggcagccggggttcctggcgctccgggggcagccgggcggccgccggtgggtccgctgggccgctgccccgctccgggtggggggtggccccgctgggcaccgctgcgccgccgccaggtcctggggcagccggggttcctggcgctccgggggcagccgggcggccgccggtgggtccgctgggccgctgccccgctccgggtggggggtggccccgctgggcaccgctgcgccgccgccaggtcctggggcagccggggttcctggcgctccgggggcagccgggcggccgccggtgggtccgctgggccgctgccccgctccgggtggggggtggccccgctgggcaccgctgcgccgccgccaggtcctggggcagccggggttcctggcgctccgggggcagccgggcggccgccggtgggtccgctgggccgctgccccgctccgggtggggggtggccccgctgggcaccgctgcgccgccgccaggtcctggggcagccggggttcctggcgctccgggggcagccgggcggccgccggtgggtccgctgggccgctgccccgctccgggtggggggtggccccgctgggcaccgctgcgccgccgccaggtcctggggcagccggggttcctggcgctccgggggcagccgggcggccgccggtgggtccgctgggccgctgccccgctccgggtggggggtggccccgctgggcaccgctgcgccgccgccaggtcctggggcagccggggttcctggcgctccgggggcagccgggcggccgccggtgggtccgctgggccgctgccccgctccgggtggggggtggccccgctgggcaccgctgcgccgccgccaggtcctggggcagccggggttcctggcgctccgggggcagccgggcggccgccggtgggtccgctgggccgctgccccgctccgggtggggggtggccccgctgggcaccgctgcgccgccgccaggtcctggggcagccggggttcctggcgctccgggggcagccgggcggccgccggtgggtccgctgggccgctgccccgctccgggtggggggtggccccgctgggcaccgctgcgccgccgccaggtcctggggcagccggggttcctggcgctccgggggcagccgggcggccgccggtgggtccgctgggccgctgccccgctccgggtggggggtggccccgctgggcaccgctgcgccgccgccaggtcctggggcagccggggttcctggcgctccgggggcagccgggcggccgccggtgggtccgctgggccgctgccccgctccgggtggggggtggccccgctgggcaccgctgcgccgccgccaggtcctggggcagccggggttcctggcgctccactgcacctggaatgcagggtgggggcgtggtcccctggaccccagccccgccgatccctcccccagggcgtacccggcttgcctggttctggggctcctctgggggtcgctgcatccgccggtagggttcgaatgggcgtggtccgcttgctctgctggcccggtacgcctggattgccggctgggggctggggtcccgggacgccccctccctgctcccacccggttccctcccccagggcgtgccccgcttgcctggtcctggagctcatccggggatgctgcatccgctagtccgacctgggtgggtgcggtccgctggccccaccctgggggtagccgccgggtctgctggtccggtgcacctggaaggcaggggggggggcagtgagggaggggcgtggtcctgggaccccgcgccgactggcagggggtccccatggcacaggcctaggggtccagggggcagccgcggcccagcgcgccccgttcacgggggaggaccgcggccgagccaccaggggcccggcgggggtggggggtgcgctcccaggccggaccctggtgccaggcagggaccccgcgccacccgcttcatggggggggaggccgccgcaaggacgccgggccggctgggaggtgtgcaccccccgagcgtctggacgacgctggcgagccgggccggctcgccttcttttatcctctttttggggtctctgtgtaatactttaaggtttgctcaggagtgggggcttcttattggttaattcaggtgtgtcattttagcccgttgggtttcattaaggtgtgtcaccaggtgggtggtacctggaggttattctattgggataacgagaggaggaggggctagaggtccgcgagatttggggtaggcggagcctcaggagggtcccctccatagggttgaaccaggagggggaggattgggctccgccccgatatacctagtgggtggagcctagaggtaggtatccatagggttccattatcctggaggtatcctaagctccgcccctatataccaggtgggtggagctaggtaggattcagctaggttcctactggggtacccccctaccctaccttaaggtgcgccacccttcctccttccgttttaatggtagaataacctataggttattaacctagtggtggaatagggtattgcagctgggtatatacctataggtatatagaacctagaggaagggaaccctatagtgtaatccctcccccccctacccccccctcccttacggttgcctgagcccatcccccaccccagcaccccggggtgacgtggcaccccgcgtgccttactgacttgtcacctttgcacatttggtcagctgaccgatgctcgccacttcctgggtcatgacctggcctgtgccttgtcccgtggacaatgtccctccagcgtggtggctgcctttgggatgcatcactttgagccactaagcccccgttgctcgccttgcctgcctcaccatgacacactaagcccctgctaatccatgagccccgcctttaggaagcaccacgtcccggggacggaaggggacttggggtgattttctatgtgggggtggaaatatgagcaagaataaggacggctccttattaacctgatcagccccggagttgcctgtttcatcactaaccccgggcctgaagaggttgacaagaagggtcaaggtttcgtctgtgtgttgaagggcaggggctgttgggtgcatctggaacggcttacctcgggtaactgtttgccattaaaaggttggggattaggtttagcccctttagctgccatttcgaaccggggtgtgcagatgcaggtctccgggtgggcaggcagtacgagatgtcacgttgtgttgtctttcctcccacccctgtcctggctgtggcaaatgcgaccctcatagagttgtgtttcaggtctgtgtcctgttttgcggtgggttatttcttccctcagtgtttgccagcttatttccccagttttcacgtactggggcctgtggacacctgagggagcggccgttggtgggtatgtgttggaattgctcccaccctcaattttcgcttgccttcttcccttgttaacctgatagcatagcctctaggtttccttgtaggtctgtttgggtttgttggttcacgtggtgctaacttgaattttttggttttctagttccctcttaattacatttgtgccagatcttgtagagcaagatggcctattcaacaagggagatactgttagccctgtgtatacgggacagtcgtgtgcatggaaatggtaccctgcatcctgtgttggagctagcagcaagagaaacacctctccgcctttcgccagaggacactgtagttctgcgttatcatgtgttgcttgaggagataattgaacgaaattcagagacatttacagaaacttggaacagatttataacacacaccgaacatgtggatctggattttaactcagtatttttagagatatttcaccgtggagacccaagccttgggcgcgcgttggcctggatggcctggtgcatgcatgcctgcaggacattgtgttgtaaccagtctactccttactatgttgtggacctgtcagttcgtgggatgttagaagccagcgaaggcctggatggttggattcatcaacagggcggctggtctacattaattgaagacaacattcctggatccagaaggtttagctggactttgtttcttgctggactgactttgagtctgttagttatatgtagttatttatttatctccagaggaagacactaatctatacattttctcagcactttatatgaatcagggtcattgggcctgcggggaactgagccagtaggatattaggcaagggtgacacagtgcccatgcattataatttaaccaaacagtggtcgtgagttttaggccggccatgggggcttacaagaataacatgccaatgacccggcccccacttttaaattctgttgcagcagatagctgatacccaatgttatcttttgcggcagaaattgaaagtgctggccatatctacaattgggtgtcctaggtgggatatacgcctgtggtgttctaacgggaagtgtgtaagcacacacgtaatttgcaagcggtgcttcacgctcttcgttaaaataacacaaggacaagatactaaagaaataactgaggtgagtgtgggaagatgggaatactatgtgttatgttaacgggtgagagcctatactgcagcccagactcggggggaggaggaaatggtaagagttatactctacttatcttttttgacactacatttaactgttatgtaacaatgtttgcttattttcatgttcaataaacgctatgttaatgatgaagaacctgtgttctttggaagtgggcccaatggggtagtaggttttgggagggtgccgtgctagatatttcaactgccacagaccccattttgtcccacctgttaccacattctaggtcctgcatccagtgggccaggtgtctcaccatggctctttctaggtggataccacagtccaggcccccaaggctaccgtgctaattacctcctcatgtccacccccaccctgtgttactgtcgcctgattatcctggcttagcagcctccaagttttacaagacgtcccattgccctgcccttggtccaagtctcgccggttttcagcagcctgttgtagcctgcccccaagtttcgcaggtttcccccatgcttccacccgttaacccaatagcatgacagccaatccaacacgaggcaagttttaagagttaaaagcaactactgtttattttccaaaatgagctgggtatagttgatgatctgtaggcgcagctcatccccacattccaggtccttgatggcctcgtagatggcatcttcgtcgacattgacagccttctcatataccgtgtctctggggctgacctttatacagaaggcgtcccctactaggtccacggccagctcgtaggtggggcctatgttttcacataacagtttcaagcaggtctctgggatgtgaagggaggtgccctggagcaggagatgcatgattaggcgcccttttccatttgtgctgaagatggggcagatggtgccacaaaagtgtccggtgaccaggtaagcgtagagaaggctgggttgggaaagtccagcctttactgcactgggagagctgctgagcagagacacatagaaggtcttgttgggtattatcttgtggacattgttgaagaaggagagctgggtggagctaaactcctgaggcacatgaacctgggacctattgatgcagatctcgcagtgagaccccagagtcaggctgtggccgaagggagacaggcgaaggcagcgcccgggggagagagtgcacagtgacagtgggagaaacacggcctctgagacatgtatgggggtgttcatctcacgcagaaaatctttgcccagctcaaagttggcagagattcccctgaagaagtcccgtagtgaaaaatgggatctgtctacaccatgtctggtgtgccgggaacatattgatcgggccacactgccaaccctttccattcttcccagctctgagcgagattttccacacctggacaccgacttcacgctatgcgccgaggcctttgaggccgtgtagtttctgtggtgcggatgcattaggcggcgcaatgcgggatctgccggtcgctgttggcgtgcattcacggcatctggggtgaccggggccatcgggtttacttttcacacgtagacctgggaagtttgataggactgtaccaggtcaaggccgtggatgcgcaggaccacgtccagttccttagtgacatccacgaggattgttttgcccactctggccacttgtgtggatttaaatatgtacacaagcgtaattaacgagtcacagaccccctgttccagattctgaccggctgcaagcgctgccttaaaggcctggaagctgggtgggtaaatctgaccaaacagcacgctcggattcgtgatgctgtggttgatggcacacagggggtcgcagaacaggtgcttgtggaagtcttgcggggtgcacatctgcagccaggcccttagcctggggcatggcacatccagcagcgtgttttgggtcttgatgaggaacacgatcctgtctaggattttgatgttgttgccgaacgagtcaagaatcaggctcttgaagcggtcaagggtgtccttggcgtccgggtgggccccgaggctctcgcagagtgggcagatggtccgtgaggcattcttgtgccttagtccaaacatgggggccaggaggcaggggccctgcgaatggtcgccagcctccggtctggtgatggccagggccaactccgccagctcatcgccgctgtattccgcgtttaaaccgatagcatggtggcctggccccccgagcaggtccgtcccctgccacgtacctaatagtagtccacagtagtcggccttggttgtaatttcaggagagagtcctcccttttcggccctgagaaatggatgctgaactcggtttctggtaggcaggtggcagcacagggcggtgtacaggcccctgccgacgtcccctgggacatcctgggaatctttgcaggttctgggtccagggagggtaagaaaagtgggggtggttctgggccacatggacttgaagcagaagttggccggggactggccggtgaggatggatttcagaaactccaatttgtagtagccgaggttggcatttctaatcatgtcagaagaggacacagggaggaagcaccggcaaatgtaaaagtgaagctggatgtcaatggcaagaatcctggagggcatgaagagggaatccaaccccccggccatggggaagtattttatcaggatgtgtaaaaagtccatgcctgtgatgaggctagagatccaggctcgtggggcatttagacagtagtagcagagcagggcatagtcctcaaagaaggccacgggggcatctgagtgattgaccagggtgtcgagcagatcacaaactcggcaggtgctggctggagagagggactcgtaggtgtggacgagtggtgggtaggctatgccttcttccgcgttggctggaagataggagtgggccatcaaaaggccgactgcctcgaactggcttttcagattgtccacggtccagggcacaaagtcctccatctttggagttctgcccgcgatctgtgccacctctgttacgccactcctcgtgagggggcagctggacagtctttttccggtcagggggtttggctcgtttgcgctcgtgactttgtgagccatgacacatctgggtggcaaggtgaggtcttctgggtttttaataccggggtcggcaccagtttctgggacaccgccacaaggacaaggtgggctagcaagttctcgagtctacgaagactccgggggcagtcttttgagtttctcgcctatgatccaccccaatctcgcccccctaattgcgccatctgcctacgcgaggctgaacctcctgaatcactgcatctttcttgaggcgtttaaagaagagaatagtggccagggcctcggtggggtccagcgtgaggtcttatttttgaaaagggatattataaaacaggtcattgctcggattgtggcagccgatagcaccctagatctagtgaatcatggcgagcccggaagagaggctcctagacgagctcaataacgtaattgtgtcatttctgtgtgactctgggtctctggaagtggagagatgctccggggcgcatgtgttctccaggggcagctcccaacccctctgcaccgtgaagctgcgccacggacagatttaccacctggagtttgtctacaagttcctggcctttaagctgaagaactgcaactacccctcctcgcccgtgtttgtgatatccaacaacggcctggccaccaccctgaggtgctttttgcacgagccgtcgggtctcagatcgggccagagcggcccttgcctgggtctctcaacggatgttgacctaccaaagaactccatcattatgctgggccaggatgacttcattaagttcaaaagccccctggtcttccctgctgagcttgatctcctgaaatctatggtggtctgccgggcctacatcacggaacaccggacgacgatgcagtttctggtgtttcaggccgccaacgcccagaaggcctcgcgggtcatggatatgattagtgatatgtctcagcaactgtctcggtctggtcaagtcgaggatacgggcgccagagtcacaggtggaggaggtcccaggcctggcgtcacgcactcggggtgtcttggggactcacacgttagggggcgcggtggttgggacttggataacttttcagaagctgagaccgaagacgaggcgagttacgctccttggagggacaaagactcgtggtcggaatccgaggcggcgccgtggaagaaggaactcgtgaggcaccccatccgcaggcaccggacacgcgagactcgccgtatgcgcgggagccattcacgggtggaacacgtgccccccgagacccgggagacggtggtggggggagcatggcgttattcttggcgcgccacaccttatctggcacgggtgctggctgtcacggccgtggccctgctcctgatgtttctgaggtggacctgacgttgcaggcccttggggagcgggggttctccaggctcctggatctggggctggcctgcctggatctgagctatgtggaaatgagggaatttgtggtttggggcaggcccccagcttctgaggcggctgtggcctctacgccaggctcgcttttccgaagccactcgtccgcctactggttgtcggaggtggagaggcccgggggccttgtccgctgggccaggtcacagaccagcccctcatccctgaccctcgcgccccatcttggcccgtccctcttgtccctttcagtggtcaccggtggtgggtgtggagccgtggccttttgcaacgcctttttcctagcttattttttggttgtgcggtctgttttccccgcgttttccgatagaatagctgcctggatctgcgaccggtcccctttctgcgaaaacacccgggccgtggccaggggttaccgaggcctcgtgaagaggttcttggcattcgtgtttgagcgtagtagctatgacccccccttgttgaggcaaaactctaggcctgtggagcgctgctttgccatcaagaattatgtcccgggcctggactcacaaagctgtgtgacggtcccgagcttctcccgctgggcccagtctcacgccagcgagctcgatccccgggagattcgcgacagagttacaccagcgactgcaccttcgttcgtggctgatcatgcctcggctctattggcctccctccagaagaaggcctccgacaccccctgtgggaatcccattcagtggatgtggtaccgcctgttggtaaactcgtgcctgaggagtgcccactgtcttctgcctatacctgccgtctctgagggggggagaaagacgggcgggggcgtaggggaggagctcgtgggggccggggggccctgcctgagccgggatgttttcgtggcgatcgtaagccgcaatgttctctcgtgtctgctgaacgtgcctgccgcgggtccccgggcctacaagtgtttcagatcccacgcctccagaccggtgtctggcccggattaccctcccttggccgtgttttgcatggactgcggttactgcttgaactttggaaagcagacaggtgtaggaggcaggctcaattcctttagacccactctccagttttatccccgtgaccagaaggagaagcatgtgctgacctgccatgccagcggccgtgtgtactgctccaactgcggctctgcggcggtgggctgccagaggctggctgagccaccgagcgcccgctcgggctggcggccccgaatccgggcagtgctgccgcacaacgcggcctacgagctcgaccgtggctcccgcctcttggatgccatcatcccctgcttgggacccgaccgcacttgcatgcggccggtggtcctgcggggggtgacggtcaggcagctcctgtatttaactttgcggacagaggccagagccgtttgctccatctgtcagcaacgccaagctccagaggacgcccgcgacgagcctcacctgttctcctcctgtttagaggtagaattgccacctggtgagcggtgtgcgggctgccgtctctatcagacgcgttatggcacgccggctgcccaagcccaccctccaggggaggctggaggcggattttccagacagtcccctgcttcctaaatttcaagagctgaaccagaataatctccccaatgatgtttttcgggaggctcaaagaagttacctggtatttctgacatcccagttctgctacgaagagtacgtgcagaggacttttggggtgcctcggcgccaacgcgccatagacaagaggcagagagccagtgtggctggggctggtgctcatgcacaccttggcgggtcatccgccacccccgtccagcaggctcaggccgccgcatccgctgggaccggggccttggcatcatcagcgccgtccacggccgtagcccagtccgcgaccccctctgtttcttcatctattagcagcctccgggccgcgacttcgggggcgactgccgccgcctccgccgccgcagccgtcgataccgggtcaggtggcgggggacaaccccacgacaccgccccacgcggggcacgtaagaaacagtagagggcacgaaacatggtgtatgcactttattaataaacaattacagatacaaaaacttgagtctctcgaggtctgcgatgaggcggtgggtggaacgctccagcttgcggcgaagctggctcacgaagcgagacagtactcggctagcctgactaagggtgaggctataacgcaggtcctgttccggggcggcggtggatagagaggagggggatccggaggggaccactaggtcgccggaggtcgaccctcctgtcaccacctccctgataatgtcttcaatagacagaaattgggtgaccactgagggagtgttccacagtaatgttgtctggtcgctagatggcgcgggtgaggccacgctttgcgaaaacgaaagtgcttgaaaaggcgcgggatagcgtgcgctaccggatggcgggtaatacatgctatccttacattttggcattttgggcagctgggaggcggcggatgggggtgcttcttttcgcacggtgtatgtttggggacccgcatgccggtactgggataggcgcacctcgggccgcgcgccaggctccgagccggaatgcattgggggcaatgggattgcgggggattgttgctgtctgctcctgacagggagagacacgcgcggcggagatgcagccgacggcggggccgcggtgggctgcccccgaggacgggcgccggccgccagcgcccccgtggcctttggcacgggcctggcacccaccgctttaattgtgggggtgggcagggcagctgcatcttggggcctttgtgcttgcgttttttgggggcgcggtgccaatgcaccaactggggtgtgcgccggggcggccaagccggaccccagggcgggtgcctgggggatgggaaagccggacggcgcttctcccgggtcgaacgctggagtagcggaggctgctgcgccggcggccaccacgggcgcacggggtcgcagcccgacggccgtggggaggcgggtggcggagggccgaatctccgcggcttcttcccggcccccctgctgtttcttctcccgttgcatgatagaatggccatagggtgggtcctgagaggaggcttgtgtgtcctggggctggagcccaaaagtcgttaaagatgccgctgatggtgtgggagctatgcctcccgtcgactggccgggcttgtagggggctgagggtggataactgggcttctgtgaaggcaccaaccctggaatctggatggtatgtttcttctgtgaccccgaggcagtcgatggtgtagagtgtggagacaatgtgtagacgatgggcccttgttcagaagcccagggacttgaggggggctgttgtggtgctggttggggaaggagctccagggaatctttgggccatggccttggggagcttcccggagaccggtctgggctctcggaagccctcgtttcggccccgaaatagggccttgccatcaatcgggggcctgggagagtgatgggggcggccaatcccggggtaactgtcacgtcccggggggaggaggtaggagacagccagtccctgggcctgccaggggccaccttctctaagagggggctctgtgggctgggagggccagaggcctcagattcagcagtagtgctccccttttccccctggtccgtctcccctcctcccaactgctggagccggtcggaggaggccggggtgttatctgctgactgaaacccgtccccgctgaccagtccgtgccccacccttggggggaaaccggagaacagctcctggacgttgcgtggattcgggggaagctggtatccaaccggcagtggaggatcttcgtgctcgtagaaggaggggttgagtacatcggtcggccatcgtgaggccccggccgcgttaaagtagaactgcacgtccggcagattgtgccgataggtgaaacacttccagatgatgttttttctgttggccaggatggccacggtggggggcctggcctccttaggtttggcggccctggcctcggtgagaagctcgcgtagccacacggcctggcgtgcaaagatggacatctctggctcgaaagactcggagtagccgtccaggtcctgcagaaaattcagcgagatggtctccaccagggaccggaagggctcagagtgcccgtcgcagtagaggaggggagcaacgaccctgacctgtcccagggtcttcaggttaaacagatattgagaggagacaaagagagttaggggccgaccgaggaaggccgccgccacggccgcctcaaaaacggagacggggatggtgtcaccggagcccctcttaggaccggtaatgggagtgccataaggcataagatttctcagggcccggccggtaacggtgccgtaggaagacggggtttcgcgggggacctcgagtccctccgccctggggagctcttctccgcgtgtataggcctgcttcacaaagtcgcgcaggtagtcctgaaatgcgaccgggccctccagcgggcgcaatgagtgccagagctgctgaagggcctcgggggcgaagcaccggcgtgcgaggagcagcatgcaggctcgggcgcgggccgtactttggttgtggaccaggcccaagaactcggggtgcggccagagggcggctcgggtatccatctcctcccaggcgtcctggaagaagatgaagccggtgggtggaccggcgatgcggtggcgggtgaggcggcgcgcgtcttccccgtcgttgctgccgcgggtggttgagggcatgccccccctcccggaggctggactcctgaccagcctgtaggtgaggaccgagtccgacaggaggtctcccaaacccccatctctcgctagagccgagaccaggccgagtcctgcgtagaacgatggggcgcccaggaaggcggcagcgtaggccggatgtgtgccgaccagcagcgccatcatctcccgttgttccaatagaataacttcccggtctgtggccggggctggataaggggggtgattcctagaggcgatgagactggcgtgcgctaaaagtgtcatggccacaatggggttgtctgccaggtcttccatcagggctttgggcgcagagacgtattcccgaagcagctccccggcgttggactccacgtcgggccaggtgtcccagtaggagtcggcggcggcggcgctgaggcgggcggaagctacactggccagggttcttctcctcctctcttggtcatcctgcgggggaccaatagcttgggggcgtccggctggggtcagggaaaaggcctctgggttctccagcacggtgggcatgacatattccagaaagttgtggtagacggggatgtagttgagcggctcctgggtgtctgcggagacgtaggccgggttaagggggtcgcagggagactctgtttccagccagagggtgccggcgtatttcgccggccctgccgccgccagaaattgtgcccgccgggtcggggctccattgccccatccagttggtggtgccgaaatcgtgatgaggaggggcaggttgttggtcaagggatgcttaacgaaaacggtaggctgggcggtctcgtaaaaagccaggaaactctgcttggccgaggcatagcgcagcagcttgtccttgaggagggcatactgggagccagccgaggccccaagcgccaggcccctggcagcctccaccacgatcttgagctggcgcgggtcggtgtggcccctggcctgggtgaccagatcctgcagcgttccctgcagctgggactcttcctgggcctcctggatgatggcctccagtcgggagaggcgccttttccagtctgcgacggtctccttgccccccgcgacccgcttggggtccaacgtggccagagccaacctcagctcctccatgccatccatggagttctgggccatgccctcgacttccaggagccgtgttagctcatgaatttcaccgtcagccgcagcggctaggttcagccaggcacccgcgcccccagctaaggccagggctccttcggaaagaccccgcacggcctcgcagatgccccggatccacttggcggctgccagggatttccggtagggccatgagccgttcccggccgctgcccgggccagggcggcctcgaggggagcctggacaggggctttgggcggggagggaagcaggctccggagttcatcgtcgggggcttcgtcgcgtgacctggagaggacggcctccagagccgtgtgaaagccccgccgagtgcttgccgccatctcgtgggccttcgccatcagggtctggctctcccggacctgctcttccagcgcccggacctcggccgcctcggcctcggtcagcagctccgagaagaagtcccccgtggcctggaggagatcgtcccgctctcgccttgtcagcagctggggcttcttaggccagagcgccgagtccgaggccagcctgggcggggcggttgcctgggggatagttggaggaggaggcaggttagcctggcctgggtcattagtggcttcgggtagcgtccgatccacgtactcgctcacgatggccgtcagggcagcctcggctgctcgtcttttttccagaagcccggccagcccccgctcgtactccgcgtagggggcctccagatccgtgttgaccaccgctgatttcatgtccggggactgcagggcctggcgcgtctgcgcgagggccgaacggatggcatcggccgccgtcctggcgcgaaagagggccccggccgcttcctccgctcctcgccctcctcctccttctttggcggtagccgcgggggtggcgggccaagcgtccagtctggccagagggccggtctcgatatccgtgaaccagccgggttccgcggcctccattctctccgccgcaccaccatcgtccacgagcagggatcgcagtctctccctcctcaccctcgttattcccaatagcatagcggcaaggatctgtgtgagggagtccaagatgtccgtgtttctggctactgccgccgctgctgccgcggctgagtccgtattgtctggcagcagggaggccagcagggtgttccagtcatcgggcgaagtgggagcgggctctgggcgtgcccccagcgccttcctaattctggcccaggcctcattcgcctctcgcgctcgccgctcctgcctctccttgtcttcctgttctcggagcttctccttttccttgcgcccggtctccataagctgccgcagcttcttctcatactgtcgcttgagctctttgttgggggcagtgtccagaaaggcctcgagctgttcctcggtggcgggcttaaagccttcggcctccaggcgccaggcctgcacctccttctgtctgagctgatcgttgttgttattcttcaatttctgcaggtaacttaggaagcgtttcttgagcttccctgggatgagcgtttgggagagctgattctgcagcccagagagtagtctcagggcatcctctggagcctgacctgtgatcgtcgcatcatagaccgccagtagacctgggagcagattcaccgccgcggccgtctcctttaaggtgctgtgagtagcaaaattctgcaaggccactaggcgcgctggctccagcgtcagccggttgcccatctcgaatgtgtgcagggcctctgagaccatggggtccaggatgcggtcaatgccatcctgcacctcagggtcaaggaccggcaagtcacgatagaggtggtctatgctctcctcgaaggaggcaatgtagttatcgatggtgtagaaggtgatggatttcaggatgttcatcaggtactttttggagcgaacaatctgctgtatagtgtcacgtaggcggatgtacgtggggttctttgcggccccgactatcgaccctgcatttgcgatgtacttttctatgacggggatggtgagggccgcggtgtcggccagcggtggcgtggcttcggggttgtcgtggttggcgggtgtcgcagagggagaggcgggagagatgggggcgcctggggccgaggccacaccggccaggcccaacattgcctcgatgtcgtccaggatggtgcggaggcgcttttcgttttctctggtggtctcgagctccttctgttttttcgcgactgtctcaaactctggaaggggggcaatgctggggtcgtcctcctcaactcgctccaggggccaggggataccgctcatatcactaagggcggtgcccaggtagaggagctcgcgatagtcccattcaatggacgtgtaccggatgtttaggagaggcagggaggcgatgatctggcatgtgtgccgcaggtgtgtcaggaggtcgtcaaaatccatcactgttgggaggcttgggtcctcaaggtaggagagataatcggaggccgccgaggccaccttgtccctgatgtccgccgtacacctgcgcacgtgcagggccgcattcttggaccggacggccacgttgtggacaaaggggggcactgaggcggcgggaggggccccatactctatcgctgtcaacagcgccaaaaagcggacgtcctcctcatctaccccagcctgttgtctggccacggccgttcgggcggcctccgccagggataggaggcgcttccagctttcgtcgtccaggaccaaggggacgtccacgtgcgggcccctgtagatggaattatcctcgggttctcctcctccttcccccgcctcctgatctccgcccgagagcaggtcggtcaggcgtctgcgggccgcctccaggtcaaattttccatcgtcgctctcggccagctggggaatttcagccagcatcttagcaccggcatctacacggaccgcgtccttcgtggccagggacggcaggcaggcctccagctttgcggccaggtgcttatggaactctcccgctcttcccttgttttctgatagcatgtttgcgaggttttggatgttaagttcggaagtgagcagttgctccaggtccagcgtggggacctgcagatgtcccgaccagtcctttaagaattccagcagatttagcacagacgatcggtccctactccttattagcccctgctcgaggaccactgtcacaagaagatagtctatcatgctcaaggcatctgcctctggcacttcccggttagaggccgggtcgtagacgatggcctgttcctggtaggtatgtccggctattctcgcaatgttgctctcgaggggcacaaagtccatctcaggagtctctatgtcaaaggtggtctgatagtattggctcctggcggtgtccagtgtgatgggggacgtgggggcactggatcccgattccaggctgttggagaacacttcatcttcgaacatgtcttcatcctctgtggtggggatatcggaggctaagtcgctctccgcttcttcagagtcggacatggataggaaaggctcctctaggtcagacaggtagcggacgaggccagaacccccagatgcatcatccccaaaggagggctgctgcccgaagggaggtgatggggatatctccgttccagccctgtcagcggccgggggatggtttttttctggttcgagtgtcgtggctgatggtgggagctgctgagcaggaggaggagccggggtagctgatggcaggggctgctgctgaggaggaagaggagaaggagcccgggcggctgatggcgggggctgctgctgaggaagaagtggagaaggagccggggcggctgatggcgtgggctgctgctgaggaagaagtggagaaggagccggggcggctgattgcgggggctgctgctgagttggaggaggagaaagagtcgtggtggtgggggctgctgctgcagtcggggaaggggatggggtggtcagagggatttttgggttcgagggagctgcctgtggcagagggatgggtatttgcaaagggaggcgaggagatggagtgactgaaggagcgatagttgagactggcgcggggtggggtgtcggggaggcgggtggtgattggtgagggatggggattactggagggggaaggcgagctgctgaaggggggcgatggggcggaacgtgggtgcgtggcagctgatcatcctctgtgtcagtggtggaggacagagggaggcggcggccggaggtgggcttcttgtgggggctatctttgcccaatccctttttcctcttgggagtctgaggcgctgcgccgctcgacgcccttggtggcgtggagggagcggggaccccgggggtgtgacctaggccggggatggggatgaagaggggagggctggaggccggggccgcggaggccggggccgcggaggccggggccgcggaggccggggccgcggaggccggggccgcggaggccggggccgcagaggccggggccgcagaggccggggccgcagaggccggagacgacggcggggagttggtctttgcaggactatacctggcggcagggaatgagtcggatgtgaaagatcgagagggcagtggcctgaggttatacggtattattcgccgttcaaacggtagcatgacgggagggctgctatcagcaccgggcgtccccgccgcctccccatcactggacacaagctcgggccccaccaggtcaaagccgctgccgttggcctcataaaagtcatacacgccatagtgttccagcataaagatgcgggggtcctctgtctcaaaggcctcgggtagaaaatagagatgcacgcaagtgtactgggcccctggtgcccccacgtactgcaggatgtcgtgcgcataggtgctgactctgacatgggcgggggtgcccggggccgcatccttctggcagtgggggtcaaacaagtagaaggagccatctgtctcgatgatgatggcccccgcgtagatgtcgcagatgtagaggatgaactgggccaccccgttgtaactgccgtgcaggacctcggccagggactgaacaactgccgagtttgcgatctgggcagggaataggacgaggccaaagatctccgccgagcggtatatgtgcacgcgcccaccgcccctcaggaccacggagctgggcacgtccgtcaactgggccatctcgtgccccttgaggatgccgctctggcgcatgagggcatccagccgcgccccctcgtccaggacctcgtccagctcagggcgggaggtcagggggcggccggccaggaagctcttgaccaggtagaggacgcagttgctgacgcactggatgccggcaaagcggccaaacttgcagtgggcctggttgcacgaggccgtgcctaggatgcggagggccgagcctccactcccgcccccgggggcattcacatccatggtcctgattccgcgcacggggccggttccccgggtgcgctggctttgcccccagtcgccgttactcatcttcggcggtggggcggggaggacgccttgtcgccccccttctggtccggggtcttacgcggctggcggcggcagccgccgagagataaggggggtacgtgtgtgcctccgcctctcctctgtctgggccgccgccgccgcttgcccgccttgaaggagagggggtagtccgcggactgcgtctgcgggggcaggaggtctcaaccttctgggctcgggccgcggtgtcgatatccgatggcctttccctgtcttcctcgtatgctccttctcctcctcctcccggcacgcccctgagatctgcctcccctccctctccctcgtcctggtcggaaaagtctgaggaggagaaggagaatggggaggagtccaaaacggcacgccacctgccgtggggcggtggtgacaggtcccggctggcccggcgcttgctcgcgttcctgccgttacccaggagaatggccgcgagttttttggcggggaggatgcggaatggcgggggcgtttgtcccacgggtgagggggaatcgtcggttagggccggcacgaggtggtgggtctggacccgggccgtgcgagcaaaggcggcgagaaccgaggggcttctgggggtgactgtgatctgttccggatttaggtccatggcgggtgtgtatgttttaataggggtggtctctggcgcggcaggatgatggtcgaggacgtccaccagggccttgcagatgctcttgcctagatacaggatgtcgtccatgctgaggggaggtggggtgtctgctcccccctgcggaagccgcctgggtgcggggatgaagacaggtggtgggcgggcgtctcgccggactatggcctcggcacgctcggcgtcgatggcgggtggctggaacaggcgggcgaatgtgtaatcccggaaccggtaggcgacgctgcgcctgagggcgcccgtcaggctgtatcccagctccagggcgtgctccacccgctcgttgagctcctcgagatccggacgcaggggctcgctggtgtgggcccagagggggtgatccgcgatgccccggctctccctgagggccggcaccaggaggcgccttctgagggtggccgtgtcggccgtggccagggcccacctggcggcggcgtcccggcacacatcctggatgccctccacgacgctctttagcgtctggaggtccgtggagtagtggcggggggaggatgaaacgctcttttccttcaccgctaccaccgcctcctcctcctcttccgtcgccagagggatctgcaccctcccggtctctgcgtcgtacaggagcgggcgggagcacagcctccaagctgcccccgtcaagcgcgagatgtcctccgagagggtctcacccgagaccagaaagcggcgggtggccaggcccaacgactccgccgtcgtgctgtatctcagggtgaagaggagtgaaaagagggaggtgggccaggcaagcggtggtgcttccgccgcccgctctgaagctgagatagtctcggagatgatgcctgagacctctcggacggcgtccatgatcctaaggactgcgtcgtgggacgacagcccccaggggcccccgccctcttcgtcttctgcaccctcggctcctgcgtccccggccttgccttccccctctaagttgagggggcgcagtccgaccgcctggggggactccccaggcatcggaggggccccgtcatagatctcccagacggtggcgtatatgagctcgagaggacggcgggcccgggtcagctcgggggaagggagggccaggtcgctgccgaaggagaccagccagcgcagggcggccagagagcgggttttgggcagctcgttggagaggacccggcgaagggcgggccagatttggaactcgatgaaggcggccgggaagaaggggctgtggacataggccggatccgcgcgcgccgtttggccggccctcagggaccggcagtatgcctcgacgtctgtccgcggggccgccgccaccgctgccgtccactgccttcttccctgctcgccggggagtagggggggcttacaggggagggccggagccggggccggggcctgccacaggcggctgtagcggacccatagcagagacctgaggagttcggatgaaaggtcccccgccacctgctcatactcggccgcgggaggggggacgatgaagatgcgcagaggggttacggcgtcccaagggtccgccgccgcccccacacccacagccgtcgcggcgggggcggcggcgggcgtagaggggccgctggtgcgccgggctcgtctgtccacggcctcggcctccgcccgcaggtaggccgcccgggccacacgggcgaagcggctcgtggggctcgcggtgggcagcagtcggaaaaagtgcagggcaaagcccgatagactctctaggagggcggcggtggcctcgagccacctccaccgcgagcgggacacccggggcacagaggccagcatcatggcgtagtcccccgccacggtctcgttgagcccggccgagagcagaaccgtggccacctgctcgatggcggctggagagaaggatgcccggctccccgccgcctcctgcacacgagcggccagggcctccatctctgccgccatcccggccaggaaggcctcgatgaccgagtctgggacgccgtaagtctggtcccagagcagggcctcgtacacatagtcgtaaaagagggcccctgagggctccaaaagccggagccgggcggcgtcaaaggccaggacgggcacagccgcgacggggggcgtttgtcccccgctggcctccgcgtacacgcccaggatctctaccgcccgccgccgggccaggggcagcgaggccaccacgctggaaagtgactcggggcggtgaaagagaccaccaccgctttcttcaccctctcccccgccggccccgcccccactgtgctccaccagctccacggccatggccttgatgtccgcggccgtgggctgaccctgccctgcagccgcccaggggtagcggttggtctccgcgtatacggtgaccagccatctccccagcgtcgttttcgccgcgttaaaagcgtagaatgacagcccctcccgcgggaaggcgtcccaccgggccagataagtgtcggccaccagctcttccacgaaggcaaaggtggccgttgggccagagaccgcgagcacctccccgctgccctcttcgatgatgcgccggtacgcggccgccagggcccgggtctctgcgatgagccgagagccgtccagcggatcgtcggtggccggagaggctgtcgtggggggcagtgaggatgccagcacgtccagggccgcctccagatggccgaggccgaagctgcgcctggaaaaggaggccgcccggagtaggtagtaggcgtggtggcggaggaccgccgccgggtaagcgtggccgctcatgagggtgagagtatttaaaaaatcgcgcaccagcaccggctgggccaaatcccccagtccaaagatccccagctccagaggcatcagcgcgcgcaggcgggcagcggggtcgtccccagacagcagcaactgacgcgtcacgcgggcgagccccccgtccacctctgccaggggtggctgggcgtctgcccctccgctaccgccgctgctgtcactctccatagcggacgccatgaaggtccaggggtccgtcgatcgccgccgtctgcaacgccgaatcgcggggctgctgccccctccggcccggcgtctaaatatttcccgggggtccgaattcacgcgggacgttcgtgggctggttgaggaacacgcgcaggcctcctcgctgagtgcggcggccgtctggcgcgcagggctgctggccccgggggaggtggcggtcgccgggggtggcagtggaggggggagcttcagctggtctgggtggcggccgccagtctttggggactttctgatacacgccagctccttcaacaacgccgaggccactggaacgccccttttccaattcaagcagagtgacccgttctcgggcgtcgacgcggtattcactcctctctccctgtttatcctaatgaatcacggccggggtgtagccgcccgggtcgaggcaggtgggggcctgacgcggatggccaacctgctgtacgacagccccgcaaccctggctgacctggtcccggactttgggcggctggtggccgaccgccgcttccacaacttcatcacccctgtgggccccctggtggagaatataaagagcacctatctgaataaaatcaccacggtggtccacgggcctgtggtcagcaaggccatccctcgcagcaccgtcaaggtgacggtgccccaggaggcctttgtggatctggacgcgtggctctccggcggcgccgggggtggcggtggagtatgcttcgtcggggggctgggcctgcagccgtgccccgccgatgcgcgcctctatgtcgctctgacctatgaggaagccgggccgcggtttacgtttttccagtcgtcccgcggccactgtcagatcatgaatatcttaagaatttattactcaccatccatcatgcaccgctatgctgtggtccagcccctacatatagaggagctaaccttcggggcggttgcctgtctggggacatttagtgctactgacggttggaggaggtctgccttcaattaccgtggctctagcctccccgtggtggagattgacagcttttattccaacgtctctgactgggaggtgattctctagacttaacgggaggaaacaggaggaggagggggacaagagcacaaaagtggttcagtggacacccaccacacagcatggcaacgaccagtcatgtcgagcatgagctcctctccaaattgattgatgagttaaaggtcaaggccaactcagaccccgaggctgatgtcctggccgggcgcctgctccaccgccttaaggccgagtcagttacacacacagtagccgaatatctggaggtcttctctgacaaattctacgatgaggaattcttccagatgcaccgggatgagctggagacccgagtctctgctttcgcgcagagcccggcctacgagcgcatcgtctccagcggctacctgtcggccctgcgctactatgacacctatctgtatgtggggcgcagcgggaagcaggagagtgtgcagcacttttacatgcggttagccggcttctgtgcctcaaccacctgcctctacgcgggtctcagggcagccctgcagcgggccaggccggagattgagagtgacatggaggtgtttgattactactttgagcacctaacctcccagacggtgtgctgctccacgccctttatgcgctttgccggggtggaaaactccactctggccagctgcatcctcaccacccccgacctcagctccgagtgggacgtgacccaggccctctataggcacctggggcgctacctctttcagcgagccggggtgggtgtaggggtgacgggggctggccaggatgggaaacacatcagcctcctgatgaggatgatcaacagccacgtggagtaccacaactatggctgcaagaggccggtcagtgtggcggcctacatggagccctggcacagccagattttcaagtttttggaaacgaagctgccggagaaccacgagaggtgcccgggcatctttacggggctctttgtccccgagctcttcttcaagctttttagggacacgccctggtcggactggtacctgtttgaccccaaggacgccggggacctggagaggctctacggggaggagtttgagcgcgagtactatcggctggtgacagcgggcaagttttgtgggcgggtctccatcaagtccctgatgttctctatcgtcaactgcgccgtcaaggccggcagccccttcatccttttgaaggaggcctgcaacgcccacttttggcgcgacctgcagggcgaggccatgaacgccgccaacctgtgcgccgaggtgctgcagccctcgaggaagtctgtggccacctgcaatctggccaacatctgcctcccgcgctgcctggtgaatgcgcctctggcggtgcgggcacagcgggccgacacgcagggggatgaactcctgctggccctccctcgactctcagtcaccctacctggagagggggcagtcggtgatggattctcgctagcccgcctcagagatgccacccagtgtgccacctttgtggtggcctgctccattcttcagggatcccccacttatgattccagggatatggcctccatgggcctcggggtgcagggcctggccgatgtctttgcggacctgggctggcagtacactgaccctccctctcgctcgttaaacaaggaaatattcgaacatatgtactttacggccctctgcaccagtagtctgattggacttcacaccaggaagatttttccgggtttcaaacagagcaagtatgccggggggtggtttcactggcacgattgggcaggaacagacctttctattcccagggaaatttggtctcgcctctctgaacgcattgtgagggatgggcttttcaattcacagtttatcgccctgatgcccacctcaggctgtgcccaggtgacgggctgttcggacgccttctaccccttctatgccaatgcgtccaccaaggtcaccaacaaggaggaggcccttaggccaaaccggtctttttggcgtcatgtgcgtctggatgacagggaagctttgaatcttgtcgggggccgtgtctcctgcctcccggaggctctgcggcagcgctacctgcgtttccaaacggcctttgattacaaccaggaggacctgattcagatgtcccgggacagggccccctttgtggaccagagccaatctcacagcctgtttttgcgtgaggaagatgccgcgcgggccagcacgctagccaacctactggtgcgcagctacgagctgggcctgaagactatcatgtactattgtcgcattgagaaggccgccgatctgggggtgatggagtgtaaggccagcgcggctctgtcggtgccgcgggaggaacagaatgagcggagtcccgctgagcagatgccgcctcgtcccatggaaccggcgcaggttgcggggccggttgacatcatgagcaagggcccaggggagggaccaggtgggtggtgtgtgcccgggggattggaagtgtgctataagtaccgtcagctcttctcagaggatgatctgttggagactgacggttttactgaacgagcctgtgaatcttgccaataaacgtttattgccatgtccaagttgttgtacgtgcgtgatcatgagggctttgcctgcctaacggtcgaaacccaccgcaaccgctggttcgcggctcacattgtcctcaccaaggactgcgggtgtctcaagctactcaatgagagggacttggagttttacaagttcctctttacgttcctggccatggccgagaagcttgtgaactttaacattgatgaactggtcaccagcttcgagagccacgacattgatcactactacaccgagcagaaggccatggagaacgtccacggggagacttatgctaacattttaaacatgctctttgatggggacagggcggcgatgaacgcctacgcagaggccatcatggccgacgaggccctgcaagccaagatttcctggctccgtgacaaggtggcggccgccgtcaccctgccggagaagattcttgtgttcctgctgattgaaggcatcttcttcattagctccttctacagcatagccctgctgcgggtccggggcctaatgcctggcatctgcctggccaataactacataagtagggatgagctgctccacacccgcgctgcctccctgttatacaatagcatgacagccaaggctgaccgaccaagggccacctggatccaggagctgtttcgcactgcggtggaggtagagactgccttcatcgaggctcgtggagagggggttaccttggtggatgtgcgagccataaagcagtttctggaggccacggccgatcgcatcctgggtgacattggtcaggctcccttgtatggcacaccaccccccaaggactgcccgctcacctacatgactagcatcaagcaaactaatttctttgagcaagagagttccgattacaccatgctggtggtagatgacctttgagtcagggtggctacttgctcaggtttctgggcataaattctcctgcctgcctctgctctggtacgttggcttctgctgctgcttgtgatcatggaaaccactcagactctccgctttaagaccaaggccctagccgtcctgtccaagtgctatgaccatgcccagactcatctcaagggaggagtgctgcaggtaaaccttctgtctgtaaactatggaggcccccggctggccgccgtggccaacgcaggcacggccgggctaatcagcttcgaggtctcccctgacgctgtggccgagtggcagaatcaccagagcccagaggaggccccggccgccgtgtcatttagaaaccttgcctacgggcgcacctgtgtcctgggcaaggagctgtttggctcggctgtggagcaggcttccctgcaattttacaagcggccacaagggggttcccggcctgaatttgttaagctcactatggaatatgatgataaggtgtccaagagccaccacacctgcgccctgatgccctatatgcccccggccagcgacaggctgaggaacgagcagatgattgggcaggtgctgttgatgcccaagacggcttcctcgttgcagaagtgggcacgccagcaaggctcaggcggcgttaaggtgacactcaatccggatctctacgtcaccacgtatacttctggggaggcctgcctcaccctagactacaagcctctgagtgtggggccatacgaggccttcactggccctgtggccaaggctcaggacgtgggggccgttgaggcccacgttgtctgctcggtagcagcggactcgctggcggcggcgcttagcctctgccgcattccggccgttagcgtgccaatcttgaggttttacaggtctggcatcatagctgtggtggccggcctgctgacgtcagcgggggacctgccgttggatcttagtgttattttatttaaccacgcctccgaagaggcggccgccagtacggcctctgagccagaagataaaagtccccgggtgcaaccactgggcacaggactccaacaacgccccagacatacggtcagtccatctccttcacctccgccacctcctaggacccctacttgggagagtccggcaaggccagagacaccctcgcctgccattcccagccactccagcaacaccgcactggagaggcctctggctgttcagctcgcgaggaaaaggacatcgtcggaggccaggcagaagcagaagcaccccaagaaagtgaagcaggcctttaaccccctcatttaacaccatgttctcgtgcaagcagcacctgtccctgggggcctgtgtcttctgtctcggcctcctggccagcacccccttcatttggtgctttgtctttgccaacctgctctctctggagatcttctcaccgtggcagacacacgtgtacaggcttggattcccgacggcatgcctaatggccgtcctctggacgctggtacccgccaagcacgcggtgagggccgtcactccagccatcatgctgaatattgccagcgccttgatcttcttctccctcagagtctactcgaccagcacgtgggtttctgccccctgtctctttctggccaacctgcctctcttatgcctgtggccccggctggccatcgagattgtttacatctgcccggctatacaccaaaggttctttgaacttgggttgctcttggcctgcaccatctttgccctgtccgtggtctccagggccctggaggtgtcggctgtcttcatgtctccatttttcatctttctggctttgggctctggaagcctggccggtgctcggcgtaaccagatttacacctcgggtctcgagcggagacgcagcattttctgcgcccggggagatcattcggtggcatccctgaaggagaccctccataaatgcccgtgggatctgctggccatctctgccttgaccgttcttgtcgtctgtgtgatgattgtgttgcatgtgcacgcagaggtgttctttggactctctagatacctgcccctctttctctgtggggcgatggcctccggggggctgtacctgggccattccagcatcattgcatgtgtcatggccaccctctgcaccctgacatctgttgtggtatatttcctccatgaaacccttggacccctgggcaagaccgtgctgtttatctcaatctttgtctattactttagcggggtagcggccctgagcgcagctatgcgctacaagcttaagaagtttgtgaacggacccctggtccatctccgtgtggtatacatgtgctgttttgtctttactttttgtgaatatctgttggtgacattcattaaatcctaacgaccggagtcctgtctctttgtgttcttgggggacttgagttagctgtctttcctcttattacattgggctaacgggaggaaatgaacccaggggtggcagtggatggggtcatttatgggcaaaactcacaggacatgtttggggagttagcattggcgtcgggaaacacagctctggcagttataaccgcaccagctaacaggacatgtttgggggagttggcattggcgtcaggagacacggctctgtcagttatcaccgtaccatgagtgccatgtgtgtccagtgcctaatcaccgttcctcattttgtgtgcctcctcaaatgttccagaagtcggccacaggggaggtggctgaattagggccttttccctcattcccccatgagacccacgtggcaggcctaggggctacattcgcctcccacgtttcccttcgcgtgaggcatccgatatgactgaattttcgcagtctcttttccctcttcccttgttattcccatagaattacagtgaggttacacaggtggagattcagtttaaccatttattgatttaatccaggaacaaaaaacagtcctagtgacccagtgcccggagagagaatggccctgacaagtcggctgcatgatgcacttcggcagtcacgtgtgtgagtctccacggcctctgtcaaaagggagcttagcgtgccagggttgtaattcttgatgtagtggcccaggaattcaacttcatcgtgtctccgtctgcagttggcgttaatgtaggctggggctactgccgcatatgctgccaagagacagaggggctgcttcacatatgagctgctcagggtctccaccaccttgttttgacgggccgtggcacaggtgatgtagaagagttgcttcacaaagttgtagtctcgcgtgttaggaaggaagcagggtgccagctctttgagcttggtcaggatcaccttgctaagactcatggcgcaggccaggaggatgtcttccgcgggagctaggggcaggtcgccgtggtaggtgatctcctggagccaaaagatggtctcttctagcatggccaccagggtgcagagccccgcgttctggatcgcctgcatgcgtgcatccagccatgtgtccttgttggttgacttggtgaaaaactcacgtagtgtcttgtagctcctgcgcagctcgtgtctgggttgcactttctgccaggctccaatctctggatgggcggccaccgccagcatcgactgtaggaacgggtcttggatgggctctagggtcagagaggccagggggctgggcaaggtgacaaatgtaatcttggagacaggcttaaccagactcatgtcaaaccacggtttgttcggcaggggcctctggctgcgttcttgcctcgcctgcttccttgtgctcctgccggcccctcgagattctgaccggggacctctggttgctctgttgcttcggggagctcttggagacctcggtgctctaggcaccctgggggcccttggggctctgggcgctcttgctcccgggggcaggtgtcggcgcttgccataactttcatcggtgcagccatggacctctccgcgtcgccttttgtggcctctggtgtaagaggagttgccagtctcctccttctcgtcctcgtccctgcacaggggtgagcgatgcaatgtgactgtcttgtcctgtaggtcccacttctttctgggaatcacaaacgatgccgaggtaggggttatgaccacgctggagggccgtgcaggtatggcgtgggccggagttggatcttcatcctcctcctctgaggatgaaatctctccatctgtggagtgttcttcgctgccctccatagggtccagatcgcagtctgtgttggtgtctgagaccgcttcgagttccagaatgtggctctctgcagaggggagacaaaaggtggagactgccttgagcacctctgtctcaggcaccggatgcccccggctccacggccccggccactggccggtgtagcttcttacctgcgggatcctcgttggaggaaatgctgctagttcgggagagtctctgagaaggaaccatcttgtctgtctctacgacgggctagctgggatgtagtgctgtcttgactggcctcagccctatttatgattctggaggcgggcacgctgatggagaaatgggcggtcggttgattggccccacagcgaccggcgaagcactgactcatgaaggtgaccgtgatggcctgtgatgtgtagtagagtaccagaaacaccctcacattcttggagctggccctgtgggtatgcctcaggcacgcaaagttcctgccccgggcatggcacacctgaactaagtttggcccggtttgctcaaacgtgacatggagaaactgggggaatttgtcttctggcacagctgttgccagggtgctcatgagcgagggccagatgcaggagctgacccaggcgacgagatccaggcccagatgtccctctatcatggcgcagacattctccacggtggggggcagggtctcgcgggtcctctggattagatagtcacgcccatcatccgcgatgtggtagcagaaggttttgggggccggccagcccacgtgcagtgagtgatgtaagaggttttgaatgttgagggcattcttaacatagctgtgcttgtcttcctcttccggatgacagacaaagaggcgcagctgccggctaagaccaccgcccctgtccaccttgtaggtatgcggcagccggatgcaccgcccggcgtgatacacgccgctgtcaaaaagcggggccccaatctctttgatcttgtgacgcatgcggcgcaggcaggccgttaggcccatgagcttctgcagcacagacacaaacccttgtactgcgcttgttcccacaatagcatggcctctaggtaggggggtgatgacgcgaaagcccagttttcccgtgcatatgcaaaaggggagcacatcttccatattatccgggtcggcgggtggacaagctgatttgaaaaaatagactgggtgggccctggacactggacccaggcggcgcatgaggcgcagtacctcacgccgcacggtccggcacaggtcatagatttcctccagcgaccagggggcccccttgatctttagatccaggtccaagaccaggttgcagaccggaagccggggattaaagtattcatgccgggagacaaagagctgctcgctcaggctgttctgtgaatagtacactggggtgtaggagagggccctggtgagacacgtgtctgggaggcggcagttggtcggggtggagacgacctccgccaggtgggatgagaaggggtcagcggctgtcattacaaagtagtgcctgtctgcaaaatggcagaggaagaccggtagccgctgcacccttcgaaggacggtgggtgggaggaattgttccttgggattccactggccccggcaggtggcctggccggccaagcatagaaacccttgaagcgtgggggggtatgtgggaccctcatccgcgtgccagcgcgcgagctccaccagctcccgggccacgtccacactgagcccggcccaggcccgcatgagtccgtcatcggggtcggggtcccacgtgtatggggccgggggctccatgcggattttcagctgctggacacgcacatgctcagccaggtaagtctcccgggtgaagtaggtgcgcatgtgctccgcaaagcccctgtccaggagcgaggggagcacgacgccccccgaaggcagacacccaatttctcccatgctcgttaactgagagtatcgcttaaaggttccctcgttgaagcactgtgcgtgggccaaatagacgtagcgcacgagatcggccgaggccaggggaaggcgccccctgtaggcgtctatcgtccttgccacagcgcggatctctcgcgagtcccgccgcagtttctcgtgtgcaaagtgggcaaaagcctcggtctgctccgcccatgccgaggagccaaagacctcccccagctcggccagggacgtgacggcggccaggctctgaccagactcggaagtaaatagctccgtgaggtgcgccagggtctcaatcgtacaaggaatgccccaaaaatagtaagcagccgtgactagcacgaactgggcctcgtgggagccaaaggtgctaatgaaccacctggccgagatgttaacgcggtagatgcggcgcagacagcccacgatcttgggacgcagccacgccacgcggcctctggcatccccctgtggctgtttcttagcgctcagtgtgagcagttccacgaggggcgtgagcgagcgcagggcccccgcgcgatctaggtaggtggatagacggtccgcggtgagcggcgtgaggccgcgcaggaaggggaaggcctcctccgccggcaggtgcagcgtcagaaccaggccgcagcggctctgtgaggtcagccgcttcttgggcaggtgaagctgcagttccacgagagaacccgccacgtggtggaggggcgaggcgttgtggcacaaacaaaacaggcggaagccctcgtcaggccgcgagaggatggcatcgaggatggcctccgcaatgtcagtgtttgaggccacaagggccttgatgacgacgggggcggacattatttaagaccgggaggccccaacggcgggctaaacagaacgatggccttctatctcccagactggtcgtgctgcgggctctggctctttggccggcccaggaatagatacagccagctccctgaggagccggagacctttgagtgcccggaccgctggcgagccgagatagatctgggcctgccccctggtgtgcaggtgggagatttgctaagaaatgagcagacgatgggctcactgagacaggtttatttgctcgctgttcaagccaatagcatcacggatcacctgaagcgctttgacgccgtccgcgtccctgagagctgtcgtggggtggtggaggcccaggtggccaagcttgaggccgtgcgctcagtcatctggaataccatgatctctctggctgtaagcggcatcgagatggacgagaatgggctcaaggccctgctggacaaacaggctggcgacagcctggccctgatggagatggagaaggtggccacggcgctcaagatggacgagaccggtgcctgggcgcaagagatctcggccgttgtctcatcggtgaccgccccctcagcctcggcccctttcatcaactccgcctttgagcccgaggtgcccacccccgtccttgcaccgcctcccgtggtgcggcagccggagcactctgggcccacggagctcgcgttaacgtagcaaccagactccacaccaaataaacattttattggtaaaacaagggatatgaaggtgtcattgacccgaggatccaaaccccctcccctgtctcccctcgagcgcctcgctcagcccactatcacccatggccaggctcggcacctcctcgaaggtgcagctggcccacctaaagagagatctggggccaaggacccccgcgtcactgtgggggctgtagaaggaggtgaggtggtgcttgtgaaggtaaacaagctgacagaagcgccggtacttgttaaggaacacggtctggtcactaaagttggtcaggctgacgtccaccccaccccggcgccacctgcagggcttcactagaataccctgcatggccaggcccgacctgccaaagattgtcggcctgtggtgagggatagaaggggggggcacggtgagtgtcactgagacggtctgatgggggaagagggccaggtcctttggcaaagagacgtccaggcccacgtccccggggtactgggggtggttgatgggacccttgtcctcctccatctggggggtggcatatctgaaggcagccaggtggattttgagctccgatggacgcagcgtggagttgtagcgccgctgattctggaggattagccggagttcccccgtgtagccgggatcgatgatgccaacatgagacgtgaccggacgggaggtgctgccccacagcatgagcccatgaccctcgggtgggcgggcatagaggcctaggtccacagttgtggtcttcatcgggcgcagcaggatggtggtcttgttgaccaaggtgagccgccctacactagcctgctggagcaacagcttgtcattctggaaggcgtagcgtatgtgtggacaggcctccatggtgatgatctaacagacagggacggcggcgctatatataagagcccaagacccggctctctttactgcgaaatggggaaggtcctaagaaagccgtttgcaaaggctgtgccactgctcttcctcgccgccacctggcttctgaccggggtgctgccggccggcgcttccagtcccacaaacgcggcggcggcttccctgactgaagcccaggaccagttctactcctacacatgtaatgcggacacattctcgccttctttgaccagctttgcctccatctgggcacttctgacgcttgtcttagtcattatagcctcagccatctacctgatgtacgtctgctttaacaagtttgtgaacacgctgctgacggattagatggggatatttaaaaggggcagcaatctcggctgtttgtacttcttctctgctcgttaaaccaatagcatgtcagctccacgcaaagtcagattgccttctgttaaggctgttgacatgagcatggaagacatggccgcccgcctggctcgcctggagtctgagaataaggctctgaagcaacaggtcctcagagggggtgcctgtgcctcgtctacctctgttccttctgctccagtgcctccgcctgagccgcttacagctcgacagcgagaggtaatgattacgcaggccacgggccgtttggcgtctcaggctatgaagaagattgaagacaaggttcggaaatctgttgacggtgtaactacccgcaatgaaatggaaaatatattgcaaaatctgaccctccgcattcaagtatctatgttgggtgcaaaaggccaacccagccctggtgagggaacacgaccacgagaatcaaacgaccccaacgccacccgacgtgcccgctcccgctcccggggacgtgaagcaaagaaagtgcaaatttctgattaataaatttttattgactttatacataggtctcggcgtcatcatatggtggggtggtgtaggtatgggatgtagacaagttacgcctaaaggcgcagtccgccatgaccagcagcagcagaagggtcagcacagccagagaggcccactgcagtactagcatggagaggtttgagaatctgggctgggacgttggcgggactggcacggtggcttgggctgtggtaaccggtgggctcgtaaaagtccagcggggccgcagtttgctagaagtgctgggaggtagataggtggtcgcattgtatctcggtcttggcgtagttgaatcaccgccgtaatctgtggtgggctctgtacttgtccgggctccatgtcctgtggtgtgctttccaccggtggtagaattggcctttccacctgttgaggtgaccgtgggaaccgccgtcttttggccactgggggcctggggcgacgttgcattttgggggggcgtgcctttggtgacattaacctcccccggttttgtggatgtggaactgtttccagggcctgacgcttggctggtggtgcctgggcggggtgctggcgaactggtggacacatgatgtgtgctgatagaggctggtgtcacctgtgttatattttcaccacctgttgggtgagcggaggttagtaaaggcatatgtgacgttgaattgtcactggtggaggggctgagtgtctctgggtttgaactgggtctcagtgacatggaagaggttgaacttgaagttatgttatgttggcctgtggtaacagcactggttgcattttttggttggctggtaactactggggtgggacttgttcctcctaaggtgtggttggtggcatttgcctgtggacttgtttctcccacagtagggccggtggcatttggggttggggtagtcactgctgaggtggggcttgtttttcccaaggtggggctggtggcatttggggttggggtagtcactgctgaggtggggcttgtttttcccaaggtggggctggtggcatttggggttggggtagtcactgctgaggtaggacttgtttttcccaaggtggggctggtggcatttggggttggggtagtcactgctggggtggggctggtggcatttggggttggggtagtcactgctggggtggggctggtggcatttggggttggggtagtcactggtgaggtggagctggtcatgtcgggggccttactttctgtgccgttgtcccatggagatggacttggtgtcaccggtgatgcgcctgacgttgtgccggctggtgttgggctggtgacatccgcggtggatacagtggggcctgtgcttgcaggtgcggtgaggttggtaggcacgtgagtagagctgggtagacctgtcgttgtattgggatcagcaaatccagttgtattcaaggtaggggaggtggtggtgctctcgggtgccttggagaatataaccttgtgggttgttgtggtggcattggtagccgttcgtgtgataatgagtgtcttgggggccgtgccaagacccgagacagtaatgtcaaatgtccgattgctcgcaaatgcaccagaaatattttcacaacccgaaggtgtccccgaggtgagagtccatttgcacttaaagtcagtttcagtgttgtttggccaggcccaaaaggcagtcactgtaacatttggcgagtttgcgtcctcagaagtgaccattggcactgaataggtagcattgtcacccacatatgtgatgtctgtggtgtttgtcggcatgtcctgtgaagctggaatctcatcagagaacacaatgttggactgaatgcagtaatctcccccgctcgccttcggtccattcccagagtaaaacacgtacaggatactgttattgccaagaaatcgtgacactggacgtggtgtcagacgcaggctgtatgcataccctgtaccaggtattggggtggccacgggactcgttgatgtgagaattccgccgctgggaacatggctctcgtatccactgcaggtgatgttaaatttgttgtctccgggcagaacttgtgaaatttcgccatcctccataatacactcaatatctatctcattaccgagcatttctgtttttacgctgaaattcgagtcttgagctgacgttggcaaacttaagggtagcgtgacatccagcccctgtgccctcactactgccgttatattggtagaattacagttatcccactttatgtatggcactgtttctggtatcaggtacacggggttttgcatttctgcatggtggcaccacatggttccaaacacatcttgaaagtagacatctacagattccaggcttacttgttgctcctctccggtggtgacgttaattggaagcttcttagaccgcatagttagagccaattctcctgcaccaaggagctccagtagaaagagattggtggcattttctgagccaccaaatgcacctcgaggttggtagacagccttcgtatggggtgtcagctggccaaagtcaagatcaagttgatgctttttgcccccgacatcgaaattgatagttacattgacatctgccgtgcaaacattgcatgtggggtaaaatgggaattccggaatctcaacattgaaaaaaccaggatcttcacccgtgagatggatcaggctctggatggtgtactgacacacaagcaaggctgcctccattgtctcggcaccgatttctaggcagcatcctctttaataggtacaaggggggtgcggtgttggtgagtcacacttttgttgcagacaaaatggacaaggacaggccgggtcccccggccctggatgacaacatggaagaagaagtcccatctacctcggttgtgcaggaacaggtatcggcgggagattgggaaaatgtcctcatagagttatcagatagcagctcagaaaaggaagcagaagatgcccacctggagccggcccagaagggtacgaagagaaagcgggtcgatcatgatgccggtgggtcagctccagcacgacccatgctcccaccccagccggatctccctgggcgagaagccattctccgcaggtttccactagatctaagaacacttcttcaagcgattggagccgcggctacggtgagcatccctatggcctaagtgtgtgatgtgtgtttttacccatcacacaacaacaaggtaagtaatttgttgccgttggtttcagcgcatcgacacacgagccatagaccagtttttcggatcccagatttcaaataccgagatgtacataatgtatgccatggccattcgacaggccattagagatcgtcggagaaatccagcttctcgtagagatcaggccaaatggagactgcaaaccctggccgccggatggcctatgggttaccaggcatacagcagctggatgtacagctacaccgatcaccagacgactcccacattcgtacatctccaggcgacacttgggtgcacaggtggccgtaggtgtcacgtgaccttttccgccggcacctttaagctgccgcgatgtacccccggggatcgccagtggttgtatgttcagagctccgtgggtaacattgtacagagctgtaatccccgctacagtattttctttgactatatggctatacacaggagcctcacgaaaatctgggaggaagttttaacacctgaccagcgtgtttcatttatggaattcctaggatttttgcagagaacggatttgtcctatatcaagagctttgtcagcgatgccctgggcaccactagtatccaaacaccgtggatcgatgacaatcctagcacggagacggcacaggcttggaatgccggctttctccggggtcgtgcgtatgggatagacttgcttagaactgaaggggaacatgtcgaaggtgctaccggtgaaacgcgagaagaaagtgaggacacggagagcgatggagatgatgaagatcttccttgtatagtgtccagaggtggacctaaggtcaaacgaccccctatatttataagacgtctgcacaggttgctgctgatgagagcgggcaaacgaacagaacagggcaaggaggtactggaaaaggcccgtgggagcacttatggcacacctaggccgcctgttccgaaaccaagaccagaggtcccacaaagcgacgagacagctaccagtcacgggtcggcgcaagtcccagaacccccaaccattcacctagcagctcagggaatggcatacccattacatgaacaacacggcatggccccgtgtccggtagcacaggccccacctacgcccttgccccctgtatctccaggggatcaactcccaggtgtttttagcgacgggcgagtggcgtgtgcaccagtacccgccccggctgggcctattgtccggccctgggagccatccctgacacaggctgcggggcaggcctttgcacccgttagaccacaacacatgccagtagaacccgtccctgtcccgacagtggcacttgagcgaccagtttaccccaagccagttcgtccggcacctcctaagattgctatgcagggccccggggaaacttctggcattagacgcgcgcgggagcgttggaggcccgcaccttggacgccaaatccaccccgttctcccagtcagatgtccgtgcgtgaccgtctggctcgtttgcgtgctgaggcacaggtcaaacaggctagtgttgaggtgcagcccccccagttgacccaagtatcccctcagcaaccaatggaggggccgttggtaccagagcagcagatgttccctggtgccccctttagccaggttgctgatgtggtccgggcacctggggtaccggcgatgcagccacagtactttgacctccccttaattcaacccattagccagggggcacccgtggccccgttgagggctagtatgggcccggtacctccggtaccggcaacacagccacagtattttgacatccccttaactgaacccattaaccagggggcatccgcggcccattttctccctcagcaaccgatggaggggccgttggtacctgagcagtggatgttcccaggtgccgccctgagccagagtgttaggccaggggtagcgcagtcacaatattttgacctccccttaactcaacccattaaccatggggcacccgcagcccatttcctccatcagccaccaatggaggggccgtgggtacccgagcagtggatgttccaaggtgccccccctagccaaggcactgacgtggtccaacatcagctggatgctttggggtatacactccatggtcttaaccatcccggggttcccgtgtctcctgccgttaaccaatatcatctcagccaggctgcctttgggttacctattgatgaggatgagagtggcgaggggtccgatacctccgagccgtgtgaagctcttgatttgtcaatccatggcaggccctgccctcaggcccccgagtggcctgttcaagaggagggtggccaggatgccaccgaggttcttgatttgtcaatccatggcaggccccgccctcggacccccgagtggcctgttcaaggggaaggtggccaaaatgtcacaggccctgaaactagaagggtggtggtgtcagctgttgttcacatgtgtcaggatgacgagtttccggatctacaagatcctccagatgaggcctaagcaaaggtgtagaagtgtgtccccctccattccacccactgataatacgcccgacaataaagttgatgatattgaattccacacctgcttgtgtttgtgattttatttcatattccatgagagagacctcgcatatttgcagaagggtcactgaaacatcttatcttaaaacagttacacctgaataatgaagaaagcgtggctcagcagagcacagcaagccgatgccgggggggcatctggctccgaggacccaccagattatggagatcaaggtaatgtgacacaggtgggatctgagcctatttcacctgagattggcccctttgaactctctgcggccagtgaggatgatcctcaatctgggccagtggaagagaatttagatgccgctgcaagagaggaagaggaacctcatgagcaggagcacaatggtggtgacgatcccttggatgtccatactcgccagcctagatttgtggatgtgaacccaacgcaggctccagtgatccaactagtccacgctgtctatgattccatgttggtaagaggcacctagaacatttccagatgtttcgcttggattttttggccagtcttaattgattgtcattggtttcagcaatcggacctccggcccctaggcagtttattccttgagcaaaacctgaacatcgaagaatttatatggatgtgcatgaccgtgcgtcacagatgtcaggccatcagaaaaaaaccattaccaattgttaagcagaggcgttggaagctcctgtcatcttgcagatcctggcgtatgggttaccgcacgcataacctcaaagtaaacagttttgagtcagggggggacaatgtccacccggtccttgtgactgctacgctaggatgtgatgagggcacgcggcatgcaacaacgtacagtgctggcattgtacagataccacgaatatcagaccaaaaccaaaagatcgaaacagccttcctgatggcacgtcgtgctaggtcactttcggcagaaagatatactttgttctttgatttagtatcctccggaaacaccctgtatgctatatggattgggctgggcacgaaaaaccgagtttcatttattgagtttgtaggatggttatgcaagaaggaccacactcatatacgcgaatggttccgccagtgcaccgggagacccaaagcagccaagccatggttaagagcgcatcctgtcgccattccttatgatgatccgttaacaaacgaggagattgatctggcctatgcccgcgggcaggccatgaatattgaggctcctagactgccagatgatcctataattgttgaggatgacgacgaaagtgaggaaattgaagctgaaagcgacgaggaggaagacaagagtggaatggaatctcttaaaaatataccgcaaacactgccgtacaatccaacagtatacggcaggcccgcggtgtttgaccgaaagtcagatgcaaaatcaaccaaaaaatgcagggccatagtaactgactttagtgtaatcaaggccattgaagaggaacacagaaagaagaaggcagccagaacagagcagccaagagccacgcctgaatcccaggcccccacagtggtcctccagcgaccacccacgcagcaagagcctggccccgtcggcccactgagtgtccaggctcggctggagccatggcaacctttgcctgggccccaagtgacagcagttctacttcacgaagaatccatgcagggtgtccaagtacatggttcgatgctagaccttcttgaaaaagacgatgaagtcatggagcagagggttatggcaaccctactgccaccagtaccacaacagccccgggctggcagaagaggcccttgtgtcttcaccggtgacctaggcatagagagtgatgagcccgcttcgacagagccggttcatgatcagctactgcctgccccaggacctgaccctcttgaaatccaaccactaacgtcccccaccacgtctcaacttagcagttcggcaccaagctgcgcacaaactccatggccggtggttcagccaagtcagactccagatgacccaacgaaacagtcccggccaccggaaacagctgccccacgccagtggccaatgcccctgcgacctatccctatgcgccccttgcggatgcagccaatcccatttaatcatccagtgggacccactccccatcagacacctcaagtggaaataacaccatataagcccacttgggctcagatagggcacattccatatcagcctacaccaacgggtcctgctaccatgctgttgcgccagtgggcacccgccaccatgcagacaccaccgagagcgcccactcccatgtcaccacctgaggtgccacccgttccccggcagaggcctcggggggcgcccactcccacgccacctcctcaggtgccgcccgttccccggcagaggcctcggggggcgcccactcccacgccacctcctcaggtgctgcccactcccatgcagctggcactaagggctcctgctggtcagcaggggccgacaaagcaaattttgcgccaattgttaacggggggcgtcaagaaagggagaccatcacttaagttacaggccgcccttgagcgtcaagccgctgcgggctggcagccttcaccagggtccggcaccagtgacaagattgtgcaggcgcctattttctacccacccgttttgcagcccatacaggttatggggcaagggggttccccaacggccatggccgcctcagcggtgacacaggcacccacggaatataccagggaaaggaggggagtggggcctatgcctcccaccgatattccgccgtctaaacgagcgaagatcgaggcctatacagagcccgagatgccgcacgggggggcctcgcactctcccgtcgttatcttggagaatgtcggccaggggcaacagcagactctggagtgcggaggaactgctaaacaggaaagggacatgttggggctgggggacattgcagtttcttccccttcctcttctgaaacatcgaacgatgagtgatttcgcccatgtaacaagaactgggatgaaccctggggcaacagactgcggggaggaggggggcagtgataagtcatgacaattttagatgaggtagaaattttgcatattttcagacccaccatggaatcatttgaaggacagggggactctagacagtcacccgacaatgagcggggagataatgtacagactaccggcgagcatgatcaggaccctgggccggggcctccatccagtggggcttctgagagattggtaccagaagagtcatactcaagagatcagcaaccttgggggcaaagcaggggtgatgaaaacagaggctggatgcagcgcatcaggcgaaggcggagaagacgggctgccttgtccggccatcttttagacacggaagacaatgtgccgccatggttgcctccacacgacatcacaccatataccgcaaggaatatcagggatgctgcctgccgggctgtcaaggtgagtatgcctctaactgggttcatgggggccatctaaggcccacgtgtgacccatgtttccattaattttagcaatcgcacctgcaagcgctatcaaacctgatactcgatagtgggttagacacacaacacatcttgtgcttcgtgatggcagccaggcagcgtcttcaggacattcgacgtggacccttggttgcggagggcggtgtgggttggcgacattggcttctaacatctcccagccaatcctggcccatgggatatcgtacagcaacactacgcacattaactcccgtgcctaacagggttggggctgacagcatcatgttaactgccacatttggatgccaaaatgcggcacgaactctaaacaccttctctgccaccgtgtggacaccaccccatgctggaccaagagagcaagaaagatacgctcgggaagccgaggtacgcttccttcgtggtaaatggcagaggcggtaccgaagaatctatgatttgatagaactgtgtggctctctgcaccacatctggcaaaacttgctccagaccgaggagaaccttttagatttcgtgcgtttcatgggtgtcatgtccagctgcaataatccagctgtgaattactggtttcacaagacaatcggaaactttaagccatattacccgtggaatgcaccacctaatgaaaatccatatcacgcgcggagaggcataaaagaacacgtaatccagaacgcatttcgaaaggcacaaatacagggtttatcaatgttagcaacgggaggtgaacccagaggtgatgctactagtgaaacgagcagtgatgaggacaccggtagacagggttcggacgtggagctagagtcctcggacgatgagctgccatatatcgatcccaatatggagccggttcagcagaggcccgtcatgtttgtgagccgtgtgcctgcaaagaaaccgaggaaactgccttggcccacgcccaagacgcacccagtgaagcgcacaaatgttaagacctctgatagatctgataaggcagaagcacaaagcacccctgaaaggccgggcccttccgaacaatcatcagtgaccgtggagcccgcccacccgaccccggtggagatgccaatggtgattctccatcaaccacctccagtgcccaaaccggttccagtcaagcctacgccaccgccttcccgtaggagaaggggagcgtgtgttgtgtacgacgatgatgtcatagaggtgattgatgttgaaaccaccgaagattcatcgtcagtgtcacagccaaataagccacatcggaaacatcaagacggctttcaacgttcaggccgacgtcaaaaacgagccgcgcctcccaccgtgagtccttcggatactgggcctcctgccgtggggcctcctgccgcggggcctcctgccgcggggcctcctgccgcggggcctcctgccgcggggcctcctgccgcggggcctcctgccgcggggcctcgcatactggcgcctctttccgctgggcctcctgccgcggggcctcacatagtgacgcctccttccgcccggcctcgtataatggcgcctcccgtcgtacgtatgtttatgagggagcgacagctcccccagtccaccggccgtaaacctcagtgcttctgggaaatgcgggctggtcgtgaaattacacaaatgcaacaagaaccaagttcacacctgcagtccgccactcagcctacaacgcctcgcccatcatgggccccatcagtctgcgccctctcggtgatggatgctggtaaggcccagcccatagaaagttcacacttgagttccatgtcgcccacacagccgatatcgcacgaagaacaaccccggtatgaggatcctgacgctcctctggatttaagtttacatccagacgttgctgctcaaccagctccccaggctccataccagggataccaggagccgccggccccccaggctccataccagggataccaggagccgccgcccccccaggctccataccagggataccaggagccgccggcccacgggctccaatcatcttcatatccaggatatgcgggtccctggaccccaaggtctcaacatccatgttataggcacccctgggcaccatggtctcaagatcctgtgcatgggcacacccagggtccatgggatcccagggcaccacatctcccacctcagtgggatggatctgcaggacatggccaggatcaggtctcccagttcccacatctgcaatcggagacaggcccaccacgtcttcaactttcattggtgccactggtctcatcctctgcaccatcatggtcatctccccagccccgagcccccatacgccccattccaacaagattcccccctccccctatgccgttacaagatagcatggccgtggggtgtgactcatcaggtacagcatgcccaagcatgccctttgccagtgattacagtcaaggtgcatttaccccactggacattaatgccaccacgccaaaaaggcctcgagtagaagaaagttctcacggacctgcccggtgttcccaagctactgctgaagcacaggagattctcagtgacaattctgagatctccgtgttcccaaaggacgcgaagcagactgactacgatgcatccactgaaagtgagctagattaaggggatccaaggtgacccctgttagctatttgatctttgactgacacataaacatggtttaaggaatgaacactcatggtgtgagactggaactgtactaaatttgctgacatatgtacaatgagagccaaaaatttgataaaccttaaaagtccccccatctaatgatgtccagttcccttctcccaccctgtacaccccgacccaaagggactcaatggcattcagatttctagttaccacaggtagaatatcgggcgttggcccataaaaataagtgcatggatatagctctgcacaggcttggaaacacccattccaggtgtgcttctttttggtgaaataaaaacagcatcctttatatgaaaatgtgtattctctggtgttgcagtatgtacagttagctttggtatagttttggggtacctgaaatgtgtgcagggtgggtgtccaatgtggcagttttacctctttgtccccatactcctgctcggccgtcttgttaaagttaaccggcggtggaggatccaccggccagacctctacatttggtttgggtacccaggtgatggccgcggctgccacccgccctcctcctcttaccctgggtggcaaaaagtatgccaggagtagaacaataacaagtgcgatggcggtaaacaatggcaccctcacctgcttaaatgaaaccatggcaaccacttcaaagagagccgacaggaagatatttattaatattccattagtaaacgaggcgtgaagcaggcgtggtttcaataacgggagttagaaatttaagagatcctcgtgtaaaacatctggtgtccgggggataatggagtcaacatccaggcttgggcacatctgcttcaacaggaggcgcagcctgtcattttcagatgatttggcagcagccacctgcggacaaaaatcaggcgtttagatggggcatttatgtttgggacgctagccgcctgggcattcgtgttagtatatactgacctcacggtagtgctgcagcagttgcttaaacttggcccggcattttctggaagccacccgattcttgtatcgctttatttctagttcagaatcgcattcctccagctgcgagcaagggaatgcgttactacaagtggtgcctagtcagttgaaacaagccccaccatccgctgccgcccctccatgagccccaccgtccgctgccgcccctccttgagcccctccttaccgattctggctgttgtggtttccgtgtgcgtcgtgccggggcagccactggtgcaggctgtggaacaccaatgtctgctagctgttgtccttggttagccccggggcaagcaaacaccactgctgctgctgtttgaacagtagaattgtctccaggttgaggtgcttctcccccggcttggttagtctgttgattctgggttatgtcggagactgggaacagctgaggtgctgcataagcttgataagcattctcaggagcaggctgaggggcagaaaaccacgacccagtcggagcggttgaaacatgataggcagttagctggccttgtggcagaggctctggcagcaccggccacagcacacaaggcaaaggagcttgcgatggccctcccaggtcctgatagactctggtagcttggtcaaaagcttgtacaaaaggcacctggtatgggtcaggtgtaaattttacatcttcagaagtcgagtttgggtccatcatcttcagcaaagatagcaaaggtggccggcaaggtgcaatgtttagtgagttacctgtctaacatctcccctttaaagccaaggcaccagcctcctctgtgatgtcatggtttgggacgtgctaaatttaggtgtgtctatgaggtacattagcaatgcctgtggctcatgcatagtttctaaaagaggaggaggcagttttcagaagtgtctaaaataagctggtgtcaaaaatagacagcccagttgaaatatgcatggcatgcagcagacattcatcatttagaaatgtatccaagatttcattaagttcgggggtcaggggggagtccagattcaaatcctctgtcatggactctagtgttgtggtcagttcgtccaaatggccacgagggggcgggtggctcaggtccatctgtccacatatggctgcttcctccttctggggaataacagtgtcagccatctcccttagggccttcacggcctgactggtttcttcatcagggtcctccaacagatgacttgcctcgggggttactgcgggggccgggtcaagtggctggggcaccggggctggcgttagggatccgaccggttcatggacaggtcctgtgggggtgggagccaaagaggcaggcaggggccggttggcccacggggatccgggtggatggaagggcctgatcctctttggctgacacacctctcgcccctcgaacacgtcagatatggcactgcccgcttccggctttggcaggaacataccttcccggctatccctgaggcccttcttccttttaacgggaggaagaaaggtgggctttgaggggtgggggaatatgggtctctcatcgctctcttggtggaccgctgctatccaaggctgttcaggttccgccgcgttggaaggacatggagtttgaccacggttgggcctggatgtccggcgcgactttggggcccgcaggcgcggggcctcggccctggcctcttcccgctcgctctgctcggtgtcactgttgcccgagtcactgctgctggaactgctgtcaccgcagtcggcgctttgggcaccgggcttcaggggcatggtcgggctcgggagactttcgagttcatctgtaaaagcatgaaactgtccggactccgagtagcgggcctcggtgtgagaggcacccccatcattccccatgagctcctcgtccatcctgtcggctccggacacgaggataggagtttccactgccttggacttggttgacagcaggcacgcgggaagcacgccgctcacgtagctcctctgtccggcgtggctggagtaggaggcccggggcagtgtcttaatcagagccctgacatccttaacatcgtccgtcagatggcctgtcttggacgagaccatagtctggaacatctcctcgaggacgggataggtgaacacccacttgcaaaaggccttgaacttggagcttaggaggccttccttctccatcctgttcaggtgttccactacctgcttgccggaggccatgatggccgcgcggtccacgcccagcaccttgctgtaggtgtaggcccgcacccgactgtgttttaggagcttgtacatagcggtgcctatggtggcaggaatcatcacccggttgctgggggcctggatgaagaatctgtcagtgaccactatcaggtggtctaacacgtagcgcatcactatagggcacgcgatggaacatgcgtcgttgccggcattctcagcccgtcttcttaccctgttgtttcggagaatggcccaaaaattgcagatgttgagcgtggccattagcccgccccattctcgcccgtgggccttggcctcatttataaatgccttgcatattttgtaggatctcagagtaatctccacactcccggctgtaaattccttgttgaggacgttgcagtagtcagagaccagagagcccagctgctttttgatttcaggagttagcctcagaaagtcttccaagccatcctttttaggcctcatggctagtagtaacagaggaaatgcccgaccattaaaatctttcctccatgagctttacctgaaacactatcccgaagtgggggatgtggtgcatctactgaacaccatcggggtcgactgcgacctcccacctagccacccactcctgacagcccagagggggctgttcctggcaagagtcttgcaggctgtacagcagcacaagctgctggaagacaccatcgtccccaagatcttaaagaagctggcttatttcttagagctgctaagctactactcccccaaggatgaacagcgtgacatcgccgaggttcttgaccacctcaagacgaatcgggacctggggctggacgacagactctgggccctgattaggaaactgcgccaagacagacaccatgcctctgtaaatgtcctcatgccaggaagcgactacacagccgtgtcgctgcagtactacgacggcatctccataggtatgaggaaggtaatcgcggatgtctgccgcagtggctatgcctccatgccctccatgacggccacgcacaacctctcccaccagctcttgatggcgtccgggcccagtgaggaaccgtgcgcctggcgcgggttctttaaccaggtcctcctctggactgtggccctctgcaagtttcgcagatgcatttactataactacattcagggatctatagccaccatctcccagcttctgcacctcgagatcaaggccctctgcagctggataatatcccaggatggcatgcgcctctttcaacacagcaggcctctcctcaccctctgggagagcgtggccgcaaatcaggaggtcacggatgccattaccctgcctgactgcgctgaatacatagacctactaaagcacacaaaacatgtcttagaaaactgttctgccatgcaatacaaataaatttctcttacctgcgtctgtttgtgtagtgaggtgttgtgtcctgtatggtattctactttaaaaaggccggctgacatggattactggtcttttatgagccattggcatgggcgggacaatcgcaatataaaaccctgaccatcacatggggcattaggcgactctgcatcagcatcgcttaagtatgagtgggcagcagagaggctcggttattttggttcctgaacatctggctggggcattaactaagcttatgagcgattttatcacaggacaagatgtcactctttctggaggaaatattgcagtcaaaattcgcgatgctataaaccagacccccgggggtggtgatgtagctatactttcttccctgtttgctttatggaatgccctcccaacatctggtagacaatcctccagggacgatttaatcccagccgccgtgcaggccttaaccacggcccacaacttatgtctgggtgttattccaggtgagacctcacacaaggacacacccgagtcattgctccgggctatcgtgacgggtctccaaaaattgtgggtggattcgtgcggatgtccagagtgcctacaatgtcttaagggattgaaggcaattaagcccggcctttatgaaatccctaggataataccacacactaagcagtgtagtcctgtcaatctcctgaacatgttggtccacaagcttgtggctttacgtggtcatgtgcagcttgcatacgacgcccgtgtcctgacgcctgactttcacgaaatccctgacctcgatgactccgatgctgttttcgcacgcaccttattggcagccttatttcacctcaatatgttctttattctcaaagattacataacacaagactccatgagcttgaagcaggccctcagtggtcattggatgtctgccacgggcaaccccctgcctgcagcaccggaaaccctgcgagactacttggaagctttccgaaattcggataatcacttttatctcccgacgacagggcctttaaacaccttccaatttcccgaagagcttctggggcgcgttgttgttattgattcctctttgtgtgccgccagtcacgttcaggacgttatcacccatggtgttggggcgggtgttcctcgtcctcggttttcggccctgcctccggccccatcccgcgagccccagcagacatgctctcagttaacgagcagagggaatgaaagctcacggcgaaacttgggccagcccggggggacctcccctgctgttcccccagtttgccccatcgtttccctgacggcctcaggggccaagcaaaaccgcgggggcatgggatccttgcacttagccaagcctgaggaaacctcccccgccgtctccccagtatgccccatcgcttccccagcggcctccaggtccaagcagcactgcggggtcactggatcctcacaggccgcacccagcttttcttccgttgccccagtagcatctctgtctggtgaccttgaagaggaagaggaggggtcccgagaatccccatccctaccgtccagcaaaaagggggacgaggaatttgaggcctggcttgaggctcaggacgcaaatcttgaggatgttcagcgggagttttccgggctgcgagtaattggtgatgaggacgaggatggttcggaggatggggaattttcagacctggatctgtctgacagcgaccatgaaggggatgagggtgggggggctgttggagggggcaggagtctgcactccctgtattcactgagcgtcgtctaataaagatgtctattgatctcttttagtgtgaatcatgtctgacgaggggccaggtacaggacctggaaatggcctaggagagaagggagacacatctggaccagaaggctccggcggcagtggacctcaaagaagagggggtgataaccatggacgaggacggggaagaggacgaggacgaggaggcggaagaccaggagccccgggcggctcaggatcagggccaagacatagagatggtgtccggagaccccaaaaacgtccaagttgcattggctgcaaagggacccacggtggaacaggagcaggagcaggagcgggaggggcaggagcaggaggggcaggagcaggaggaggggcaggagcaggaggaggggcaggaggggcaggaggggcaggaggggcaggagcaggaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggggcaggaggggcaggaggggcaggagcaggaggggcaggagcaggaggaggggcaggaggggcaggaggggcaggagcaggaggggcaggagcaggaggggcaggagcaggaggggcaggagcaggaggggcaggaggggcaggagcaggaggggcaggaggggcaggagcaggaggggcaggaggggcaggagcaggaggaggggcaggaggggcaggagcaggaggaggggcaggaggggcaggagcaggaggggcaggaggggcaggagcaggaggggcaggaggggcaggagcaggaggggcaggaggggcaggagcaggaggaggggcaggagcaggaggggcaggagcaggaggtggaggccggggtcgaggaggcagtggaggccggggtcgaggaggtagtggaggccggggtcgaggaggtagtggaggccgccggggtagaggacgtgaaagagccagggggggaagtcgtgaaagagccagggggagaggtcgtggacgtggagaaaagaggcccaggagtcccagtagtcagtcatcatcatccgggtctccaccgcgcaggccccctccaggtagaaggccatttttccaccctgtaggggaagccgattattttgaataccaccaagaaggtggcccagatggtgagcctgacgtgcccccgggagcgatagagcagggccccgcagatgacccaggagaaggcccaagcactggaccccggggtcagggtgatggaggcaggcgcaaaaaaggagggtggtttggaaagcatcgtggtcaaggaggttccaacccgaaatttgagaacattgcagaaggtttaagagctctcctggctaggagtcacgtagaaaggactaccgacgaaggaacttgggtcgccggtgtgttcgtatatggaggtagtaagacctccctttacaacctaaggcgaggaactgcccttgctattccacaatgtcgtcttacaccattgagtcgtctcccctttggaatggcccctggacccggcccacaacctggcccgctaagggagtccattgtctgttatttcatggtctttttacaaactcatatatttgctgaggttttgaaggatgcgattaaggaccttgttatgacaaagcccgctcctacctgcaatatcagggtgactgtgtgcagctttgacgatggagtagatttgcctccctggtttccacctatggtggaaggggctgccgcggagggtgatgacggagatgacggagatgaaggaggtgatggagatgagggtgaggaagggcaggagtgatgtaacttgttaggagacgccctcaatcgtattaaaagccgtgtattcccccgcactaaagaataaatccccagtagacatcatgcgtgctgttggtgtatttctggccatctgtcttgtcaccattttcgtcctcccaacatggggcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagctggtcatcagccgctgcgcaaacggactcaacgtggtctccttctttatctccatcctgaagcgaagcagctccgccctcacgggccatctccgtgagttgttaaccaccctggagactctttacggttcattctcagtggaagacctgtttggtgccaacttaaacagatacgcatggcatcgcgggggctagacctctggctggatgagcacgtgtggaagaggaaacaggagattggtgtgaaaggagaaaatctgcttctccccgacttatggctagatttcctacaactcagccccatcttccagcgcaagcttgctgccgttattgcctgtgtccgacgcctgcggactcaggccaccgtctacccagaggaggacatgtgcatggcctgggcccgcttttgcgacccctctgatattaaggtggttattttgggccaggacccctatcacgggggtcaagcaaacggcctggcattcagcgtcgcatacggctttccagttccccccagcctgaggaacatctacgcggagctgcaccggagcctgccggagttttctcccccagatcacggctgtctagacgcgtgggcctcccagggggtgttgctactcaacaccatcctgaccgtgcaaaagggcaagcccggctcgcacgcagacattggctgggcgtggtttactgaccacgtaatttcattgctctctgagcggttaaaagcgtgcgtgtttatgctgtggggtgcgaaggcgggagacaaagcttcactaatcaactccaagaagcatctggttctgacctctcagcatccctctcccctggcccagaacagcacccgaaagagtgcccagcagaagttcctgggcaacaaccactttgtcctcgctaacaactttttgcgtgagaaggggctcggtgagatagattggaggctgtagaggggtcatcactatggccatgtttctgaagtcgcgtggggtccggtcttgcagggaccggcgcctcttgtcggacgaggaggaagagacttcacagagcagcagctacactctggggtctcaggcctcccagtctatccaggaggaggacgtgagtgacactgatgagtctgactactcagatgaagacgaggagattgatttggaggaagagtaccccagtgacgaagacccatctgagggcagtgatagcgacccctcgtggcatccttcagattcagacgagtctgactacagcgagagcgacgaggatgaagcaacccccggctctcaggcctcacgatcttcaagagtctcgccatctacccaacagtcttcaggtctgacacccacgccttcgttctcccgaccacgcacccgggcacctccgaggccgccggctcccgcgccggtcaggggacgggcctcagcacctcccaggccaccagccccagttcagcaatccaccaaagacaagggtccccatagacctacgcgacctgtacttagaggcccagctccacgccgcccccctccaccttcaagtcccaatacatacaataaacacatgatggaaaccaccccccccattaagggcaataacaactacaattggccatggctgtaaataaaatgtcataacctggagtctgcatgtctgttgttttattcagtaaaccagtagtgcgcgtgagttctttagggcatccacgatgtagccgctcgcggggttcccctccccagtgatcatctcggataggggattcctgtccatgaccacgcaattagagtgccgggcccgggacagcgccacatacacatggccgggtttgatgtttctgtggctgccgaagcagatggcgactttgtttagggacagaccctgggccttggctatggtcatggccagctttgagctaatgccatagtcacggatgctgcagaggttcagggacttgtcctctatcgtctcatacagtttgttagtattgtgttccaggcagcacacgaagcctgcctcatccttgaccattagcctgggcatgcgtgaactgccagcgtcctgaggctgctgctttcctcggatgccaaagaagacgttgagatgcgtgtagcccagaagcgtgtagttctcggtggtggaggcgtagtccaggaggccgtgaaggagaggctcgtctgaggtgaactctatgttgtcgcgaatcagcatgttgttggtaaatgtgcagaaggggaggtccctgaactcccttccgccatagcggacggccacatccaggcattgcctgaaataggccctgaggtcattatatatgtttaacagggagcagaggggggcagaatttgcggccgggggagccagtactcgggcatagaagacagcggcggggctccgctccccatcccaggcaacctccagcggcagttcgcccagctccatcccagcagtcacctccggatcccacgtacgcccgggcaggggcacagcaccaagctccgccacgtattccccgttttcacagagagaatgtcctccgtggctaaaagcgtagatgcctccgtagatgagtcgggccaggaagctgtagacatactcgggctgctcatgcccgtgggcctccacgaagctgtccgcctcgagcgtgtccataaagtccccgaaggtgccggtatagccacagatggactttttggtcttgcagttgaccgacaccgagctgtgcttgacgtaggtgacattgtaggtgaccttgacccgttcttcgtcctgctcggtgcccaccgggaccatgtcttggtcggcgaactgcgagtagttaccgaggcgtgcataattcttttggagccaggtgtgggccgtgaggcccggaagcccgaccagggtcttgtactgggccaggggatcgaggaagacctcgcactccaccgggcaggtaaacatggtcaccccgcccccatctcccccggttccccgcgcggcacgcccctgcccggcagtcttgagcgtggcatggagggtggtgaggaaggtcttgacctcggcgtgggagaggaagagccgggtccagcccacgtactgcgcggggtccattatggccgccctggggacgacgaagcggtcgacgtaggccaggatgtccggcgagagctcgaggccgtactcgagggtcttcatgaggtgtccaaactggacgtcggtgcagcgcttgttgttgatgaagagggcccagttgcgggccacgtccacgtaggtcgcggccctggggttgcccaccaggaaggtgaggatgttgtcgcactcgcgaatcttgtttacctgggtctcgtggctaaaggaggactgaaaggcgtctgtctgggtgggagagcccacgcagacgatgcagggaatgcggccccggcggtagagtggggtacgcagccaggcgttgaagaaccagtagcaaaagaccacggctgttagaatgtgcacggaaagcgttccagcttcgtccaccacgatcacattggtggtccatagctgcccctggtgcatgtctctcaggacctcaaaggcggggccagagactcccgagtagagccccctgggcttggttcgcctgaactcggcggcaatgtcggagagtaccggccagtatttggccaggtcccgccgctggagttcctctagggcggcgtccgtagagcgaccatgactgctgacccgctgcgtcatatttatgtggcggctcttgaacccaaaggcgctatagacggttgggcagtaggctcggagtgtctgggagaggttctgtgcggccacggttgtggctcccgtgaccaggcagtccatcgtgtggtggaggcagctaacgctggtgctcttgccagcccccgccgttcccgtaattacataggctgaaaagggcaggaaggggggctccgagagctccgggtcaaactcgggggagaacgtctccatatccgggagttgttggacgcggcgcctagccagggtccctatcctcctgactatacgcctcacggaggcgtctgaggtcatgttcaacatgaacgtggacgagagcgcctctggcgccctcggctcctcggccattcctgttcaccccacgccggcctcggtccgactttttgagatcctgcagggaaagtacgcctacgtccagggacagaccatctacgccaacctccgcaaccccggagtcttctcgaggcaggtgtttacccatttgtttaaacgagccatctctcattgcacgtacgatgacgtgctacatgactggaacaagttcgaggcctgcatccagaagcgatggccgagcgatgactcgtgtgcgagccggtttcgtgagtccaccttcgagtcgtggtccacgaccatgaagctgaccgtgcgtgacctgctgaccaccaacatctaccgagtgctacacagccgctccgtgctctcctatgagcgttatgtggactggatctgcgccaccggcatggtgcccgccgttaagaagcccataacccaagagctccactccaagataaagagcctgagggacaggtgcgtctgtcgggaattggggcacgagaggaccatcaggagtatcgggacggaattatatgaggcaacgaaggaaataatagagtcgctcaactccacgttcatcccccagtttacggaggtgaccatcgagtaccttccgaggagcgacgagtatgtggcctactactgtggccgccgcatcaggctgcatgtgctcttccccccggccatctttgccggaacggtgaccttcgacagcccggtgcagcgcctctaccagaacattttcatgtgctaccgcacgctggagcatgccaagatctgccagctcctgaacacggcccctctcaaggccatcgtgggccacggggggcgagacatgtacaaggacatcctggcccatctggagcagaactcacagcgcaaggaccccaagaaggagctgctgaacctgctggtcaagctctcggagaacaagaccatcagcggggtcacggacgtggtggaggagttcataacggatgcctccaacaacctggtggaccgcaaccgtctatttggccagcccggggagacagctgcacagggcctaaagaaaaaggtctccaacacggtggtcaagtgtctgactgatcagataaacgagcaatttgaccagattaatggcctagagaaggagagggagctctatctaaagaagatccgctccatggagtctcagctgcaggcctccctgggtcccggcggcaacaacccagcggcgtcagcccccgccgcagttgcggcagaagccgcgtctgtagatatactgacgggcagcaccgcctccgcaatcgaaaagctgttcaactccccgtccgccagcctgggtgccagggtgtctggtcacaatgaaagcatcctaaacagtttcgtttctcaatacatccccccttcgcgggaaatgactaaggatctgactgaactttgggaaagcgagctgtttaacaccttcaagttaacacccgtggttgataatcaggggcagcgtctctacgtcagatactcgtcagacacgatctctatattattgggccccttcacctatctggtggcagagctttcaccggtggaactcgtgacagatgtctacgccaccctaggcatcgtggagatcatcgacgagctctaccggagcagtcgcctggccatctacatcgaggacctcggtcgaaaatactgccccgcgagcgcgaccgggggagatcatggcatccggcaagcaccatcagcccggggggacacggagcctgaccatgcaaaaagtaagcctgcgcgtgaccccccgcctggtgctggaagttaaccgccataacgccatctgcgtggccaccaacgtccctgagttctacaatgccaggggggaccttaacatccgagacctccgggcccacgtcaaggcccggatgatctcgtcccagttttgcggctacgtcctcgtgagtctgctggactccgaggaccaggtcgaccacctcaacatattcccccacgtgttctccgagaggatgatcctgtacaaacccaacaatgtgaaccttatggagatgtgcgccctgctctcgatgattgagaatgccaagagcccctccataggcctctgccgggaggtgctgggtcgcctgaccctcttgcactccaagtgcaacaatctggactctctgtttctgtacaatggggccaggacgctgctgtccaccctggtcaagtaccacgacctggaggagggggctgccacccccgggccgtggaatgagggcctgagtctctttaagctgcacaaggagctgaagcgcgccccatccgaagcccgggacctcatgcagagcctctttctgacctcggggaagatggggtgcctggccaggtcacccaaggattactgcgcggatctaaacaaggaggaagatgccaactcgggcttcacatttaacctgttttatcaagattctttattgaccaagcatttccagtgccagaccgtcctccagaccttgagacgcaagtgcctcgggagtgacacggtctcaaaaataattccctagaataaactgagaacagtcatcagtaaatctgtctctcgcgtgatttccataggaatggtgtagccggggtggagggccgatatcacatcaagcagaaaggccataatctctcgaaagtaggcggtggggctgagaccatgctcagtggccgtctggcagggggccgggcgcgctccgtccttgtccaggagacacacgtggcttccagagaggcgcagcccagccctccgcagccgctgaagccaggctcgcggaagagcccaaaacctgtttcggcgccgcccgggggccagtctccgggtcaggtcgcggaccagggtcaacaggtggtcgtgggatggcggggccttgtctgcctcgggtctcgccgctagttggtccagggtccaggagaaggcttcgtgccaaaccaaaaagggccccgagtgctccctacatccacccacgtaaagatccccctgaaagatggccatcagtaggcacccgggcccgcgtcgagccttcacccgaatgtgtctgcgggccacggtggcctctccacccatcacatcccggtcgagccggctggcatcctccgagtctttcacgccttgcaggaaagcctaggagatacagcaacagaaagctattagccggtggttcccccaccatcattcttcctgttaacgggaagaataagagttgggcaaaccccgggggccgcgctctcccacccagccccgcttctcacctgtgctagtggctcctctgaaggatgggcggaggttggtgccacaaagcccaggatgaactcgtctgcataagcccaggtcagtcctaggtcagcggccgcgtgtaggagaacccgggtgacggcggtgtagaggcccccgagtgcccgtcgcgtgtctgaggtgccatagcggtgaagggcccgcagccaggtttgcgcgtcccgcgcctgccctccgccatcaggcgttcccacgggggcgcccctggcagagaggtggcagcgggccaattcgtagagccaccaagtggcatcagcctcaaggatggctgtggcctccgcgcgcccgaccaccgtcgtctcgtcctccccccctccctcgccgccttcccgcgtgcaaacgtggcgagggttaatctcctttcgggtcgggggccagatttgttgtaggagcagcgagccgcgtcgttgccctgaccgcgcgtcgaggcccaggagggcgtctgccaggggcgtcccagagactcccaggttcaggtccagtagcaggagaccctcgctgtgtggcgcccggtgccagaaggccggcctcgcccgtcccacataatggatgggcaggaagggaaagcccgggacatagggctggaaatctgagccccctgggcagagttcggggtccaggaggtagaagatgggcttggtgcctctgtggttggcgtagcaggaggcatagatactgcggaggaaggcgtagagcccgcccccggccatactccaagagttgacaagccaggactcgaatcccccagccggctcaagaattttcaggctgacgcggtgccgtcgggcgtccccaccacggccggtggccccgtcggacgacaccagatctacttcataagtgaccggtcgcaggatgtccctaaaggggacgggagaggggtcgtcgggagtctcggtggaataggtgaaaacatccccacgcggtgtcctgatgtatacgtccaactgtccgggagactcagagtgcctctgagcatgggggcatgtctgttccccctccatctcggacccgaagccatcaacaggtgggggttgttggtcccgcccatcatcccccgagcagctttggcagaccacctgtgctggaaagagaggctggaagatgaggccctgctcatcctccaccctggcggcggacaagagtctgcggtctcgggttctaaatgaaaggtcaaataggtccttctcggcggcatcggcgagcatagcaatgagccccccgctgcgcctgagctcccgctcccatcgcaaaaagttgagttcggtagtcgagggcgcgttgaccacggggggctccagggagcctccaagcggcggctggcaggcctgcaccacgatcagagtctcaacgtcctcccttttgatgggcacgatgcccacgacccaaatcgcccaccaccgccctgcggtctgggtaacattataaaaggtaaccgagctgacgcgggccctgacgctctccgcgggtgtttccatcattgtttgagatctgaggaggactggaccctttaaaacatccggtcacgccctttgcaaattatttaaaaggtgaatgctcaactgagaccatcgcaatcatgaagtcctccaagaatgacacgttcgtctatagaacgtgggtcaaaacgcttgttgtgtactttgtgatgtttgtcatgtcggcggtggtccccatcaccgccatgttccccaacctggggtacccctgctactttaacgcactggttgattacggggcacttaacctgaccaattacaacctggcccaccacctgacccccacgctctatctggagccgccggagatgtttgtctacatcacactggtctttatcgcggactgcgtggctttcatctactacgcctgcggcgaggtggcgctaatcaaggcccgaaaaaaggtctcgggtcttacagacctctcggcctgggtctcggcagtgggctccccaaccgtgctgtttttggccatcctcaagctctggtccatacaggtcttcatccaggtcctttcctacaagcacgtctttctctcggcctttgtgtactttttgcactttctggcctcagttctacacgcctgcgcatgtgtgacccgcttctccccggtctgggtggtcaaggcccaggacaactctattccccaggacaccttcttgtggtgggtggtcttctacctgaagcccgtagttacaaacctgtacctggggtgccttgccctggagacgctggtcttctcgctcagcgtgttcctggccctgggcaacagcttttactttatggtgggggacatggtgctgggagccgtgaacctcttcctcatcctgcccatcttctggtacattctgacggaggtgtggctggcctccttcctgcggcacaactttggcttctactgcggcatgttcatcgcctccatcatcctgatcctgcccttggtcaggtacgaggccgtctttgtctccgccaagctgcacaccactgtggccatcaatgtggccatcatacctatcctgtgctcggtggccatgctcatcaggatatgccggattttcaaaagcatgcgccagggcactgactatgtccctgtctcggagacggtggaactggagctagagtcagagccgaggcctaggccctcgcgcacgccatcacccgggcgcaaccgccgccgctcttctacgtcctcatcttcctccaggtcaaccaggagacagaggcccgtctctacccaagccctcgtctcctccgttttaccgatgacgacggacagcgaggaggagatcttcccctaatgcaataaaaacttaaaacactgaggttactttcccgtcattctttcgggggaacgaggggaggcgggaattgggttaagataggggcgaagggtgggggtgggtgcaagaattggggctgggaatggagaggggagtgggctaggtgccgacaccggggtgccaagataatggattgagtaagcatggggctctgatcgggtccgccgggttctcaggggtgtagtgggtgggcattgcatatttttgccgcggtgctgttgggccttggactcggggtgatcatccgtaccatcacccgcacccgcaccccagtccacagccaccggccaaggtcctgggcctcccaccaccgttatgcctccccctttacccattaattacaagagatgttagtttggttttttatttggcaaaaacagcaattcatcattttcagagtcctcatcatattcgagcccctcgttggtttccccgcaggccctcccttcttcggccgctattagcttagtagtctccaggttaaactcctcatagtcattatacaggttgattattcccccgtccacgtcgcctatggagttgactcgtcgtcggcaaagagaccagagggcacccatggcgcggtgtcaaaagtattgtctgcgtacgctttccaggagccagccgcggtgctcaaggtcttacggatgacagagtccggcaggaccacgggtgtcaccagcaccgccacgggaatctccaccgaggcgtccagaagcaggtctgagccgagcgtgcaggtcgccgggtctagaggcgaccgttttcgaaagaaggccgtcacaatgttcacccggggtgagcagtctctcccgggcttgccacccccactgtggcggacgtagtctccaacaattttgtattggaggagcacctggtagaagtagttgtgccgtggattgatgaagatgttgactgggacccggtctttaataccaatgcgccccgcattttcgcttgggtccgtcattacgtagagcatagactccacccccctgttggcagctaggctgtctgccaccaggtcatgaccggggcccagtttgcgcttacggacatctttaagattccaggcctcatcctgcgtcaacagatagtcaccctccgagggcaaccgcccatccgggacgtactccacggtaggacgagctatagaattgataaatctgataaatgacctcttgcatggcctcttgtaaagcgcagtgtaggatgggtagatggggtcaaattctgacttggaaaagaggtacttgaagcggcacttaatctcataaatgcagctccggtcggtgaacagtataaagtctccctgtgactccacattgacgcaaagatccagagacaccccaaaaatgccatccgtgggactaatcataaagccaaattgacggttggcggatgcgtccccgcagatgagcttacagacaatgtccttgaccgtgtcctcacaccgcaggccaaaggccacaggtcccccaaagtagtgatttgtggagatgggagctggctcaaacaccttggtgggtccattcttaatggtggagagcagcttggaagaggaaattatgccatttcgcaatatgtcccacatcaggttctcagactgccccctggtcatggactccacgtacgagcagagaacagtcctctgctcgtcggtggcctcctgtagcccccagtaaatggatttcagggagggaccgtccttgctgtcattctcttggactaacgaggagacaaagtcacagaagccagtttcaccagagaactcttgtatttgtttacagaggcaatagagatagacaaagcgcatggccggcatctgaggtggacggtcaaggttacggacaaaggcctcagtctccggactgcggaggaagcgggcaaacgtgtaggaggtcatctcctccatgggatcctcgagctcatccacgtcggccatctggaccaaagaagtcgtctgccaagagttcagctaccagacctggaagatgagggtgctcaaaccgtgggcgacagttgaagaagtagctctccttgaacctctttttaaggctccggcaccactgcaagaattgactcatatgctccgccgtgacatccacgcacggactctcgccacacgaggtcaggcccatgtctaagttcaggttccacatctgcgacagcacctccaacagcaccacctttggggctgcaaattgcaaaaagtagagcgggtcggatcggtcaaatcccatgtcagggttggggtaggggattttgtgggtggagtcagcgaggtgcatgataccatagagcagcgagtagccgagcgactgcagatccaggcgaagggccgtctgcgcccccacggggccacacgccgaggggtcagggatgtgcccagcccccctcaagatgtagcacttgctcaaaaggcagaggggcttataggtgtccttggctatagaaaatggttccctctggcaatagaggcgatagagctgccggcccttagaagactttagccgcacatccagcatcttgttgcggtcgtggagggaagcagtcccataatcagtcaggaccagcctacccatgccccacatggtgtctgtgaaatccaccaggatgttgctggggctaatgtccgaatggaagaggccgcagtgccgattcagaaagtaaacggcatctttgaggccctgaaagccccgcaccaggggctcaatactaccatcatgccagtggccataatcctggagactgcatctgaactggggcataaacagggcgtggcaggacgtgcaggccgacaggtagtccaccagggccttgtcctgcccatcctcggccgtggccttcccaatctgaatcatgtcacacaccatgagctcgtgatacagctccgtcacagagtcatagagtttgaccgtggcattatctgcatgtgcatacacggccccgtagctcccccgccccagcagatactcgcaggtaatggggaggtgatcacagcgcgtcatgttctccggcagctttacatagagggtctccgtcatgtcatcaatgttggtcaccttcaggtgtttgtgctgaaaggtgaagtaatcaatgacagtcaccttccccaaaaaggcctgggtctctcgagggggttctggggagacactcaactcgccactgctggaggagttcgtcgggctcaactccgcagccatattcacatccatgttcctcaaatggctcgagggcctgtcgcagctcgtctctggcctcaagctcctgctcacggagctcctccacccgctctagctgcttgtagttgatttttggaaattgagtcttggtcgcggtgaccaccctctgataggtagaaattagctgtttggactcaaacgtctcccttgcgtggcgcagggactctaaggcaccccgagcagatgtaaactgtgtttcaaacagagcgtggtccctcccaaatctgtcacgtgcgctcacagccgctctcttttctaccgaggctcttagttgctgggccaccagatctcgcttagaactactcatcttcataagtcaccatgtccgcaactatggagcccagatcatacgtggggtagagtacggtagttccagtggaggcttcccggtaatttcccacagcgtccaccatatatctttctgcctctcccgttagaattaggcaaggatcatacgtgtccaccggccttttatactgagcgtttaggttttgtttatgtagcaagcacaaaaggcacacacgagtgatgcaaaagggttcctgaggcagcaggcagagctgttttgccattttattcaggcggctaacgtcaaagggaggagctatatcctcacccttccagtcacgcacgtccaagtacagggcatacacacacctggtgaggtgtgccaggaatgcctctatgttggcacatggtgtataaaccgcagtgggtagcagaatagggccccttttgccccgtgccgcagcgtaaacgcagtgacgctcttcgcagtgggacctggggccgtagaagagggcccacatccaagggagtgggtcttcaggcaccagggaggtccaggtttgggagtgggccaatatttgcaaggcctgacctataacctcatctttgttccaggccagcgcaattcgcataaggtccccatcaaacacctcaaaacacagacccatgcccatttcaggctgagagggctccatccggctcgaccaaccttgtccaccaaactgccattcttctggtaaacgggggttgaggggcaagagctccaaagccaggctcgagaagtcatagtcatcctcggccacacggccggagctccgggcctcgtgccagggcctgttgtcctgggggaggatattggacacgagcaggaagctcttgagtggcgtctccaccagcttaaattgctcgggcgtgtcctggcaggcctccagtgccagttccagacactgcccatacctgcgggcgagcatcgggtcatcgggcatatcggccttgaccgcgttgaacatgctgtatgcctcgcagcgcggccgtctgaccgagaacctaagaaacgcccttcagcaggacagcaccacgcaaggctgcctgggtgccgagaccccgagtattatgtacacaggggccaagtcagacaggtgggctcaccctctggtgggcacaattcacgccagtaatttatattgcccaatgcttcgagcatactgccgccactatggccccaggcccgtgtttgtagcttctgatgaatcattacccatgttcggtgcgagccctgcccttcacaccccagtccaggtccagatgtgcctactaccagagctacgcgacacgttacagcgcctgctgccaccacccaatcttgaagactccgaggccttgacggaattcaagaccagcgtgtcctctgcccgtgccatccttgaggaccccaactttttggagatgagagagtttgtcaccagcctggccagcttcctgagtggtcagtacaagcacaagcccgcccgcctagaagcattccagaaacaagtagtgttacattctttttattttctgatctcaatcaaatctttagagattacagacaccatgtttgacatctttcaaagtgctttcggattggaagaaatgacgctggagaagctgcacatttttaagcaaaaagccagcgtgtttcttatccccaggcgccacggcaagacctggatagtcgtggccatcatcagcctcatcctctcgaatctctccaacgtgcaaataggctacgtggctcaccagaaacatgtcgcgtccgccgttttcactgaaattattgacaccttgaccaagagcttcgactccaagcgtgtagaggtcaacaaggagaccagcaccatcacgtttaggcacagtgggaaaatctccagcaccgtaatgtgtgccacctgcttcaataagaatgtaagacctgacgtttcagtacttggcaattgtagagcatagcccggctgtaaaggtcagaaaatcgcagcagggtccaaggttgtgctgtacatgggacctctttcccattagcaagaaccccctgcaggacacgtgacatgtccgggtgcattttgggtgggttaaatctcagtcccaccacaaagggggcatcctccggtttgaacatcagacccaacaaagcccgatgcccagttatgggtacgtagtcgttgttcagggccgtgcatggcagcagacaaggacaggtgccagatgtgcctgggctatcgtcctccgtccagccacgcaggatgttcacgtgggccccggcaccatagcatgtcacacattccccgttatcacatctggttagcaggttgataaaatgggtcagtgatggaaaggttggcatattggggcagcacatcagcatgtccatgttaacgaaaaacatgtacagggccccttctgcataccaggcaccaccccgtcccagtgggatgatctccgagggtgtgatatcttgcagttcttctactgttttaacggcggttgaggtggtaaagacgtgggccgtggtcagatctgtgcaggtgactacagggttacccctaatctccacaggcaccgcctcacccactgcatctgagaataccccaaagtacatgagagtcaggctgtgtggcccctggactgccttagtgaagagaacctcgggcctggccacggtggctagggttccattgatgtagacggtcacataggtgggcttcttcttgggcttcagcacaatgagggtaacattcatgtaggttttaggaggtccggctatctgaggcacgtacacagctgacacggcggttgtggccgtatagactttcatctggggcgtagaggcatcgctcagcacccagaggcactccttgttgaggaacttgcgaagctgttcccggctactgttcgcggcggatgccatgacgtgccagaatatatcccctctcctcgggggtgagtgccaattggcctttaataacaaagcccccaggcagcaccaaaaatgcctgcccgtccgatgtggtggccaggtggacgcagtgcccgtcagttccaagggctactagctgggaagcagccccaaccagcccacccgggggcctggagtcgatcaccttaccccaggccgaggccccttcctcatacagcgggtggctatctatccataggcaggcatccggcgtctttggtgcattggagatagtagctttcacccaacaactttcccaactaacccgtgtctggacagtgaagaacgcttccctgatcaggtctgaatttttatagatacgggagtaggaggtgggaataacaactgggatttcttgttgtgctgtccaggcctgcatggccagtttttccctgaagctagcagaaattctgagggccactgaaatgaggaagcgaaactccctctctggagctcccaaaattgaaacctcagcaagatctgttgctggggaggcatgggtgacagctgtcatcctgtgcagtctgccctgggcactcagctctggatatgtgacaacatagagagcgtgggggctaaaaatatgagcaattcccctgaccagggccctggactcacgaatggcccgacgggtcttagagaaagaaacaggcaccctcgagagtgcccccgacccgacccccacagtgccgccagtccctgctcggcctccgccgccttccccaccggcgctgccccggatgttgctggggttctcgagggctgggtggtgcttggacacagaggtctcagcagccgccttggtctcggccccggccctaagtctgagccccaggcaaagggccggactcccagcgtggcccaacctctgctcccctctattctcctcttgcgttatctccaatagaatttgcttgaggtcatacgttttagggtgctcgacctgggccgcggccaccggcatatgctctatacccgcccctccggggggcccaggatctataggtatgggctgcatagccgcagcagactcctggaccccagaggcctctctgataagatgcccgtcggtcagagcccttttggccccctcaaagagagacaggtaataaatctgtagctccccaaccagccctccttcatcgtaaaatcgaagggcggccacgtggaaggggttgtagagctctggaaggccctcatcgcagtacactggcacactggtaaacgtgccccgatggctaggccgtccgggcagcatgccccgagcagcaaacacgcggcagaccctcgtgagacccgtccggtcactgaagagagtctggcaccaggccccctcgcagtttggcacgcgattggggcaaagctctgccataaccgtgtcgggaacaaataggtgcacgaggaggggggtcccgaggccactcaacacttggttgtcaatgtggacatccatagctctctcatgcgtttggctacagcatcatagcgcttgtttctggtggatttaaataacagggccccgtagacagtcttttgtgagtaaatagagatgatgacatggatgtagagactgaggaccacatccaccaccttctcggaggaggcccccctaaacagcatcaggcagcaagggaacacaaaggaaaccagggccgggatgtgaggcctcagcgccccctcctgatcaaagagggcctcgctgaccccggagatgacattctcattcagaaagtagtgatagaggtgattgaccacagtcttaaccaggccctggacttgttcaggctcccacttgtcccgctggtcctgtgtgtcttgtcggatctcggtccagggcctcagcgccggctggaaatgcggccccatgtagttgcctgtaagggcgcacaccactccctcatgggtctcaatcagggtgcactcgctggatccatcacatacgtggtactcgccacagccccagcaggcaaacacggaggccatgctctcaggtaacgggagatggaactccagcttactatacgagcacaggtggcgaggattgggctcatccgtgcccccctccccccgcgggaggctcaatcggccttggtctgacattccaccccggccaggtccaggagggtgcaaatattctccaggcgctgcacctcagagacctcctgctcaaagagacctcccaccgccacgtagacgcgggccaccgtccggggaaggtcagtggggtcccagctcagcaattctccaaattctctctccccaatagtgcctcgcttcttatcctgtctttcagagcatccgggggcagacatttcacctcttgtttgtggacgaggctaactttatcaagaaggaggccctgccggcgatcctgggctttatgcttcagaaggatgccaagattatcttcatctcgtctgtgaactcggctgaccaggccaccagctttctttataagctgaaggatgctcaggagcggctgctgaacgtggtaagttatgtgtgtcaggagcatcggcaagattttgacatgcaggacagcatggtctcatgcccctgctttcgcctgcacatcccgtcctacatcaccatggacagcaacatccgagcaaccaccaacctctttctggacggggcctttagcaccgagctgatgggtgacacctcctcgctgagccagggtagcctgagccgcactgtgcgtgacgatgccatcaaccagctggagctctgccgggttgacaccctcaacccccgagtagccggacgcctagcctcctccctctacgtgtacgttgatccggcctataccaacaacacatccgcatcaggcaccggaatcgccgccgtgactcacgacagggcggaccctaacagggtcatcgtcctgggcctggaacacttcttcctcaaggacctaacaggggacgctgccctccagatcgccacctgcgtcgtggccctcgtctcctcgatcgtcaccctgcacccccacttggaggaggtgaaggtagccgtggagggcaacagcagtcaggactctgcggtggccattgcctcaatcattggggaatcctgccccctcccctgcgccttcgtgcacaccaaggacaagacgtccagcctgcagtggcccatgtacctcctgactaatgagaagtccaaggcctttgagaggctcatctacgcagtgaacacggccagcctttctgccagtcaggtcaccgtctccaacaccatccagctctccttcgatccggtcctctatctcatctcccagatcagggccatcaagcccatccctctccgcgacggtacctacacctacaccggcaagcagcgcaacctctctgacgacgtgctggttgcgctagtcatggctcattttctcgcaacaacacagaagcacacgttcaagaaagttcattaaactttattgactacaccagtcccttgtaaagcgacgggtctcgcgtgacggcattcgtgagcagggcttcgtccaggggcttgttcttggcggacatcattagcccagccgcaaatatcagaattagcatcagaaaagtgagccccacaaacaccagtgtccagagaggaagaccgtaagataaagatggctgcctctcatctggaacggtgggaagctcagcagttgtttttgtggcattggacgtccctttggaggacagcgtgggggccaaggtggtagcgttggtaatacgggtagtagcactggtggtggaggaggacctggtggtgacattgctagtcacacccgtggaggttcctgttccggcctcggtggcagtgatgttctgtgcagtaaccttagtggtgacattgatggtggatgcgttggaagttgttgggactggtgtgacagttgtcccagtgaatgtcaccgtggttgtgttggtgctcagaatagcagttgtggttataggggcgctagtcgtggtcaaggtcgtagactggtttgtgctaggacccgatgccgacggtgatggtgtagtcacagccgttgtgcctgtcacgttccccgccgaggccgtcgaactgccactagatgtccaaataaggcttgtctcacagatgagtatcatggccataacagcgcctgccttgtctctggcgtgtgccatcgcgtctggacgcagaaggcctcccggcctcttttatagctagtctccacacccaatactctactgaaccatcacatacatgacctcctcgaggtatgcagggaatgagcggtccgtgagccggtcaacacgacattgcttccgtttcatgcctccagctgcccctgaccagttaggacccttgacggatgtctttaacggcgcggtgcagttggtcaccaatgacggcctaaaggccaacacatccttgaagcagggcgtaggaatggtaccaaactcggggcccaccccatcaaagacataatatgtctcatagtggcagtgatgatgcatcaccaccacagcactcgccaggaccctctgcatatcttgtacaaggcgcctttcaactcggccactggctctggtgacgttaaatgtcctgttcctattagtcacagcctgtagatttgggcacccagactcaaaaagtgcagctacatgaagggcagccgcctcaaatccaccatgacccccatggctgtccgtgttgttggggtaataagtcacattgttaatgaccacggccgggataagggtgtaaaccttgcagaatggattggtcggacacccataagacaggggcaccccaaaatcacgccccttaccccgaagcaccttggcccccaccggcataaagctgggcaaaaagagtgggttaaaaccaaaggcgagtagggccaggaacgccaaatagcagcagtaatagatgaaaacaaagctcagcatgaaacagcgtggaggctcagctagggtctctgcctctccatcatagacatcttccttgaatctcattctctcaccgcatacctcgctcttcatccaggagggggccatggctgccattctaccagttaacgaggagagagagagtaggtccgcggaaattggtgcccctctctgccctcctgacgaggccatggtgtcatccatctccgcagtccgttcttcagctttggcattggtccgggtccgggtggtctgattttgattctgatcctgggtattggtcttggtctctcctcccccattggcatggattggcataggtgggtgtggctcaggctcaggttccggccctgggacggcagcagccgccgggacggtgaagtcgtggaaggtagaggcccgtccctcccgaggtcgtggggccggagccttataaaagacttccaccctctccccgctggccaagacacgccgctcgtggaccacgccatcttcctcccggctgattgtgtggctgacggtgccgtgttccaccgccacttgttcatcgaccatggtaccccctttatcttaaccagcaagtggccgtcagggtctcttgagagtatgccgctgtggccaagcgaggccccaaattaaatagtgatgccaaagactgtaggtaggtcatcatcacacgcatgcgtgataaatcatccgccactgacaggtcatccaggtctatccgggctatctcatccggcaccatttcctggaagagattcaagaggtcgtgatgctcatgccggataaggcctcggaccaggcgcatactggccctgggcagcagggtcaccatgatgcaaaagtagagactcagattgtccagcagggccaagccaaggggccctggcacctccgggagggccaactcgtagtggtgccccaggtatgaaacagagccaagatgcatgtgtacatcgagcatgtctgcgttcccgggagcctgcatgacaacccgggagtacacgttaaacaggagaatcttctgcagcacctcctctgctatgggcgtaggcagcaccatggggaaaacaatgtccacatcattggactctaacttcacggtggcatgctctcgtccaaataccgggggcataacactgaggctcccggtcccatgccactggaaaaagggctggtacttgttcttaatggcgtaggtctgacctggaacaatcttggtgagtatcaaactgtccacgctaacctcatccagcacggccagggtgcaatcagacaggtagttgtacatggacacgtagtccgggaccgtctctagagagtacacctgacccaagcccaatccctgcacattctgcgtcccgtgagtggaagccaggggtaagatgcagccaatcctctgttgcatcttggcaatctcatcggtatacagacgagaggagagagacactaccactttcaaatccatctttattgacaattatcaaaaaaccaccttatttccaaactttaatattcttcgtaccggcgccacctcttcaattatatagtgtccgtaatggatgggggcgtgggtctgtttgacagacataaactcatcgatgagtgcccgggaggaggctgagagtgcggggaatgcctcctgcagaaagctgcagggctgctccagaaacacgtcagtgccagcaatcactacaaactgcacctctgtgttgctggtggctgggtgccctccaagtcgctggctgtactcgttgaccatgttgtagagtcccctgttgttgcgcagaagctcctccttgttgaaaaatgcccggcaggggctgtagaggcccgggacggccgtctggcgataggaggagttgtacatgatgtcacccagagaacccagctgagatgcccagggattcacagtgctccggtattcataggcggcatccgggcgagaatggtcatagatgagcccctcggcaacctcctgattgtagttttcacaggagaccacacaggcggcccgtccccttggagagttggacttttgaaaataagccacgtctgccgtgaccggtgttacgataatctcacaggtggcctgctggccgtggcagagtcctggagctccattaacattagtcatacctgccaggtatgtcctggggtcccgaagcagcgtcccattgcgctgagcgcccaccttggccttgatgtagtcattgacttgctggttgccaaaggcctcggccggaaagacgctaaagaagtcttgggtgtggatacccatgtcagtagtgatggccgccaccctggccggagtcatggtcgagctataactaagcccggtgtcgatggaggccatctcgtgatgcacctcaaaggttaccgcgtccaccctggcctcccggcggctaacatttggggtcccaatgaacatggatgttgaggccctggagctaaacaatatgttttcagagaggatctcatcggtcctgaccacggtcatggccacccctgggtggatcttgagcttggcctgggcaatataggccatgggggacatcttgatgtgcatggcggtcattccactgattgaaacgagggaaggaagacattcggccgcgtatttgcccatgggcgagcggtgccactcccggtactctgcaaagagctgctctggccggttgaaggcttccacggcccgctgctgaggattgcgcataacaaaggtggcaacatcctggtgcatggtggcagccactcgcgggtccccgtaaaacatatggaaaggaatggcgtgaaagagacactgggtgacggcccgggtcctctcggagaaggcaaaggccaccagcccgttcaccaaaacagtctgctctgtccgcttgtcggcgggattcggggccagctgctgcgtaacgtcattgtccaccgacacacgcacggcacgggtgaaagtggggcaggtcatgaatgaggcgctgaggtccctgatcatgcccacggtggggcggaggtcggagatctccagcagatccctgagcgtcccattctccaaattgtcgaggatgtcctcgtccctggtaaaatggtggctgaaggctggcccgttgtaggccagggtctgggccacgtgctgaaagtccaccccgaggccgcacatgtgggcattggtgcaggttgggaggaaaacgtagtaaaagatcttttccagcacatccgcatgcccctcatctacataagggcctaggtgcagacggaaatcgtggtcgtggtctccgttaacccggtagccgtacaaggccacaaattgggcagccatctcatccatgtttccaaccctctcaataaactggggcgcggccagggtgtcagcgtaaacctcatttccgataataatctggggggcccggtcactaacggtgagaagatgggtgaaaatgtctgtgtaggccaccggggggagcaggttagggtccaggagagcgcagacatactgacccacgctctcatcccccacaacatctgacccggccaggcgcatcagggcctgctctagggctataagttccccatagatttttctatacatggaataggcctccttggagatggcgttatttcccaggtggcggcagatgaacttgatcatggaaaagctgttcacaaaggcaagcctccctgaccgttcccagtaggtgttgatgcacagggacaccaaaggcacgttcatgacaaacttttcctcaaacccgtggatcatagcctcgactacgtagaagaaggctggataggcagtgtcataggcagtatcctgcacagtctcaataacggcctgatccaccacgtgggccagagatgtggcggtctcaaactgctgcccccgggcctcttggaatgcagctggggccaggggagtcggcaggttacccaccattagccggtgcacagccctgtgcctggccctctccccggcatccctgccaatgtaaatatcataaagggggtgcagctccagccgcagcaggtcataattggacgggtggaggaagtcttcggtgggcagcccgcacttgagagctatatctgtcacgggggctgcatacttgttatcatagaactcgtccacaataacaagcacattcatgtgattgggcctcctgtgttgcagggagtaggtctcgcgcctgtctcgcggggccggggccgcgttgaggctgtttagggtatgggcgggtgtgtggagtcgggggtgacagagaaccttgagagcattctgtaggttaaacgcgaggagaaggttattcttgtttacgatccatgcctccaccggtagctgctgtgtggggttgtccagcattttgatggcggcggaggtcgtgtacttgggattgggcataaacaggcccactgggaaatagtagctgtactgcattcttctgttgagggggtatggggactgagtgtcattgtacatcttttgcaggctttccacggccaccgcgtggttgcccagcttgatgacggcggctgagatcggcacccggggctgatcctcgacccctgcggccacagccggcaggtcagacttggtgcttccggctttttccggtgagtccacgatcctagccatgaaatgctcaaacgtacgcatcacgcgcccgtagctcacggcagtgaccaggttctccccccgtaccacaaaagaagcatagctcgagggccccataatctggttgtcggcctcctcacccaggaaggtcaagagctggcgcagaacgttgtcggtgacaataaacaccccccccactggctctccccccttggcggtcgtgtaggtactgacccccttgagcacgctctccccggacacggccgctaccatctcagagagacggcttcgcacgtactgagaaaacccggagcccatgttctcggcccggtccaggaagaaggagtgctccagcagatgcctcttgaacatggcaatgaggtcagacttgacagtcttggagaaccccctctcagtgaaggtgggatccgccagggtctgcaggataaacatgggaggggcatggcgaagcttcacactcaggacggtgttaatgaggcccctctccagggcatcgaccccaaactgtagggccgaggccacggtcttgacagcccccacgtactctgcgtactcgaccggggtctcggggatactatgcaggatctccagatccagcatggacagttccatttccgtactaatgtggtgtttgtggcaatttttgaccacaatgaatgtccgctgcttgctgggtctccttccgtccccgtgagcaatggtggggacggagattcgaaattgaatcttgccatccgtcatacgactcaggtctttgaattccgtgttcacacaggacacggccagtgccgtctccaggaagcgaacatattggatggcgttcgtgtagaccccgagtagcacctcaaacttgatgcccgcctctctggcatccttgcccaccagcaggtcaaagctatgaaacaacccctcagccgctgactgccgcaggttcgagagcaggtcggcatccaccgtcagataggggaagggtctgttttccacaccctcatttgaggccatgacacaaggtaagagggagatggggggaggtctcgagggcttctcttcacagctgggtctcttttacgccctggcctgcaaccgcagcccacccacacttcccgaggatgctacccttctaatcaaatggttggacacggccctgggcagggaggccaccttttacgcgtgtcgggctatgcgtcggcttctactcggcgttatccgaatgaatgactgccaggagctgccacccggtttaataattctgagtccgggcaccgtccctggcccccttggagtccagagtctggagcatacagactgcgaaatatggtcctctgcccaccctgaccacgctgcccacctcccggtgcccagggtcatcacatacaccgactgcccgggttccataaacacgagctcaatgtttcgccttatcatccgctacttgtctcatcaccaatttgagcgctgcttcgagcagttctgccgcgtggtcccgcgtcgcttcctagggacctgtaagcgaaactctgcaaagatgctggctcatctgaatcaggttaccaggatccccccctgtccgcccttcagcgggcgggaggccagactcaagttccacttcttctcctggagcacattcatgctgtcatggccaaacaatgccacactccgggagatcaggacgagggccgccaccaacctcacccaccacccacatctagtggatactctgtaccacgcctctccgcagaccccatttctgacacgcagcggtgctctataccgcttcgtcacctgttgcaactgcaccctgcccaatatctccatccagcagtgcaaggccggggacagaccgggggacctggagatcattctacagagtaacggcggagggaggcccgcgagcttccagttcccctcctccccaactggctccctattgcgatgcatagttgctgcgtccctgctgccggaggtgtccgtggggcaccaggagctgtctccgctgcggtccagaagccagggagggcagacggatgtcaggtcgggcccggacccggcccggagactggtggccctcctgcgaagggaagatggggcacctaaagacccccctctgggaccgtttggacacccccgggggcccggcccggccaagagcgaagacgaggagtctgagcgtcgagacgcccctccacccccgctcgattccagcttccaagcttcccggttggtgcccgtggggcctgggtttcgcctgctcgtgttcaacaccaatcgggtgatcaacactaaattggtgtgctcagagcccctggtgaagatgcgagtttgcaatgtcccccgcctcatcaacaactttgtagcccgcaagtacgtggtgaaagagacggcgttcaccgtcagtctattctttacggacggggtgggggccaacctagccatcaatgtcaatatcagtggcacctatctgagcttcctattggccatgacgtcactgcggtgcttcctgcctgtggaggctatttatcccgcggccgtgtcaaactggaactcgactctagatctccatgggctggaaaatcagagcctagtcagagagaaccgaagcggggtcttttggactaccaactttccctcggtggtgtcctgccgggacggtctcaacgtgtcctggtttaaggccgcaactgccaccatatctcgagtgcacgggcagacattggagcagcacctgatccgtgaaatcacccccatcgtgacgcatcgagaggcaaaaatctcccggattaaaaaccggctctttaccctgctagagctacgcaatcggagtcagattcaagtgctgcacaagcgtttcctggaaggcctgctagactgcgcctccctcctgcgcctggatcccagctgtatcaaccgaatcgcctccgagggcctgtttgatttctccaagagaagcatcgcccactccaaaaaccgacacgagtgcgcgcttctgggtcacagacattcggcgaacgtgacaaagctggtggtaaacgagcgcaagacccgcctggacatactgggccgtaacgctaactttttaacgaggtgtaagcatcaggttaatctaagacagtcacctattttcctgaccctcctgaggcacatccgccgacgtctgggcctgggccgtgcttccgtaaaacgagagattacccttctcctggcccacctgcgcaaaaagacagcccccatccactgccgtgatgctcaagtgtaagcagcccggggcccgcttcattcacggggccgtgcacctgccatcgggacagattgtcttccacaccatccacagccccactcttgcctcggcgctgggactgcctggggaaaatgtacccatcccggccctcttccgtgcctcgggcctcaacgtccgtgagagcctacccatgaccaacatgagggcaccgatcatctcgctggctcgcctcatcctggcccccaacccctatatcctagagggacagctgacggtgggcatgacacaggacaacggcattcccgtgctttttgccaggcctgtcattgaggtaaaaagcgggcctgagtccaacattaaagcctcctcgcaacttatgatagcagaagactcctgcctgaatcagatcgcccccttttccgcatcagagcaccccgccttctccatggttgagtccgtaaaacgagtccgggtcgatgagggagcaaacacccggcgcaccatccgggatattctggagatccccgtgactgtgctctcatccctgcaactgtctcccaccaagtccatcctgaaaaaggcaccggagcccccacctccggagccccaagccaccttcgatgccaccccctatgcccgcatcttttacgacatcgggcgacaggtgcccaagctgggcaatgcccccgccgcgcaggtcagcaacgtgctcatcgccaaccgctcccacaactctctaaggctggtgcccaatccggacttgctgcctctccagcatttgtacctcaagcacgtagtgctaaagagtctgaatctggagaatatagtgcaggactttgaggccatcttcacctccccgtctgataccatcagtgaggctgaaaccaaggcctttgagaagctggtggagcaagccaaaaacaccgtagagaacatagtcttttgcctcaacagcatctgttccacctctacactcccagatgtcgtccccgatgtcaataacccaaacattagcctggctctagagaagtattttctcatgttccctccctcaggcaccattatgagaaatgtcagattcgccacccccatcgtccggctcttgtgccaaggggctgagcttggcaccatggcacagtttctaggaaagtacatcaaggtcaagaaggaaactggaatgtacacactggtcaagctttattacctgctgcgcatctaaaggaaaaacataacaatcttgtgaaccagaaagatacccagagcaaaagcaataaagtacaggattattgccaaaacaacgtgtgctctttcttcatacaggcccgcaatttccatgacagtcccgttggtggtcagcagcagatagtgaacgtggaggttgtcaaaatcaaagtagttggagctcaagatggagttttggacttcctgggaggtgatgtaggttgtagtttccaggccttccttttcatcataactgagcagggcaaagccacaaaaaatgcaggatttctgcgtcctggtaaaattctggatctttggaatctggcggggctccccagccacagcaccctgcgaacatttattcattataacgggggagagaaagagagagctgctgagataggtggtgctggcctcgtatagcgccgagcctcggacctcacggtcactagagattatgaatgtcacattgatgagcgggataatcatcagaactttgtcgagcctgtccacgcatttgtaggcggggagatgccacgcatccctgtcttctcgctccaaagagagccgcccaagaaacccatccacagcatttgaaacggccgcctggtccagcgttgcctcctggggggccatgctcagcagcttgtctcgtgtgaggtcaaatcgtaggctgaggtagcacggtgagaagagcccgctctctgtccccagggctagcccccgcaaaacctccccaatctctagggccgagcacagggcggtggacagcagttggtatagggcaaggttgggcccctgggtagtcacgttcagccgcaactcgcgtagcaccacgtggctgccgataaacagggtctctctcatcacggtatgcaggggctggaaaagggggtggcggttgtaggccgagagaagcacagatgtggcgcctccaatgaggccactgtaaaccccggccttggggtagccgacggtggctaacctcagcgcgtactcctgtttctcagtagtcaggtgacccagctcctccatcttgaccgtggccatcagcatggcggccaagcgctccagcccgtaggattgcatgcccttgacagtggccccataacatatgccgatgatgtctttcaggacagtcagctcaaagaagctcttggccaaccagcggaggtccacgcagccattgccagtctcacccacagcatgacccaccttaaagaaggccacagaaacctcaaacatggtagtcagcgtttccgtgtccagttccggctcccggcagcctcccttcatctccagcaggaccagtttctggagaacgtagcgagcgtagctggcggctgtcatggtgacggctcgggaaaacatatccttcaggttgggtacaaagtagttgtgaaagttggcataatgcacaaaggttgtaacaatcaccagggaatagtccccgctttgggcactggttaaggatgggtaactaaaaggccccctcagatccggcaggtccttcgtcttgccaaagaccaggctcaacacatgctcatctcccttctcggtcactcgcttgtaggtgcccatcagaaatttagaagtcatggcccccgtgtactgaaacttgtccccgttgatggacagggccacataagacaagtgacagcgcagctgataaaagacatagctgtgtggccgcgtgttgggcagcatggtgccaatatagtagaagagctgcttctcaaggggggcactaagcatgcaggcaggggaattcaggccgctaatgactccgggatggaccttagatgcatccacttgcatggatccttcagagacagcagggatatcgacaggctcggccagcgcaataccaagggtaccagacgtcttgtaaattaacttgtagcggttaagcatagacgccaaatcttcggtgacatttgcctctctccacagcgcctctgggctaaggcctgggacctttgccatcagttcggtccatgggatggtgtaatgcgaagcatgcccctctatgtccaggtgcagcttaacctcgctgaggctggcagcccccacctcccatagcaacaccaggcaaaaaacacagagcaactgcatcctagtcccgatttcccctctcaaaatcagagatcaccttgctcagaccagcccaatcgaaaaactgagatcgtattgccggattcttcaatgcctgcatgtaaatctccgtccagcatccaggtaaatcgtcctgaaactctgagaggtccacaagcacaaactgaaggtaggctagcgttcgggtgaacgcaagacaaacttccaacaacaccgcgtcggctcggaaaggctgtatgacttccttaagtacactaaagatgctgttcttatacagcttctcggccacaccacttcgaattatgggggtgtggctttgatgacatactgtcgtgattgttgttagaccggcacataccttcacaatgtcctcgggggcaaaatactgtgttaggagccaggcacagtaaacggcgtgatatgcatcgttgacactcttcaggtagccagcatccagtcctgactcatgtttcctccctcgcttcttcaggcggcgcatgttctcctccacgtttaacttcatccagactatggtgtcccccgggtctgcggtaaacgtggccaaaacttgaataaagtcactataggagagaagctggctccggagcagcattagagggaaaaccacggaggccgacagcaaatggcgatcatgcaaaatccaacaatccaggggcgcgactgacctggcaccagactcggtaaccagcaagctccgcttcctagaggccaagactctgaaaggggtggtaaatttcatctggcatgctaaaacctcagccgacgtgtcttcccttccatgcctcgcccgagtcacattcttgtgcatggccttaatggcattttcatacacatgagtccagtaccgcatcggttcagggactacaatggtcaggtccccaaagacagccttcaaatgattcagcatagtagtctttcccacaccaggggcaccttccaaaaatagggaacaggcaggtttgattactggtacatgatttgttaggtgggtcacaattggaacccgcatgctctccttcctctgagccttggcctggcgggtgtcttgggcatcatccagattcagaacattcatcacactcccacttagccgcttcagctgggcagcatgcttggataacttactaaactcgcgcccatgggcggccaggtgttcgaagagaccagaaggcttacccttgccaccattcttttgttttaacgcggaatgagaagagggcctgcggaaattagactcatcctcagactcacagtcagatttgtcatcgagcccaaggccggccaggccctcctcaaagcctttctggtacatgaagctccggctcgtggagtccgcacctccttctgtgcacgaagttttgcggaaccaggagaaggggtctggcgtcttgctggggccacactcccggctacggggcttcggggtaggggcagtaggcttttggtgtgcgggtgctggtggctgggctcccctgggcagggtaaaggggcacgatgtgtgccggctacccggagagtttccagtattagatgtcacggcagcctgggtccggcacggcaccctctccccagacagtccggtcggagccatcaaggggggccagtgggtgggcacctggtagaggccgtcgtcatcttcctcacctgcccctgagtcactaccggttggggtaagaactgagggggcaaagtcatcaatctcagcgtaaaagttttcgtgtctttcgttttcaggggactcatcctcctgacattttcgccagccgccgggcgggccggcctcctttcctggaaatccagccatggatcccacccggggtctgtgtgccctctccacacacgacctggcaaaatttcacagtcttcccccggctagaaaggcggcaggtaagcgagcgcaccttcggtgttactccaagctgctctctcttaagagctgggagcaactggcctcttttttgtctctgcccccgggacccacgtttacagactttagactatttttcgaagtcaccctgggtcggagaatcgcagattgcgttgtggtagctctgcagccttatccccggtgttatattgtagaatttaagacggccatgagcaacacggccaacccgcaaagcgttactcgcaaggcacagaggctagagggcaccgcccagttgtgtgactgtgccaattttcttcgcacgtcctgcccccccgtgctgggcagtcagggcctggaagtcttggcggcgttggtatttaaaaaccagcgatccctgagaacgctccaggtagagtttccagccctgggccaaaagaccctccccacctccaccaccggcctgctaaacctcctctcccgctggcaggatggcgctctccgggcacgtcttgatagaccccgcccgactgcccagggacacaggccccgaactcatgtgggccccaagccttcgcaactcactgcgcgtgtcccccgaagcgctcgagctggcagagcgggaggccgaaagggccaggtcggagcggtgggacaggtgtgcccaggtgctcaaaaataggctgctccgcgtggagctggacggcatcatgcgtgaccacctggccagggcggaggagatccgccaggacctggatgctgtagtggccttctctgatggcctggagagcatgcaggtcaggtccccctccacgggagggcgctctgcgccagccccgccctccccatccccagcccagccgttcactcggctcaccgggaacgcccagtatgcagtctcaatctctcccacggacccccctctgatggtggccggcagcctggctcaaacgctgcttggtaatctgtacgggaacatcaaccagtgggtaccgtccttcggaccctggtacaggaccatgtcggctaatgccatgcagcggcgcgtgttccctaagcagctgaggggcaacctgaactttaccaactccgtctccctaaagctgatgacagaagtggtggcggtgcttgagggcaccacccaggactttttctcagacgtcaggcacctgccagacctccaggctgccctgatcctctcggtggcctacctgctactccaggggggctcctcacaccagcagcgccccctccctgcctcacgggaagagctgctggagctgggcccggagagcctagagaaaatcatcgccgacctcaaggccaagtcacccggcggaaattttatgattttaacaagcggaaacaaggaagcgcgccagtcaatagcccctctcaaccgacaggcggcatatccacccggcacattcgcggacaataagatttacaacctgtttgtgggagcgggactactgcccacgacggccgcgctgaacgtgcccggggcggcgggtcgggaccgggacctggtgtaccggatcgccaaccagatctttggggaggatgtgccccccttctcatctcaccagtggaacctgcgcgtaggtttagccgcactcgaggccctgatgctcgtctacacgctctgcgagaccgccaacctggccgaggcggccacccggcgtctacacctatcgtccctgctcccccaggcaatgcagcggcgcaagcctgccatggcgtcagctggtatgccgggcgcctatccagtccagacgcttttccgccacggggagctcttccgcttcatctgggcccactacgtgaggcccacggtggcggcagacccccaggcctccatcagctctcttttccccgggctggttttgctggccctggagctgaagttgatggatgggcaggctccctcccattatgccataaacctgaccggacaaaagtttgacaccctctttgagattatcaaccagaagcttttatttcacgacccggctgccatgctggcggcgcgcacacagctgcgtctagccttcgaggacggcgtcggtgttgccctggggcgcccctcgcccatgcttgcggcgcgggagatcctggagcgtcagttctcagcctcggatgactacgaccggctgtacttcctgacgctgggctacctggcctccccggtggccccaagctgagccagttcctcgcactggagtgggtcattggcaaaaaggtaaataaactcatcgcacgggggttttgcctccttctcgtctcttgtttcgggtaggggagtaaggccgtgccaggccgccatgctcagggccacggcgtgccagaggccctcgtagtcgtgcgcatccgagaggatggcacggtccagaagcagatagccggccaggcagaggaaggccacaaagagggggcgaaggcgtgcccgaacccgggtttcatgctcgtctgcaccccagtggacaaggcagtagaggacacccaccaccaggcggttagggaggacactgccaaggttgaagagcagatttccgtcagccagggtgacctggctcaggtccggcgccctgcgcagtccaagctgcgcccacacacatgcacagacggcccctgtgacatcaggccggtcatgcaaaaacagacaaagagaccgtgagcggttaccggggcgcagggcctctgccgggaagcccacccgggccagggcccggtaaagcaggtaccagtattcatccggcaccttgcgtgccaacacacgattcgtgcggtttccagtatttatcacggcttcccgccacaggtaaaagttaacacttagggtcagcagcttggtcagggataggtgcaaaaacctgagctcgtcctcgcgcagagcgcaaagcggccagttctttagcatcttcaggaggagcccgtgaatcccaggtgtcattcgcgcgtcatccccgcgcacccccagtcccattaacatagcgggcacaatggtgcaggcaccgtctgtatacgtctgcggcttcgtggagcgcccggacgccccacccaaggacgcctgccttcacctggatcccctcaccgtcaagagccagctccctctgaagaagcccttgccactcacggtggaacacctgccggatgctccggtcggctcagtctttggcctttaccagagccgagcgggtctctttagcgcagcctcgattacctctggggacttcctgtccctgctggactcaatttaccacgattgcgatattgcacagagtcagcgcctgcccctccctcgagaacccaaggtggaggctctgcacgcctggctcccctcactgtcactggcctccctccacccagacataccccaaaccaccgcagatggaggcaagctgtccttctttgaccacgtgtctatctgtgccctgggtcgtcggcgcggcaccacggcagtctacggtacagaccttgcgtgggtcctgaagcactttagtgacctggaaccgtctatcgccgcccagattgagaatgacgccaatgccgcaaagcgtgaatccggatgcccggaagaccaccctctgcccctcacgaagctcatagctaaggcaatcgatgctggatttctgagaaaccgcgtggagactctgaggcaggacaggggtgtggccaatatcccagccgagtcgtatttaaaggccagcgacgccccggacctacaaaagccggacaaggcacttcagagcccaccaccggcctccacagacccagccaccatgctatcaggtaacgcaggagaaggagcaacagcctgcggaggttcggccgccgcgggccaggacctcatcagcgtcccccgcaacacctttatgacactgcttcagaccaacctggacaacaaaccgccgaggcagaccccgctaccctacgcggccccgctgccccccttttcccaccaggcaatagccaccgcgccttcctacggtcctggggccggagcggtcgccccggccggcggctactttacctccccaggaggttactacgccgggcccgcgggcggggacccgggtgccttcttggcgatggacgctcacacctaccacccccacccacacccccctccggcctactttggcttgccgggcctctttggcccccctccacccgtgcctccttactacggatcccacttgcgggcagactacgtccccgctccctcgcgatccaacaagcggaaaagagaccccgaggaggatgaagaaggcggggggctattcccgggggaggacgccaccctctaccgcaaggacatagcgggcctctccaagagtgtgaatgagttacagcacacgctacaggccctgcgccgggagacgctgtcctacggccacaccggagtcggatactgcccccagcagggcccctgctacacccactcggggccttacggatttcagcctcatcaaagctacgaagtgcccagatacgtccctcatccgcccccaccaccaacttctcaccaggcagctcaggcgcagcctccacccccgggcacacaggcccccgaagcccactgtgtggccgagtccacgatccctgaggcgggagcagccgggaactctggaccccgggaggacaccaaccctcagcagcccaccaccgagggccaccaccgcggaaagaaactggtgcaggcctctgcgtccggagtggctcagtctaaggagcccaccacccccaaggccaagtctgtgtcagcccacctcaagtccatcttttgcgaggaattgctgaataaacgcgtggcttgaaagtaaactttattgcgtgttagtacctgtccattcacaggggtatccagcccttgcgccgcctcccccagcccgccagccaccccagacaggagatgataatgatgaggagcaccggagccaccacagcacaagtgattaggagcagggcccagtgcacccaggtggtcttagggcgccagggatcgattggaaaagggcccagggtcactggcttatgcgtgggacgtttagaaacaggccgcctatggggcctgtgactggtgcttgtggtgtgggagactaatgtggtgggggctatggtagtggctgggataacagtaagatgcatacgctgagtgagggtccggttggcatggtattggtcgtcttcttcccctgcagagtaattgcagtggaccccggaggccacactgcaatttctcagtgtcacattgcacgtgtagtaacctgcatgcgcaagggtcacattggggattatcagagagacggaggtgttggagtcatttacccattctagggtaaggctataattgtaacccccgttagttatatgagttccgttgttggaagtagctacggccaagggcagttgtccatccccgggagtgtatccccggcccaactcgatccgagagaccgactcattgctaggaacgctgcaggtgagattcactctagcacctgcatgggcggtgacattttcaaatttaaccagatctgagaaaaatgcacaaacagaccccacacagcagcacaatagaagcactaaatgagtcattcctaaactgtcagttttaaaactccctgcttctcaggcctaaatatgtggtggggtgtgcttaggatcactttcatattctgcaacaacagccatacccggaagaggagctgccggttgccatttttcaagctgctaaaccacgagtggcagcaggcctaagaagctcctcagcaacatggagacctcgaagggaaactggcaggagcagggagtcacgtaggcactagcctcttcatgtgaggtaagagatcgctaaaaatgggatcagggtatgtaaaccgagttttgcgggggatggtgagccagacacggcgggtgggggaaggagctgacacgattgagtagaaagggccaaaaatacaccagctataaggaattgctcaggccaaagttgttcctcaggtggctttaggcctaatgtaggcaattgcgtgcctagaacattgctaatgtgccctgggtttcctgccttcatgcaaatattctacctcccccggcctggtgcaaaatgtctgcctcagaatactaacagctaatccaagctaacattctatcagtaaacgggcagaaaactgataaggaccgcggagtttggccctccgcggtgtccggtggtcctcacacgtgccctccccccgggccgatggctgaggcccggaatatgcaagtgcatctttctaaccagtaggggcctccacctaggtgctttgttaatctttagtgggaactagtgggagtgctgtgcctcgggtacccctatcctataggtcctaccggagctccttgtcttgataatccctgtaaacacacaccacctaagaacaaggcattgttaacctttggtggaacctagtgttagtgttgtgctgtaaataagtgtccagcgcaccactagtcaccaggtgtcaccggaggctacttgcctcagtgccacttttaccttctcaaatctatacggggggggggggggctctgtaacatttggtgggacctgatgctgctggtgtgctgtaaataagtgcctagcacatcacgtaggcaccaggtgtcaccagggctacttgcctcggcatctcctcaccggagaaggggttaacaaacccgtggggggtcttagtggaagtgacgtgctgtgaatacaggtccatagcaccgctatccactatgtctcgcccgggctatatgtcgccttacctcccctatatagtcacgaccccaccgaaccaggcatgatgtagaataaaattttatgcatcatcttctaatctgtgccgcttggagggaaacatgaccacctgaagtctgttaaccaggtcagtggttttgtttccttgatagagacacaaggactgccagccccattggggagggggggtgggtacgggagagtttgggctcgtttaaacaaagtctcatctgatgctctgtggcacctcaaggtgaatatagctgcccatcgacgtatcgctggaaaccggtgggccgctgttcacctaaagtgacgcaaggtctgtcagccgccagggtccgtttaccaggctttcaggtgtggaatttagatagagtgggtgtgtgctcttgtttaattacaccaagatcaccaccctctatccatatcccacaattgataaacctccgcatgtccaaccaccacgttgaacaggatgtggcaccctaagaggacgcaggcatacaaggttattacccagtccttgtatgcctggtgtccccttagtgggacgcaggcctaggtagcatcatttacactaaaagcagtgaccttgttggtactttaaggttggtccaatccataggctttttttgtgaaaacccggggatcggactagccttagagtaactcaaggccaagcatttcacacctgcaaatgcaccatgtaaccacagatctaaactgaaagttgcagctttagatggcaaggaaacttgggtttcaggcatagaaagcctggctcactatagcagcccatgtttgttccagggtgggggaaaggcacgtgcccttagaaaacttagctgcaaaaattctattgtgttgggagagcctctatatctaaaggcctttcctcacaatacaaatgttactaacgtctgccctctggagacctgctatgtggctagacgtatggcctacccaagacgttgggggtctcgggtaggccatgattcttccaggcataggttacaaccagtcactgctatcaagcctactcagttcccaacgcagcacataccccccgcctctcctgccatgaggacttatggcagtgtttactgttctgcttttactcttggaccaggctgtcattctatcagaataacaggggaagcaaggccccctgcttcagcgggacacgtgtttctagaatctcggagccaataactacctgcccctctaatctgtatgctgcatgaaaaaccacatacacgtgatgtaagtttagccagtttattgttacaccaatgccccgaaagtctccccctgtccctttgggtctcaggacccagccctggagctcgggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggatgggggtgcgctcccaggccggaccctggtgccaggcagggaccccgcgccacccgcttcatggggggggaggccgccgcaaggacgccgggccggctgggaggtgtgcaccccccgagcgtctggacgacgctggcgagccgggccagctcgccttcttttatcctctttttggggtctctgtgcaataccttaaggtttgctcaggagtggggggcttctcattggttaattcaggtgtgtgattttagcccgttgggttacattaaggtgtgtaaccaggtgggtggtacctggaggtcattctattgggataacgagaggaggaggggctagaggcccgcgagatttggggtaggcggagcctcaggagggtcccctccatagggttgaaccaggagggggaggatcgggctccgccccgatatacctagtgggtggagcctagaggtaggtatccatagggttccattatcctggaggtatcctaagctccgcccctatataccaggtgggtggagctaggtaggattcagctaggttcctactggggtacccccctaccctaccttaaggtgcgccacccttcctccttccgttttaatggtagaataacctataggttattaacctagtggtggaatagggtattgcagctgggtatatacctataggtatatagaacctagaggaagggaaccctatagtgtaatccctcccccccctacccccccctcccttacggttgcctgagcccatcccccaccccagcaccccggggtgacgtggcaccccgcgtgccttactgacttgtcacctttgcacatttggtcagctgaccgatgctcgccacttcctgggtcatgacctggcctgtgccttgtcccatggacaatgtccctccagcgtggtggctgcctttgggatgcatcactttgagccactaagcccccgttgctcgccttgcctgcctcaccatgacacactaagcccctgctaatccatgagccccgcctttaggaagcaccacgtcccggggacggaagctggattttggccagtcttcaattttggggagtggttttgtgtgagccggaagttggcaatggggtgagggtggcgctggttaagctgacgacctcccaaggtctctcaccctgggtacacaggtggggcggcagcctctaactttggctgtggcctctatttcctccctttcctagccagggccatgtgttcctgcatgtctacttgcctcctgtggtggcagagcttggccctgggcccaacccccgccttgggagcctgtaggggccaacacccttggtttgtttgtgttcctgtttgctggcaacttactggcagccgagcagattctaatgggcgcccgccttctttctctcttgttttattaatagaatctcagccaggacctatacctgagacttcaaagtctggtcctgggttctgagacccccaagatttgtcatgcacacctgcacacctgttggtattgggtttctattcttgagtgtgaaagtttgtaaaaaaattcataaaatgtcactaattcctcttacctgtttagggtattgtgcaattcttcagcctgcctattttcaatttgcctaaggtggcaatttaagatgtggttaattaaccattttcctgtctgacaccactgcatgggcaaccgggttccatggcacatttagagataaacatagatgtcttgtcttgctcatgtgcagaggagggggtgttggtgtgcaatatagtttctggattccaaattgagttgggggtgctattttcactatggaattaaattactgacattagacagtggacaccgggctatatgtggggatgtctgtggcttgtcatttcctcttagaaggtaatcccccatcttaacttccctttaaattgtgatgcaagccctgggttatttatagaatgattatctaggtttgatagtctgaaggctgggcagagaatgtttgtaatttttattcaccttctttaccccccacgagtatccagttctagaagatctcctgatatcccgggctgccattattcccttgagtgttatagcttcctcttaacttaagcaagagctccaggatgttagcttttttggtggggctggttgtcaggaagaggttccagtgttgtcctttatttttagatgttagctttgtgttaggttagtatgggctgggtattcactagtgaaggcaactaacacagttagacgtgctagttgtgcccactggtgtttatccggtcccaaatgtcaccacagaacacagggggctggatttggcagcagcacttgtgcttttgttgatttttacccgtgtatcagagtgggggatgctagccaatttagcttcccctccccttaacagggggtctcgcggggtgccaattgtcgcctgccttcccccgcttccccttgttaacttatagcatgataggtaggtcacctaacgtggaagcctggtgggtgatccttcctcggtagggagcgcttagggctgttgagctcaacagccccacctgggtaaaatgtatgttctaaagagttacccaattataacaaaactgttgtagggtaacgaagacctgatggaagtggtattgttgccgttgaaagacgggtgtcctggctcaagttcgcacttcctatacagtgttaaagccttgtatcggaagtttgggcttcgtcccagtgtactcgataatgtcgactgctgcgaaaggtttggaccgtcttccagtaggtgttgggggtcccaaatcacgaggttaggcaggtgcacttggctctttaggagggacccttaagccagacaatgtagtgccccttttttttgcaaattggccttattattaatttcttgttaacactaattctgttctatgaccctgtgtttttcagatgccgttgaacgtgtcactgagctgaatttggacgcagctacttgacctttgcccccgtgcctccagcgctgataagtgctgcgtccactttgtgttacaggtgggccaaacctccagaatatcaattggtggggccttggtgggctgcataaggcagtaggtttgaggtgacctacttggaccatgtggatccagtgtcctgatcctggaccttgactatgaaacaattctaaaaaaatgcatcatagtccagtgtccagggacagtgcactcggaagtctcatcatctccgtttgtgtgtttagtgtggccagtacggccacccctgtgccacgccctggcatgctgctgacatctggccgccaatttcagcgggcccttttcccccttgttcaccccatagcaagaagggtaggttacatgggtattttcccatcagcacctgactggccggtgcaattagaggagagggcaacaacgcaaggctgttgttttatttgggttacaagagctgcggcggtcgatgggttcactgattacggtttcctagattgtacagatgaactagaactgtcacaatctatggggtcgtagacagtgtgcttaccagacttccatggaagatgtgaatttgctgctagctatatgggtggtgctatgggctccctagggactcatgtagtggggctttgtgatagctaatgaatgtggcagctgttgtttgtactggaccctgaattggaaacagtaacttggattctgtaacacttcatgggtcccgtagtgacaactatgctgaatatcttgaatatgggaggaggggggctttgggttccattgtgtgccctttcctggccaacgtgagggtcctagtgttatagggcgtggcagttttcttgagggctaataacccgggtgaggcggttgtcacaggtgctagaccctggagttgaaccagtaccactcggttacaaagtcatggtctagtagttgtgaccctgcaaagctacgtggggatgagcagccagggactttggttggcaagcagacaggcggcgcattggaaccccagaggagtgtcccggggccacctctttggttctgtacatattttgttattgtacataaccatggagttggctgtggtgcactccatctggtaagggggctggtgcggacgcctgtgtttagtctatgccaatgtttacctgccttgggttactattccaaacgaccacacctttgaggacacctggagccctgatcattctcggcttttactgccacctggcttctgttgggtcagacagtttggtgcgctagttgtgtgcttagcagcaacgcacaccaggctgactgccttagcagtgtggccctttattgtggcatcctaaggagggattctggagtgcctttcgcgtgaagcatgccctgagacgtactcgagttaggacttaatcgctcctgtgccgctggatgagggagcgccaatttgtacatcctagctctggccatagagttagcccacccttgtgtctccctttggcctttgcggtgccaatttccggtggtttcccttttccgcccgtttatccaatagcatgtaagagaggttgcctagatttggcaactttgagggaacgttccgtgtagctggtgacctaacacccgcccatcaccaccggacagattctgaacttgtcctgtggtgtttggtgtggttttggggtacgcaggagtacgttggaatgctttggagccgagagggatgggcccgcttgtgcgcttatgtgttacacggtgccaataaccggcccggtgcggctgccccgtgacccgtgggccttaccttcctggccatcgggggaccctggtgctagggtcccttgtgttgctttctgccataggggggaaagcatcgccttcagaattggctgctccgttggaacatttgaggcctactgtatccgtgtcctgacaacattccccgcaaacatgacatgggttaatttaaacatgttttgtttgcttgggaatgctcttagggcctggaagcttgtcattggattcatcgtttcctgaactacaggcgtagggcctattgtagcaggcatgtcttcattcctgcgtaccgaatggcatgaaggcacagcctgttaccattggcaccttttttccatgtaaacctccgtgatcctgggtcctttggagactcaagtgtgaatttgttttggtgttcggcgccagggtatctcgacgttggaatgtcaactcaacttgggcacctcgataaccggctcgtggctcgtacagacgattgtttggctctgtaacttgccagggacggctgacgatgtgtttagtctgccacttgcatccggcgctttggttactcgggagactaatggggggtgtggtatggcacaggctgggggtgagtctggggatgtccctgggcgttgctgcagcccattcgccctctggggatgagatgttcaggggtggccggtaccctacgctgccgatttacataatataaattgtaaatgctgcagtagtagggatctggacgcgcgacctgctactcttcggaaacgccaacccaggagcgtcgcctctggccccatactcccgccatgcgactgctcgccccctcccaggcctccctggtgagcccttgccgctccccgcattcctgctttcggcgcccctgcggatcccgatgacagcaggcctttccttcccccgttaatgaaaagaatgacagtgaggttgtgacagaaggacagctttattcagtttacagagtgccctcggaggctacgatattcccgttaaatgtcttgttgattctctcaaaggtggggagggaggagctctccacaacaatgttccctggcagcgtgagcgcgcagccctgccgttggatgtatcttctcatgatggtgctgatagaggggtctccggcgtagatgaaaaaggcctgggccatgctctggccggtcacgatcgttatggggttgttggaaatgttccggaccgtcagcttgagggtctggcccggcttccactcctgtgggtagacgtagaagaccgggttggaggagtgggacacgacaacggccgtaatcttggagctcaggggggcctcgtaggtgttgttgtattccagctccgtgatgaaattaggaggaataatcacaggggagccaaagtagcggatgtctgtggattccccgtcccagcgccagtggctcttagggtaggggttgtaacggaaggcaataatcacatcatccaatagggtcatgcccaccttgacgttcagcgggccctctcgtttcaggtccggcgtgtccacggagactcggacgtagcccttaccgcggcgtatggcgtttaccggacacaccttccccgggaatgtgtgaatacgggcgtatgactttagaaatgggggcgtgtgctgcgccagcaggtaaggcaggcactcgtcctggctggtgacgggagagccactgaggaagatctggggctcgctggtgtttagcttgtccccgctctgggtgcaggagcgtgtcagctgaatgtcgctctgcccgggcagaatctgcaggtagaggtaggggttcttgaccaatctgatgggcacaatgtaccaggtaaacttccctttctctatgaacaggctgcgcggattcaggacgcttagcacgatgtcctggtcagagtgcataacgaagaagggcttgaggaatacctcgttgtcttccgctccaaagaacaaaaacgcgaccgtaaagtagcggctgccgtaggtggtcgtgttgaaggagaaagaaggtaacttgaagctgagtatctggcccaccgaggggcagggaggcagctcttggcactgcgcgtccagctgcaatacctgcttgttggtgacgcggacgtatgaggggaagatctcgtacttccacacgcctctcatgaacgacgtgtctggtttttcagtgggccgcaggcggcggaggctgttcctgaacgacgagcgccgggacgctagtgctgcatgggctcctccggggtaagcttcggccatggccggagctcgtcgacgggcaaggtgagagtcggggggcgggcgacggtgcggccccaatacaactctccgctcgttagctggtagaatatccgcccggcgtctaggttgtcacttcgctcggccggccagaagagcgcaagtccaagtctggtgctggggccgatgtgcagcggtttgtgcccgcagttgtagactgtcatttttatgggcgagtgggcggtccacacgcgcgggcgcagcacccattggtcgcacgccgcctcctggaatgtaaacccccagagagagggcgtgccgccctggagatggccctgtgccatcacatgtatttcctccttgggtggaacaacggcgtcgtgctccgggtggagggggaatagcgtccaggcatctttcagggtcacgagaccggggtccatgctcagagaacagccctcccgggcggtgggcggcccgggctccagcagaacgtcgcagacccagccctcctcggccctgtccacctgtatgtccaggtgcacggacccggaggctgcgtctcgtgacatggccaggcctggtgccagccgaccacgtcccgtgtcccagccgaggccgcgccagagcagagcccgggactgactcagggccacatcccctcggcccgcggacgccgcctcgccagcccccgggccttcatgggcccgctttctacctctctccggcaccccagcctggtcagccgcagaggaagcatgaccttggggtgggacggggcaggcgtgatcctgggcgcaatctttgccgatccccacaccttcactccttgttaggttgatagaatgtcggtaccacgccacggggggcgggcccgcatagggaaaagccagggagagcgatgtgggcgaggatgggctcaggcggccccagacacgcaatttgcccccctgggcggccgcagcctgcccctcggcggcccgtgccccagctccgtcacggggggcgcataggaggggtatatctaggatagccgcacctacacaaatgagacacagacacaggtcgtgaggatttaggcaacgcaggcttgtctttatagttacaaacatgggagcgtgcacctggaagatgcagctggggtagatctttacatctttacagggcgcagcggccgccagacactgaagggcagagttcacggcgggcacctcccagagggagcccaccagcccgtacctggccacggccagggcctcgtaggccgagacgggcagccggagcttgtggtactgtccctccggcaggtggagtgggacacagttagagaacattagtcctctggtccctatctccacccgccaggcctgtgtgtcagtttgcagggccatcctcgcactcaggtggactggctaggcacccttctgaagtatctggcggtgactgtcacctggttcttgagagagtccataaaatggctgaagctccaggcgtatagtataatgagcaacagggccaaacaggcggcggggcctgggtagtagcgggcaacgagagactctgtgcaatcaaaccccaggctcccggcctcacccaggaagagcagcggcagggacagcataaaccaggagaaggcgcaaatgagtccggtgaaggtgacgttgcatatcaggcgcggcttccttccgaattttgtgcgcaaaagtttccagatgatgataactgtgaggaggacgatcaggactgccgccagtaggtagcagccggctttcagtccttggacggccgtgtgcatgcctttggtggggccttccctgcacatgttggggcctctgttgagattggcgtcggggcccatggtaatgaggaggatgataatcagcaggagtaccagacaaaacacgcccatcaggtacaggcacacatttctgtgggaggttctcttgggcgttcggctgaacaatgctagggtcttctccaacgccatacccaagtgagtccatacggagcacatcaggcccaagaacatcatgttctgggtcaaaaggcagagaccggtagacgagaactcctgaatcatttttcccagcacccagagcagcagttctatgagaagagctatcagccagacatccattcggtgaaccaattttcttacaaagatgataaacaagatgccagccagtgttagcagaatcagcaggacgagcagcaggcttgtcatgccgctgaggaaggcgctgtaggatttagtgcacgcatcttccgttgcattgacggaagtcatgttggccaccagggtccccacggtggacccgggggccatggtggagagcatcttgctggtcagagccagactgggtggtgtctgcagcaaaagaggaacttgcccaggcagtcagttattttgcatgccacctccctgcctggtggacttccagactattttctgcattcgcccttgcgtgtccattgttgcaaggagcgatttggagaaaataaactgtgagtttcacagatccacgggccacgctcccctgggggcttcatgatcccaccgcctttcccgatgatgatgacaaccgcggctgtctgaagcggctgacgaaatcggttgagattctgatgagaggcttgggggggtctttgccctcaaggcgaggctccttctcctaggaatgccgagccccctgcactagcttcgctccactggggatctttgccagccttcatactagattcagcgatcccccggttgggaatcttcgccagccccccgtcctgctatcccgctcgtcgccgcgcctcccatgctaagggcccccttcctttcccttgactttggggatattcggagtctgctctcgccgctctcttctctcgtttaaacgagagaatagtagtagggtccagtctcaggccccctcactttgggtcttagaatggtggccgggctgtaaaattctggaggacggagagggcggccccggagttgttatcaaagaggcactggaggatgttggccgctccttggagcagcttgtcgaaataatgatccacggccacgggaacgccgtgccgctcggcgtaggccgggtcctcggccatctccgtctttctcgcccccttcactccccccttgggctccacaaagacgtactggatgcggtcgtggatctggggcagttcctcgttgcgctcgacgaacttctggtagacggccaggtgaggcatctgggtgctcttgtaggctgagagcttgcggctgagctccgttgaaaagcagagctcccccatggggaccctgccttcacggaggtctgtgtaggcctggtttaggatgtcaatgacgggcaaaaagcccacaggtagcccttgtgtaaatgactcttggaagggccggtgggagaggaggctggccgcctcctttacccgggcatccgccagcaccaggtcgagcacgcgccggcagcgtgtctgcacaaacttgcaggccgtcttccggacgagctccacccccttcatcagggtcttgccgtccgtcagcacccccacatatctcttctttgtaatcagcatcaggcaggagaaggtcttctcggcctccagggagatgggggccacaaacaggctccgggtggtgtgggcggccagggcatcggcaaagcgcagggtctcgctctctgaaaacccccggcactcgataaacagcgagtccgtgtccccgtagatgactcgaagctggccctcggggttgaggggcgcccaggcgtccggggagggggccagggcctgcaggttggcggggctcagggcctccacgaaggccttggcccgctccaacatcgtgcggccctgcagcgtcaccgtctcggcgatggagaggcagggaaagaggccgttggccaccccggtgaagccgtagacggcgttgcacgtgcacttgatggccagctgctgcttgtcgaggatggtcctttggcgcggatcctcgcaggccgccagcagcttcttgatggccttgcgcttggccagccaggaggtcaacagactagccaagaaggactcgtgcacgtgcttctttacaaagtggtagacgccccccgtgagcctgaaggactcatagtcttctcccgggcgcaggccggctagcctgtgctcttctcccggcgttatcatggtagaataacagagattatgagcctgaatgatgctcgggtagaggctggcaaagtccaccaccagaaccggggagttgtagaatccggacaggggctggatgacggtggccccctggtagccgtcccggtcagaggccgagggcatgggcaggataaagttttccttttgggcggccgccaggaggcaggagaacacgcggatctgctgcccatcgtccagcacccgcctgcaggggatgtgagcgatcttggcaatctctgccacctccacgtggatcacgaaatggtttagcagatccatgaccagggccgagtcctgcacgcagtacatgccgagccgcctgcgcccctcggggcccgctgcaaagaggcgaggaatctccttgtaatgcacatcctccttcttggcccccagtaggtgcctggctactgtgtccagcttgtagtctgagaggctgagcttgtcccggcacacggcgtacatgtcgatggggatgaggccggtgatgcggaccttggtgttggcccgcaagaagcccttgcccgcatcatggggtcgcctgacctcgcagacgcccccagccctaattttgcccagagaggctgggttgatgctgtagatgtgcctggctctgtccagaatgtagggccagtcaaagttggccacgttgtagccggtcacaatctccacgctgaggtctctgatgagctggaagaaggcgtagagcatgtccagctccgatgggaactcgtagacctcaaccccctctatgtcttcgcaggtgcccagcgtcagcaggatgcgcctatagcgcccggcctcctcccctgtcgaccagaggacgcaggatatctgcaggatcaggtcagcctcgttggtggccgtggggaagccctcctcccccagacactcgatatcgaaggccagggcctggtaggagggccaggagctgtcttcacgccggaccgagaggtcgcccacctcacagtcgtactcgagctcggcgtacgagtcccggtgctggaggcgggggatggcgcggcggcagctgtaccagccaaaggtgacaaagtcattgtccaggacaaagcggcgcgtggcatccacgttggcctcaaagatccgacacccgtgcttgtcttgcagccacgtggccacgtgacacacactgttgggatgggagagggtgatcttgtggtagtcgccggcatggttgccgtagcccataatggaacggcgcgtgaccttctccaccgagacccggcagggggtcctgcggtcgaaggtgctggccttgagggcgctgaggactgcaaactccacgtccagaccctgaggcgcgctggcgtagaagtaggcctgctgcccaaacacgttcacacacacgctggccccatcggccttgcgccggcccagtagcttgatgacgatgccacatggcaccacatacccctgtttatccgatggaatgacggcgcatttctcgtgcgtgtacaccgtctcgagtatgtcgtagacatggaagtccagagggcttccgtgggtgtctgcctccggccttgccgtgccctcttgggcacgctggcgccaccacatgccctttccatcctcgtcaccccccaccaccgtcagggagtcttggtagaagcacagggggggctgaggcccccgcacatccaccacccctgcggcgcctggtgtctggaaacacttgggaatgagacgcaggtactccttgtcaggctttttcagaaggcctttattaggtcttaggaaagggttatagaagagtcccccagacatggttaaaactcagtctctgcctccccaagcagtgcggcggcggtctctggatcgtgatagcgtcttctgcgtaggcctggaaaacggtccctggctgcctgcaatgctctgctggccactgagggtccggccgccctctgagctgctctcttttgctcctggttttgctcatgcagcgctaacatgatggcttgtaattctgtcttactaatgggattaatgcctggaccctcaccagaggcatgttgctgagcgagctcgtcgatcccggggtagagcatctgcaccggctgctgcgacatctggcgcgtgcgcctcgtgagggaaataaccaggatcaccacccccgccaccaggaccagaatgagcatgccgccgaaggggtttttgaagaaggagatgaaaccagagaccaggctgctaaacaaaccccccaccgtgctgactaggttggtgatggactgacccacgctacccagactgtccataagttcccccaggccgtccacgaattgatttcttccgtttgacactgcattgtccaaatccttccgcaggccggcgatgttttgcgcctggaagttgtactcccggaagatgccctccaggtcaaagacgttggaggcacgctgttcgtcccgtgagtacagctccagggaggcaaagtcaatgttctcgatgagggaggtgtttagtgagatgaaggtctgcagggtggcaatgccgtccagctcgatggttttaaagtggtggtagtcgttgtagacgtggatctcgttgccggactggaagtagtactggctggtcgcctggcacacctccgtcatcttttttgtgaggaagatctcgttgtcggtgcccagctgtccctcgtaggtcttggtgtcgttgataaagctgaaggacaccagggggcgcgagtagcacatggtctcggagccagggaccctcatgctcttgcgcagggtgacggtggcctggttaacgggcacgcactgggagactgagatgacatcccccaggcgcttggccgccaccgccttaccgtagatgctggacatgacggtggttggattaatcttggttagttctctcagcaccatgttctgcctcttctgctccaggcaccaggcccgcgcaaggtctcccagcatgcggttgatctggcggcgcagggagtcgtaggcaaattggatctggacggtggcgggattgttgagggtgcccagggacttcccgggggccgtggggggcaccggtgtggtggcgttccccgcatcccgcctccgacgcctcagaacggcggcgggggtgctcccgcgggccgcggatggggctgggggcgatggactgctggggggtgaggaagtcggagtggtaagctccgtcaggttcttgacggtggccaacgagcgcggggtcagaggtagccaagctaataacaatcctccgctcgttataaaatatgtaatggcttcctggcccttcgtgtaacgatcctggacggcctcgtacttctcatgcatggtcttgttcacctgctcttcgatgcacttgaaggcgtccgggagctctatgcccacggttgtgttggtcacgaagctagaggtgccctcgtcagtcacaaaatgtattgacttccctgtttctgtggcgatggtcgagtcaaaggtttgccagtgttgaagcgggcagtaggctgtcctgttctcgagcttccaagatagcgtgtaagtgcccttgtccaggaaggctcggcgttcgccttgcgggttcgtccctcggttgtcgtagtccactatcttgtagttagttctcacgtggaaggagtctgcccgctcatggaaggtttccttatttttcccgtcatagaaaggggacatttccacagtctgcccggtggtggtcacaaagaagtcgaaggggctgttggacttggccatcatgtcagttatcaggcagttgacggtagttcttgttctgtaagtccatatcaaccacccgggggcgtcatagagctccgtctggctggcgtagcggcgcaccccgttggccaggcccccggtgggctttaggttgacggtgatgttaactccgtcgcggtctacatacacgcgcgtcagcccatcttttgtcatcttgaccgcgttgtagcactggtagatggtatccatctggtcagtttcgtagctgtcaacggagaacttctcctcgtgccggttggtcacggagtccgcgtaccagccattgtagatgagaatgttggtcactatcttggtgtaggagcggaccttaaacgagtagggaataatgttgtctttaaacaccatcaacaggccctccgtgtgattctcccgcgtgccaaacgagggacactggatgtccgaggagaagcggaacaggtcgccgtggctggagagctcgcagactcggaaaggaaagctggtttgctgacgcgtggcggtaggctgcaccgtggtggcggggggtgcgggctgctctggggtctgcgcaccgagacggcacgccagggcggctagcagcacgaccacgcttagcaccctacgccgagtcatctctcatttggaggtgcaggtagagaagggcatatagatccttaaatacccaccccctgcccttatacagaagaattagggggcggtcagagtcgtacgtgaggtaaagcccatccgggggcagggcctggccggggctgaccgcgtccgcccggcgcaggatcaaggaccgcccccaggtcttgttgtagagggacacggttaggacggcctcgcgcagcgcccggcacagaatttgctggctagatgccagtgagcccccgggtacgctgtagaagctgttgaaggaggtctctatccagtcgctcggctcgatgcctggccatatcagggaagtcaggaatgccttctggtggggcagcgtacctgcggcgtcacagcagcgagccagggccacgttgctgggtgggggaaagagcccgctctcctccgccaggggccccgtgatgaaggtgtacaggctgtgcgtcagcgcgtgcaggtgctccgagctcagggtctgggtaaacaggtgtgttttgatgtacttggaattctcaaaggcggcaccctcgccggcgcgcctgtcctcccagggacccgagacgaaggcccgtctgtagaggaagtggttgcgcatgcgggccagctcccagtagaccacgtccccccagacgcgcaggcacagggtctcggtcagggtctcgctctgttgcgccaggcaggactgcagcttggccagaccctcggtggccacctggcgcaggtactgctccttgcgcttgagcgcgtccgagagggcgccggacgggccgggctctcgtgccccagccggccggggcacctccgggctctcccgggacgcctcctcctcgcctcggcccaaccgctgcatggctcggttgagccgcgtgtacagctcgttcctcttttgcaggatggcccggtactgggggtgcgccgtgaaggcggcggcgcagtccgccttcagcgcctccaccgcgtcgcccgaggagctgtagaccccgccgcagaagagccgctccgtggccccgggagccacggcgtcaaacaggtgagtcagccttgcccccgccagcgcctcctcgcaggccccccgcaccagggccaggcgacgctcccgggcaaacagggcagagaggcgggaatggccgccaccctccccctgccccgttgcaccgatagcatggccgccagagttccaatagaggagctccgagagctccgccacctccgggggcactgtcgagaagacgttgtaggtgtccagcgctctggtcgccccctctgcctccggccgccccgggcccgggaccgcgccctcctctgggccgcccggcctcgccttctcctcagcctccaacaggtgcccgagccccgcctggcggacttcattctcaaacagtcccgagaccggctccggattcaccggcaccgccaggtggttacaggagacgtgggtcccctctgccgtggaagggttgccgtggttgggcagaaccatcagctcgcccacacagcgccagcagggcacagaggtgatgtagaggcgcgggtctgggatgggacttacgccccgaaagcggcccagcagatccagggcccgttccaggctctccagccccatggtgtgagacatgcaataaaacacgctattgattctcttcattaaaatctctatgtcatttattaggcacaaacttacatcgactttatgccccccgtaaaactccacagagtacgcgactgagggggtacggagaggcgggacccgggtaccctttctaccaggggcgagcagcgcggcagaggcctctctcgagttctctagcaggtgcaccagctccagggacagggcgctgcatgcacggtcattctgccgtctcaaacggggaaggaggatggcctccagctcggccagcaggccggcgttgcgcaccaccgcagccacgtccagactccgggggtccagccgggtgcacacgctcagctcaaccgccagggcgtacacctggctgtacgccgccgccagcagccccgacatcgccgccccaggggtctctagacctcgagtccggggagaacggtggccagacggcgcttgcgtctgcccccggagccctgccctcctccacccagcagcagcccggccgaggcctgcgacgcggtgctgaccggctcggccacgctgataaagttgtcctgggctgccccgggcccaccccacactccctccagaaagtcccgagcggcctccgccgtccactctatcccgctggaggcaatggtcgccagggtttctaggacgctgtccgccaggacggagaagcggcccaataagtactccgcgtcgtccctagtcagcgaggcgcatgcctcgcccatggcatccacaaggttgcacaccacatcaaacacacagtcttcctcctgtttttgtgatataatggcctccaggccagccctgatgttctcaatctcatatgtggtcgcggcttgggtccggcgcttcacggtcaaccctagggtgggggtggcaaagacaaacttcttccgcatggaagagcccccggcctgcttgcgcagcccagccccgggggcctgcagcaggttcctgtccacgccccggcccataaagtatcccaggttcccggcctggaatatctggttgttgccgttgacccccgtgtacttgttgatggtcactggcagcgtgacaaccggacgggccttgcagacctggctaagacagtctgtggccgcgcagaccaccgtggtcgcagtaagggaggaggtggcctccgcgtaggccgctgccgactccaccgcccgcgtgcccagtacgtgggggtagtcacgggcgggcaccgactgcgtcctcggcaccagtccctgaatcaggctgatgtagaactgggtctggccgcacgccttcaggatggcgttgttgagcctctgcttggcgtaagtgaccaggttgccaggcaccacatctatgacgttgctctcttcgtgggcccgggagcccccgtccacaaagagggccaggtcagagtactcctccgcgctggccccgctggggacagggaccgagcgccgcctggaaaagttgtgccacaggtacaggcttgagagcttagtgtccgggaatagggtcttgtggtaggtgttgaggaatttcatgtagggcccgttgatgatgtagttctccctcctggtagtggacttgatgaagctgttctggagggcggcattctcccccgtgaagaccaccctgttcttgatcttgatgttcctggggcacagcatcagcaccttggacatgcgcacaggcagccgccggccgtacacccggccctgcagggccgcgtccaggtctggcaggtcgcaggtgggctccccatgcaccaccttggcctccttggccgtgaggacccccttgtcgatggccaggctcctaaagttggtgcacagcgtctggtagtgaccctttagccactctggggggctctggccaagcccggggttgtcattctcatagcacatacagatgggcagggagatgtcctgcaggatggtcagcagtgagcggtaaaacagctgggtgaagatggggcaggcgggctgcgcaaaggggttgcacgagtactgcatcacgtggtagcagctcttgaccaggtccttgtaggtgatgttgttcttggccatgctgttcataaactggaccacttcggcgtccaccgccgcatccacgtccttgaacatcttgacaaagtcacgcgggccatggggctccttctctagctttccttcagcgtctatgcccagccgagacagccgctccagcaggttctggttcagctgccagtaggtgtagcggggctcgtcgtccggccgctgcccgtcgtcctccttatcgatgaagttgagaaagttgcccaaaaagtccgtctcgttgtaggagcccgaggcccccgagatcacataggggtccctccgctgcgtggacatgacgggggggaagcggtccctcagcctaaagaagagcgtgttcaggcacacggccggggcccggccctcgcagagcgagcacatgggactggcggccgcccccgccacgtagctgcccgtctccggcaccggggtcagagagctcttctgtccctggcaaaactgcaggtagtaggcatagcgggcaagaaggttgggcgagaaggaggccgcatagaccaggtgctccacagcgtagtttcccggaccgttggttccggtcacgtctggcccaccccagcccgagaagcagggtcggcggcaggggtcccaggtcccctcctgcagggtccccaggccgtgggtcatgtagaaactgttaaagagactctccttgccctgaccggttgacttcgagacccccgagacgtagaggacggaattggtggcaaagatctgcgtggacacgtggggggccaggctggcattatatcggtgtaacgcagccacacgggcctctggaccctcacagtcggcaaacaggggccacgagtcgtagttgaggctggccggggtctcgtgcgaggcctccagcatggcgggtgcgtagctcaccgccagctcgcatgccgcgctgtccacaatcattaaggctcccgagtccgggtgactgatggttgaggctgggaactccttgaggggggccaccttggccaccttggcctggtcctgcaggctctgcttctccagcagctccaccagcttgcccacccgtcggacgcgcagcgcctgcgccagcccggtgtacagcgcctcgtgcatgcagcggctgaggtccgagttgtaaaactggcggagctggggcacgccctctgggaacacctccttgtcgtagagcgggaccctaacgctcgcagactgccccaccgctacctcctgttttaacgatggaatggccaccaggtttccgctgtagagtcgctccttgaaggcctcggttattgccaccgcccccaggtaggcagagggatctagcccttcggggaagaagtcccccggcttggagctttccctcggtagggcgctgtaggcgtcgtacccaaacacctccctggtctcgccacagagggcctcgagacccggcccctcaaagatggggggaaccatatgggcattgtggaacacgtagatgtccctgtgataggaggtagcgcgtaggagcccgcagttggggtcgggcctcctgtgcagagccttgacattgatgctgaagcccggctccacggtgatgccgcaaaggagcggcaccgtcaggcacctgtggcccgcgtagccggtccccagtgtggccacctccctaagagggtaggtggccagggggtaaaagtagatgtagccgcacggacccggctggctctggctgcccagattatcctcgctagtctgtgcaccctgcatgatgcccaaggtatcgccccggcctcccagtcccacattaaatgttacactttactcatcacgcaacacccactgtttattcatttacaaagatttcaggaagtcagtcaggctggccagggcccacgtcacggggaactgacgtctcagcgatcttggcatgccgcccagcctcgcaaaccagagtctgcgatagagggccaggtagtgggcgattgcccccagcacgaaggcggcgctcttgtggtcatccaggtagtttcgcaccgcaaacaccactgtgtagcacagcaccaccctgagccgcgaccagtagtcgtagtggtcgttgtacactgcgcgcaggacgctgatgatgagccgtacgtgcgtgtctttgcccccgatgtcggctgtcctgcaggccagctccgcgtacagcttcctatccttcctcagggaggccttgatgagccggcagaggaccagggctggcaaaggcaggtctttctcatcccgggtgaacaccgcgtacatggccctgaacatgaggtagctggactcagccaccttgtcgtccggcggcgagggcgcgacccacgcctcgaccggggtcctcacaaacacagaatctgtagacttggctggcctcatggtctcgtcaggccagctcacgggcttcaggcttatatgataaaatgggcgtggcagaatagtataagacgcgaggcctgggtgaggagagtccagagcaatggccaggttcatcgctcagctcctcctgttggcctcctgtgtggccgccggccaggctgtcaccgctttcttgggtgagcgagtcaccctgacctcctactggaggagggtgagcctcggtccagagattgaggtcagctggtttaaactgggcccaggagaggagcaggtgcttattgggcgcatgcaccacgatgtcatctttatagagtggcctttcaggggcttctttgatatccacagaagtgccaacaccttctttttagtagtcaccgctgccaacatctcccatgacggcaactacctgtgccgcatgaaactgggcgagaccgaggtcaccaagcaggaacacctgagcgtggtgaagcctctaacgctgtctgtccactccgaaaggtctcagttcccagacttctctgtccttactgtgacatgcaccgtgaatgcatttccccatccccacgtccagtggctcatgcccgagggcgtggagcccgcaccaactgcggcaaatggcggtgttatgaaggaaaaggatgggagcctctctgttgctgttgacctgtcacttcccaagccctggcacctgccagtgacctgcgttgggaaaaatgacaaggaggaagcccacggggtttatgtttctggatacttgtcgcaataaacgcacttgcctatttcaccttgttttagtgtggcattgggggggtggcattgcgggtggatagcctcgcgactcgtgggaaaatgggcggaagggcaccgtgggaaaatagttccaggtgacagcagcagtgtgtgaagattgtcacagctgctggtttggagaaaacgggggtgggcggtgatcagggagaacaattccccggggacacctgcacgagacccctgggctctcaggaactccgcccaggtcttgccaattggggtgatcctgtagcgccgcggtttcagcatcacaggttattttgcctgaagcttgctggggcgtaaatccctctcgccttgtttctcagagagcatttcaggccggttttgcagtcgctgctgcagctatggggtccctagaaatggtgccaatgggcgcgggtccccctagccccggcggggatccggatgggtacgatggcggaaacaactcccaatatccatctgcttctggctcttctgggaacacccccaccccaccgaacgatgaggaacgtgaatctaatgaagagcccccaccgccttatgaggacccatattggggcaatggcgaccgtcactcggactatcaaccactaggaacccaagatcaaagtctgtacttgggattgcaacacgacgggaatgacgggctccctccccctccctactctccacgggatgactcatctcaacacatatacgaagaagcgggcagaggaaggtaagagtgccatctatctgtacttttatttattgcatcacaagtcacatcaataataagggcgccatctagcgggagatgttatccacaccatcccaattcacatctcagggacaacaggtcaaagttctttgttgacacccccagcgctggctccagggggtggaagcgttggatgcagtcctccgcatcggggcggacgcctcctcccaacgcgtttctgcggatcagtcgctggctggtgggcatcggagtcggtgggcggtcctccacggggacacgctccttcttggccttgttctttgaccttttggacattcttctgaaggaacggcggagagtagcgtagaatccagccagtggtctacccggtcgcatggtggcttcttagatgaggagcaggcataaaagtccaaacaggacacagagtaccaccaggagtagtcttagtctgctgacgtctgggtcctcggggcaggggtggctaggcctggtctccgtagaagagccgggcaggccgcaggcagaggactgctgctctagcaaagcacgctccaggacgtgtaccatctcgagagtgaggcacagctgttttcgtggacttttatacagtaaggacaaggaaagaaggccagaggaatgtggaaagatgagcgaggacaggtgtggaggttttgggctagctcttagtttctgggtgtgagagagggattaaagtgcttatgcgcaaagaatgtgtcaacaacaggtgttcctgcctctgctggcatgagttaggtgtggcttgggctgaatccaaatgtgtattggcacaagatggaaagcaaagttgctggagttactgggtgggagacagggatgtatgtggtcccccgctggtatgccagtaccctgtggaagtaaggggcctcatctgcctggtagttgtgttgtgcagaggtctgatgtgtgtaggaggggtgggttcaacgcaggggcgttggtggcggagtctggcaacgcccgggtccttgctacctgtgtggtgtgttaagggctgggtaaaggtgtctgccaattctcgcatgtcctcctttccccttgttttgaaatagaatatgaatgtggcttttcagcctagacagacagtgtggctaagggagtgtgtgccagttaaggtgattagctaaggcattcccagtaaatggagggagagtcagtcaggcaagcctatgacatggtaatgcctagaagtaaagaaaggttagtcatagtagcttagctgaactgggccgtgggggtcgtcatcatctccaccggaaccagaagaacccaaaagcagcgtaggaaggtgtggatcaccgccgccatggccggaatcatgactatgaccgccgcctccgtctgtcatcaaaggcgggccctggtcacctcctttgttttcaacctcttccgtcaattgtggagggcctccatcatttccagcagagtcgctagggctatgaggcagcgggtcatgtgggccattgtcatcagtgttgtcagggtcctgtgggccattgtcatcagtgttgtcagggtcctgaggcagcgggtcatgtgggccattgtcatcagtgttgtcagggtcctgtgggccattgtcatcagtgttgtcagggtcctgtgggccattgtcaggaccacctccaggtgcgcctaggttttgagagcagagtgggggtccgtcgccggctccactcacgagcaggtggtgtctgccctcgttggagttagagtcagattcatggccagaatcatcggtagcttgttgagggtgcgggagggagtcatcgtggtggtgttcatcactgtgtcgttgtccatggtaatacatccagattaaaatcgccagaaacaggaggagccaaaggagatcaaccaatagagtccaccagttttgttgtagatagagagcaataatgagcaggatgaggtctaggaagaaggctaggaagaaggccaaaagctgccagatggtggcaccaagtcgccagagcatctccaataagtagatccagatacctaagactgcgttgaaaaaagagtgttagggttggaaaagtgggggtgtggtaaataattcctagggaatgttagatcttaccaagtaagcacccgaagatgaacagcacaattccaaggaacaatgcctgtccgtgcaaattccagagagcgatgagcaggagggtgactggggaaagaggagaaagtgcgttagagaaggaagagtaagggaaagggggtgtggggcaaagggtgtaatacttactcatcagtaggagtatacaaagggctccaagtggacagagaaggtctcttctgaagataaagatgatcaaaattataattataagcatgagagcaaaggaatagaggacaaggagggctcctccagtccagtcactcataacgatgtacagccaaaacagtagcgccaagaggaggagaaggagagcaaggcctagggaagaggagagggggggtcctcgagggggccgtcgcgggcccggtgggcccctctcaaggtcgtgttccatcctcagggcagtgtgtcaggagcaaggcagttgaggaaagaagggggcagagcagtgtgagaggcttatgtagggcggctacgtcagagtaacgcgtgtttcttgggatgtaggcccggggggatttgcggggtctgccggaggcagtacgggtacagatttcccgaaagcggcggtgtgtgtgtgcatgtaagcgtagaaaggggaagtagaaagcgtgtgtttgtgttagaaaagcgggtccccggggggcaagctgtgggaatgcggtggccaagtgcaacaggaaatggaaaggcagtgcggcaatcagaagggggagtgcgtagtgttgtgggaagcggcagtgtaatctgcacaaagaggcgcggggcgcgcaacgttgggaggtcgttggcggcaggcgggaggccgtgctttaggggggttcaggtgaggcaaggctgtggggtaaccgtaggggaggcgggtgaggcggctaagagggctaagggtcggcgggtgacgaagcagcagacggcggatatgggaatttcagaatgaggtggcggattcaggcgaaaagggtgtgggctgtgcgagtgtcatgaggcaggcgcggaaagtcgctgcggcttgctggggcatggggggccgcgcattcctggaaaaagtggagggggcgtggccttcccccgcggccccccagcccccccgcacagagcggcgctacggcgggcgggcggcggggggtcggggtccgcgggctccgggggctgcgggcggtggatggcggcggacgttccggggatcgggggggtcggggggcgccgcgcgggcgcagccatgcgtgaccgtgatgagggggcagggtcgcagggggtgtgtctggtgggggcgggagcggggggcggcgcgggagcctgcacgccgttggagggtagaatgacagggggcggggacagagaggcggtcgcgcccccggccgcgccagccaagcccccaaggggggcggggagcgggcaatggagcgtgacgaagggccccagggctgaccccggcaaacgtgacccggggctccggggtgacccagccaagcgtgaccaaggggcccgtgggtgacacaggcaaccctgacaaaggccccccaggaaagacccccggggggcatcggggggtggggcatggggggccgcgcattcctggaaaaagtggagggggcgtggccttcccccgcggccccccagcccccccgcacagagcggcgctacggcgggcgggcggcggggggtcggggtccgcgggctccgggggctgcgggcggtggatggcggcggacgttccggggatcgggggggtcggggggcgccgcgcgggcgcagccatgcgtgaccgtgatgagggggcagggtcgcagggggtgtgtctggtgggggcgggagcggggggcggcgcgggagcctgcacgccgttggagggtagaatgacagggggcggggacagagaggcggtcgcgcccccggccgcgccagccaagcccccaaggggggcggggagcgggcaatggagcgtgacgaagggccccagggctgaccccggcaaacgtgacccggggctccggggtgacccagccaagcgtgaccaaggggcccgtgggtgacacaggcaaccctgacaaaggccccccaggaaagacccccgtggggcatggggggccgcgcattcctggaaaaagtggagggggcgtggccttcccccgcggccccccagcccccccgcacagagcggcgctacggcgggcgggcggcggggggtcggggtccgcgggctccgggggctgcgggcggtggatggcggcggacgttccggggatcgggggggtcggggggcgccgcgcgggcgcagccatgcgtgaccgtgatgagggggcagggtcgcagggggtgtgtctggtgggggcgggagcggggggcggcgcgggagcctgcacgccgttggagggtagaatgacagggggcggggacagagaggcggtcgcgcccccggccgcgccagccaagcccccaaggggggcggggagcgggcaatggagcgtgacgaagggccccagggctgaccccggcaaacgtgacccggggctccggggtgacccagccaagcgtgaccaaggggcccgtgggtgacacaggcaaccctgacaaaggccccccaggaaagacccccggggggcatcggggggtggggcatggggggccgcgcattcctggaaaaagtggagggggcgtggccttcccccgcggccccccagcccccccgcacagagcggcgctacggcgggcgggcggcggggggtcggggtccgcgggctccgggggctgcgggcggtggatggcggcggacgttccggggatcgggggggtcggggggcgccgcgcgggcgcagccatgcgtgaccgtgatgagggggcagggtcgcagggggtgtgtctggtgggggcgggagcggggggcggcgcgggagcctgcacgccgttggagggtagaatgacagggggcggggacagagaggcggtcgcgcccccggccgcgccagccaagcccccaaggggggcggggagcgggcaatggagcgtgacgaagggccccagggctgaccccggcaaacgtgacccggggctccggggtgacccagccaagcgtgaccaaggggcccgtgggtgacacaggcaaccctgacaaaggccccccaggaaagacccccggggggcatcggggggggtgttggcgggggcatgggggggtcggatttcgcccttattgccctgttt

SEQ ID NO: 3 - DNA, 16 bp, synthetic construct
  source 1..16: mol_type = other DNA
gcgacctgccagacct

SEQ ID NO: 4 - DNA, 20 bp, synthetic construct
  source 1..20: mol_type = other DNA
cacagcatcagcaccttgga

SEQ ID NO: 5 - DNA, 15 bp, synthetic construct
  source 1..15: mol_type = other DNA
cagggccggttgtac

SEQ ID NO: 6 - DNA, 22 bp, synthetic construct
  source 1..22: mol_type = other DNA
ggtcagcagtgagcggtaaaac

SEQ ID NO: 7 - DNA, 21 bp, synthetic construct
  source 1..21: mol_type = other DNA
caaggacctggtcaagagctg

SEQ ID NO: 8 - DNA, 18 bp, synthetic construct
  source 1..18: mol_type = other DNA
ctgggtgaagacggggca

SEQ ID NO: 9 - DNA, 19 bp, synthetic construct
  source 1..19: mol_type = other DNA
tgtttgccgactgtgaggg

SEQ ID NO: 10 - DNA, 24 bp, synthetic construct
  source 1..24: mol_type = other DNA
ggctggcattatatcggtgtaacg

SEQ ID NO: 11 - DNA, 17 bp, synthetic construct
  source 1..17: mol_type = other DNA
agaggcccgtatggctg

SEQ ID NO: 12 - DNA, 15 bp, synthetic construct
  source 1..15: mol_type = other DNA
cagggccgggtgtac

SEQ ID NO: 13 - DNA, 483 bp, synthetic construct
  source 1..483: mol_type = other DNA
caccctgttcttgatcttgatgttcctggggcacagcatcagcaccttggacatgcgcacaggcagccgccggccgtacacccggccctgcagggccgcgtccaggtctggcaggtcgcaggtgggctccccatgcaccaccttggcctccttggccgtgaattctcatagcacatacagatgggcagggagatgtcctgcaggatggtcagcagtgagcggtaaaacagctgggtgaagacggggcaggcgggctgcgcaaaggggttgcacgagtactgcatcacgtggtagcagctcttgaccaggtccttgtaggtgagtagaggacggaattggtggcaaagatctgcgtggacacgtggggggccaggctggcattatatcggtgtaacgcagccatacgggcctctggaccctcacagtcggcaaacaggggccacgagtcgtagttgaggctggccggggtctcgtgcgaggcct

SEQ ID NO: 14 - DNA, 483 bp, synthetic construct
  source 1..483: mol_type = other DNA
caccctgttcttgatcttgatgttcctggggcacagcatcagcaccttggacatgcgcacaggcagccgccggccgtacaaccggccctgcagggccgcgtccaggtctggcaggtcgcaggtgggctccccatgcaccaccttggcctccttggccgtgaattctcatagcacatacagatgggcagggagatgtcctgcaggatggtcagcagtgagcggtaaaacagctgggtgaagatggggcaggcgggctgcgcaaaggggttgcacgagtactgcatcacgtggtagcagctcttgaccaggtccttgtaggtgagtagaggacggaattggtggcaaagatctgcgtggacacgtggggggccaggctggcattatatcggtgtaacgcagccacacgggcctctggaccctcacagtcggcaaacaggggccacgagtcgtagttgaggctggccggggtctcgtgcgaggcct

(19) United States
(12) Patent Application Publication
(10) Pub. No.: US 2025/0340958 A1
(43) Pub. Date: Nov. 6, 2025

(54) Methods and Reagents for Detection, Quantitation, and Genotyping of Epstein-Barr Virus

(71) Applicant: The Board of Trustees of the Leland Stanford Junior University, Stanford, CA (US)

(72) Inventors: Benjamin Pinsky, Palo Alto, CA (US); Jacob Miller, Redwood City, CA (US)

(21) Appl. No.: 18/998,005

(22) PCT Filed: Jul. 26, 2023
(86) PCT No.: PCT/US2023/070994

§ 371 Date: Jan. 23, 2025

Related U.S. Application Data
(60) Provisional application No. 63/369,450, filed on Jul. 26, 2022.

(51) Int. Cl. C12Q 1/70

(52) U.S. Cl. CPC C12Q 1/701; C12Q 2600/118; C12Q 2600/156; C12Q 2600/172

(57) ABSTRACT

Methods and oligonucleotide reagents for genotyping Epstein-Barr virus are disclosed. In particular, genetic profiling is used to detect BALF2 variants in the genome of Epstein-Barr virus in an infected individual to predict the risk of an individual developing nasopharyngeal carcinoma. Primers and allele-specific probes are provided for performing nucleic acid-based diagnostic assays to determine which alleles are present at single nucleotide polymorphisms (SNPs) in the BALF2 gene of Epstein-Barr virus in biological samples from potentially infected subjects. These primers and allele-specific probes can be used for amplifying target sequences to allow rapid detection of a single mutation or multiple mutations in the BALF2 gene simultaneously in a single assay. In addition, methods are provided for identifying individuals at high risk of developing nasopharyngeal carcinoma who are in need of further screening and treatment.

### INCORPORATION BY REFERENCE OF A SEQUENCE LISTING

A Sequence Listing is provided herewith as a Sequence Listing XML file, “STAN-2005WO_S22-281” created on Jul. 7, 2023 and having a size of 187,286 bytes. The contents of the Sequence Listing XML file are incorporated by reference herein in their entirety.

### BACKGROUND OF THE INVENTION

Epstein-Barr Virus (EBV)-associated nasopharyngeal carcinoma (NPC) is unusually restricted to certain regions and populations despite nearly ubiquitous EBV infection early in life (Abeynayake et al. (2014) J Clin Microbiol. 52 (10): 3802-3804). NPC is the second-leading cause of head/neck cancer mortality worldwide, and has no definite modifiable risk factors (Le et al. (2013) Clin. Cancer Res. Off. J. Am. Assoc. Cancer Res. 19 (8): 2208-2215). Without biomarker-based screening, most patients present with NPC at an advanced stage and have worse prognoses despite treatment intensification (Miller et al. (2021) J. Natl. Cancer Inst. 113 (7): 852-862).

Screening high-risk populations can detect most NPC cases at an early stage, but existing serologic and molecular diagnostics are limited by low positive predictive value (PPV) secondary to benign EBV reactivation (Xu et al. (2019) Nat. Genet. 51 (7): 1131-1136; Lam et al. (2020) Clin. Chem. 66 (4): 598-605). These false positives result in excess screening imaging, endoscopies, biopsies, and/or repeated laboratory testing which increase screening costs and visits. Ancillary triage testing with nasopharyngeal EBV PCR and plasma EBV next-generation sequencing (NGS) can increase PPV but have limitations (Hui et al. (2019) Int. J. Cancer 144 (12): 3031-3042; Bray F, Colombet M, Mery L, et al. Cancer Incidence in Five Continents, Vol. XI. Lyon: International Agency for Research on Cancer, 2017).

There remains a need for better methods to screen for high-risk EBV variants associated with NPC.

### SUMMARY OF THE INVENTION

Methods and oligonucleotide reagents for genotyping EBV are disclosed. In particular, genetic profiling is used to detect BALF2 variants in the genome of Epstein-Barr virus in an infected individual to predict the risk of an individual developing nasopharyngeal carcinoma (NPC). Primers and allele-specific probes are provided for performing nucleic acid-based diagnostic assays to determine which alleles are present at single nucleotide polymorphisms (SNPs) in the BALF2 gene of EBV in biological samples from potentially infected subjects. These primers and allele-specific probes can be used for amplifying target sequences to allow rapid detection of a single mutation or multiple mutations in the BALF2 gene simultaneously in a single assay. In addition, methods are provided for identifying individuals at high risk of developing nasopharyngeal carcinoma who are in need of further screening and treatment.

In one aspect, a method for genotyping one or more polymorphisms in a BALF2 gene of Epstein-Barr virus (EBV) using a nucleic acid amplification assay is provided, the method comprising: (a) obtaining a biological sample suspected of containing EBV nucleic acids from a subject; (b) amplifying the EBV nucleic acids, if present, with a set of primers comprising: (i) a forward primer comprising or consisting of the nucleotide sequence of SEQ ID NO:3 and a reverse primer comprising or consisting of the nucleotide sequence of SEQ ID NO:4, (ii) a forward primer comprising or consisting of the nucleotide sequence of SEQ ID NO:6 and a reverse primer comprising or consisting of the nucleotide sequence of SEQ ID NO:7; (iii) a forward primer comprising or consisting of the nucleotide sequence of SEQ ID NO:9 and a reverse primer comprising or consisting of the nucleotide sequence of SEQ ID NO:10; (iv) a forward primer and a reverse primer comprising at least one nucleotide sequence that differs from the corresponding nucleotide sequence of the forward primer and the reverse primer of a set selected from the group consisting of (i)-(iii) in that the forward primer or the reverse primer has up to three nucleotide changes compared to the corresponding nucleotide sequence, wherein the forward primer and the reverse primer are capable of hybridizing to and amplifying the EBV nucleic acids in the nucleic acid amplification assay: (v) a forward primer and a reverse primer that are complements of the corresponding nucleotide sequences of the forward primer and the reverse primer of a set selected from the group consisting of (i)-(iv); or (vi) any combination of (i)-(v); and (c) genotyping the BALF2 gene by detecting the presence of one or more alleles at the one or more polymorphisms of the BALF2 gene in the amplified nucleic acids using one or more detectably labeled allele-specific probes selected from: (i) a detectably labeled allele-specific probe comprising or consisting of the nucleotide sequence of SEQ ID NO: 5, (ii) a detectably labeled allele-specific probe comprising or consisting of the nucleotide sequence of SEQ ID NO:8, (iii) a detectably labeled allele-specific probe comprising or consisting of the nucleotide sequence of SEQ ID NO:11, (iv) a detectably labeled allele-specific probe comprising or consisting of the nucleotide sequence of SEQ ID NO:12, (v) a detectably labeled allele-specific probe comprising a nucleotide sequence having up to three nucleotide changes in a nucleotide sequence selected from the group consisting of SEQ ID NO:5, SEQ ID NO:8, SEQ ID NO:11, and SEQ ID NO:12, wherein the probe retains allele specificity of a probe selected from (i)-(iv), (vi) a detectably labeled allele-specific probe having a nucleotide sequence that is complementary to the corresponding nucleotide sequence of a detectably labeled allele-specific probe selected from the group consisting of (i)-(v); or (vii) any combination of (i)-(vi); and wherein detection of binding of the detectably labeled allele-specific probe comprising or consisting of the nucleotide sequence of SEQ ID NO: 5 to the EBV nucleic acids or an amplicon thereof produced by the forward primer comprising or consisting of the nucleotide sequence of SEQ ID NO:3 and the reverse primer comprising or consisting of the nucleotide sequence of SEQ ID NO:4, if present, indicates the BALF2 gene has an adenine (A) at nucleotide position 162215, wherein detection of binding of the detectably labeled allele-specific probe comprising or consisting of the nucleotide sequence of SEQ ID NO:8 to the EBV nucleic acids or an amplicon thereof produced by the forward primer comprising or consisting of the nucleotide sequence of SEQ ID NO:6 and the reverse primer comprising or consisting of the nucleotide sequence of SEQ ID NO:7, if present, indicates the BALF2 gene has a cytosine (C) at nucleotide position 162476, wherein detection of binding of the detectably labeled allele-specific probe comprising or consisting of the nucleotide sequence of SEQ ID NO:11 to the EBV nucleic acids or an amplicon thereof produced by the forward primer comprising or consisting of the nucleotide sequence of SEQ ID NO:9 and the reverse primer comprising or consisting of the nucleotide sequence of SEQ ID NO:10, if present, indicates the BALF2 gene has a thymine (T) at nucleotide position 163364, wherein detection of binding of the detectably labeled allele-specific probe comprising or consisting of the nucleotide sequence of SEQ ID NO:12 to the EBV nucleic acids or an amplicon thereof produced by the forward primer comprising or consisting of the nucleotide sequence of SEQ ID NO:3 and the reverse primer comprising or consisting of the nucleotide sequence of SEQ ID NO:4, if present, indicates the BALF2 gene has a cytosine (C) at position 162215, and wherein the nucleotide positions are numbered relative to the reference nucleotide sequence of SEQ ID NO:2.

In certain embodiments, the set of primers comprises the forward primer comprising or consisting of the nucleotide sequence of SEQ ID NO:3 and the reverse primer comprising or consisting of the nucleotide sequence of SEQ ID NO:4; and the one or more detectably labeled allele-specific probes comprise the detectably labeled allele-specific probe comprising or consisting of the nucleotide sequence of SEQ ID NO:5. In some embodiments, the one or more detectably labeled allele-specific probes further comprise the detectably labeled allele-specific probe comprising or consisting of the nucleotide sequence of SEQ ID NO:12.

In certain embodiments, the set of primers comprises the forward primer comprising or consisting of the nucleotide sequence of SEQ ID NO:6 and the reverse primer comprising or consisting of the nucleotide sequence of SEQ ID NO:7; and the one or more detectably labeled allele-specific probes comprise the detectably labeled allele-specific probe comprising or consisting of the nucleotide sequence of SEQ ID NO:8.

In certain embodiments, the set of primers comprises the forward primer comprising or consisting of the nucleotide sequence of SEQ ID NO:9 and the reverse primer comprising or consisting of the nucleotide sequence of SEQ ID NO:10; and the one or more detectably labeled allele-specific probes comprise the detectably labeled allele-specific probe comprising or consisting of the nucleotide sequence of SEQ ID NO:11.

In certain embodiments, the set of primers comprises: the forward primer comprising or consisting of the nucleotide sequence of SEQ ID NO:3 and the reverse primer comprising or consisting of the nucleotide sequence of SEQ ID NO:4, the forward primer comprising or consisting of the nucleotide sequence of SEQ ID NO:6 and the reverse primer comprising or consisting of the nucleotide sequence of SEQ ID NO:7; and the forward primer comprising or consisting of the nucleotide sequence of SEQ ID NO:9 and the reverse primer comprising or consisting of the nucleotide sequence of SEQ ID NO:10; and the one or more detectably labeled allele-specific probes comprise: the detectably labeled allele-specific probe comprising or consisting of the nucleotide sequence of SEQ ID NO:5, the detectably labeled allele-specific probe comprising or consisting of the nucleotide sequence of SEQ ID NO:8, the detectably labeled allele-specific probe comprising or consisting of the nucleotide sequence of SEQ ID NO:11, and the detectably labeled allele-specific probe comprising or consisting of the nucleotide sequence of SEQ ID NO:12.

In certain embodiments, the method further comprises using a nucleic acid comprising or consisting of the nucleotide sequence of SEQ ID NO: 13 as a risk allele control.

In certain embodiments, the method further comprises using a nucleic acid comprising or consisting of the nucleotide sequence of SEQ ID NO:14 as a non-risk allele control.

In certain embodiments, a method for genotyping one or more polymorphisms in a BALF2 gene of Epstein-Barr virus (EBV) using a nucleic acid amplification assay is provided, the method comprising: (a) obtaining a biological sample suspected of containing EBV nucleic acids from a subject; (b) amplifying the EBV nucleic acids, if present, with a set of primers comprising: (i) a forward primer comprising or consisting of the nucleotide sequence of SEQ ID NO:3 and a reverse primer comprising or consisting of the nucleotide sequence of SEQ ID NO:4, (ii) a forward primer comprising or consisting of the nucleotide sequence of SEQ ID NO:6 and a reverse primer comprising or consisting of the nucleotide sequence of SEQ ID NO:7; and (iii) a forward primer comprising or consisting of the nucleotide sequence of SEQ ID NO:9 and a reverse primer comprising or consisting of the nucleotide sequence of SEQ ID NO:10; and (c) genotyping the BALF2 gene by detecting the presence of one or more alleles at the one or more polymorphisms of the BALF2 gene in the amplified nucleic acids using a set of detectably labeled allele-specific probes comprising: (i) a detectably labeled allele-specific probe comprising or consisting of the nucleotide sequence of SEQ ID NO:5, (ii) a detectably labeled allele-specific probe comprising or consisting of the nucleotide sequence of SEQ ID NO:8, (iii) a detectably labeled allele-specific probe comprising or consisting of the nucleotide sequence of SEQ ID NO:11, and (iv) a detectably labeled allele-specific probe comprising or consisting of the nucleotide sequence of SEQ ID NO:12; wherein detection of binding of the detectably labeled allele-specific probe comprising or consisting of the nucleotide sequence of SEQ ID NO: 5 to the EBV nucleic acids or an amplicon thereof produced by the forward primer comprising or consisting of the nucleotide sequence of SEQ ID NO:3 and the reverse primer comprising or consisting of the nucleotide sequence of SEQ ID NO:4, if present, indicates the BALF2 gene has an adenine (A) at nucleotide position 162215, wherein detection of binding of the detectably labeled allele-specific probe comprising or consisting of the nucleotide sequence of SEQ ID NO:8 to the EBV nucleic acids or an amplicon thereof produced by the forward primer comprising or consisting of the nucleotide sequence of SEQ ID NO:6 and the reverse primer comprising or consisting of the nucleotide sequence of SEQ ID NO:7, if present, indicates the BALF2 gene has a cytosine (C) at nucleotide position 162476, wherein detection of binding of the detectably labeled allele-specific probe comprising or consisting of the nucleotide sequence of SEQ ID NO:11 to the EBV nucleic acids or an amplicon thereof produced by the forward primer comprising or consisting of the nucleotide sequence of SEQ ID NO:9 and the reverse primer comprising or consisting of the nucleotide sequence of SEQ ID NO:10, if present, indicates the BALF2 gene has a thymine (T) at nucleotide position 163364, wherein detection of binding of the detectably labeled allele-specific probe comprising or consisting of the nucleotide sequence of SEQ ID NO:12 to the EBV nucleic acids or an amplicon thereof produced by the forward primer comprising or consisting of the nucleotide sequence of SEQ ID NO:3 and the reverse primer comprising or consisting of the nucleotide sequence of SEQ ID NO:4, if present, indicates the BALF2 gene has a cytosine (C) at position 162215, and wherein the nucleotide positions are numbered relative to the reference nucleotide sequence of SEQ ID NO:2.

In certain embodiments, each allele-specific probe is detectably labeled with a fluorophore. In some embodiments, each allele-specific probe is labeled with a different fluorophore. In some embodiments, the allele-specific probes are detectably labeled with a 5′-fluorophore and a 3′-quencher. In some embodiments, the 3′-quencher is a black hole quencher (BHQ) or tetramethyl rhodamine (TAMRA).

In certain embodiments, the allele-specific probes comprise one or more propynyl-modified bases.

In certain embodiments, the amplifying comprises performing polymerase chain reaction (PCR) or isothermal amplification. In some embodiments, the PCR is quantitative PCR. In some embodiments, the isothermal amplification is loop-mediated isothermal amplification (LAMP), helicase-dependent amplification (HDA), or recombinase polymerase amplification (RPA).

In certain embodiments, the biological sample comprises blood, plasma, B cells, or epithelial cells.

In certain embodiments, the method further comprises determining whether the subject has a BALF2 haplotype associated with nasopharyngeal carcinoma (NPC), wherein detection of a cytosine (C) at nucleotide position 162215, a cytosine (C) at nucleotide position 162476, and a thymine (T) or a cytosine (C) at nucleotide position 163364 indicates the subject has a BALF2 haplotype associated with nasopharyngeal carcinoma (NPC) and is at risk of developing nasopharyngeal carcinoma.

In certain embodiments, the method further comprises performing further screening of the subject for nasopharyngeal carcinoma if the subject is identified as having a BALF2 haplotype associated with nasopharyngeal carcinoma (NPC). For example, further screening may comprise performing an endoscopy or magnetic resonance imaging (MRI). In some embodiments, the method further comprises treating the subject for nasopharyngeal carcinoma if the subject is identified as having nasopharyngeal carcinoma based on said genotyping and further screening.

In another aspect, a composition for genotyping one or more polymorphisms in a BALF2 gene of EBV in a biological sample using a nucleic acid amplification assay is provided, the composition comprising a set of primers and allele-specific probes comprising: (a) a forward primer comprising or consisting of the nucleotide sequence of SEQ ID NO:3, a reverse primer comprising or consisting of the nucleotide sequence of SEQ ID NO:4, an allele-specific probe comprising or consisting of the nucleotide sequence of SEQ ID NO:5, and an allele-specific probe comprising or consisting of the nucleotide sequence of SEQ ID NO:12; (b) a forward primer comprising or consisting of the nucleotide sequence of SEQ ID NO:6, a reverse primer comprising or consisting of the nucleotide sequence of SEQ ID NO:7, and an allele-specific probe comprising or consisting of the nucleotide sequence of SEQ ID NO:8; (c) a forward primer comprising or consisting of the nucleotide sequence of SEQ ID NO:9, a reverse primer comprising or consisting of the nucleotide sequence of SEQ ID NO:10, and an allele-specific probe comprising or consisting of the nucleotide sequence of SEQ ID NO:11: (d) a forward primer, a reverse primer, and an allele-specific probe comprising at least one nucleotide sequence that differs from the corresponding nucleotide sequence of the forward primer, the reverse primer, and the allele-specific probe of a set selected from the group consisting of (a)-(c) in that the forward primer, the reverse primer, or the allele-specific probe has up to three nucleotide changes compared to the corresponding nucleotide sequence, wherein the forward primer and the reverse primer are capable of hybridizing to and amplifying the EBV nucleic acids in the nucleic acid amplification assay, and wherein the allele-specific probe retains allele-specificity; (e) a forward primer, a reverse primer, and an allele-specific probe comprising nucleotide sequences that are complements of the corresponding nucleotide sequences of the forward primer, reverse primer, and the allele-specific probe of a set selected from the group consisting of (a)-(i); or (f) any combination of (a)-(e).

In certain embodiments, the set of primers and allele-specific probes comprises: (a) a forward primer comprising or consisting of the nucleotide sequence of SEQ ID NO:3, a reverse primer comprising or consisting of the nucleotide sequence of SEQ ID NO:4, an allele-specific probe comprising or consisting of the nucleotide sequence of SEQ ID NO:5, and an allele-specific probe comprising or consisting of the nucleotide sequence of SEQ ID NO:12; (b) a forward primer comprising or consisting of the nucleotide sequence of SEQ ID NO:6, a reverse primer comprising or consisting of the nucleotide sequence of SEQ ID NO:7, and an allele-specific probe comprising or consisting of the nucleotide sequence of SEQ ID NO:8; and (c) a forward primer comprising or consisting of the nucleotide sequence of SEQ ID NO:9, a reverse primer comprising or consisting of the nucleotide sequence of SEQ ID NO:10, and an allele-specific probe comprising or consisting of the nucleotide sequence of SEQ ID NO:11.

In certain embodiments, the allele-specific probes are detectably labeled. In some embodiments, each allele-specific probe is detectably labeled with a fluorophore. In some embodiments, each allele-specific probe is labeled with a different fluorophore. In some embodiments, the allele-specific probes are detectably labeled with a 5′-fluorophore and a 3′-quencher. For example, the 3′-quencher may be a black hole quencher (BHQ) or tetramethyl rhodamine (TAMRA).

In another aspect, a kit comprising a composition described herein and instructions for genotyping one or more polymorphisms in a BALF2 gene of Epstein-Barr virus (EBV) in a biological sample is provided. In some embodiments, the kit further comprises Taq polymerase and deoxyribonucleotide triphosphates.

### BRIEF DESCRIPTION OF THE DRAWINGS

FIGS. 1A-1J. Multiplex EBV BALF2 genotyping qPCR design, validation, and association studies with nasopharyngeal carcinoma in endemic and non-endemic populations. FIG. 1A) Analytical sensitivity for each of the four BALF2 qPCR targets. The 95% lower limit of detection with 95% confidence interval is reported for each target in units of EBV copies/mL plasma. In conjunction with the LLODs, the corresponding plasma viral load for 34 screen-detected preclinical NPC cases is presented to indicate likelihood of genotyping success. FIG. 1B) Analytical linearity for each of the four BALF2 qPCR targets, plotting cycle threshold (Ct) against nominal dsDNA control concentration in units of log 10 copies/μL template. FIG. 1C) Mixing studies at fixed total template concentration (100 copies/μL template) combining high-risk and low-risk dsDNA controls, demonstrating detection of minor allele fractions as low as 10% for each of the four targets. Measured concentration is plotted against nominal concentration. In the presence of mixed alleles, the assay is approximately linear as allele fraction decreases. FIG. 1D) Study overview and experimental workflow. First, the multiplex BALF2 genotyping assay was analytically validated using synthetic dsDNA controls and wild-type B95-8 whole virus control. Next, our non-endemic cohort of 24 NPC cases and 155 non-NPC controls contributed to BALF2 qPCR/NGS validation, longitudinal BALF2 genotyping, and BALF2-NPC association. Finally, our non-endemic cohort and three predominantly endemic cohorts contributed to a meta-analysis of 755 EBV+ NPC cases and 981 non-NPC controls. This validated the association between BALF2 haplotypes and NPC in multiple cohorts, further defined regional EBV genomic diversity, and was used to develop a variant-informed screening model. FIG. 1E) Prevalence of I613V and V317M between EBV+NPC cases and non-NPC controls in the present study and in the three prior EBV GWAS cohorts. FIG. 1F) Log-transformed odds ratios with 95% confidence intervals for association between BALF2 high-risk haplotypes (C-C-T, C-C-C. or both) and EBV+ NPC or other EBV-associated diseases in the current cohort and in the three prior EBV GWAS cohorts. FIG. 1G) Individual patient characteristics from current study of 24 NPC cases and 155 non-NPC controls. BALF2 haplotypes are defined by presence or absence of V700L, I613V, and/or V317M, which are associated with clinical phenotype. FIG. 1H) Plasma EBV viral load (log 10 IU/mL plasma) across phenotypes of 155 patients included in current study, demonstrating no significant difference between plasma viral load and phenotype. FIGS. 1I) and 1J) Association between NPC and other BALF2 single nucleotide variants identified by next-generation sequencing. Log-transformed P-value from association test and log-transformed odds ratios with 95% confidence intervals are presented for three variants of interest (V700L=162215C>A, I613V=162476C>T, V317M=163364C>T) and 13 additional variants differentially associated with NPC. V700L is mutually exclusive with I613V and V317M, was rare in this population, and was not associated with NPC risk. Only one other variant (163287G>A, synonymous) exceeded the Bonferroni-corrected P-value threshold.

FIGS. 2A-2F: Longitudinal EBV BALF2 genotyping and modeled variant-informed NPC screening strategies in 12 high-risk endemic populations. FIG. 2A) A subset of 16 patients with serial EBV-positive plasma specimens were subject to BALF2 genotyping by qPCR and NGS to assess temporal haplotype stability. Variant allele fraction (VAF) is plotted against time from first specimen collection for the three qPCR targets (V700L, I613V, V317M). The sample's viral load in log 10 EBNA-1 IU/mL is plotted below the allele frequencies. Two patients had one specimen each with temporally-discordant haplotypes. Patient #2 was a lung/liver transplant recipient with I613V detected in only the third of seven plasma specimens collected over 8.7 months. Patient #10 was a kidney transplant recipient with large-cell lymphoma who had V700 detected only in the first of five specimens collected over 7.8 months, whereas the subsequent four specimens harbored V700L, possibly indicating mutagenesis. FIG. 2B) Map of east/southeast Asia with 12 included high-risk populations. Shading represents the national NPC incidence rate. Each bubble indicates a single population with size proportional to incidence rate. Bubble color indicates the cost-effectiveness of variant-informed screening at variable willingness-to-pay thresholds. FIG. 2C) Modeled survival in a hypothetical cohort of 50-year-old patients in southern China. Survival differs with no screening (black line), seven variant-agnostic screening strategies (red solid lines), and seven variant-informed screening strategies (blue dashed lines) due to weighted stage distributions dictated by effective screening sensitivity. FIG. 2D) Cost-effectiveness of variant-agnostic and variant-informed screening strategies across variable screening frequencies. Box plots indicate median with interquartile range. FIG. 2E) Resource utilization after initial biomarker screening for variant-agnostic (A₀-G₀) and variant-informed screening strategies (A_{BALF2}-G_{BALF2}). Bar charts indicate absolute number of screening endoscopies and MRIs per 100,000 screened subjects. Referrals for endoscopy/MRI decrease after triage with BALF2 qPCR. FIG. 2F) NPC deaths per 100,000 screened individuals with variable screening frequencies and initial screening ages.

FIG. 3: A schematic of the multiplex qPCR assay for genotyping EBV BALF2. Three sets of forward and reverse primers are combined with allele-specific hydrolysis probes to detect the presence of BALF2 V700L, I613V, and/or V317M. Two synthetic dsDNA fragments harbor either the risk-associated or non-risk-associated BALF2 alleles and serve as assay controls. Nucleic acid from the B95-8 cell line serves as an additional wild-type whole-virus control. Nucleic acids are extracted from human plasma, combined with oligonucleotides, DNA polymerase, MgCl2, and dNTPs, then subject to real-time PCR. The results are analyzed to determine presence of absence of EBV nucleic acids and the associated BALF2 haplotype.

FIGS. 4A-4B: Multiplex EBV BALF2 genotyping qPCR design and example amplification curves. FIG. 4A) Regions within EBV BALF2 reference sequence (GenBank NC_00706.1:162397-163444) amplified by BALF2 qPCR. Three sets of conserved primers flank the three variants (V700L, I613V, and V317M) which define low-risk and high-risk BALF2 haplotypes. Four allele-specific hydrolysis probes targeting V700, V700L, I613V, and V317M and cleaved during primer extension. FIG. 4B) Example multiplex BALF2 qPCR amplification plots demonstrating detection or absence of V700, V700L, I613V, and V317M from EBV-negative plasma, dsDNA low-risk and high-risk controls, wild-type EBV DNA from B95-8 cell line, and clinical specimens with A-T-C, C-C-C, and C-C-T BALF2 haplotypes. Abbreviations: FWD, forward; REV, reverse; EBV, Epstein-Barr Virus; LLOD, lower limit of detection; dsDNA, double-stranded DNA; Ct, cycle threshold.

FIG. 5: Schematic of natural history Markov model of endemic nasopharyngeal carcinoma (NPC). Healthy adults could develop Epstein-Barr Virus-associated (EBV) stage I NPC and progress without detection to more advanced stages of undetected disease. This is the cohort of patients with prevalent undiagnosed NPC that can be screen-detected. Each undetected stage of NPC could present symptomatically or be detected by screening. A subset of healthy adults and those with each stage of preclinical NPC are biomarker positive or negative (for each of ten biomarker combinations). At the time of screening, participants who are biomarker positive (whether healthy or those with preclinical NPC) test positive and are found to be true positives or false positives. Similarly, participants who are biomarker negative are true negatives or false negatives. Each detected (incident) case of NPC is managed with radiotherapy and/or chemotherapy, and can either be cured or develop locoregional recurrence (LRR) or distant metastasis (DM) after treatment and die from their disease. Patients with stage IVC NPC receive chemotherapy until dying from their disease. In each state, there is also the risk of remaining within the same state or dying from other causes.

FIGS. 6A-6C: Sensitivity analysis of modeled variant-informed screening strategies in endemic populations. FIG. 6A) The modeled number of NPC deaths averted per screening test per 100,000 screened individuals in a modeled population of men and women in southern China, screened with variant-agnostic or variant-informed screening strategies starting from ages 40-60 with screening frequency ranging from every 1-5 years. While absolute mortality reduction is greatest with more frequent screening, the relative mortality reduction per test is lower with more frequent screening due to the lower prevalence of undiagnostic NPC with each successive screen. Per-test mortality reduction is similar irrespective of initial screening age. FIG. 6B) Prevalence of known and unknown BALF2 haplotypes in endemic population as the number of plasma PCR screens increase. Patients with low-risk haplotypes discontinue future screening. FIG. 6C) Results of deterministic sensitivity analysis indicating proportional change in cost-effectiveness (ICER/GDPppp) as model parameters vary within ranges of uncertainty. Top 18 parameters that impact cost-effectiveness are plotted, with upper/lower limits denoted by black/gray bars. Deterministic sensitivity analysis identified parameters that most impacted cost-effectiveness (FIG. 4, Tables 12 and 20). Within the studied parameter ranges, variations in stage-specific recurrence/survival rates, most health utilities, and the costs of imaging, workup, radiotherapy, and chemotherapy modestly impacted ICERs (±1.0-7.0%). Screening was more cost-effective (16.4% ICER decrease) in the setting of 2D/3DCRT owing to decreased survival, higher recurrence rates, and late toxicities that most impact patients with advanced-stage disease. Screening performance and costs were the principal determinants of cost-effectiveness. A 5.0% absolute decrease in sensitivity or compliance increased median ICER by 9.5% and 12.3%, respectively. A 0.05 increase or decrease in the long-term utility after definitive radiotherapy alone for stage I NPC decreased or increased median ICER by 16.0-23.6% Discount rate (0-5%) had the largest impact upon median ICER (±55%). Due to uncertainty in population-specific costs, we studied a broad range (50-200%) of screening costs. Doubling reagent/consumables costs or laboratory technician costs increased median ICER by 66.3% and 10.4%, respectively. We also studied uncertainty in the WHO-CHOICE regression estimates of healthcare costs in each economy, which had a <10% impact on median ICER. Because BALF2 variant-informed screening strategies were typically cost-neutral compared with variant-agnostic screening, varying the prevalence of high-risk haplotypes above or below the 60.5% obtained from meta-analysis only impacted median ICER by 2.5%. Abbreviations: ICER, incremental cost-effectiveness ratio; GDPppp, purchasing power parity-adjusted per-capita gross domestic product; MRI, magnetic resonance imaging; PCR, polymerase chain reaction; ELISA, enzyme-linked immunosorbent assay; NED, no evidence of disease; IMRT, intensity-modulated radiotherapy; 2D/3D CRT, 2D/3D conformal radiotherapy; WHO, World Health Organization; OS, overall survival.

### DETAILED DESCRIPTION OF THE INVENTION

Methods and oligonucleotide reagents for genotyping Epstein-Barr virus are disclosed. In particular, genetic profiling is used to detect BALF2 variants in the genome of Epstein-Barr virus in an infected individual to predict the risk of an individual developing nasopharyngeal carcinoma. Primers and allele-specific probes are provided for performing nucleic acid-based diagnostic assays to determine which alleles are present at single nucleotide polymorphisms (SNPs) in the BALF2 gene of Epstein-Barr virus in biological samples from potentially infected subjects. These primers and allele-specific probes can be used for amplifying target sequences to allow rapid detection of a single mutation or multiple mutations in the BALF2 gene simultaneously in a single assay. In addition, methods are provided for identifying individuals at high risk of developing nasopharyngeal carcinoma who are in need of further screening and treatment.

Before the present compositions, methods, and kits are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a nucleic acid” includes a plurality of such nucleic acids and reference to “the primer” includes reference to one or more primers and equivalents thereof, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about,” particularly in reference to a given quantity, is meant to encompass deviations of plus or minus five percent.

The terms “polymorphism,” “polymorphic nucleotide,” “polymorphic site” or “polymorphic nucleotide position” refer to a position in a nucleic acid that possesses the quality or character of occurring in several different forms. A nucleic acid polymorphism is characterized by two or more “alleles,” or versions of the nucleic acid sequence. Typically, an allele of a polymorphism that is identical to a reference sequence is referred to as a “reference allele” and an allele of a polymorphism that is different from a reference sequence is referred to as an “alternate allele,” or sometimes a “variant allele.” As used herein, the term “major allele” refers to the more frequently occurring allele at a given polymorphic site, and “minor allele” refers to the less frequently occurring allele, as present in the general or study population.

The term “single nucleotide polymorphism” or “SNP” refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. Single nucleotide polymorphisms can also arise from a deletion of a nucleotide or an insertion of a nucleotide relative to a reference allele.

SNPs generally are described as having a minor allele frequency, which can vary between populations, but generally refers to the sequence variation (A,T,G, or C) that is less common than the major allele. The frequency can be obtained from dbSNP or other sources, or may be determined for a certain population using Hardy-Weinberg equilibrium (See for details see Eberle M A, Rieder M J, Kruglyak L, Nickerson D A (2006) Allele Frequency Matching Between SNPs Reveals an Excess of Linkage Disequilibrium in Genic Regions of the Human Genome. PLOS Genet 2 (9): e142. doi: 10.1371/journal.pgen.0020142; herein incorporated by reference).

The term “single nucleotide variation” or “SNV” refers to a DNA sequence variation, wherein a single nucleotide (adenine, thymine, cytosine, or guanine) in the genome sequence is altered.

As used herein, the term “Epstein-Barr virus”, “human gammaherpesvirus 4”, “human herpesvirus 4”, or “EBV” refers to a human herpesvirus belonging to the Herpesviridae family of enveloped viruses having a linear double-stranded DNA genome (see, e.g., Epstein Barr Virus, One Herpes Virus: Many Diseases, Volumes 1 and 2 (part of Current Topics in Microbiology and Immunology), edited by Christian Münz, Springer International, 2015). The term EBV may include any strain of EBV of any subtype, such as EBV Type 1 and EBV Type 2, which is capable of causing disease in a human subject. In particular, the term encompasses any strain of EBV that causes nasopharyngeal carcinoma in humans, including strains comprising SNVs in the EBV BALF2 gene such as V700L [162215C>A], I613V [162476T>C], and/or V317M [163364C>T]). A large number of EBV isolates have been partially or completely sequenced. See, e.g., the National Center for Biotechnology Information (NCBI) database, which contain complete sequences for various EBV strains.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, peptide nucleic acids (PNAs), morpholino nucleic acids, locked nucleic acids (LNAs), glycol nucleic acids (GNAs), threose nucleic acids (TNAs) and hexitol nucleic acids (HNAs). and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA: RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

An EBV polynucleotide, oligonucleotide, nucleic acid and nucleic acid molecule, as defined above, is a nucleic acid molecule derived from EBV. The molecule need not be physically derived from the particular isolate in question, but may be synthetically or recombinantly produced. Nucleic acid sequences for a number of EBV isolates are known. Representative EBV sequences are known and are presented in SEQ ID NO:1 and SEQ ID NO:2 of the Sequence Listing. Additional representative sequences, including BALF2 sequences from various EBV isolates are listed in the National Center for Biotechnology Information (NCBI) database. See, for example, NCBI entries: Accession Nos. NC_007605, KT273949, AB850658, AB850654, AB850647, MH883784, MH883768, MH883766, MH883765, MH883759, MH883758, MG298927, MG298926, MG298925, MG298924, MG298923, MG298918, MG298917, DQ279927, MT648662, MT648661, MT648660, MT648659, MT648643, MT648642, MH837524, MH837518, MG298916, MG298915, MG298913, ALV83281. ALV83211, ALV83141, ALV83074, ALV83005, ALV82937, ALV82867, AGL80696, BAQ20414, BAQ20350, AXY93549, AXY93436, AXY93191, AXY92982, AXY92861, AXY92789, AXY92441, AWG93767, AWG93697, QZL10856, QZL10798, QZL10059, QZL10005, and QZL09831; all of which sequences (as entered by the date of filing of this application) are herein incorporated by reference. See also Choi et al. (2018) J. Microbiol. 56 (8): 525-533, Zanella et al. (2019) Sci. Rep. 9 (1): 9829, Xu et al. (2019) Nat Genet. 51 (7): 1131-1136, Miller et al. (2022) Mol Cancer 21 (1): 154, for sequence comparisons and a discussion of genetic diversity and phylogenetic analysis of Epstein-Barr viruses.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

As used herein, a “solid support” refers to a solid surface such as a magnetic bead, latex bead, microtiter plate well, glass plate, nylon, agarose, acrylamide, and the like.

As used herein, the term “target nucleic acid region” or “target nucleic acid” denotes a nucleic acid molecule with a “target sequence” to be amplified. The target nucleic acid may be either single-stranded or double-stranded and may include other sequences besides the target sequence, which may not be amplified. The term “target sequence” refers to the particular nucleotide sequence of the target nucleic acid which is to be amplified. The target sequence may include a probe-hybridizing region contained within the target molecule with which a probe will form a stable hybrid under desired conditions. The “target sequence” may also include the complexing sequences to which the oligonucleotide primers complex and extended using the target sequence as a template. Where the target nucleic acid is originally single-stranded, the term “target sequence” also refers to the sequence complementary to the “target sequence” as present in the target nucleic acid. If the “target nucleic acid” is originally double-stranded, the term “target sequence” refers to both the plus (+) and minus (−) strands (or sense and anti-sense strands).

As used herein, the term “probe” refers to a polynucleotide that contains a nucleic acid sequence complementary to a nucleic acid sequence present in the target nucleic acid analyte (e.g., at EBV BALF2 SNV location). The polynucleotide regions of probes may be composed of DNA, and/or RNA, and/or synthetic nucleotide analogs. Probes may be labeled in order to detect the target sequence. Such a label may be present at the 5′ end, at the 3′ end, at both the 5′ and 3′ ends, and/or internally. The “probe” may contain at least one fluorescer and at least one quencher. Quenching of fluorophore fluorescence may be eliminated by exonuclease cleavage of the fluorophore from the oligonucleotide or by hybridization of the oligonucleotide probe to the nucleic acid target sequence. Additionally, the oligonucleotide probe will typically be derived from a sequence containing a selected SNV that lies between the sense and the antisense primers when used in a nucleic acid amplification assay.

An “allele-specific probe” hybridizes to only one of the possible alleles of a SNP under suitably stringent hybridization conditions.

The term “primer” as used herein, refers to an oligonucleotide that hybridizes to the template strand of a nucleic acid and initiates synthesis of a nucleic acid strand complementary to the template strand when placed under conditions in which synthesis of a primer extension product is induced, i.e., in the presence of nucleotides and a polymerization-inducing agent such as a DNA or RNA polymerase and at suitable temperature, pH, metal concentration, and salt concentration. The primer is preferably single-stranded for maximum efficiency in amplification, but may alternatively be double-stranded. If double-stranded, the primer can first be treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA or RNA synthesis. Typically, nucleic acids are amplified using at least one set of oligonucleotide primers comprising at least one forward primer and at least one reverse primer capable of hybridizing to regions of a nucleic acid flanking the portion of the nucleic acid to be amplified.

An “allele-specific primer” matches the sequence exactly of only one of the possible alleles of a SNV, hybridizes at the SNV location, and amplifies only one specific allele if it is present in a nucleic acid amplification reaction.

The term “amplicon” refers to the amplified nucleic acid product of a polymerase chain reaction (PCR) or other nucleic acid amplification process such as isothermal nucleic acid amplification (e.g., loop-mediated isothermal amplification (LAMP), helicase-dependent amplification (HDA), recombinase polymerase amplification (RPA), or nicking enzyme amplification reaction (NEAR)).

As used herein, the term “capture oligonucleotide” refers to an oligonucleotide that contains a nucleic acid sequence complementary to a nucleic acid sequence present in the target nucleic acid analyte such that the capture oligonucleotide can “capture” the target nucleic acid. One or more capture oligonucleotides can be used in order to capture the target analyte. The polynucleotide regions of a capture oligonucleotide may be composed of DNA, and/or RNA, and/or synthetic nucleotide analogs. By “capture” is meant that the analyte can be separated from other components of the sample by virtue of the binding of the capture molecule to the analyte. Typically, the capture molecule is associated with a solid support, either directly or indirectly.

The terms “hybridize” and “hybridization” refer to the formation of complexes between nucleotide sequences which are sufficiently complementary to form complexes via Watson-Crick base pairing. Where a primer “hybridizes” with a target (template), such complexes (or hybrids) are sufficiently stable to serve the priming function required by, e.g., a DNA polymerase to initiate DNA synthesis.

It will be appreciated that the hybridizing sequences need not have perfect complementarity to provide stable hybrids. In many situations, stable hybrids will form where fewer than about 10% of the bases are mismatches, ignoring loops of four or more nucleotides. Accordingly, as used herein the term “complementary” refers to an oligonucleotide that forms a stable duplex with its “complement” under assay conditions, generally where there is about 90% or greater homology.

The term “sample” as used herein relates to a material or mixture of materials, typically, although not necessarily, in liquid form, containing one or more analytes of interest.

As used herein, a “biological sample” refers to a sample of cells, tissue, or fluid isolated from a subject, including but not limited to, for example, blood, plasma, serum, fecal matter, urine, bone marrow, bile, spinal fluid, lymph fluid, samples of the skin, external secretions of the skin, respiratory, intestinal, and genitourinary tracts, tears, saliva, milk, blood cells (e.g., peripheral blood mononuclear cells, B cells, T cells, NK cells), organs (e.g., liver, kidney, lung, spleen, thymus, tonsil, or lymph node), biopsies and also samples of in vitro cell culture constituents including but not limited to conditioned media resulting from the growth of cells and tissues in culture medium, e.g., recombinant cells, and cell components.

The term “assaying” is used herein to include the physical steps of manipulating a sample to generate data related to the sample. As will be readily understood by one of ordinary skill in the art, a sample must be “obtained” prior to assaying the sample. Thus, the term “assaying” implies that the sample has been obtained. The terms “obtained” or “obtaining” as used herein encompass the act of receiving an extracted or isolated sample. For example, a testing facility can “obtain” a sample in the mail (or via delivery, etc.) prior to assaying the sample. In some such cases, the sample was “extracted” or “isolated” from an individual by another party prior to mailing (i.e., delivery, transfer, etc.), and then “obtained” by the testing facility upon arrival of the sample. Thus, a testing facility can obtain the sample and then assay the sample, thereby producing data related to the sample.

The terms “obtained” or “obtaining” as used herein can also include the physical extraction or isolation of a sample from a subject. Accordingly, a sample can be isolated from a subject (and thus “obtained”) by the same person or same entity that subsequently assays the sample. When a sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating a sample.

In some embodiments, the step of obtaining comprises the step of isolating a sample (e.g., a pre-treatment sample, a post-treatment sample, etc.). Methods and protocols for isolating various samples (e.g., a blood sample, a serum sample, a plasma sample, a biopsy sample, an aspirate, etc.) will be known to one of ordinary skill in the art and any convenient method may be used to isolate a sample.

It will be understood by one of ordinary skill in the art that in some cases, it is convenient to wait until multiple samples have been obtained prior to assaying the samples. Accordingly, in some cases an isolated sample is stored until all appropriate samples have been obtained. One of ordinary skill in the art will understand how to appropriately store a variety of different types of samples and any convenient method of storage may be used (e.g., refrigeration) that is appropriate for the particular sample. In some cases, samples are processed immediately or as soon as possible after they are obtained.

“Diagnosis” as used herein generally includes determination as to whether a subject is likely affected by a given disease, disorder or dysfunction.

“Prognosis” as used herein generally refers to a prediction of the probable course and outcome of a clinical condition or disease. A prognosis of a patient may be made, for example, based on genotyping to determine the presence of one or more SNVs which are indicative of the risk of developing a disease, disorder or dysfunction. Determining the prognosis of a patient may further involve evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with nasopharyngeal carcinoma) as well as those in which prevention is desired (e.g., those with a genetic predisposition to developing nasopharyngeal carcinoma, those with an environmental exposure to a carcinogen, or who otherwise have an increased susceptibility or increased likelihood of developing cancer, those suspected of having nasopharyngeal carcinoma, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including human and non-human mammals such as non-human primates, including chimpanzees and other apes and monkey species; laboratory animals such as mice, rats, rabbits, hamsters, guinea pigs, and chinchillas; domestic animals such as dogs and cats; and farm animals such as sheep, goats, pigs, horses and cows.

A “therapeutically effective dose” or “therapeutic dose” is an amount sufficient to effect desired clinical results (i.e., achieve therapeutic efficacy). A therapeutically effective dose can be administered in one or more administrations.

“Providing an analysis” is used herein to refer to the delivery of an oral or written analysis (i.e., a document, a report, etc.). A written analysis can be a printed or electronic document. A suitable analysis (e.g., an oral or written report) provides any or all of the following information: identifying information of the subject (name, age, etc.), a description of what type of sample(s) was used and/or how it was used, the technique used to assay the sample, the results of the assay (e.g., the BALF2 genotype, presence or absence of any BALF2 SNVs associated with risk of developing EBV-associated nasopharyngeal carcinoma such as V700L [162215C>A], I613V [162476T>C], V317M [163364C>T]), the assessment as to whether the individual is determined to have a BALF2 genotype associated with risk of developing nasopharyngeal carcinoma (e.g., high-risk haplotype such as C-C-C or C-C-T at positions 162215, 162476T, and 163364, respectively), a recommendation for further screening or treatment, etc. The report can be in any format including, but not limited to printed information on a suitable medium or substrate (e.g., paper); or electronic format. If in electronic format, the report can be in any computer readable medium, e.g., diskette, compact disk (CD), flash drive, and the like, on which the information has been recorded. In addition, the report may be present as a website address which may be used via the internet to access the information at a remote site.

Detecting and Genotyping Single Nucleotide Variants Associated with Risk of Developing EBV-Associated Nasopharyngeal Carcinoma

Reagents and methods are provided for genotyping an individual to determine the risk of developing EBV-associated nasopharyngeal carcinoma. The pathogenesis of nasopharyngeal carcinoma is associated with EBV infection. In particular, single nucleotide variants (SNVs) in the EBV BALF2 gene, including V700L [162215C>A]. I613V [162476T>C], and V317M [163364C>T] are associated with a high-risk of developing nasopharyngeal carcinoma and can be used as genetic markers for evaluating the risk of an individual of developing EBV-associated nasopharyngeal carcinoma (the foregoing numbering is relative to the reference amino acid sequence of SEQ ID NO: 1 of the EBV (human gammaherpesvirus 4) BALF2 single-stranded DNA-binding protein and the reference EBV genomic sequence of SEQ ID NO:2). Especially high-risk haplotypes include C-C-C and C-C-T at positions 162215, 162476T, and 163364, respectively, which indicate an individual is at high risk of developing nasopharyngeal carcinoma. Accordingly, methods are provided for detecting these BALF2 SNVs and high-risk haplotypes in individuals, who have been infected with EBV, to evaluate the risk of developing EBV-associated nasopharyngeal carcinoma.

The methods use oligonucleotide reagents (e.g., oligonucleotide primers and allele-specific probes) or a combination of reagents capable of detecting one or more EBV BALF2 SNVs in a single assay. In one format, primer pairs and allele-specific probes capable of detecting one or more of the EBV BALF2 SNVs associated with high risk of developing EBV-associated nasopharyngeal carcinoma are used. For example, certain primers and allele specific probes capable of detecting more than one SNV associated with high risk of developing EBV-associated nasopharyngeal carcinoma may be used, such as any combination of two or more SNVs (e.g., both V700L [162215C>A] and I613V [162476T>C], both I613V [162476T>C] and V317M [163364C>T], both V700L [162215C>A] and V317M [163364C>T], or V700L [162215C>A], I613V [162476T>C], and V317M [163364C>T]).

For genetic testing, a biological sample containing EBV viral nucleic acids is collected from an individual. The biological sample is typically a blood or plasma sample, but can be any sample from bodily fluids, tissue, or cells (e.g., infected B cells, T cells, NK cells, or epithelial cells) that contains EBV genomic DNA. In some embodiments, the EBV DNA is from viral particles of infected B cells or epithelial cells or circulating cancer-derived EBV DNA from a nasopharyngeal carcinoma tumor. In certain embodiments, EBV nucleic acids from the biological sample are isolated, purified, and/or amplified prior to analysis. See, e.g., Green and Sambrook Molecular Cloning: A Laboratory Manual (Cold Spring Harbor Laboratory Press; 4th edition, 2012); and Current Protocols in Molecular Biology (Ausubel ed., John Wiley & Sons, 1995); herein incorporated by reference in their entireties.

Representative EBV sequences are known and are presented in SEQ ID NO:1 and SEQ ID NO: 2 of the Sequence Listing. Additional representative sequences, including BALF2 sequences from various EBV isolates are listed in the National Center for Biotechnology Information (NCBI) database. See, for example, NCBI entries: Accession Nos. NC_007605, KT273949, AB850658, AB850654. AB850647, MH883784, MH883768, MH883766, MH883765, MH883759, MH883758, MG298927, MG298926, MG298925, MG298924, MG298923, MG298918, MG298917, DQ279927, MT648662, MT648661. MT648660, MT648659, MT648643. MT648642, MH837524, MH837518, MG298916, MG298915, MG298913, ALV83281, ALV83211, ALV83141, ALV83074, ALV83005, ALV82937. ALV82867, AGL80696, BAQ20414, BAQ20350. AXY93549, AXY93436, AXY93191, AXY92982, AXY92861, AXY92789, AXY92441, AWG93767. AWG93697, QZL10856, QZL10798, QZL10059, QZL10005, and QZL09831; all of which sequences (as entered by the date of filing of this application) are herein incorporated by reference. See also Choi et al. (2018) J. Microbiol. 56 (8): 525-533. Zanella et al. (2019) Sci. Rep. 9 (1): 9829, Xu et al. (2019) Nat Genet. 51 (7): 1131-1136, Miller et al. (2022) Mol Cancer 21 (1): 154, for sequence comparisons and a discussion of genetic diversity and phylogenetic analysis of Epstein-Barr viruses.

Primers and allele-specific probes for use in the assays herein are derived from these sequences and are readily synthesized by standard techniques, e.g., solid phase synthesis via phosphoramidite chemistry, as disclosed in U.S. Pat. Nos. 4,458,066 and 4,415,732, incorporated herein by reference; Beaucage et al., Tetrahedron (1992) 48:2223-2311; and Applied Biosystems User Bulletin No. 13 (1 Apr. 1987). Other chemical synthesis methods include, for example, the phosphotriester method described by Narang et al., Meth. Enzymol. (1979) 68:90 and the phosphodiester method disclosed by Brown et al., Meth. Enzymol. (1979) 68:109. Poly (A) or poly (C), or other non-complementary nucleotide extensions may be incorporated into oligonucleotides using these same methods. Hexaethylene oxide extensions may be coupled to the oligonucleotides by methods known in the art. Cload et al., J. Am. Chem. Soc. (1991) 113:6324-6326; U.S. Pat. No. 4,914,210 to Levenson et al.; Durand et al., Nucleic Acids Res. (1990) 18:6353-6359; and Horn et al., Tet. Lett. (1986) 27:4705-4708.

A set of allele-specific probes is provided for detecting the EBV BALF2 SNVs: V700L [162215C>A], I613V [162476T>C], and V317M [163364C>T]. Each allele-specific probe hybridizes to only one of the possible alleles at the specified polymorphic sites under suitably stringent hybridization conditions. Individual probes comprise a nucleotide sequence derived from the nucleotide sequence of the target SNV sequences or complementary sequences thereof. An allele-specific probe can specifically hybridize under either stringent or lowered stringency hybridization conditions to a region of the EBV target BALF2 sequence containing the specified SNV, to the complement thereof, or to a nucleic acid sequence (such as a cDNA) derived therefrom.

The typical probe oligonucleotide is in the range of between 10-100 nucleotides long, such as 10-60, 15-40, 15-30, 15-25, and so on, and any length between the stated ranges such as 10, 11, 12, 13, 14, 15, 16, 17, 18, 19 20, 21, 22, 23 24, 25, 26, 27, 28, 29, 30, 35, 40, 45, 50, 55, 60, 65, 70, 75, 80, 85, 90, 95, or 100 nucleotides in length. In certain embodiments, a probe oligonucleotide comprises or consists of a sequence selected from the group consisting of SEQ ID NO:5, SEQ ID NO: 8, SEQ ID NO:11, and SEQ ID NO:12; or a fragment thereof comprising at least about 6 contiguous nucleotides, preferably at least about 8 contiguous nucleotides, more preferably at least about 10-12 contiguous nucleotides, and even more preferably at least about 15-20 contiguous nucleotides; or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, wherein the oligonucleotide probe retains allelic specificity and is capable of hybridizing to and detecting a particular EBV target nucleic acid comprising a BALF2 SNV of interest. Changes to the nucleotide sequences of SEQ ID NO:5, SEQ ID NO:8, SEQ ID NO:11, and SEQ ID NO:12 may be introduced corresponding to genetic variations in particular EBV strains. In certain embodiments, up to three nucleotide changes, including 1 nucleotide change, 2 nucleotide changes, or three nucleotide changes, may be made in a sequence selected from the group consisting of SEQ ID NO:5, SEQ ID NO: 8, SEQ ID NO: 11, and SEQ ID NO: 12, wherein the oligonucleotide probe retains allelic specificity and is capable of hybridizing to and detecting a particular EBV target nucleic acid comprising a BALF2 SNV of interest.

An allele-specific probe can comprise DNA, RNA, RNA or DNA mimetics, or combinations thereof, and can be single-stranded or double-stranded. Thus, the probes can be composed of naturally occurring nucleobases, sugars and covalent internucleoside (backbone) linkages as well as probes having non-naturally occurring portions which function similarly. Such modified or substituted probes may provide desirable properties such as, for example, enhanced affinity for a target gene sequence and increased stability.

In certain embodiments, a set of allele-specific probes is provided, the set comprising: (i) an allele-specific probe comprising or consisting of the nucleotide sequence of SEQ ID NO:5, (ii) an allele-specific probe comprising or consisting of the nucleotide sequence of SEQ ID NO:8, (iii) an allele-specific probe comprising or consisting of the nucleotide sequence of SEQ ID NO:11. (iv) an allele-specific probe comprising or consisting of the nucleotide sequence of SEQ ID NO:12, (v) an allele-specific probe comprising a nucleotide sequence having up to three nucleotide changes in a nucleotide sequence selected from the group consisting of SEQ ID NO:5, SEQ ID NO:8, SEQ ID NO: 11, and SEQ ID NO:12, wherein the probe retains allele specificity of a probe selected from (i)-(iv), (vi) an allele-specific probe having a nucleotide sequence that is complementary to the corresponding nucleotide sequence of a detectably labeled allele-specific probe selected from the group consisting of (i)-(v); or (vii) any combination of (i)-(vi).

In certain embodiments, a set of allele-specific probes is provided, the set comprising: (i) an allele-specific probe comprising or consisting of the nucleotide sequence of SEQ ID NO:5, (ii) an allele-specific probe comprising or consisting of the nucleotide sequence of SEQ ID NO:8, (iii) an allele-specific probe comprising or consisting of the nucleotide sequence of SEQ ID NO:11, and (iv) an allele-specific probe comprising or consisting of the nucleotide sequence of SEQ ID NO:12.

In addition, a set of primers is provided for amplifying EBV nucleic acids. For use in amplification reactions such as polymerase chain reaction (PCR), a pair of primers is used for detection of a SNV sequence. Each pair of primers is designed with sequences flanking a selected BALF2 SNV to generate an amplicon comprising a sequence containing the selected SNV. The pairs of primers are usually chosen so as to generate an amplification product of at least about 50 nucleotides, more usually at least about 100 nucleotides. These primers may be used in combination with allele-specific probes in standard quantitative or qualitative PCR-based assays for SNV genotyping of subjects.

Typically, the primer oligonucleotides have a length in the range of between 10-100 nucleotides in length, such as 15-60, 20-40, 20-30, and so on, more typically in the range of between 20-40 nucleotides long, and any length between the stated ranges. In certain embodiments, a primer oligonucleotide comprises or consists of a sequence selected from the group consisting of SEQ ID NO: 3, SEQ ID NO:4, SEQ ID NO:6, SEQ ID NO:7, SEQ ID NO:9, and SEQ ID NO:10; or a fragment thereof comprising at least about 6 contiguous nucleotides, preferably at least about 8 contiguous nucleotides, more preferably at least about 10-12 contiguous nucleotides, and even more preferably at least about 15-20 contiguous nucleotides; or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto. Changes to the nucleotide sequences of SEQ ID NO:3, SEQ ID NO:4, SEQ ID NO:6, SEQ ID NO: 7, SEQ ID NO:9, and SEQ ID NO:10 may be introduced corresponding to genetic variations in particular EBV strains. In certain embodiments, up to three nucleotide changes, including 1 nucleotide change, 2 nucleotide changes, or three nucleotide changes, may be made in a sequence selected from the group consisting of SEQ ID NO:3, SEQ ID NO:4, SEQ ID NO:6, SEQ ID NO:7, SEQ ID NO:9, and SEQ ID NO:10, wherein the oligonucleotide primer is capable of hybridizing to and amplifying a particular EBV target nucleic acid comprising a BALF2 SNV of interest.

In certain embodiments, a set of primers is provided, the set comprising: (i) a forward primer comprising or consisting of the nucleotide sequence of SEQ ID NO:3 and a reverse primer comprising or consisting of the nucleotide sequence of SEQ ID NO:4, (ii) a forward primer comprising or consisting of the nucleotide sequence of SEQ ID NO:6 and a reverse primer comprising or consisting of the nucleotide sequence of SEQ ID NO:7; (iii) a forward primer comprising or consisting of the nucleotide sequence of SEQ ID NO:9 and a reverse primer comprising or consisting of the nucleotide sequence of SEQ ID NO:10; (iv) a forward primer and a reverse primer comprising at least one nucleotide sequence that differs from the corresponding nucleotide sequence of the forward primer and the reverse primer of a set selected from the group consisting of (i)-(iii) in that the forward primer or the reverse primer has up to three nucleotide changes compared to the corresponding nucleotide sequence, wherein the forward primer and the reverse primer are capable of hybridizing to and amplifying the EBV nucleic acids in the nucleic acid amplification assay: (v) a forward primer and a reverse primer that are complements of the corresponding nucleotide sequences of the forward primer and the reverse primer of a set selected from the group consisting of (i)-(iv); or (vi) any combination of (i)-(v).

In certain embodiments, a set of primers is provided, the set comprising: (i) a forward primer comprising or consisting of the nucleotide sequence of SEQ ID NO:3 and a reverse primer comprising or consisting of the nucleotide sequence of SEQ ID NO:4, (ii) a forward primer comprising or consisting of the nucleotide sequence of SEQ ID NO:6 and a reverse primer comprising or consisting of the nucleotide sequence of SEQ ID NO:7; and (iii) a forward primer comprising or consisting of the nucleotide sequence of SEQ ID NO:9 and a reverse primer comprising or consisting of the nucleotide sequence of SEQ ID NO:10.

A label can be attached to or incorporated into an allele-specific probe or primer to allow detection and/or quantitation of a target SNV. The target SNV may be detected in genomic EBV DNA, expressed RNA, a cDNA copy thereof, or an amplification product derived therefrom, and may be the positive or negative DNA strand, as long as the SNV can be specifically detected in the assay being used. In certain multiplex formats, the labels on allele-specific probes used for detecting different SNVs may be distinguishable.

The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g., biotin-avidin or streptavidin). Many labels are commercially available in activated forms which can readily be used for such conjugation (for example through amine acylation), or labels may be attached through known or determinable conjugation schemes, many of which are known in the art.

There are several means known for derivatizing oligonucleotides with reactive functionalities which permit the addition of a label. For example, several approaches are available for biotinylating probes so that radioactive, fluorescent, chemiluminescent, enzymatic, or electron dense labels can be attached via avidin. See, e.g., Broken et al., Nucl. Acids Res. (1978) 5:363-384 which discloses the use of ferritin-avidin-biotin labels; and Chollet et al., Nucl. Acids Res. (1985) 13:1529-1541 which discloses biotinylation of the 5′ termini of oligonucleotides via an aminoalkylphosphoramide linker arm. Several methods are also available for synthesizing amino-derivatized oligonucleotides which are readily labeled by fluorescent or other types of compounds derivatized by amino-reactive groups, such as isothiocyanate, N-hydroxysuccinimide, or the like, see, e.g., Connolly, Nucl. Acids Res. (1987) 15:3131-3139, Gibson et al. Nucl. Acids Res. (1987) 15:6455-6467 and U.S. Pat. No. 4,605,735 to Miyoshi et al. Methods are also available for synthesizing sulfhydryl-derivatized oligonucleotides, which can be reacted with thiol-specific labels, see, e.g., U.S. Pat. No. 4,757,141 to Fung et al., Connolly et al., Nucl. Acids Res. (1985) 13:4485-4502 and Spoat et al. Nucl. Acids Res. (1987) 15:4837-4848. A comprehensive review of methodologies for labeling DNA fragments is provided in Matthews et al., Anal. Biochem. (1988) 169:1-25.

For example, oligonucleotides may be fluorescently labeled by linking a fluorescent molecule to the terminus of the molecule. Guidance for selecting appropriate fluorescent labels can be found in Smith et al., Meth. Enzymol. (1987) 155:260-301; Karger et al., Nucl. Acids Res. (1991) 19:4955-4962; Guo et al. (2012) Anal. Bioanal. Chem. 402 (10): 3115-3125; and Molecular Probes Handbook, A Guide to Fluorescent Probes and Labeling Technologies, 11th edition, Johnson and Spence eds., 2010 (Molecular Probes/Life Technologies). Fluorescent labels include fluorescein and derivatives thereof, such as disclosed in U.S. Pat. No. 4,318,846 and Lee et al., Cytometry (1989) 10:151-164. Dyes include, but are not limited to, 3-phenyl-7-isocyanatocoumarin, acridines, such as 9-isothiocyanatoacridine and acridine orange, pyrenes, benzoxadiazoles, and stilbenes, such as disclosed in U.S. Pat. No. 4,174,384. Examples of dyes include, without limitation, SYBR green, SYBR gold, a CAL Fluor dye such as CAL Fluor Gold 540, CAL Fluor Orange 560, CAL Fluor Red 590, CAL Fluor Red 610, and CAL Fluor Red 635, a Quasar dye such as Quasar 570, Quasar 670, and Quasar 705, an Alexa Fluor such as Alexa Fluor 488, Alexa Fluor 546, Alexa Fluor 555, Alexa Fluor 594, Alexa Fluor 647, and Alexa Fluor 784, a cyanine dye such as Cy3, Cy3.5, Cy5, Cy5.5, and Cy7, an ATTO dye such as ATTO 532, ATTO 542, ATTO 550, ATTO 565, ATTO 590, ATTO 594, ATTO 540Q, ATTO 575Q, ATTO 580Q, and ATTO 612Q; fluorescein, 2′, 4′, 5′, 7′-tetrachloro-4-7-dichlorofluorescein (TET), carboxyfluorescein (FAM), fluorescein isothiocyanate (FITC), 6-carboxy-4′,5′-dichloro-2′,7′-dimethoxyfluorescein (JOE), hexachlorofluorescein (HEX), rhodamine, carboxy-X-rhodamine (ROX), N′,N′,N′,N′-tetramethyl-6-carboxyrhodamine (TAMRA); and Texas Red, Yakima Yellow, Texas Red, 3-(E-carboxypentyl)-3′-ethyl-5,5′-dimethyloxa-carbocyanine (CYA); 5,6-carboxyrhodamine-110 (R110); 6-carboxyrhodamine-6G (R6G): Dragonfly orange; and BODIPY dyes. These dyes are commercially available from various suppliers such as Thermo Fisher Scientific (Waltham, MA), Life Technologies (Carlsbad, CA), Biosearch Technologies (Novato, Calif.), and Integrated DNA Technolgies (Coralville, Iowa).

Oligonucleotides can also be labeled with a minor groove binding (MGB) molecule, such as disclosed in U.S. Pat. Nos. 6,884,584, 5,801,155; Afonina et al. (2002) Biotechniques 32:940-944, 946-949; Lopez-Andreo et al. (2005) Anal. Biochem. 339:73-82; and Belousov et al. (2004) Hum Genomics 1:209-217. Oligonucleotides having a covalently attached MGB are more sequence specific for their complementary targets than unmodified oligonucleotides. In addition, an MGB group increases hybrid stability with complementary DNA target strands compared to unmodified oligonucleotides, allowing hybridization with shorter oligonucleotides.

Additionally, oligonucleotides can be labeled with an acridinium ester (AE). Current technologies allow the AE label to be placed at any location within the probe. See, e.g., Nelson et al., (1995) “Detection of Acridinium Esters by Chemiluminescence” in Nonisotopic Probing, Blotting and Sequencing, Kricka L. J. (ed) Academic Press, San Diego, Calif.; Nelson et al. (1994) “Application of the Hybridization Protection Assay (HPA) to PCR” in The Polymerase Chain Reaction, Mullis et al. (eds.) Birkhauser, Boston, Mass.; Weeks et al., Clin. Chem. (1983) 29:1474-1479; Berry et al., Clin. Chem. (1988) 34:2087-2090. An AE molecule can be directly attached to the probe using non-nucleotide-based linker arm chemistry that allows placement of the label at any location within the probe. See, e.g., U.S. Pat. Nos. 5,585,481 and 5,185,439.

In certain embodiments, molecular beacon probes may be used for detection of EBV target nucleic acids containing BALF2 SNVs. Molecular beacons are hairpin shaped oligonucleotides with an internally quenched fluorophore. Molecular beacons typically comprise four parts: a loop of about 15-30 nucleotides, which is complementary to the target nucleic acid sequence; a stem formed by two oligonucleotide regions that are complementary to each other, each about 5 to 7 nucleotide residues in length, on either side of the loop; a fluorophore covalently attached to the 5′ end of the molecular beacon, and a quencher covalently attached to the 3′ end of the molecular beacon. When the beacon is in its closed hairpin conformation, the quencher resides in proximity to the fluorophore, which results in quenching of the fluorescent emission from the fluorophore. In the presence of a target nucleic acid having a region that is complementary to the strand in the molecular beacon loop, hybridization occurs resulting in the formation of a duplex between the target nucleic acid and the molecular beacon. Hybridization disrupts intramolecular interactions in the stem of the molecular beacon and causes the fluorophore and the quencher of the molecular beacon to separate resulting in a fluorescent signal from the fluorophore that indicates the presence of the target nucleic acid sequence. See, e.g., Guo et al. (2012) Anal. Bioanal. Chem. 402 (10): 3115-3125; Wang et al. (2009) Angew. Chem. Int. Ed. Engl. 48 (5): 856-870; and Li et al. (2008) Biochem. Biophys. Res. Commun. 373 (4): 457-461; herein incorporated by reference in their entireties.

Representative EBV primers and allele-specific probes derived from the BALF2 gene for use in the various assays are shown in in Table 1 of Example 1. The oligonucleotides labeled as V700, I613, V317 are oligonucleotides that selectively amplify, detect, and/or hybridize to EBV nucleic acids comprising the specified BALF2 allele and can therefore be used to specifically identify individuals infected with a EBV strain having a SNV associated with a high risk of developing EBV-associated nasopharyngeal carcinoma using the assays described herein. Accordingly, for example, V700-specific oligonucleotides could be used in combination with I613-specific oligonucleotides in order to test for the presence of BALF2 SNVs at either V700 or 1613 in a single assay. Similarly, V700-specific oligonucleotides could be used in combination with I613-specific oligonucleotides and V317-specific oligonucleotides in order to test for the presence of SNVs at V700, I613, or V317 in a single assay. Also shown in Table 2 are EBV DNA fragments comprising risk alleles (SEQ ID NO:13) and non-risk alleles (SEQ ID NO:14) useful as controls in assays. It is to be understood that the primers and probes described herein are merely representative, and other oligonucleotides derived from various pathogenic EBV strains will find use in the assays described herein.

When utilizing a hybridization-based detection system, a nucleic acid probe is chosen that is complementary to a target nucleic acid sequence. By selection of appropriate conditions, the probe and the target sequence “selectively hybridize,” or bind, to each other to form a hybrid molecule. An allele-specific probe that “selectively hybridizes” to an EBV BALF2 sequence comprising a particular SNV under hybridization conditions described below, denotes an oligonucleotide that binds to a EBV BALF2 sequence comprising that particular SNV, but does not bind to a EBV BALF2 sequence not comprising the particular SNV.

In certain embodiments, an oligonucleotide (e.g., a primer or allele-specific probe) is capable of hybridizing selectively to a target sequence under moderately stringent hybridization conditions. Hybridization conditions useful for probe/target hybridization where the probe and target have a specific degree of sequence identity, can be determined as is known in the art (see, for example, Nucleic Acid Hybridization: A Practical Approach, editors B. D. Hames and S. J. Higgins, (1985) Oxford: Washington, D.C.; IRL Press). Hybrid molecules can be formed, for example, on a solid support, in solution, and in tissue sections. The formation of hybrids can be monitored by inclusion of a reporter molecule, typically, in the probe. Such reporter molecules or detectable labels include, but are not limited to, radioactive elements, fluorescent markers, and molecules to which an enzyme-conjugated ligand can bind.

With respect to stringency conditions for hybridization, it is well known in the art that numerous equivalent conditions can be employed to establish a particular stringency by varying, for example, the following factors: the length and nature of probe and target sequences, base composition of the various sequences, concentrations of salts and other hybridization solution components, the presence or absence of blocking agents in the hybridization solutions (e.g., formamide, dextran sulfate, and polyethylene glycol), hybridization reaction temperature and time parameters, as well as, varying wash conditions. The selection of a particular set of hybridization conditions is well known (see, for example, Sambrook, et al., Molecular Cloning: A Laboratory Manual, 3rd Edition, 2001).

As explained above, the primers and probes may be used in polymerase chain reaction (PCR)-based techniques, such as real-time PCR or quantitative PCR, to genotype BALF2 SNVs in EBV strains in biological samples. PCR is a technique for amplifying a desired target nucleic acid sequence contained in a nucleic acid molecule or mixture of molecules. In PCR, a pair of primers is employed in excess to hybridize to the complementary strands of the target nucleic acid. The primers are each extended by a polymerase using the target nucleic acid as a template. The extension products become target sequences themselves after dissociation from the original target strand. New primers are then hybridized and extended by a polymerase, and the cycle is repeated to geometrically increase the number of target sequence molecules. The PCR method for amplifying target nucleic acid sequences in a sample is well known in the art and has been described in, e.g., Innis et al. (eds.) PCR Protocols (Academic Press, NY 1990); Taylor (1991) Polymerase chain reaction: basic principles and automation, in PCR: A Practical Approach, McPherson et al. (eds.) IRL Press, Oxford; Saiki et al. (1986) Nature 324:163; as well as in U.S. Pat. Nos. 4,683,195, 4,683,202 and 4,889,818, all incorporated herein by reference in their entireties.

In particular, PCR uses relatively short oligonucleotide primers which flank the target nucleotide sequence to be amplified, oriented such that their 3′ ends face each other, each primer extending toward the other. The polynucleotide sample is extracted and denatured, preferably by heat, and hybridized with first and second primers that are present in molar excess. Polymerization is catalyzed in the presence of the four deoxyribonucleotide triphosphates (dNTPs: dATP, dGTP, dCTP and dTTP) using a primer- and template-dependent polynucleotide polymerizing agent, such as any enzyme capable of producing primer extension products, for example, E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs). This results in two “long products” which contain the respective primers at their 5′ ends covalently linked to the newly synthesized complements of the original strands. The reaction mixture is then returned to polymerizing conditions, e.g., by lowering the temperature, inactivating a denaturing agent, or adding more polymerase, and a second cycle is initiated. The second cycle provides the two original strands, the two long products from the first cycle, two new long products replicated from the original strands, and two “short products” replicated from the long products. The short products have the sequence of the target sequence with a primer at each end. On each additional cycle, an additional two long products are produced, and a number of short products equal to the number of long and short products remaining at the end of the previous cycle. Thus, the number of short products containing the target sequence grows exponentially with each cycle.

A PCR reaction will generally be carried out by cycling the reaction mixture between appropriate temperatures for annealing, elongation/extension, and denaturation for specific times. Such temperature and times will vary and will depend on the particular components of the reaction including, e.g., the polymerase and the primers as well as the expected length of the resulting PCR product. In some instances, e.g., where nested or two-step PCR are employed the cycling-reaction may be carried out in stages, e.g., cycling according to a first stage having a particular cycling program or using particular temperature(s) and subsequently cycling according to a second stage having a particular cycling program or using particular temperature(s).

In addition, one or more PCR additives or enhancing agents may be included to improve the yield of the amplification reaction, for example, by reducing secondary structure in a nucleic acid or mispriming events. Such additives or enhancing agents include, but are not limited to, dimethyl sulfoxide (DMSO), N,N,N-trimethylglycine (betaine), formamide, glycerol, nonionic detergents (e.g., Triton X-100, Tween 20, and Nonidet P-40 (NP-40)), 7-deaza-2′-deoxyguanosine, bovine serum albumin, T4 gene 32 protein, polyethylene glycol, 1,2-propanediol, and tetramethylammonium chloride.

In some instances, the method further comprises monitoring the amplification of a target DNA molecule such as is performed in real-time PCR, also referred to herein as quantitative PCR (qPCR). In real-time PCR, fluorescence is measured after each PCR cycle, wherein the intensity of the fluorescent signal is used to calculate the amount of DNA amplicons produced. Real-time PCR methods can be used, for example, to measure EBV viral load in a patient. For a description of suitable real-time PCR methods that can be used for detecting and quantitating EBV nucleic acids, see, e.g., Abeynayake et al. (2014) J Clin Microbiol. 52 (10): 3802-3804, Le et al. (2013) Clin Cancer Res. Off. J. Am. Assoc. Cancer Res. 19 (8): 2208-2215; herein incorporated by reference in their entireties.

In some embodiments, real-time PCR is performed with an allele-specific probe comprising a fluorophore on one end and a quencher on the other end. When the probe is intact, the distance between the reporter and the quencher is close enough to allow fluorescence resonance energy transfer (FRET) between the fluorophore and the quencher resulting in absorption of the light emitted by the fluorophore (i.e., quenching of the fluorescent signal). During amplification, a Taq polymerase with 5′->3′ exonuclease activity cleaves the fluorophore from the probe when the probe is bound to the DNA template to produce an unquenched fluorescent signal.

The fluorogenic 5′ nuclease assay, known as the TaqMan™ assay (Perkin-Elmer), is an example of such a real-time PCR method. Primers and allele-specific probes can be used in TaqMan™ analyses to detect the presence of EBV BALF2 SNVs (e.g., V700L [162215C>A], I613V [162476T>C], V317M [163364C>T]) in a biological sample containing EBV nucleic acids. Analysis is performed in conjunction with thermal cycling by monitoring the generation of fluorescence signals. The assay system dispenses with the need for gel electrophoretic analysis, and is capable of generating quantitative data allowing the determination of target copy numbers. For example, standard curves can be produced using serial dilutions of previously quantified EBV nucleic acids against which sample unknowns can be compared.

The fluorogenic 5′ nuclease assay is conveniently performed using, for example, AmpliTaq Gold™ DNA polymerase, which has endogenous 5′ nuclease activity, to digest an internal oligonucleotide probe labeled with both a fluorescent reporter dye and a quencher (see, Holland et al., Proc. Natl. Acad. Sci. USA (1991) 88:7276-7280; and Lee et al., Nucl. Acids Res. (1993) 21:3761-3766). Changes in fluorescence are measured during amplification cycles as the fluorescent probe is digested, uncoupling the dye and quencher labels and causing an increase in the fluorescent signal that is proportional to the amplification of target nucleic acid.

The amplification products can be detected in solution or using solid supports. In this method, the TaqMan™ probe is designed to hybridize to a target sequence comprising a BALF2 SNV within the desired PCR product. The 5′ end of the TaqMan™ probe contains a fluorescent reporter dye. The 3′ end of the probe is blocked to prevent probe extension and contains a dye that will quench the fluorescence of the 5′ fluorophore. During subsequent amplification, the 5′ fluorescent label is cleaved off if a polymerase with 5′ exonuclease activity present in the reaction. Excision of the 5′ fluorophore results in an increase in fluorescence that can be detected. For a detailed description of the TaqMan™ assay, reagents and conditions for use therein, see, e.g., Holland et al., Proc. Natl. Acad. Sci, U.S.A. (1991) 88:7276-7280; U.S. Pat. Nos. 5,538,848, 5,723,591, and 5,876,930, all incorporated herein by reference in their entireties.

A class of quenchers, known as “Black Hole Quenchers” such as BHQ1, BHQ2, BHQ3, BHQplus, and BHQnova can be used in the nucleic acid assays described above. Black Hole quenchers are described in, e.g., Johansson et al., J. Chem. Soc. (2002) 124:6950-6956 and are commercially available from Biosearch Technologies (Novato, Calif.). Other quenchers include, without limitation, TAMRA, DABCYL, and ATTO quenchers such as ATTO 540Q. ATTO 575Q, ATTO 580Q, and ATTO 612Q.

The subject methods are also applicable to digital PCR techniques. For digital PCR, a sample containing nucleic acids is separated into a large number of partitions before performing PCR. Partitioning can be achieved in a variety of ways known in the art, for example, by use of micro well plates, capillaries, emulsions, arrays of miniaturized chambers or nucleic acid binding surfaces. Separation of the sample may involve distributing any suitable portion including up to the entire sample among the partitions. Each partition includes a fluid volume that is isolated from the fluid volumes of other partitions. The partitions may be isolated from one another by a fluid phase, such as a continuous phase of an emulsion, by a solid phase, such as at least one wall of a container, or a combination thereof. In certain embodiments, the partitions may comprise droplets disposed in a continuous phase, such that the droplets and the continuous phase collectively form an emulsion.

The partitions may be formed by any suitable procedure, in any suitable manner, and with any suitable properties. For example, the partitions may be formed with a fluid dispenser, such as a pipette, with a droplet generator, by agitation of the sample (e.g., shaking, stirring, sonication, etc.), and the like. Accordingly, the partitions may be formed serially, in parallel, or in batch. The partitions may have any suitable volume or volumes. The partitions may be of substantially uniform volume or may have different volumes. Exemplary partitions having substantially the same volume are monodisperse droplets. Exemplary volumes for the partitions include an average volume of less than about 100, 10 or 1 mL, less than about 100, 10, or 1 nL, or less than about 100, 10, or 1 pL, among others.

After separation of the sample, PCR is carried out in the partitions. The partitions, when formed, may be competent for performance of one or more reactions in the partitions. Alternatively, one or more reagents may be added to the partitions after they are formed to render them competent for reaction. The reagents may be added by any suitable mechanism, such as a fluid dispenser, fusion of droplets, or the like.

In some embodiments, nucleic acids are amplified by emulsion PCR to compartmentalize the amplification reactions of individual DNA molecules. An aqueous PCR mixture with forward and reverse primers is mixed with an oil to create the emulsion. Preferably, each droplet of water in the oil emulsion contains one bead and one molecule of template DNA (e.g., a single assembled nucleic acid of the sequencing library), such that individual molecules are amplified in separate emulsion droplets. After amplification, the emulsion is broken, e.g., using isopropanol and detergent with vortexing. In some embodiments, the gene fragment library and the sequencing library are bound to magnetic beads or superparamagnetic beads prior to amplification, wherein amplification and breaking of the emulsion is followed by magnetic separation of the beads. For a description of emulsion PCR, see, e.g., Kanagal-Shamanna et al. (2016) Methods Mol Biol. 1392:33-42, Zhu et al. (2012) Anal Bioanal Chem. 403 (8): 2127-43, Zhang et al. (2020) Lab Chip 20 (13): 2328-2333, Siu et al. (2021) Talanta 221:121593, Zheng et al. (2011) Nat. Protoc. 6 (9): 1367-1376, and Kojima et al. (2015) Methods Mol. Biol. 2015; 1347:87-100; herein incorporated by reference.

After PCR amplification, nucleic acids can be quantified by counting the partitions that contain PCR amplicons. Partitioning of the sample allows quantification of the number of different molecules by assuming that the population of molecules follows a Poisson distribution. For a description of digital PCR methods, see, e.g., Hindson et al. (2011) Anal. Chem. 83 (22): 8604-8610; Pohl and Shih (2004) Expert Rev. Mol. Diagn. 4 (1): 41-47; Pekin et al. (2011) Lab Chip 11 (13): 2156-2166; Pinheiro et al. (2012) Anal. Chem. 84 (2): 1003-1011; Day et al. (2013) Methods 59 (1): 101-107; herein incorporated by reference in their entireties.

In some instances, amplification may be carried out under isothermal conditions, e.g., by means of isothermal amplification. Methods of isothermal amplification generally make use of enzymatic means of separating DNA strands to facilitate amplification at constant temperature, such as, e.g., strand-displacing polymerase or a helicase, thus negating the need for thermocycling to denature DNA. Any convenient and appropriate means of isothermal amplification may be employed in the subject methods, including but not limited to, recombinase polymerase amplification (RPA), loop-mediated isothermal amplification (LAMP), strand displacement amplification (SDA), helicase-dependent amplification (HDA), nicking enzyme amplification reaction (NEAR), and the like.

LAMP generally utilizes a plurality of primers, e.g., 4-6 primers, which may recognize a plurality of distinct regions, e.g., 6-8 distinct regions, of target DNA. Synthesis is generally initiated by a strand-displacing DNA polymerase with two of the primers forming loop structures to facilitate subsequent rounds of amplification. LAMP is rapid and sensitive. In addition, the magnesium pyrophosphate produced during the LAMP amplification reaction may, in some instances be visualized without the use of specialized equipment, e.g., by eye.

RPA combines isothermal recombinase-mediated primer targeting with strand-displacement DNA synthesis (Piepenburg et al. (2006) PLOS Biology. 4 (7): e204; herein incorporated by reference). The technique uses two primers together with a recombinase, a single-stranded DNA-binding protein, and a strand-displacing polymerase for amplification. Unlike PCR, heat is not required for melting of the DNA strands. Instead, a recombinase-primer complex is used for localized strand exchange to place oligonucleotide primers at homologous sequences of the DNA template. The single-stranded DNA-binding protein binds to the displaced template strand to prevent the primers from being ejected by branch migration. Dissociation of the recombinase leaves the 3′-end of the primer accessible to the strand displacing DNA polymerase (e.g., the large fragment of Bacillus subtilis Pol I), which catalyzes primer extension. Cyclic repetition of this process results in exponential amplification.

SDA generally involves the use of a strand-displacing DNA polymerase (e.g., Bst DNA polymerase, Large (Klenow) Fragment polymerase, Klenow Fragment (3′-5′ exo-), and the like) to initiate at nicks created by a strand-limited restriction endonuclease or nicking enzyme at a site contained in a primer. In SDA, the nicking site is generally regenerated with each polymerase displacement step, resulting in exponential amplification.

HDA generally employs: a helicase which unwinds double-stranded DNA unwinding to separate strands; primers, e.g., two primers, that may anneal to the unwound DNA; and a strand-displacing DNA polymerase for extension.

NEAR generally involves a strand-displacing DNA polymerase that initiates elongation at nicks, e.g., created by a nicking enzyme. NEAR is rapid and sensitive, quickly producing many short nucleic acids from a target sequence.

In some instances, entire amplification methods may be combined or aspects of various amplification methods may be recombined to generate a hybrid amplification method. For example, in some instances, aspects of PCR may be used, e.g., to generate the initial template or amplicon or first round or rounds of amplification, and an isothermal amplification method may be subsequently employed for further amplification. In some instances, an isothermal amplification method or aspects of an isothermal amplification method may be employed, followed by PCR for further amplification of the product of the isothermal amplification reaction. In some instances, a sample may be preamplified using a first method of amplification and may be further processed, including e.g., further amplified or analyzed, using a second method of amplification. As a non-limiting example, a sample may be preamplified by PCR and further analyzed by quantitative PCR (qPCR).

In certain embodiments, the target nucleic acids are separated from non-homologous nucleic acids using capture oligonucleotides immobilized on a solid support. Such capture oligonucleotides contain nucleic acid sequences that are complementary to a nucleic acid sequence present in the target EBV nucleic acid analyte such that the capture oligonucleotide can “capture” the target nucleic acid. Capture oligonucleotides can be used alone or in combination to capture EBV nucleic acids. For example, multiple capture oligonucleotides can be used in combination, e.g., 2, 3, 4, 5, 6, etc. different capture oligonucleotides can be attached to a solid support to capture target EBV nucleic acids. In certain embodiments, one or more capture oligonucleotides can be used to bind EBV target nucleic acids either prior to or after amplification by primer oligonucleotides and/or detection by probe oligonucleotides.

In certain embodiments, the biological sample potentially carrying target nucleic acids is contacted with a solid support in association with capture oligonucleotides. The capture oligonucleotides, which may be used separately or in combination, may be associated with the solid support, for example, by covalent binding of the capture moiety to the solid support, by affinity association, hydrogen binding, or nonspecific association.

The capture oligonucleotides can include from about 5 to about 500 nucleotides of a conserved region from a EBV BALF2 gene, preferably about 10 to about 100 nucleotides, or more preferably about 10 to about 60 nucleotides of the conserved region, or any integer within these ranges, such as a sequence including 18, 19, 20, 21, 22, 23, 24, 25, 26 . . . 35 . . . 40, etc. nucleotides from the conserved region of interest. In certain embodiments, the capture oligonucleotide comprises a sequence selected from the group consisting of SEQ ID NOS: 3-14 or a complement thereof. The capture oligonucleotide may also be phosphorylated at the 3′ end in order to prevent extension of the capture oligonucleotide.

The capture oligonucleotide may be attached to the solid support in a variety of manners. For example, the oligonucleotide may be attached to the solid support by attachment of the 3′ or 5′ terminal nucleotide of the probe to the solid support. More preferably, the capture oligonucleotide is attached to the solid support by a linker which serves to distance the probe from the solid support. The linker is usually at least 10-50 atoms in length, more preferably at least 15-30 atoms in length. The required length of the linker will depend on the particular solid support used. For example, a six atom linker is generally sufficient when high cross-linked polystyrene is used as the solid support.

A wide variety of linkers are known in the art which may be used to attach the oligonucleotide probe to the solid support. The linker may be formed of any compound which does not significantly interfere with the hybridization of the target sequence to the probe attached to the solid support. The linker may be formed of a homopolymeric oligonucleotide which can be readily added on to the linker by automated synthesis. The homopolymeric sequence can be either 5′ or 3′ to the virus-specific sequence. In one aspect of the invention, the capture oligonucleotides include a homopolymer chain, such as, for example poly A, poly T, poly G, poly C, poly U, poly dA, poly dT, poly dG, poly dC, or poly dU in order to facilitate attachment to a solid support. The homopolymer chain can be from about 10 to about 40 nucleotides in length, or preferably about 12 to about 25 nucleotides in length, or any integer within these ranges, such as for example, 10 . . . 12 . . . 16, 17, 18, 19, 20, 21, 22, 23, or 24 nucleotides. The homopolymer, if present, can be added to the 3′ or 5′ terminus of the capture oligonucleotides by enzymatic or chemical methods. This addition can be made by stepwise addition of nucleotides or by ligation of a preformed homopolymer. Capture oligonucleotides comprising such a homopolymer chain can be bound to a solid support comprising a complementary homopolymer. Alternatively, biotinylated capture oligonucleotides can be bound to avidin- or streptavidin-coated beads. See, e.g., Chollet et al., supra.

Alternatively, polymers such as functionalized polyethylene glycol can be used as the linker Such polymers do not significantly interfere with the hybridization of probe to the target oligonucleotide. Examples of linkages include polyethylene glycol, carbamate and amide linkages. The linkages between the solid support, the linker and the probe are preferably not cleaved during removal of base protecting groups under basic conditions at high temperature.

The solid support may take many forms including, for example, nitrocellulose reduced to particulate form and retrievable upon passing the sample medium containing the support through a sieve; nitrocellulose or the materials impregnated with magnetic particles or the like, allowing the nitrocellulose to migrate within the sample medium upon the application of a magnetic field; beads or particles which may be filtered or exhibit electromagnetic properties; and polystyrene beads which partition to the surface of an aqueous medium. Examples of types of solid supports for immobilization of the oligonucleotide probe include controlled pore glass, glass plates, polystyrene, avidin-coated polystyrene beads, cellulose, nylon, acrylamide gel and activated dextran.

In one embodiment, the solid support comprises magnetic beads. The magnetic beads may contain primary amine functional groups, which facilitate covalent binding or association of the capture oligonucleotides to the magnetic support particles. Alternatively, the magnetic beads have immobilized thereon homopolymers, such as poly T or poly A sequences. The homopolymers on the solid support will generally be complementary to any homopolymer on the capture oligonucleotide to allow attachment of the capture oligonucleotide to the solid support by hybridization. The use of a solid support with magnetic beads allows for a one-pot method of isolation, amplification and detection as the solid support can be separated from the biological sample by magnetic means.

The magnetic beads or particles can be produced using standard techniques or obtained from commercial sources. In general, the particles or beads may be comprised of magnetic particles, although they can also include other magnetic metal or metal oxides, whether in impure, alloy, or composite form, as long as they have a reactive surface and exhibit an ability to react to a magnetic field. Other materials that may be used individually or in combination with iron include, but are not limited to, cobalt, nickel, and silicon. A magnetic bead suitable for use with the present invention includes magnetic beads containing poly dT groups marketed under the trade name Sera-Mag magnetic oligonucleotide beads by Seradyn, Indianapolis, Ind.

Next, the association of the capture oligonucleotides with the solid support is initiated by contacting the solid support with the medium containing the capture oligonucleotides. In the preferred embodiment, the magnetic beads containing poly dT groups are hybridized with the capture oligonucleotides that comprise poly dA contiguous with the capture sequence (i.e., the sequence substantially complementary to a EBV nucleic acid sequence) selected from the conserved single stranded region of the dengue genome. The poly dA on the capture oligonucleotide and the poly dT on the solid support hybridize thereby immobilizing or associating the capture oligonucleotides with the solid support.

In certain embodiments, the capture oligonucleotides are combined with a biological sample under conditions suitable for hybridization with target EBV nucleic acids prior to immobilization of the capture oligonucleotides on a solid support. The capture oligonucleotide-target nucleic acid complexes formed are then bound to the solid support. In other embodiments, a solid support with associated capture oligonucleotides is brought into contact with a biological sample under hybridizing conditions. The immobilized capture oligonucleotides hybridize to the target nucleic acids present in the biological sample. Typically, hybridization of capture oligonucleotides to the targets can be accomplished in approximately 15 minutes, but may take as long as 3 to 48 hours.

The solid support is then separated from the biological sample, for example, by filtering, centrifugation, passing through a column, or by magnetic means. The solid support maybe washed to remove unbound contaminants and transferred to a suitable container (e.g., a microtiter plate). As will be appreciated by one of skill in the art, the method of separation will depend on the type of solid support selected. Since the targets are hybridized to the capture oligonucleotides immobilized on the solid support, the target strands are thereby separated from the impurities in the sample. In some cases, extraneous nucleic acids, proteins, carbohydrates, lipids, cellular debris, and other impurities may still be bound to the support, although at much lower concentrations than initially found in the biological sample. Those skilled in the art will recognize that some undesirable materials can be removed by washing the support with a washing medium. The separation of the solid support from the biological sample preferably removes at least about 70%, more preferably about 90% and, most preferably, at least about 95% or more of the non-target nucleic acids present in the sample. I

In some embodiments, the subject methods include providing an analysis of the risk of an individual developing EBV-associated nasopharyngeal carcinoma. The analysis may further provide the results of the assay (e.g., the BALF2 genotype, presence or absence of any BALF2 SNVs associated with risk of developing EBV-associated nasopharyngeal carcinoma such as V700L [162215C>A], I613V [162476T>C], V317M [163364C>T]), the assessment as to whether the individual is determined to have a BALF2 genotype associated with risk of developing nasopharyngeal carcinoma (e.g., a high-risk haplotype such as C-C-C or C-C-T at positions 162215, 162476T, and 163364, respectively), a recommendation for further screening or treatment, etc. As described above, an analysis can be an oral or written report (e.g., written or electronic document). The analysis can be provided to the subject, to the subject's physician, to a testing facility, etc. The analysis can also be accessible as a website address via the internet. In some such cases, the analysis can be accessible by multiple different entities (e.g., the subject, the subject's physician, a testing facility, etc.).

As is readily apparent, design of the assays described herein is subject to a great deal of variation, and many formats are known in the art. The above descriptions are merely provided as guidance and one of skill in the art can readily modify the described protocols, using techniques well known in the art.

### Kits

The above-described assay reagents, including the primers and allele-specific probes, and optionally capture oligonucleotides, a solid support with bound probes, and/or reagents for performing nucleic acid amplification, such as by PCR or isothermal amplification can be provided in kits, with suitable instructions and other necessary reagents, in order to conduct the assays as described above. The kit will normally contain in separate containers the primers and probes, control formulations (positive and/or negative), and other reagents that the assay format requires. The kit can also contain, depending on the particular assay used, other packaged reagents and materials (i.e., wash buffers, and the like). The reagents may be provided independently in liquid or solid form or provided in mixtures. Standard assays, such as those described above, can be conducted using these kits.

In certain embodiments, the kit comprises written instructions for detecting the presence of EBV BALF2 SNVs, including V700L [162215C>A], I613V [162476T>C], V317M [163364C>T]). In some embodiments, the kit comprises at least one set of primers for genotyping one or more polymorphisms in a BALF2 gene of EBV using a nucleic acid amplification assay, wherein the set of primers comprises: (i) a forward primer comprising or consisting of the nucleotide sequence of SEQ ID NO: 3 and a reverse primer comprising or consisting of the nucleotide sequence of SEQ ID NO:4, (ii) a forward primer comprising or consisting of the nucleotide sequence of SEQ ID NO:6 and a reverse primer comprising or consisting of the nucleotide sequence of SEQ ID NO:7; (iii) a forward primer comprising or consisting of the nucleotide sequence of SEQ ID NO:9 and a reverse primer comprising or consisting of the nucleotide sequence of SEQ ID NO:10; (iv) a forward primer and a reverse primer comprising at least one nucleotide sequence that differs from the corresponding nucleotide sequence of the forward primer and the reverse primer of a set selected from the group consisting of (i)-(iii) in that the forward primer or the reverse primer has up to three nucleotide changes compared to the corresponding nucleotide sequence, wherein the forward primer and the reverse primer are capable of hybridizing to and amplifying the EBV nucleic acids in the nucleic acid amplification assay; (v) a forward primer and a reverse primer that are complements of the corresponding nucleotide sequences of the forward primer and the reverse primer of a set selected from the group consisting of (i)-(iv); or (vi) any combination of (i)-(v). In some embodiments, the kit comprises at least one set of detectably labeled allele-specific probes for genotyping one or more polymorphisms in a BALF2 gene of EBV, wherein the set of allele-specific probes comprises (i) a detectably labeled allele-specific probe comprising or consisting of the nucleotide sequence of SEQ ID NO: 5, (ii) a detectably labeled allele-specific probe comprising or consisting of the nucleotide sequence of SEQ ID NO:8, (iii) a detectably labeled allele-specific probe comprising or consisting of the nucleotide sequence of SEQ ID NO:11, (iv) a detectably labeled allele-specific probe comprising or consisting of the nucleotide sequence of SEQ ID NO:12, (v) a detectably labeled allele-specific probe comprising a nucleotide sequence having up to three nucleotide changes in a nucleotide sequence selected from the group consisting of SEQ ID NO:5, SEQ ID NO:8, SEQ ID NO:11, and SEQ ID NO:12, wherein the probe retains allele specificity of a probe selected from (i)-(iv), (vi) a detectably labeled allele-specific probe having a nucleotide sequence that is complementary to the corresponding nucleotide sequence of a detectably labeled allele-specific probe selected from the group consisting of (i)-(v); or (vii) any combination of (i)-(vi); and wherein detection of binding of the detectably labeled allele-specific probe comprising or consisting of the nucleotide sequence of SEQ ID NO: 5 to the EBV nucleic acids or an amplicon thereof, if present, indicates the BALF2 gene has an adenine (A) at nucleotide position 162215, wherein detection of binding of the detectably labeled allele-specific probe comprising or consisting of the nucleotide sequence of SEQ ID NO:8 to the EBV nucleic acids or the amplicon thereof, if present, indicates the BALF2 gene has a cytosine (C) at nucleotide position 162476, wherein detection of binding of the detectably labeled allele-specific probe comprising or consisting of the nucleotide sequence of SEQ ID NO:11 to the EBV nucleic acids or the amplicon thereof, if present, indicates the BALF2 gene has a thymine (T) at nucleotide position 163364, wherein detection of binding of the detectably labeled allele-specific probe comprising or consisting of the nucleotide sequence of SEQ ID NO:12 to the EBV nucleic acids or an amplicon thereof, if present, indicates the BALF2 gene has a cytosine (C) at position 162215, wherein the nucleotide positions are numbered relative to the reference nucleotide sequence of SEQ ID NO: 2.

In certain embodiments, the kit comprises a set of primers comprising: (i) the forward primer comprising or consisting of the nucleotide sequence of SEQ ID NO:3 and the reverse primer comprising or consisting of the nucleotide sequence of SEQ ID NO:4, (ii) the forward primer comprising or consisting of the nucleotide sequence of SEQ ID NO:6 and the reverse primer comprising or consisting of the nucleotide sequence of SEQ ID NO:7; and (iii) the forward primer comprising or consisting of the nucleotide sequence of SEQ ID NO:9 and the reverse primer comprising or consisting of the nucleotide sequence of SEQ ID NO:10; and a set of detectably labeled allele-specific probes comprising (i) a detectably labeled allele-specific probe comprising or consisting of the nucleotide sequence of SEQ ID NO:5, (ii) a detectably labeled allele-specific probe comprising or consisting of the nucleotide sequence of SEQ ID NO:8, (iii) a detectably labeled allele-specific probe comprising or consisting of the nucleotide sequence of SEQ ID NO:11, and (iv) a detectably labeled allele-specific probe comprising or consisting of the nucleotide sequence of SEQ ID NO: 12.

In certain embodiments, the kit further comprises an EBV DNA fragment comprising a risk allele (SEQ ID NO:13) and/or an EBV DNA fragment comprising a non-risk allele (SEQ ID NO:14) for use as a control.

Kits may comprise one or more containers of the compositions described herein. Suitable containers for the compositions include, for example, bottles, vials, syringes, test tubes, and microwell plates. Containers can be formed from a variety of materials, including glass or plastic.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, Blu-ray, flash drive, and the like, on which the information has been recorded. Yet another form of these instructions that may be present is a website address which may be used via the internet to access the information at a removed site.

It will be apparent to one of ordinary skill in the art that various changes and modifications can be made without departing from the spirit or scope of the invention.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-30 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method for genotyping one or more polymorphisms in a BALF2 gene of Epstein-Barr virus (EBV) using a nucleic acid amplification assay, the method comprising:
(a) obtaining a biological sample suspected of containing EBV nucleic acids from a subject;
(b) amplifying the EBV nucleic acids, if present, with a set of primers comprising:
   (i) a forward primer comprising or consisting of the nucleotide sequence of SEQ ID NO: 3 and a reverse primer comprising or consisting of the nucleotide sequence of SEQ ID NO: 4,
   (ii) a forward primer comprising or consisting of the nucleotide sequence of SEQ ID NO: 6 and a reverse primer comprising or consisting of the nucleotide sequence of SEQ ID NO: 7;
   (iii) a forward primer comprising or consisting of the nucleotide sequence of SEQ ID NO: 9 and a reverse primer comprising or consisting of the nucleotide sequence of SEQ ID NO: 10;
   (iv) a forward primer and a reverse primer comprising at least one nucleotide sequence that differs from the corresponding nucleotide sequence of the forward primer and the reverse primer of a set selected from the group consisting of (i)-(iii) in that the forward primer or the reverse primer has up to three nucleotide changes compared to the corresponding nucleotide sequence, wherein the forward primer and the reverse primer are capable of hybridizing to and amplifying the EBV nucleic acids in the nucleic acid amplification assay;
   (v) a forward primer and a reverse primer that are complements of the corresponding nucleotide sequences of the forward primer and the reverse primer of a set selected from the group consisting of (i)-(iv); or
   (vi) any combination of (i)-(v); and
(c) genotyping the BALF2 gene by detecting the presence of one or more alleles at the one or more polymorphisms of the BALF2 gene in the amplified nucleic acids using one or more detectably labeled allele-specific probes selected from:
   (i) a detectably labeled allele-specific probe comprising or consisting of the nucleotide sequence of SEQ ID NO:5.
   (ii) a detectably labeled allele-specific probe comprising or consisting of the nucleotide sequence of SEQ ID NO:8.
   (iii) a detectably labeled allele-specific probe comprising or consisting of the nucleotide sequence of SEQ ID NO:11,
   (iv) a detectably labeled allele-specific probe comprising or consisting of the nucleotide sequence of SEQ ID NO:12,
   (v) a detectably labeled allele-specific probe comprising a nucleotide sequence having up to three nucleotide changes in a nucleotide sequence selected from the group consisting of SEQ ID NO:5, SEQ ID NO:8, SEQ ID NO:11, and SEQ ID NO:12, wherein the probe retains allele specificity of a probe selected from (i)-(iv),
   (vi) a detectably labeled allele-specific probe having a nucleotide sequence that is complementary to the corresponding nucleotide sequence of a detectably labeled allele-specific probe selected from the group consisting of (i)-(v); or
   (vii) any combination of (i)-(vi); and
wherein detection of binding of the detectably labeled allele-specific probe comprising or consisting of the nucleotide sequence of SEQ ID NO:5 to the EBV nucleic acids or an amplicon thereof produced by the forward primer comprising or consisting of the nucleotide sequence of SEQ ID NO: 3 and the reverse primer comprising or consisting of the nucleotide sequence of SEQ ID NO:4, if present, indicates the BALF2 gene has an adenine (A) at nucleotide position 162215,
wherein detection of binding of the detectably labeled allele-specific probe comprising or consisting of the nucleotide sequence of SEQ ID NO:8 to the EBV nucleic acids or an amplicon thereof produced by the forward primer comprising or consisting of the nucleotide sequence of SEQ ID NO: 6 and the reverse primer comprising or consisting of the nucleotide sequence of SEQ ID NO:7, if present, indicates the BALF2 gene has a cytosine (C) at nucleotide position 162476,
wherein detection of binding of the detectably labeled allele-specific probe comprising or consisting of the nucleotide sequence of SEQ ID NO:11 to the EBV nucleic acids or an amplicon thereof produced by the forward primer comprising or consisting of the nucleotide sequence of SEQ ID NO: 9 and the reverse primer comprising or consisting of the nucleotide sequence of SEQ ID NO: 10, if present, indicates the BALF2 gene has a thymine (T) at nucleotide position 163364,
wherein detection of binding of the detectably labeled allele-specific probe comprising or consisting of the nucleotide sequence of SEQ ID NO:12 to the EBV nucleic acids or an amplicon thereof produced by the forward primer comprising or consisting of the nucleotide sequence of SEQ ID NO: 3 and the reverse primer comprising or consisting of the nucleotide sequence of SEQ ID NO:4, if present, indicates the BALF2 gene has a cytosine (C) at position 162215, and
wherein the nucleotide positions are numbered relative to the reference nucleotide sequence of SEQ ID NO:2.

2. The method of aspect 1, wherein the set of primers comprises the forward primer comprising or consisting of the nucleotide sequence of SEQ ID NO:3 and the reverse primer comprising or consisting of the nucleotide sequence of SEQ ID NO:4; and the one or more detectably labeled allele-specific probes comprise the detectably labeled allele-specific probe comprising or consisting of the nucleotide sequence of SEQ ID NO:5.

3. The method of aspect 2, wherein the one or more detectably labeled allele-specific probes further comprise the detectably labeled allele-specific probe comprising or consisting of the nucleotide sequence of SEQ ID NO:12.

4. The method of any one of aspects 1-3, wherein the set of primers comprises the forward primer comprising or consisting of the nucleotide sequence of SEQ ID NO:6 and the reverse primer comprising or consisting of the nucleotide sequence of SEQ ID NO:7; and the one or more detectably labeled allele-specific probes comprise the detectably labeled allele-specific probe comprising or consisting of the nucleotide sequence of SEQ ID NO:8.

5. The method of any one of aspects 1-4, wherein the set of primers comprises the forward primer comprising or consisting of the nucleotide sequence of SEQ ID NO:9 and the reverse primer comprising or consisting of the nucleotide sequence of SEQ ID NO:10; and the one or more detectably labeled allele-specific probes comprise the detectably labeled allele-specific probe comprising or consisting of the nucleotide sequence of SEQ ID NO:11.

6. The method of aspect 1, wherein the set of primers comprises: the forward primer comprising or consisting of the nucleotide sequence of SEQ ID NO:3 and the reverse primer comprising or consisting of the nucleotide sequence of SEQ ID NO:4, the forward primer comprising or consisting of the nucleotide sequence of SEQ ID NO:6 and the reverse primer comprising or consisting of the nucleotide sequence of SEQ ID NO:7; and the forward primer comprising or consisting of the nucleotide sequence of SEQ ID NO:9 and the reverse primer comprising or consisting of the nucleotide sequence of SEQ ID NO:10; and the one or more detectably labeled allele-specific probes comprise: the detectably labeled allele-specific probe comprising or consisting of the nucleotide sequence of SEQ ID NO:5, the detectably labeled allele-specific probe comprising or consisting of the nucleotide sequence of SEQ ID NO:8, the detectably labeled allele-specific probe comprising or consisting of the nucleotide sequence of SEQ ID NO:11, and the detectably labeled allele-specific probe comprising or consisting of the nucleotide sequence of SEQ ID NO:12.

7. The method of any one of aspects 1-6, further comprising using a nucleic acid comprising or consisting of the nucleotide sequence of SEQ ID NO:13 as a risk allele control.

8. The method of any one of aspects 1-7, further comprising using a nucleic acid comprising or consisting of the nucleotide sequence of SEQ ID NO:14 as a non-risk allele control.

9. The method of any one of aspects 1-8, wherein each allele-specific probe is detectably labeled with a different fluorophore.

10. The method of any one of aspects 1-9, wherein each allele-specific probe is detectably labeled with a 5′-fluorophore and a 3′-quencher.

11. The method of aspect 10, wherein the 3′-quencher is a black hole quencher (BHQ) or tetramethyl rhodamine (TAMRA).

12. The method of any one of aspects 1-11, wherein said amplifying comprises performing polymerase chain reaction (PCR) or isothermal amplification.

13. The method of aspects 1-12, wherein the PCR is quantitative PCR.

14. The method of any one of aspects 1-13, wherein the biological sample comprises blood, plasma, B cells, or epithelial cells.

15. The method of any one of aspects 1-14, further comprising measuring EBV viral load in the biological sample.

16. The method of one of aspects 1-15, further comprising determining whether the subject has a BALF2 haplotype associated with nasopharyngeal carcinoma (NPC), wherein detection of a cytosine (C) at nucleotide position 162215, a cytosine (C) at nucleotide position 162476, and a thymine (T) or a cytosine (C) at nucleotide position 163364 indicates the subject has a BALF2 haplotype associated with nasopharyngeal carcinoma (NPC) and is at risk of developing nasopharyngeal carcinoma.

17. The method of aspect 16, further comprising performing further screening of the subject for nasopharyngeal carcinoma if the subject is identified as having a BALF2 haplotype associated with nasopharyngeal carcinoma (NPC).

18. The method of aspect 17, wherein said performing further screening comprises performing an endoscopy or magnetic resonance imaging (MRI).

19. The method of aspect 17 or 18, further comprising treating the subject for nasopharyngeal carcinoma if the subject is identified as having nasopharyngeal carcinoma based on said genotyping and further screening.

20. A method for genotyping one or more polymorphisms in a BALF2 gene of Epstein-Barr virus (EBV) using a nucleic acid amplification assay, the method comprising:
(a) obtaining a biological sample suspected of containing EBV nucleic acids from a subject;
(b) amplifying the EBV nucleic acids, if present, with a set of primers comprising:
   (i) a forward primer comprising or consisting of the nucleotide sequence of SEQ ID NO: 3 and a reverse primer comprising or consisting of the nucleotide sequence of SEQ ID NO: 4,
   (ii) a forward primer comprising or consisting of the nucleotide sequence of SEQ ID NO: 6 and a reverse primer comprising or consisting of the nucleotide sequence of SEQ ID NO: 7; and
   (iii) a forward primer comprising or consisting of the nucleotide sequence of SEQ ID NO: 9 and a reverse primer comprising or consisting of the nucleotide sequence of SEQ ID NO: 10; and
(c) genotyping the BALF2 gene by detecting the presence of one or more alleles at the one or more polymorphisms of the BALF2 gene in the amplified nucleic acids using a set of detectably labeled allele-specific probes comprising:
   (i) a detectably labeled allele-specific probe comprising or consisting of the nucleotide sequence of SEQ ID NO:5.
   (ii) a detectably labeled allele-specific probe comprising or consisting of the nucleotide sequence of SEQ ID NO:8.
   (iii) a detectably labeled allele-specific probe comprising or consisting of the nucleotide sequence of SEQ ID NO:11, and
   (iv) a detectably labeled allele-specific probe comprising or consisting of the nucleotide sequence of SEQ ID NO:12;
wherein detection of binding of the detectably labeled allele-specific probe comprising or consisting of the nucleotide sequence of SEQ ID NO:5 to the EBV nucleic acids or an amplicon thereof produced by the forward primer comprising or consisting of the nucleotide sequence of SEQ ID NO: 3 and the reverse primer comprising or consisting of the nucleotide sequence of SEQ ID NO:4, if present, indicates the BALF2 gene has an adenine (A) at nucleotide position 162215,
wherein detection of binding of the detectably labeled allele-specific probe comprising or consisting of the nucleotide sequence of SEQ ID NO:8 to the EBV nucleic acids or an amplicon thereof produced by the forward primer comprising or consisting of the nucleotide sequence of SEQ ID NO: 6 and the reverse primer comprising or consisting of the nucleotide sequence of SEQ ID NO:7, if present, indicates the BALF2 gene has a cytosine (C) at nucleotide position 162476,
wherein detection of binding of the detectably labeled allele-specific probe comprising or consisting of the nucleotide sequence of SEQ ID NO:11 to the EBV nucleic acids or an amplicon thereof produced by the forward primer comprising or consisting of the nucleotide sequence of SEQ ID NO: 9 and the reverse primer comprising or consisting of the nucleotide sequence of SEQ ID NO: 10, if present, indicates the BALF2 gene has a thymine (T) at nucleotide position 163364,
wherein detection of binding of the detectably labeled allele-specific probe comprising or consisting of the nucleotide sequence of SEQ ID NO:12 to the EBV nucleic acids or an amplicon thereof produced by the forward primer comprising or consisting of the nucleotide sequence of SEQ ID NO: 3 and the reverse primer comprising or consisting of the nucleotide sequence of SEQ ID NO:4, if present, indicates the BALF2 gene has a cytosine (C) at position 162215, and
wherein the nucleotide positions are numbered relative to the reference nucleotide sequence of SEQ ID NO:2.

21. The method of any one of aspects 1-20, wherein the allele-specific probes comprise one or more propynyl-modified bases.

22. A composition for genotyping one or more polymorphisms in a BALF2 gene of Epstein-Barr virus (EBV) in a biological sample using a nucleic acid amplification assay, the composition comprising a set of primers and allele-specific probes comprising:
(a) a forward primer comprising or consisting of the nucleotide sequence of SEQ ID NO:3, a reverse primer comprising or consisting of the nucleotide sequence of SEQ ID NO:4, an allele-specific probe comprising or consisting of the nucleotide sequence of SEQ ID NO:5, and an allele-specific probe comprising or consisting of the nucleotide sequence of SEQ ID NO:12;
(b) a forward primer comprising or consisting of the nucleotide sequence of SEQ ID NO:6, a reverse primer comprising or consisting of the nucleotide sequence of SEQ ID NO:7, and an allele-specific probe comprising or consisting of the nucleotide sequence of SEQ ID NO:8;
(c) a forward primer comprising or consisting of the nucleotide sequence of SEQ ID NO:9, a reverse primer comprising or consisting of the nucleotide sequence of SEQ ID NO:10, and an allele-specific probe comprising or consisting of the nucleotide sequence of SEQ ID NO:11;
(d) a forward primer, a reverse primer, and an allele-specific probe comprising at least one nucleotide sequence that differs from the corresponding nucleotide sequence of the forward primer, the reverse primer, and the allele-specific probe of a set selected from the group consisting of (a)-(c) in that the forward primer, the reverse primer, or the allele-specific probe has up to three nucleotide changes compared to the corresponding nucleotide sequence, wherein the forward primer and the reverse primer are capable of hybridizing to and amplifying the EBV nucleic acids in the nucleic acid amplification assay, and wherein the allele-specific probe retains allele-specificity;
(e) a forward primer, a reverse primer, and an allele-specific probe comprising nucleotide sequences that are complements of the corresponding nucleotide sequences of the forward primer, reverse primer, and the allele-specific probe of a set selected from the group consisting of (a)-(i); or
(f) any combination of (a)-(e).

23. The composition of aspect 22, wherein the set of primers and allele-specific probes comprises:
(a) a forward primer comprising or consisting of the nucleotide sequence of SEQ ID NO:3, a reverse primer comprising or consisting of the nucleotide sequence of SEQ ID NO:4, an allele-specific probe comprising or consisting of the nucleotide sequence of SEQ ID NO:5, and an allele-specific probe comprising or consisting of the nucleotide sequence of SEQ ID NO:12;
(b) a forward primer comprising or consisting of the nucleotide sequence of SEQ ID NO:6, a reverse primer comprising or consisting of the nucleotide sequence of SEQ ID NO:7, and an allele-specific probe comprising or consisting of the nucleotide sequence of SEQ ID NO:8; and
(c) a forward primer comprising or consisting of the nucleotide sequence of SEQ ID NO:9, a reverse primer comprising or consisting of the nucleotide sequence of SEQ ID NO:10, and an allele-specific probe comprising or consisting of the nucleotide sequence of SEQ ID NO:11.

24. The composition of aspect 22 or 23, wherein the allele-specific probes are detectably labeled.

25. The composition of aspect 24, wherein each allele-specific probe is labeled with a different fluorophore.

26. The composition of aspect 25, wherein each allele-specific probe is detectably labeled with a 5′-fluorophore and a 3′-quencher.

27. The composition of aspect 26, wherein the 3′-quencher is a black hole quencher (BHQ) or tetramethyl rhodamine (TAMRA).

28. The composition of any one of aspects 22-27, wherein the allele-specific probes comprise one or more propynyl-modified bases.

29. A kit comprising the composition of any one of aspects 12-28 and instructions for genotyping one or more polymorphisms in a BALF2 gene of Epstein-Barr virus (EBV) in a biological sample.

30. The kit of aspect 28, further comprising Taq polymerase and deoxyribonucleotide triphosphates.

### Experimental

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Multiplex Epstein-Barr Virus BALF2 Genotyping Detects High-Risk Variants in Plasma for Population Screening of Nasopharyngeal Carcinoma

### Introduction

Epstein-Barr Virus (EBV)-associated nasopharyngeal carcinoma (NPC) exhibits unusual geographic restriction despite ubiquitous lifelong infection. Screening programs can detect most NPC cases at an early stage, but existing EBV diagnostics are limited by false positives and low positive predictive value (PPV), leading to excess screening endoscopies, MRIs, and repeated testing. Human genome-wide association studies (GWAS) have previously identified susceptibility loci which are associated with NPC risk.8 However, the effect sizes are modest relative to the marked variation in NPC incidence worldwide. In contrast, several recent EBV GWAS have identified viral polymorphisms with much greater attributable risk.⁹⁻¹¹ In particular, two non-synonymous polymorphisms within the EBV BALF2 gene (I613V, V317M) may contribute more than 80% of attributable risk in southern China. Because humans typically establish a single lifelong latent EBV infection, BALF2 genotyping could serve as an adjunctive tool for lifetime screening triage.⁹ We therefore hypothesized that a noninvasive molecular diagnostic could detect high-risk EBV BALF2 variants in plasma and could serve to triage individuals for further screening work-up while remaining cost-effective in high-risk populations.

### Methods

### Multiplex BALF2 Genotyping Assay Design

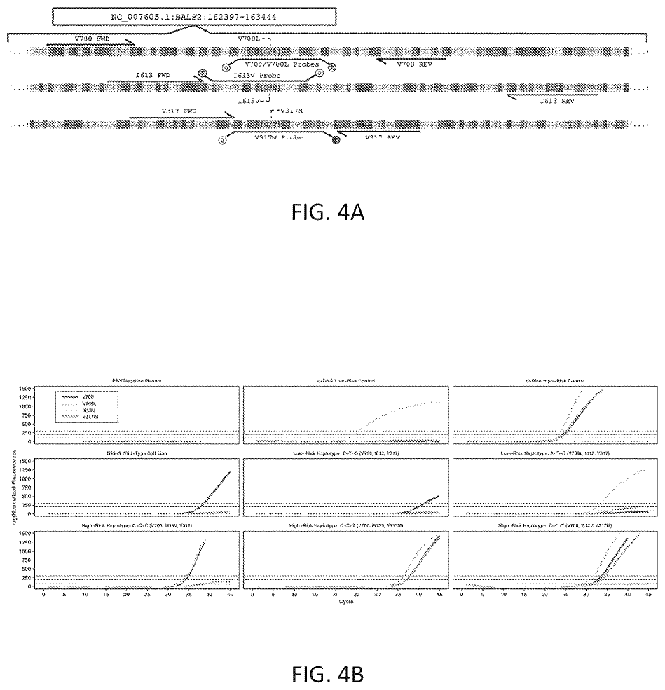

We designed a multiplex allele-specific real-time polymerase chain reaction (qPCR) genotyping assay to detect three non-synonymous polymorphisms in the EBV BALF2 gene (NCBI RefSeq NC_007605.1 Aug. 2018: V700L [162215C>A], I613V [162476T>C], V317M [163364C>T]). To permit single reaction multiplexing, we designed three conserved primer sets flanking the single nucleotide variants (SNVs), with one allele-specific propynyl-modified dual-labeled hydrolysis probe for each SNV (Biosearch Technologies, Petaluma, USA). A fourth allele-specific probe detecting the wild-type V700 allele (162215C) served as an additional internal control for samples lacking these polymorphisms (FIG. 4, Table 1).

Recognizing the potential for off-target polymorphisms in primer/probe regions, on Nov. 23, 2021 we identified 1,050 EBV GenBank sequences aligning to the EBV BALF2 region of interest (NC_007605.1:162115-163464) with ≥98% coverage. Each primer was conserved in ≥98.7% of sequences. The 162215C, 162215C>A, 162476T>C, and 163364C>T alleles were present in 78.3%, 20.9%, 37.2%, and 29.2% of sequences, respectively.

Two synthetic dsDNA gene fragments (gBlocks, Integrated DNA Technologies, Coralville, USA) served as either the NPC risk-associated (V700, I613V, V317M) or non-risk-associated (V700L, I613, V317) controls (Table 2). Supernatant from the EBV-infected B95-8 cell line served as an additional wild-type whole-virus control (ATCC, Catalog #VR-1492). Further methodological details are available in the Supplementary Methods and Tables 1-3. Assay interpretation and example amplification curves are presented in Table 4 and FIG. 4.

BALF2 Genotyping qPCR Analytical Validation

The 95% lower limit of detection (LLOD) was assessed in replicates of 20 from 0.1-5.0 copies/μL template (1.0-50.0 copies/reaction) using the risk and non-risk dsDNA controls. Any amplification crossing the fluorescence threshold was regarded as detection. Linearity was assessed from 0.0 to 6.0 log 10 copies/μL template in replicates of three. Because a minority of individuals may be latently infected with multiple distinct EBV variants, we evaluated the assay's performance with mixed risk and non-risk dsDNA controls ranging from 0-100% allele frequency at a fixed total template concentration of 100 copies/μL in replicates of three.

### Clinical Specimens

This study included human plasma specimens collected between Jul. 1, 2019 and Nov. 1, 2020 as part of routine clinical care for detection of EBV EBNA-1 by qPCR. Clinical EBV DNA qPCR was conducted as previously described.^{12, 13} Approximately 3 mL whole blood was collected in EDTA tubes, centrifuged, and at least 1.25 mL plasma aliquoted into separate tubes within six hours of collection. Total nucleic acids were extracted from 1000 μL plasma using the QIAsymphony DSP Virus/Pathogen Midi kit and eluted into 60 μL buffer AVE. After development and analytical validation of our genotyping qPCR, we retrospectively genotyped specimens meeting the following criteria: 1) EBV positive by EBNA-1 qPCR (Cₜ≤45), 2)≥20 μL residual extracted nucleic acid, and 3) highest viral load for a given patient within the study period. No diagnoses or indications for testing were excluded. Specimens were collected from patients with a range of benign and neoplastic EBV-associated disorders (Table 5).

NGS Validation of BALF2 Genotyping qPCR

We validated the genotyping qPCR assay with targeted NGS using a subset of specimens from NPC cases and controls. We sequenced a region of the BALF2 gene (NC_007605.1.162126-163483) spanning the three non-synonymous polymorphisms of interest (Supplementary Methods). Sequences with a depth of at least 10 reads at the three SNV positions of interest were accepted for interpretation. We filtered out variants with the parameter ‘QUAL<30|MQ<40|DP<10|MQOF>4|DV<3’. Specimens selected for sequencing were either the highest viral load specimen for a given patient or were specimens with residual extracted nucleic acid included in the longitudinal sequencing subset described below.

### Within-Host Longitudinal Genotyping

To assess whether EBV BALF2 haplotypes persisted over time, we longitudinally genotyped plasma specimens collected over the study period from a subset of individuals with multiple EBV-positive specimens.

Modeled NPC Mortality and Resource Utilization with Variant-Informed Screening Strategies

We estimated population-level NPC mortality reduction, resource utilization, and cost-effectiveness of BALF2 variant-informed screening strategies using a previously-validated time-inhomogeneous decision-analytic cohort model (Table 6).¹⁴ This analysis was limited to high-risk populations with endemic NPC in southern China and southeast Asia. First, we conducted a meta-analysis of three prior EBV GWAS to model BALF2 haplotype prevalence among NPC cases and non-NPC controls.⁹⁻¹¹ Thereafter, we compared variant-agnostic screening strategies from prospective studies to variant-informed d screening strategies which triage positive plasma/nasopharyngeal EBV DNA with the BALF2 genotyping qPCR. Full details regarding the model framework, population selection, screening strategies, and sensitivity analyses are detailed in the Supplementary Methods and Tables 6-11.

### Statistical Analysis

Positive percent agreement (PPA) and negative percent agreement (NPA) were reported with Clopper-Pearson score 95% binomial confidence intervals using NGS as the reference method. The 95% LLOD was calculated using probit regression for each target. Linear regression was used to fit Cₜ values against nominal concentrations. Odds ratios for high-risk haplotypes (C-C-T and/or C-C-C at positions 162215-162476-163364) were calculated using the common low-risk haplotypes as reference (sum of A-T-C and C-T-C). For EBV-positive NPC cases, the reference group includes all non-NPC controls for each individual study (present cohort, Xu et al., Hui et al., Lam et al.).⁹⁻¹¹ Fisher exact tests were used to calculate p-values for SNV and haplotype associations with NPC. For targeted NGS, the p-value threshold for statistical significance was adjusted for the number of evaluated positions using the Bonferroni correction (α=3.68×105). Analyses were conducted using the R statistical software package.

### Results

### High-Risk EBV Variants are Readily Detected in Plasma Via a Single-Reaction Genotyping Assay

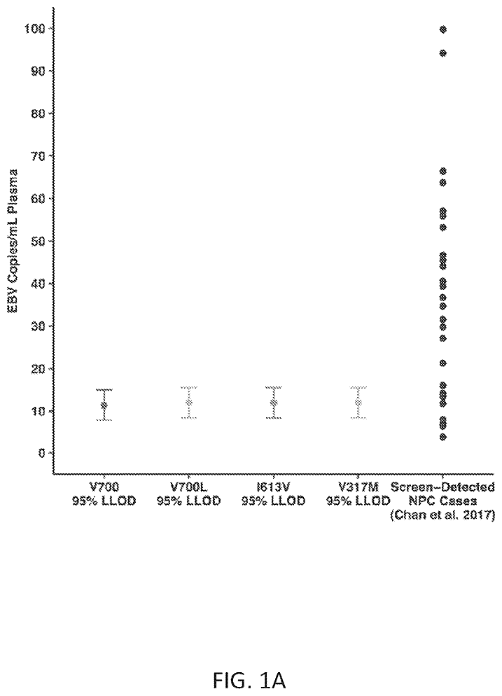

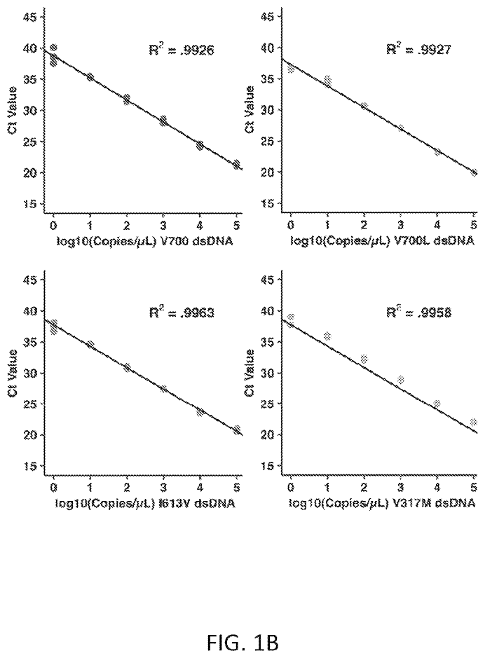

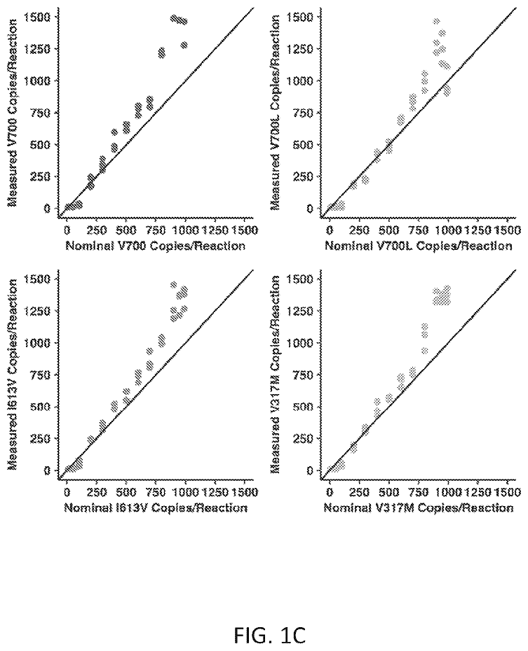

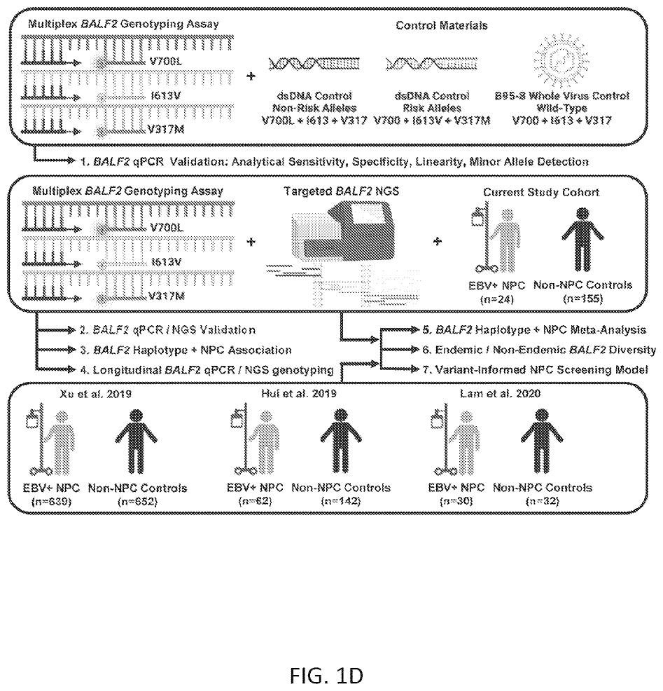

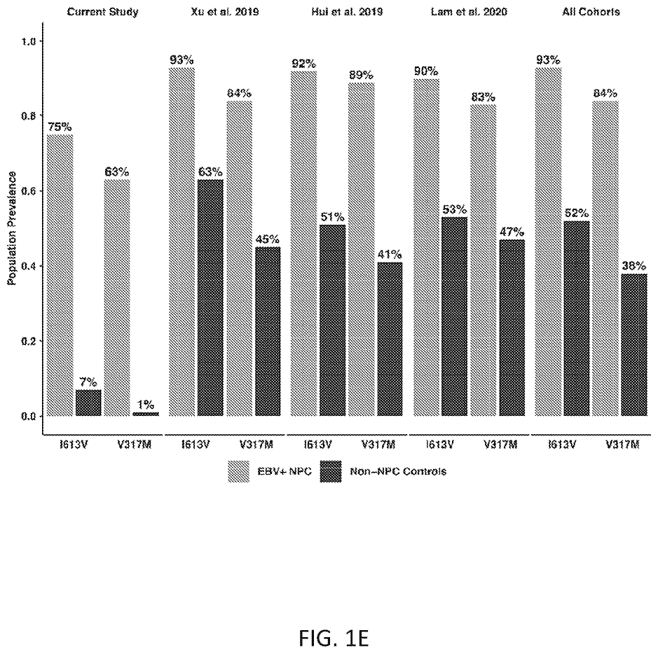

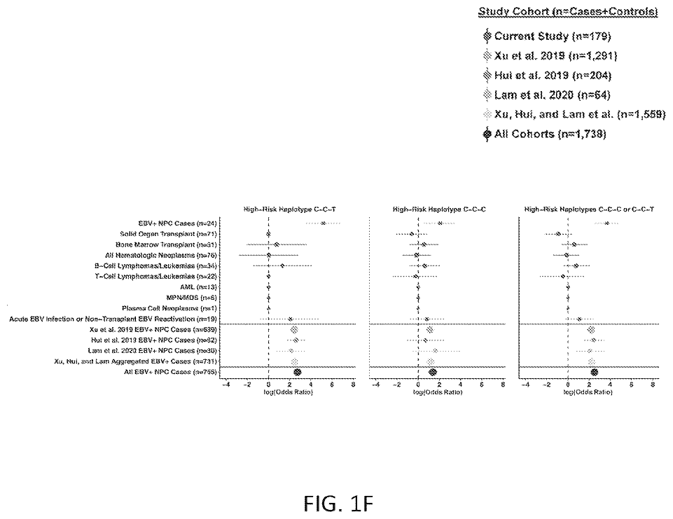

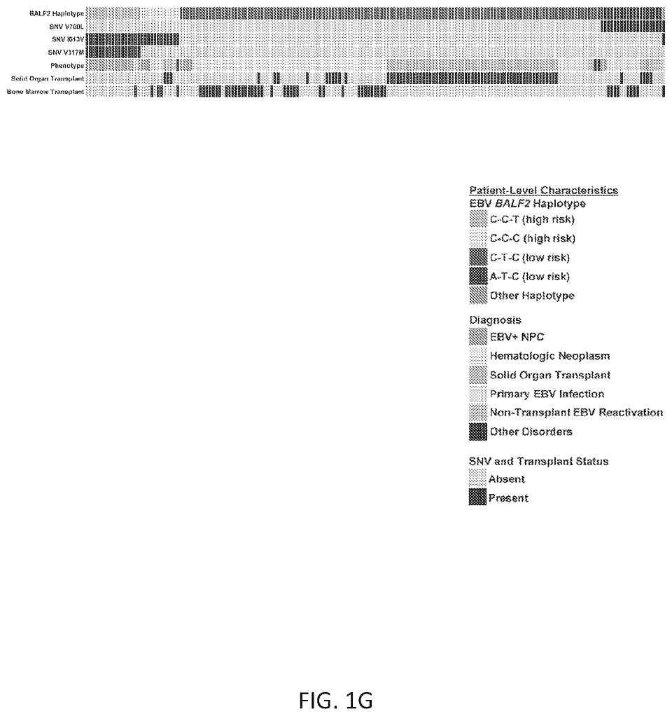

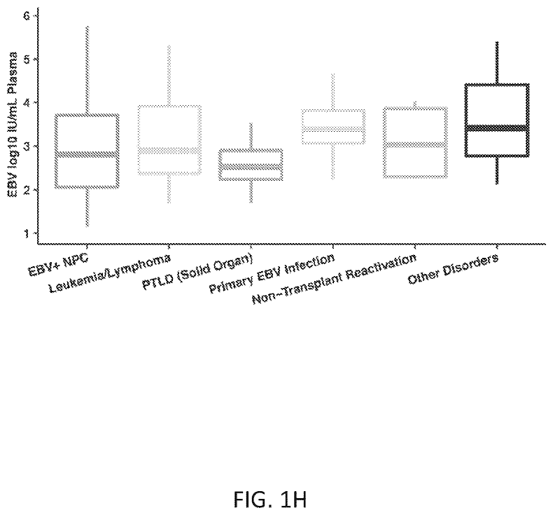

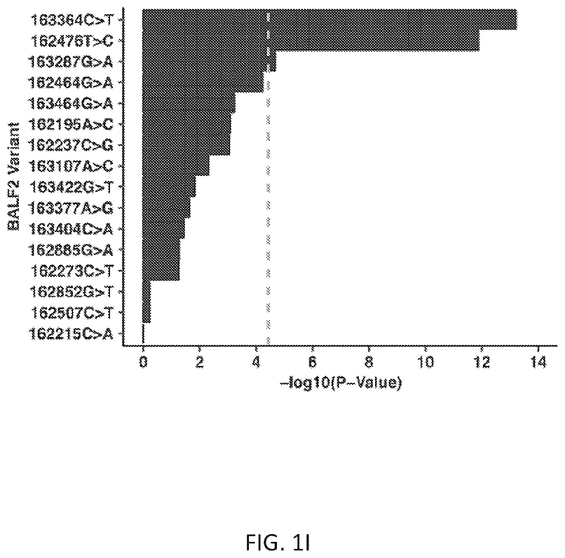

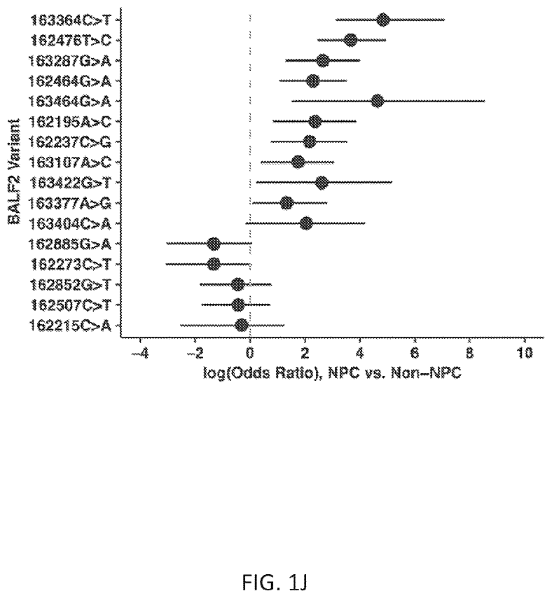

We designed and validated a multiplex allele-specific real-time polymerase chain reaction (qPCR) genotyping assay to detect three EBV BALF2 variants (V700L, I613V, V317M; Supplementary Methods, FIG. 4, Tables 1-4). The wild-type V700 allele was selected as an internal control for samples lacking these polymorphisms. The assay's 95% lower limit of detection was 2.0 copies/reaction (95% CI 1.4-2.6) with <20% coefficient of variation across six orders of magnitude (R²≥0.992, FIGS. 1A-1B, Tables 12-13). Non-specific amplification was not observed for off-target alleles, and replicates of the B95-8 wild-type whole-virus control also confirmed specificity. In mixing experiments ranging from 0-100% allele frequency, the assay detected allele frequencies as low as 10% for each of the four targets, below the host heterozygosity threshold (FIG. 1C, Table 14).⁹

Multiplex BALF2 Genotyping qPCR has Near-Perfect Concordance with Next-Generation Sequencing

We sequenced the BALF2 region in 258 clinical plasma specimens genotyped by qPCR, and 152 had adequate sequencing depth and coverage (Supplementary Methods). Samples with adequate sequencing depth and coverage had higher viral load (median 1,600 vs. 201 IU/mL, p<0.01). There was a single discordant genotyping call between qPCR and NGS. In a 43-year-old immunosuppressed woman with heart/lung transplantation, the sixth of six plasma specimens collected over 4.9 months showed qPCR loss of I613V which was detected on all five prior specimens. The specimen was sequenced and revealed the I613V mutation in 35/36 (97.2%) reads, reflecting false negative qPCR, possibly due to low viral load (EBNA-1<100 IU/mL). Positive and negative percent agreements for V700L, I613V, and V317M were otherwise 100%, and overall haplotype concordance between qPCR and NGS was 99.3% (151/152, Table 15).

BALF2 Haplotypes are Associated with NPC in a Non-Endemic Cohort

We genotyped plasma specimens from 179 unique patients in a non-endemic population, including 155 non-NPC controls and 24 EBV-positive NPC cases (Table 5, FIG. 1G). Among controls, the most common indication for plasma EBV PCR was monitoring after solid organ transplant (44%) or bone marrow transplant (33%). Seventy-six control patients (49%) had hematologic neoplasms with (66%) or without (33%) prior bone marrow transplant, including EBV-positive lymphomas/leukemias. Nineteen patients (12%) had no history of transplant or neoplasm, including ten patients with primary EBV infection. There was no significant association between plasma EBV EBNA-1 viral load and disease phenotype (FIG. 1H).

High-risk BALF2 haplotypes, defined by the presence of I613V with or without V317M, were rare among non-NPC controls (FIGS. 1D-1E, Table 16). The C-C-C and C-C-T high-risk haplotypes were present in 5.8% and 1.3% of controls, compared with 12.5% and 62.5% of NPC cases. Using the low-risk A-T-C and C-T-C haplotypes as reference, both the C-C-C (odds ratio [OR] 7.9 95% confidence interval [CI] 1.7-37.1) and C-C-T (OR 178.8, 95% CI 33.1-965.3) haplotypes were highly associated with NPC in this non-endemic population (FIG. 1F, Table 16). We observed no association between these haplotypes and other diseases, including hematologic neoplasms.

BALF2 Haplotypes are Associated with NPC in a Meta-Analysis of Endemic and Non-Endemic Cohorts

In a meta-analysis of 755 NPC cases and 981 non-NPC controls from this study and three previously-published EBV GWAS, the NPC odds ratios for the C-C-C and C-C-T haplotypes were 4.0 (95% CI 2.6-6.0) and 15.4 (95% CI 11.2-21.0), respectively (FIGS. 1D-1F, Table 16). While I613V and V317M were common (>75%) in NPC cases across cohorts, they were uncommon in non-endemic controls (7.1%) relative to endemic controls (60.5%), suggesting that variable NPC incidence could be explained by underlying BALF2 haplotype prevalence.

We also evaluated the association between clinical phenotypes and other BALF2 SNVs. For example, the previously-described 162507C>T and 162852G>T synonymous polymorphisms have been rarely observed (3%) in NPC cases but are common in endemic controls (41-43%). Among 108 unique patients with sequenced specimens, neither mutation was significantly associated with NPC. Beyond I613V and V317M, only the synonymous 163287G>A SNV reached statistical significance (FIGS. 1I-1J, Table 17). We observed no BALF2 SNVs which were significantly associated with EBV-positive leukemias/lymphomas or post-transplant lymphoproliferative disorders. We also assessed whether other SNVs were associated with high-risk BALF2 haplotypes, and identified multiple variants which were significantly correlated with I613V and V317M. For example, seven BALF2 SNVs occur with 100% frequency in the I613V/V317M haplotype and with 0-2% frequency in low-risk haplotypes (p≤1.31×10⁻⁷). This supports the hypothesis that high-risk EBV variants are transmitted locally rather than developing de novo after primary infection (Table 18).

Longitudinal Genotyping within Hosts Confirms Temporal Stability of BALF2 Haplotypes

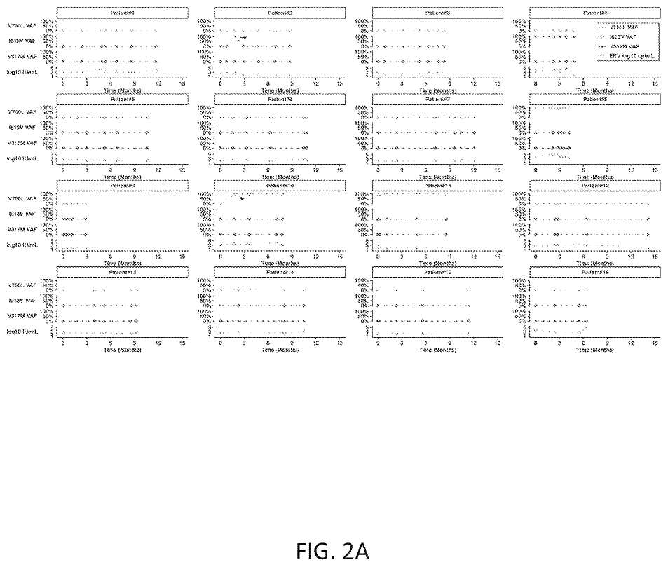

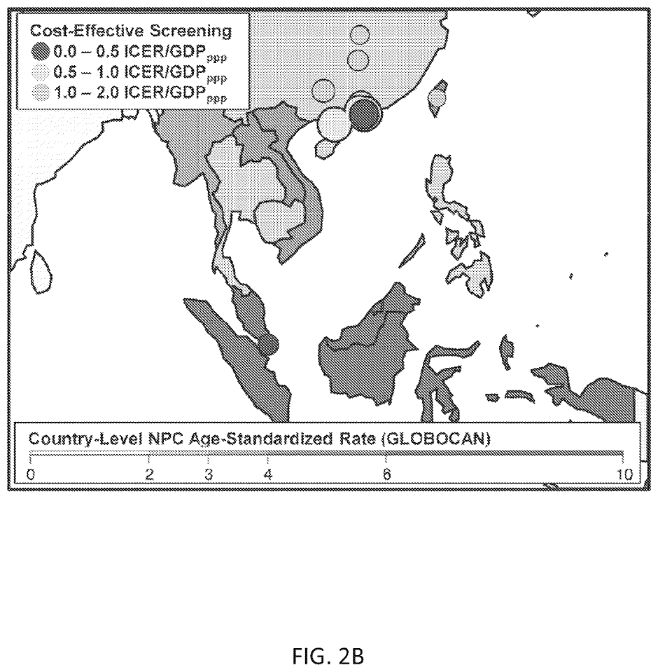

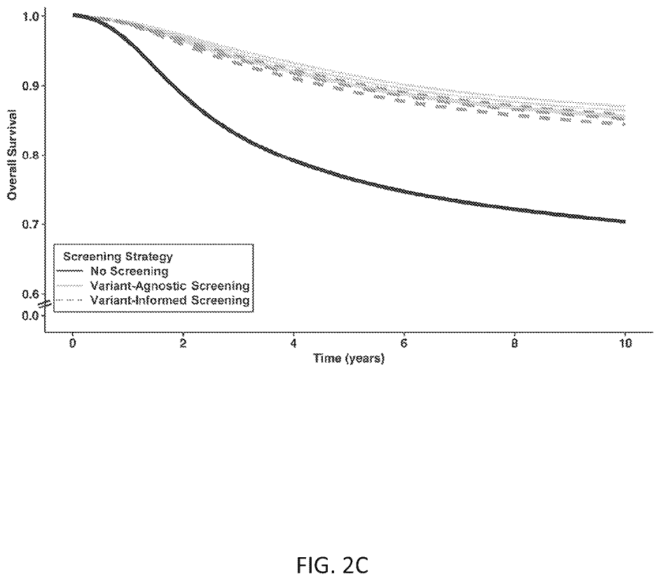

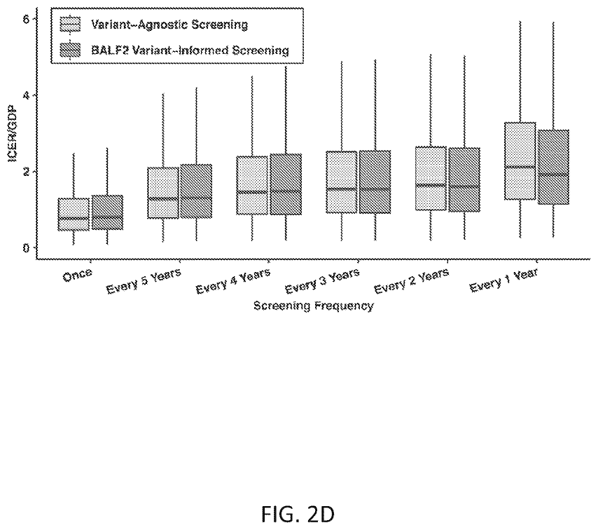

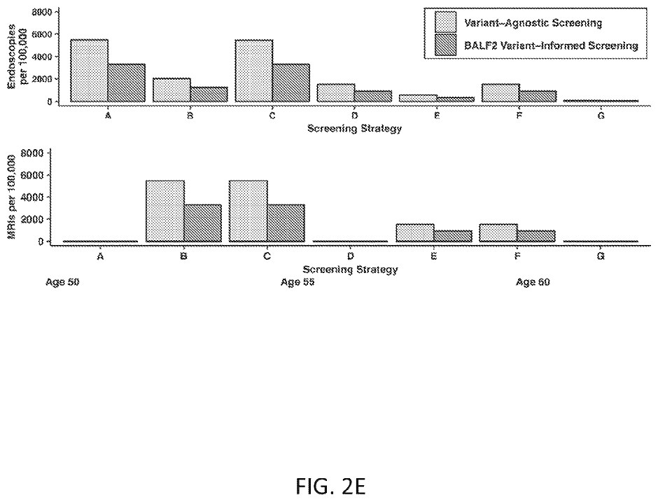

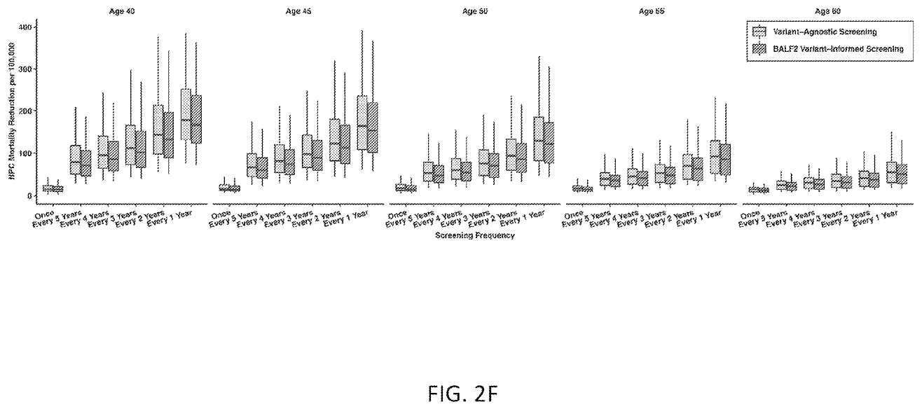

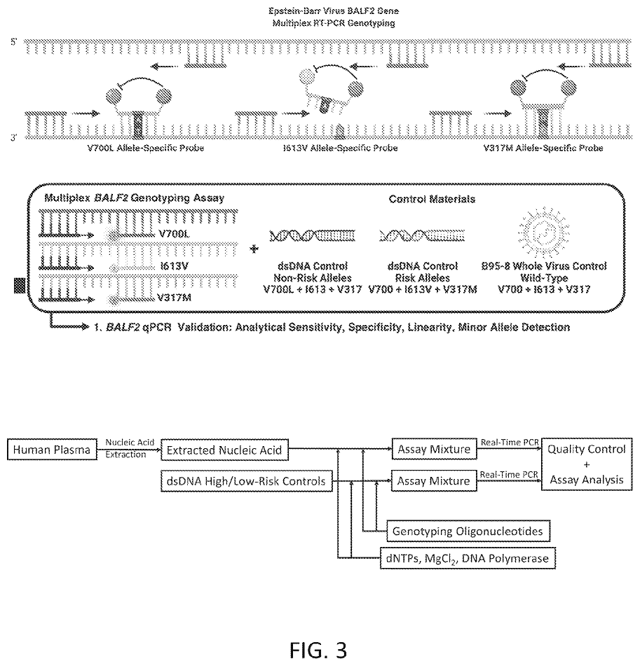

Because EBV establishes lifelong latent infection, BALF2 genotyping could facilitate once-lifetime screening triage. To assess whether EBV BALF2 haplotypes persisted over time, we genotyped 90 EBV-positive plasma specimens collected from a subset of 16 patients. These patients had a median of 5 (range, 2-7) specimens genotyped over a median period of 8.6 months (range, 2.8-13.9). Among the 90 genotyped specimens, 88 (97.8%) haplotype calls were concordant within a given individual over time (FIG. 2A). The two discordant specimens both occurred in individuals with solid organ transplantation (FIG. 2A, Patients #2 and #10) and may represent mutagenesis under immunosuppression or reactivation of distinct latent infections from the host and donor tissue.

### Variant-Informed NPC Screening Strategies Reduce False Positives and Unnecessary Procedures

We estimated population-level NPC mortality reduction, resource utilization, and cost-effectiveness of BALF2 variant-informed screening strategies using a previously-validated time-inhomogeneous decision-analytic cohort model (FIG. 4).¹⁴ Full details regarding the model framework, population selection, screening strategies, and sensitivity analyses are provided in the Supplementary Methods and Tables 6-9.

First, we conducted a meta-analysis of three prior EBV GWAS to model endemic BALF2 haplotype prevalence among NPC cases and non-NPC controls.⁹⁻¹¹ Thereafter, we compared seven variant-agnostic screening strategies from prospective studies to seven variant-informed strategies wherein positive plasma/nasopharyngeal EBV PCR are triaged using the BALF2 genotyping qPCR (Table 10). Twelve high-risk populations in southern China, Hong Kong SAR, Macao SAR, Republic of China, and Singapore met inclusion criteria (FIG. 2B, Table 11).

Variant-informed screening increased PPV by a median of 46% (range, 26-51%) with an absolute decrease in screening sensitivity of 7%. Variant-informed screening reduced referrals for endoscopy and/or MRI by approximately 40% relative to the corresponding variant-agnostic strategy (Table 10). This reduction in referrals for further screening steps averted a median of 2,969 screening visits per 100,000 subjects (Table 19). For a hypothetical cohort of 50-year-old men and women who develop NPC in southern China, 10-year survival improved from 70.4% (95% CI 68.1-72.5%) in an unscreened cohort to a median of 85.7% (range, 85.4-87.0%) with variant-agnostic screening and 85.2% (range, 84.3-85.9%) with variant-informed screening (FIG. 2C, Table 19). In the highest incidence region, the small reduction in screening sensitivity after BALF2 triage resulted in approximately 3.4 excess NPC deaths and 600 fewer false-positives requiring endoscopy/MRI per 100,000 subjects screened.

### Variant-Informed NPC Screening is Cost-Effective and Facilitates Once-Lifetime Testing

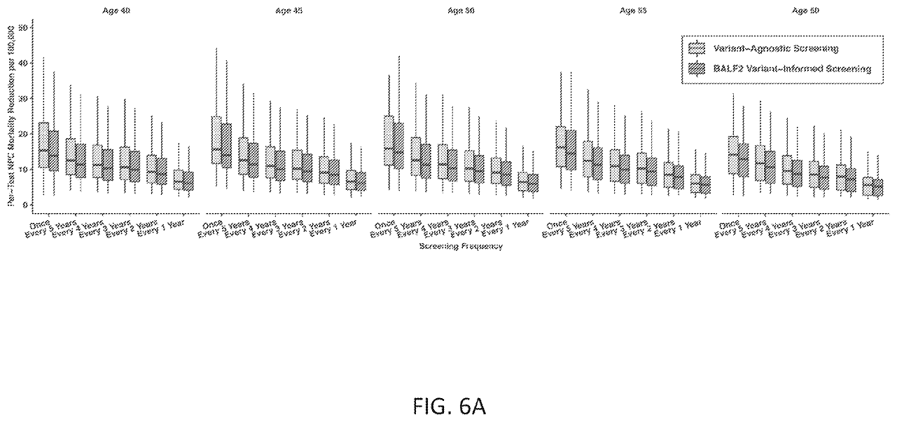

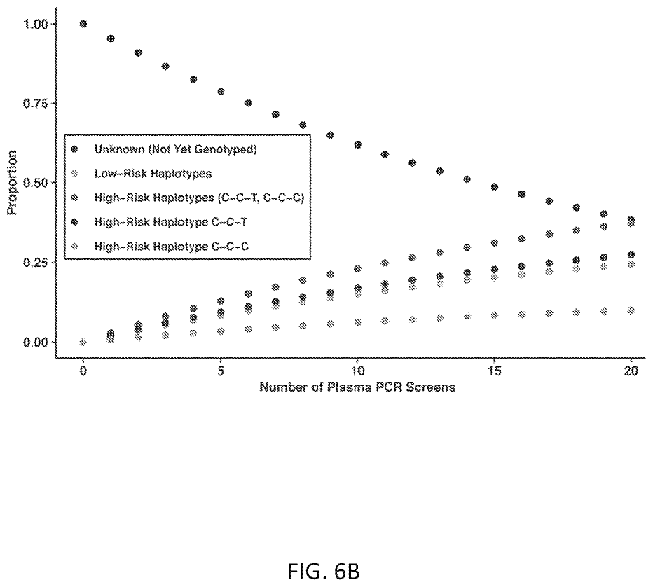

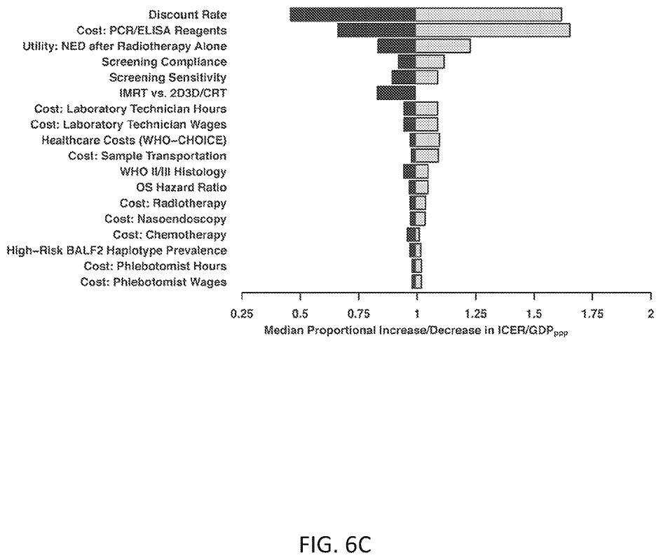

The base case screened adult men and women once at age 50 years (FIG. 2D, Tables 19-20). Variant-informed screening was cost-effective in all populations except Hengdong, China (due to lower NPC incidence). Across the 12 populations and 14 screening strategies, an initial screening age of 40-45 tended to be most cost-effective irrespective of screening interval (Table 21). Screening intervals as short as every two years could be cost-effective. Variant-informed screening became more cost-effective as the number of lifetime screens increased due to the increasing proportion of subjects known to have low-risk BALF2 haplotypes that were never subsequently screened (FIG. 6). Sensitivity analysis identified parameters that most impacted cost-effectiveness (FIG. 6, Tables 22-23).

### DISCUSSION

Existing NPC screening strategies typically utilize EBV serology or plasma PCR as the initial screening assay.^{4-7, 10} These programs achieve PPV ranging from 2-16% and do not currently leverage EBV genotyping to mitigate false positives. In light of existing laboratory screening infrastructure, we developed and validated an inexpensive single-reaction molecular diagnostic to detect high-risk EBV BALF2 haplotypes. High-risk variants were readily detectable in human plasma and were longitudinally stable within hosts. This assay had excellent analytical performance, near-perfect NGS concordance, and typically offered higher sensitivity than NGS. While prior EBV GWAS have genotyped tumor tissue or oropharyngeal swabs, our study demonstrates the feasibility of genotyping plasma EBV DNA. Modeled variant-informed screening strategies remained highly cost-effective with a 7% absolute decrease in screening sensitivity (<1% decrement in 10-year survival) and an approximate 40% decrease in referrals.

At least three recent endemic EBV-NPC GWAS have been conducted. Notably, each study observed an association between NPC and the two high-risk BALF2 haplotypes, with similar effect sizes (odds ratio 7.9-11.1) and proportion of NPC cases (90-93%). The largest study was conducted by Xu et al. and served as the basis for selection of I613V and V317M as qPCR targets.⁹ In Southern China, these variants account for 83% of the overall NPC risk, far exceeding loci in the human genome.⁸ Susceptibility loci in EBV EBER2 have also been recently identified in the Hong Kong population, but these did not meet statistical significance in Xu et al and were not evaluated in our study.^{10, 11} In contrast, a recent study of 47 Japanese patients with EBV-positive NPC reported that none harbored the I613V+V317M haplotype, whereas 21% were positive for I613V alone.¹⁵ Seven SNVs were specifically associated with non-endemic Japanese NPC. While I613V and V317M are common (60%) in endemic non-NPC controls, they are rare (12.6%) in non-endemic East Asia, Africa, and Western countries, which was replicated in our non-endemic population (7.7%) and in the Japanese cohort (9.6%). Collectively, these studies indicate that high-risk BALF2 variants are common in endemic regions but are not necessarily required for NPC carcinogenesis, and that regional EBV genomic diversity may explain differential NPC risk. Thus far, in vitro studies have implicated BALF2 variants in facilitating lytic reactivation; further functional studies are warranted to better understand the impact of I613V/V317M on NPC carcinogenesis.^{9, 15}

Modeling approaches can aid local healthcare policymakers and epidemiologists to determine the optimal balance between screening resources, complexity, and performance. If BALF2 genotyping were incorporated into screening algorithms, laboratories could consider screening with qPCR detecting BamHI-W, BALF2 V317M, BALF2 I613V, and a single-copy conserved target. This approach would require minimal additional laboratory costs and could decrease referrals for subsequent screening steps by approximately 40%. While a multiplex genotyping qPCR is advantageous in its low cost/complexity, we anticipate superior discrimination with more complex NGS-based variant panels.¹⁰ Although not evaluated in our study, once-lifetime EBV genotyping using oropharyngeal specimens warrants further evaluation.

There are multiple limitations to our study. First, we validated our assay using specimens from a non-endemic population that had few healthy controls. Although we observed no association between BALF2 haplotypes and other non-NPC diseases, it is possible that non-NPC controls had different haplotype prevalence relative to the healthy population. Second, the ability to triage individuals once in their lifetime with BALF2 genotyping is predicated on haplotype stability over time and absence of multiple EBV co-infections. Because longitudinal specimens were not available over a years- or decades-long period, it is uncertain whether haplotypes may change over longer time scales. Third, BALF2 haplotype distributions for NPC cases and controls were derived from a meta-analysis of three studies which predominantly included subjects in southern China. The degree to which these distributions vary within southeast Asia is unknown, and would impact effective screening sensitivity.

### CONCLUSIONS

Approximately 93% of endemic nasopharyngeal carcinoma harbors high-risk EBV BALF2 haplotypes. These haplotypes are stable over time within hosts and readily detectable in plasma using an inexpensive single-reaction multiplex genotyping assay. The BALF2 I613V and V317M polymorphisms are rare in non-endemic controls, supporting the hypothesis that regional EBV genomic diversity contributes to differential NPC risk worldwide. Triaging subjects who test positive for plasma/nasopharyngeal EBV DNA using BALF2 genotyping could substantially reduce referrals for more complex and expensive endoscopy/MRI. Across seven prospectively-evaluated screening strategies in 12 high-risk endemic populations, these variant-informed strategies maintain high screening sensitivity while averting 40% of referrals for endoscopy/MRI. In suitable populations, this may be a low-cost and readily accessible alternative to higher-complexity triage algorithms, and could identify low-risk individuals who require no further lifetime screening.

### REFERENCES

1. Dunmire S K, Verghese P S, Balfour H H. Primary Epstein-Barr virus infection. J Clin Virol Off Publ Pan Am Soc Clin Virol. 2018; 102:84-92. doi: 10.1016/j.jcv.2018.03.001
2. Sung H, Ferlay J, Siegel R L, et al. Global Cancer Statistics 2020: GLOBOCAN Estimates of Incidence and Mortality Worldwide for 36 Cancers in 185 Countries. CA Cancer J Clin. 2021; 71 (3): 209-249. doi: 10.3322/caac.21660
3. Pan J J, Ng W T, Zong J F, et al. Proposal for the 8th edition of the AJCC/UICC staging system for nasopharyngeal cancer in the era of intensity-modulated radiotherapy. Cancer. 2016; 122 (4): 546-558. doi: 10.1002/cncr.29795
4. Chan K C A, Woo J K S, King A, et al. Analysis of Plasma Epstein-Barr Virus DNA to Screen for Nasopharyngeal Cancer. N Engl J Med. 2017: 377 (6): 513-522. doi: 10.1056/NEJMoa1701717
5. Ji M F, Sheng W, Cheng W M, et al. Incidence and mortality of nasopharyngeal carcinoma: interim analysis of a cluster randomized controlled screening trial (PRO-NPC-001) in southern China. Ann Oncol. 2019; 30 (10): 1630-1637. doi: 10.1093/annonc/mdz231
6. Lam W K J, Jiang P, Chan K C A, et al. Methylation analysis of plasma DNA informs etiologies of Epstein-Barr virus-associated diseases. Nat Commun. 2019; 10 (1): 3256. doi: 10.1038/s41467-019-11226-5
7. Chen Y, Zhao W, Lin L, et al. Nasopharyngeal Epstein-Barr Virus Load: An Efficient Supplementary Method for Population-Based Nasopharyngeal Carcinoma Screening. PloS One. 2015; 10 (7): e0132669. doi: 10.1371/journal.pone.0132669
8. Bei J X, Su W H, Ng C C, et al. A GWAS Meta-analysis and Replication Study Identifies a Novel Locus within CLPTM1L/TERT Associated with Nasopharyngeal Carcinoma in Individuals of Chinese Ancestry. Cancer Epidemiol Biomark Prev Publ Am Assoc Cancer Res Cosponsored Am Soc Prev Oncol. 2016; 25 (1): 188-192. doi: 10.1158/1055-9965.EPI-15-0144
9. Xu M, Yao Y, Chen H, et al. Genome sequencing analysis identifies Epstein-Barr virus subtypes associated with high risk of nasopharyngeal carcinoma. Nat Genet. 2019; 51 (7): 1131-1136. doi: 10.1038/s41588-019-0436-5
10. Lam W K J, Ji L, Tse O Y O, et al. Sequencing Analysis of Plasma Epstein-Barr Virus DNA Reveals Nasopharyngeal Carcinoma-Associated Single Nucleotide Variant Profiles. Clin Chem. 2020: 66 (4): 598-605. doi: 10.1093/clinchem/hvaa027
11. Hui K F, Chan T F, Yang W, et al. High risk Epstein-Barr virus variants characterized by distinct polymorphisms in the EBER locus are strongly associated with nasopharyngeal carcinoma. Int J Cancer. 2019; 144 (12): 3031-3042. doi: 10.1002/ijc.32049
12. Abeynayake J, Johnson R, Libiran P, et al. Commutability of the Epstein-Barr virus WHO international standard across two quantitative PCR methods. J Clin Microbiol. 2014; 52 (10): 3802-3804. doi: 10.1128/JCM.01676-14
13. Le Q T, Zhang Q, Cao H, et al. An international collaboration to harmonize the quantitative plasma Epstein-Barr virus DNA assay for future biomarker-guided trials in nasopharyngeal carcinoma. Clin Cancer Res Off J Am Assoc Cancer Res. 2013; 19 (8): 2208-2215. doi: 10.1158/1078-0432.CCR-12-3702
14. Miller J A, Le Q T, Pinsky B A, Wang H. Cost-Effectiveness of Nasopharyngeal Carcinoma Screening With Epstein-Barr Virus Polymerase Chain Reaction or Serology in High-Incidence Populations Worldwide. Natl J Cancer Inst. 2021: 113 (7): 852-862. doi: 10.1093/jnci/djaa198
15. Kondo S, Okuno Y, Murata T, et al. EBV genome variations enhance clinicopathological features of nasopharyngeal carcinoma in a non-endemic region. Cancer Sci. Published online Apr. 29, 2022. doi: 10.1111/cas. 15381

### Supplementary Methods

### Multiplex BALF2 Genotyping Assay Design

We designed a multiplex allele-specific real-time polymerase chain reaction (qPCR) genotyping assay to detect three non-synonymous polymorphisms in the EBV BALF2 gene (NCBI RefSeq NC_007605.1 Aug. 2018: V700L [162215C>A], I613V [162476T>C], V317M [163364C>T]). We selected the wild-type V700 allele (162215C) to serve as an additional internal control for samples lacking any of these polymorphisms.

To permit single reaction multiplexing, we designed three conserved primer sets flanking the single nucleotide variants (SNVs), with one allele-specific propynyl-modified dual-labeled hydrolysis probe for each SNV (Biosearch Technologies. Petaluma, USA). A fourth allele-specific probe detecting V700 served as an internal control (FIG. 4). Each of the four allele-specific probes was designed to maximize the mismatch ΔTm while maintaining probe specificity and a sufficiently high annealing temperature (Table 1). Recognizing the potential for off-target polymorphisms in primer/probe regions, on Nov. 23, 2021 we identified 1,050 EBV GenBank sequences aligning to the EBV BALF2 region of interest (NC_007605.1:162115-163464) with ≥98% coverage. Each primer was conserved in ≥98.7% of sequences. The 162215C, 162215C>A, 162476T>C, and 163364C>T alleles were present in 78.3%, 20.9%, 37.2%, and 29.2% of sequences, respectively.

Two synthetic dsDNA gene fragments (gBlocks, Integrated DNA Technologies, Coralville, USA) served as either the NPC risk-associated (V700, I613V, V317M) or non-risk-associated (V700L, I613, V317) controls (Table 2). These controls were diluted in Tris-EDTA buffer (10 mM Tris, 1 mM EDTA). Supernatant from the EBV-infected B95-8 cell line served as an additional wild-type whole-virus control (ATCC, Catalog #VR-1492).

Real-time PCR was performed using 12.5 μL FastStart TaqMan Probe Master Mix (Roche, Switzerland), 2.0 μL primer/probe mix, 0.5 μL nuclease-free water, and 10.0 μL template (FIG. 4, Table 3). All experiments were conducted using a BioRad CFX96 real-time PCR instrument (BioRad, Hercules, CA, USA). Cycling conditions were 95° C. for 4:00 and then 45 cycles of 95° C. for 00:30, 63.0° C. for 00:30, and 72° C. for 00:30. All qPCR experiments included a no-template control (nuclease-free water), a wild-type whole-virus control (3.98×10⁴ IU/mL B95-8 supernatant extracted into AVE buffer), synthetic dsDNA risk control (10⁴ copies/μL template), and synthetic dsDNA non-risk control (10⁴ copies/μL template). Annealing temperature was optimized with a temperature gradient. Fluorescence was collected in all channels. Fixed fluorescence thresholds of 200 relative fluorescence units ([RFU], V700-FAM), 100 RFU (V700L-CAL560), 300 RFU (I613V-CAL610), and 300 RFU (V317M-Q670) were used to determine each target's threshold cycle (Cₜ). Assay interpretation and example amplification curves are presented in Table 4 and FIG. 4.

BALF2 Genotyping qPCR Analytical Validation

The 95% lower limit of detection (LLOD) was assessed in replicates of 20 from 0.1-5.0 copies/μL template (1.0-50.0 copies/reaction) using the risk and non-risk dsDNA controls (Table 12). Any amplification crossing the fluorescence threshold was regarded as detection. Linearity was assessed from 0.0 to 6.0 log 10 copies/μL template in replicates of three (Table 13). Because a minority of individuals may be latently infected with multiple distinct EBV variants, we evaluated the assay's performance with mixed risk and non-risk dsDNA controls ranging from 0-100% allele frequency at a fixed total template concentration of 100 copies/μL in replicates of three (Table 14).

### Clinical Specimens

This study included human plasma specimens collected between Jul. 1, 2019 and Nov. 1, 2020 as part of routine clinical care for detection of EBV EBNA-1 by qPCR. Clinical EBV DNA qPCR was conducted as previously described.^{1, 2} Approximately 3 mL whole blood was collected in EDTA tubes, centrifuged, and at least 1.25 mL plasma aliquoted into separate tubes within six hours of collection. Total nucleic acids were extracted from 1000 μL plasma using the QIAsymphony DSP Virus/Pathogen Midi kit and eluted into 60 μL buffer AVE. Nucleic acid extraction and clinical testing were conducted at the Stanford Clinical Virology Laboratory, which serves tertiary-care academic hospitals and affiliated outpatient facilities in the San Francisco Bay Area.

After development and analytical validation of our genotyping qPCR, we retrospectively genotyped specimens meeting the following criteria: 1) EBV positive by EBNA-1 qPCR (Cₜ≤45), 2)≥20 μL residual extracted nucleic acid, and 3) highest viral load for a given patient within the study period. No diagnoses or indications for testing were excluded. Specimens were collected from patients with a range of benign and neoplastic EBV-associated disorders (Table 5). This study was conducted with Stanford University institutional review board approval.

NGS Validation of BALF2 Genotyping qPCR

We validated the genotyping qPCR assay with targeted NGS using a subset of specimens from NPC cases and controls (Table 15). We sequenced a portion of the BALF2 gene (NC_007605.1.162126-163483) spanning the three non-synonymous polymorphisms of interest. Specimens selected for sequencing were either the highest viral load specimen for a given patient or were specimens with residual extracted nucleic acid included in the longitudinal sequencing subset described below.

We designed 28 conserved primers to generate 14 small overlapping amplicons for targeted enrichment. Given the small insert size (median 153 bases, range 93-219). fragmentation was not performed and adapter sequences were included at the 5′ end of each primer. Each of the 28 primers was separately synthesized with read 1 and read 2 adapters to facilitate paired-end sequencing. Even- and odd-numbered primer sets were pooled separately.

Extracted nucleic acid was amplified by PCR in two reactions using 12.5 μL LongAmp Taq 2× Mastermix Hot Start (New England BioLabs, Ipswich, MA), 0.3125 μL of 2 μM read 1 odd or even-numbered primer sets (25 nM each primer), 0.3125 μL of 2 μM read 2 odd or even-numbered primer sets (25 nM each primer), 1.875 μL nuclease-free water, and 10.0 μL template. Target enrichment PCR conditions were 94° C. for 0:30, and then 45 cycles of 94° C. for 00:30, 57.0° C. for 00:30, and 65° C. for 01:00 prior to final 65° C. extension for 10:00.

Libraries were prepared using NEBNext library preparation reagents for Illumina sequencing instruments (New England BioLabs, Ipswich, MA). Half of the products from the two PCR reactions (25 μL total) were pooled and purified with 45 μL (1.8× ratio) AMPure XP beads (Beckman Coulter, Brea, CA) and 80% ethanol, then resuspended into 25 μL AVE elution buffer. Libraries were then indexed using dual index primers in a 50 μL reaction containing 25 μL NEBNext® Ultra™ II Q5® Master Mix (M0544), 1 μL index 1 (i7) primer, 1 μL index 2 (i5) primer, and 23 μL amplified DNA. Thermocycler settings for indexing were: 98° C. for 00:30, then 12 cycles of 98° C. for 00:10 and 65.0° C. for 01:15 prior to final extension at 65.0° C. for 05:00.

Indexed libraries were then purified with 90 μL (1.8× ratio) AMPure XP beads and 80% ethanol, then resuspended into 40 μL AVE elution buffer. Individual indexed specimens were then pooled and normalized (1-20 uL each) based on EBNA-1 qPCR viral load. The pooled library was then purified with a 1:1 ratio of AMPure XP beads and 80% ethanol, then resuspended into 40 μL AVE elution buffer. Pooled indexed library fragment size was measured with a BioAnalyzer 2100. The library was then quantified using the Qubit dsDNA broad range assay and diluted to 15 pM. Each sequencing run contained one no template control, one wild-type B95-8 control, and up to 94 clinical specimens. Libraries were sequenced on an Illumina MiSeq using paired-end 150-cycle sequencing using the MiSeq Nano reagent kit V2.

Trimmed reads were assembled using the Burrows-Wheeler Aligner and variants were called using BCFTools using NC_007605.1 as reference. Sequences with a depth of at least 10 reads at the three SNV positions of interest were accepted for interpretation. We filtered out variants with the parameter ‘QUAL<30|MQ<40|DP<10|MQOF>4|DV<3’.

Modeled NPC Mortality and Resource Utilization with Variant-Informed Screening Strategies

We estimated population-level nasopharyngeal carcinoma (NPC) mortality reduction, resource utilization, and cost-effectiveness of BALF2 variant-informed screening strategies using a previously-validated time-inhomogeneous decision-analytic cohort model (Table 6).³ This was limited to high-risk populations with endemic NPC in southern China and southeast Asia.

First, we conducted a meta-analysis of three prior EBV genome-wide association studies (GWAS) to model BALF2 haplotype prevalence among NPC cases and non-NPC controls (Table 16).⁴⁻⁶ Thereafter, we compared variant-agnostic screening strategies from prospective studies to variant-informed screening strategies which triage positive plasma/nasopharyngeal EBV DNA with the BALF2 genotyping qPCR (Table 10).

### Study Populations and Incidence Data

We selected 12 populations to compare variant-informed and variant-agnostic screening (Table 23). These populations satisfied the following criteria:
Populations with endemic EBV-associated NPC.
Populations with known EBV BALF2 haplotype distributions from association studies.
Populations in regions with ongoing or previously-conducted NPC screening programs.
Populations with available age and sex-specific incidence rates published in national cancer registries or in the World Health Organization's Cancer Incidence in Five Continents (CI5 X-XI).⁷⁻¹²
Populations with available megavoltage radiotherapy services as reported in the IAEA DIRAC database. ¹³

Each population had economic characteristics and sufficiently high NPC incidence such that NPC screening was likely to be cost-effective as estimated in a prior study.³ Populations in southern China (Hong Kong SAR, Macao SAR, Guangdong, Guangxi, Hunan), Singapore, and the Republic of China met these criteria. The most recent incidence rates for these 12 populations were obtained from CI5 X-XI, the Hong Kong Cancer Registry, the Singapore Cancer Registry, and the Republic of China Cancer Registry^{7, 8, 10-12, 14}, which were adjusted for the proportion of incident NPC cases in each region which were WHO type II/III (Table 11). In Singapore, ethnicities with high NPC incidence were studied separately from pooled national data. For men and women, the age-standardized rates for these 12 populations ranged from 3.6-16.6 cases/100,000 life-years.

### Decision Analytic Model

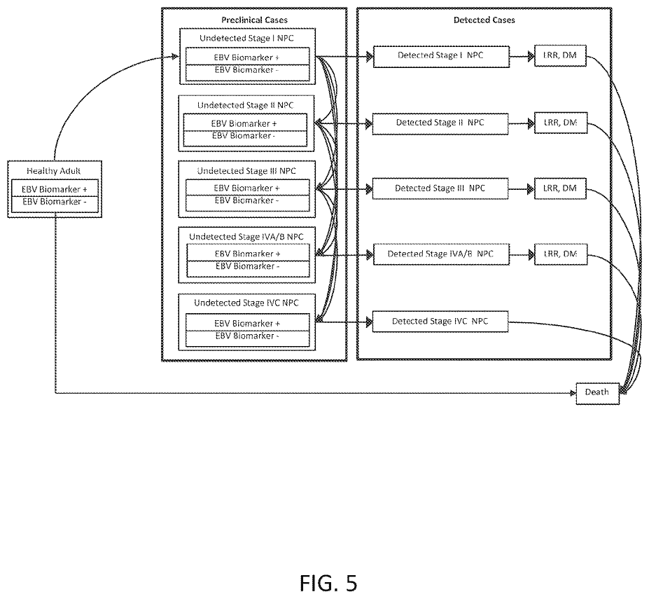

We utilized a previously-validated time-inhomogeneous decision-analytic cohort model to study NPC mortality reduction and resource utilization of BALF2 variant-informed screening strategies.³ Full details regarding development and validation of this model are available from the original publication and reports from prospective screening trials. Briefly, subjects enter model at the age of a single screening intervention and are followed until death from NPC or other causes over a lifetime horizon. Heath states include perfect health, five separate stages of undetected NPC (AJCC 7 stages I, II, III, IVA/B, IVC), five separate stages of detected incident NPC (AJCC 7 stages I, II, III, IVA/B, IVC), remission with no evidence of disease after definitive (chemo) radiotherapy, locoregional recurrence or distant metastasis under treatment with indefinite palliative chemotherapy, and death (FIG. 5).

Each AJCC 7 nonmetastatic stage of NPC (I-IVB) undergoes definitive therapy with radiotherapy or chemoradiotherapy, while de novo metastatic disease (IVC) undergoes treatment with indefinite palliative gemcitabine/platinum chemotherapy with or without one-time palliative radiotherapy. Transition probabilities between states were calibrated using epidemiologic estimates from incidence databases and from prospective screening studies identified in a systematic literature review.³

Patients diagnosed with nonmetastatic NPC undergo treatment and enter the remission state, and then could either die from background mortality or develop a locoregional and/or distant recurrence prior to death from NPC. Using the R heemod package, subjects who developed NPC enter the state of undiagnosed stage I disease and could progress to more advanced stages of undetected disease.¹⁵ Alternatively, each undiagnosed stage of NPC could instead present symptomatically with detected NPC, which is identical to observed stage-specific incidence rates in unscreened populations. At the time of onetime screening, a true positive (based on strategy-specific sensitivity) results in immediate detection at that stage. False negatives and unscreened cases resulted in continued stage progression until usual symptomatic detection. For each screening step, a positive result (whether true positive or false positive) results in testing at the next screening step (e.g., nasoendoscopy after positive serology). There was also the usual risk of death or remaining in the same state for each given state.

For each population, background mortality was derived from WHO global health observatory country-, age- and sex-specific mortality rates.¹⁶

For the base case, men and women are screened once at age 50 years, which was closest to the median age in most screening studies. Interval screening, variable initial screening ages, and the exclusive screening of men were studied in sensitivity analyses. For interval screening, all screening ended after age 70.

### Recurrence and Survival Estimates

Among patients with detected NPC (whether by screening or symptomatic presentation), stage-specific time-dependent transition probabilities to LRR, DM, or death were derived from survival models trained using extracted individual patient data from 9,864 individuals included in 17 studies and treated in China with standard-of-care intensity-modulated (chemo) radiotherapy (stages IVA/B) or gemcitabine/platinum chemotherapy (stage IVC) after MRI staging.³ These stage-specific recurrence and survival models were then externally validated using individual patient data (n=729) extracted from nine studies from patients treated with IMRT and MRI staging outside of China. These 26 total studies were identified in a previously-conducted systematic review.³

### Health State Utilities

Time-dependent health utilities during and after radiotherapy alone were derived from the experimental (IMRT) arm of a randomized trial of patients with NPC undergoing definitive radiotherapy alone (Table 6).^{17, 18} Disutilities for the addition of chemotherapy to radiotherapy were derived from a series of patients with NPC treated in Taiwan who completed the EORTC QLQ-C30 questionnaires.¹⁹ Health utilities for patients undergoing indefinite chemotherapy for locoregional or distant recurrence were derived from the control arm of the Checkmate 141 (week 15).²⁰ Responses from the EORTC QLQ-C30 were mapped to EQ-5D values using a model derived from a population of patients with head and neck cancers.²¹

For the base case, we assumed that subjects who screen as false positives by any strategy had a health state of 1.000 based on literature from lung cancer screening.²² However, we also modeled the impact of a one-month decrement in healthy utility to 0.900 for false positives based on quality of life from the breast cancer screening literature.²³ For true positives (detected NPC cases), we assigned a one-month pre-treatment decrement in health utility to 0.886 based on prior literature.²⁴ For the probabilistic sensitivity analysis, we created joint uncertainty distributions for health state utilities to respect preference order for certain health states over others based on base case values and clinical experience, as previously described.³

### Workup, Staging, and Treatment Assumptions

Initial workup and management were based upon the National Comprehensive Cancer Network 2021 head and neck cancer guidelines (Tables 7-9).²⁵ We assumed that patients who developed NPC underwent clinical evaluation by a physician, endoscopic examination, needle biopsy, pathology review, CT and MRI of the head and neck, and CT of the chest to evaluate for metastatic disease (Table 7).²⁵ Given its limited availability, we assumed that PET/CT was not used for staging, but studied its use in sensitivity analysis. For patients who underwent MRI of the head and neck as part of screening, this was not repeated during diagnostic workup. Clinical staging was defined by the American Joint Committee on Cancer (AJCC) 7^{th} edition staging system used by Chan et al. to stage screen-detected cases.²⁶

We assumed that patients with stage II-IVB disease were treated with concurrent chemoradiotherapy followed by adjuvant chemotherapy, acknowledging clinical equipoise and practice pattern differences in the use of concurrent, concurrent/adjuvant, and induction/concurrent chemoradiotherapy.²⁷⁻³⁰ Patients with cT1N0M0 NPC were treated with definitive radiotherapy alone. We extracted country-level data from the IAEA DIRAC database of radiation therapy facilities (as of June 2020).¹³ Given widespread availability of intensity-modulated radiotherapy in the 12 included populations, we assumed that all radiotherapy was intensity-modulated and image-guided to a total dose of 70 Gy in 35 daily fractions (Table 7).²⁵ We studied a range of fractionation schemes (30-40 fractions) in sensitivity analysis. While undergoing radiotherapy, patients were evaluated by a radiation oncologist once weekly for side effect management. Multidisciplinary consultation, restaging imaging, endoscopic exam, and tissue confirmation were performed for patients who developed local or distant recurrence after definitive therapy (Table 8).

For patients receiving chemotherapy, a separate medical oncology consultation was included, with one follow-up visit per cycle (Table 9). For patients with stage II-IVB disease, concurrent chemotherapy was cisplatin 100 mg/m² every three weeks, and adjuvant chemotherapy was cisplatin 80 mg/m² (day 1) with continuous-infusion fluorouracil 1000 mg/m²/d (days 1-4) every four weeks for three cycles.²⁷ Patients with either recurrent or de novo metastatic (stage IVC) disease were treated with chemotherapy alone (cisplatin 80 mg/m² day 1 with gemcitabine 1000 mg/m² days 1 and 8, every three weeks).³¹ Additionally, we assumed that half of patients who developed a locoregional recurrence underwent palliative 2D/3D conformal radiotherapy to a dose of 30 Gy in 10 fractions (with an associated short-term decrement in health utility), and studied a range of palliative radiotherapy utilization (10-90%) in sensitivity analysis. Although highly-selected patients in high-resource settings are clinically treated with definitive-intent re-irradiation and/or surgical resection, we did not incorporate these salvage therapies into our model due to the small number of patients eligible for these treatments. We furthermore assumed that radiotherapy was not delivered in the de novo metastatic setting. Basic labs were drawn and supportive care (antiemetics, hydration) was administered with each cycle of chemotherapy.

### Variant-Agnostic and Variant-Informed Screening Strategies

### BALF2 Variant-Agnostic Strategies

We studied a set of seven prospectively-evaluated screening strategies and assessed the impact of adding EBV BALF2 genotyping to triage samples positive for EBV DNA (Table 10).^{4-6, 32-36} These seven strategies were selected due to dependance upon plasma or nasopharyngeal EBV DNA PCR, as residual extracted nucleic acid could be subjected to BALF2 genotyping. These strategies included combinations of single-antigen serology, plasma or nasopharyngeal EBV DNA PCR, and nasoendoscopy/MRI. We did not study the impact of BALF2 genotyping on strategies relying only on serology. Screened participants from these trials resided in Zhongshan, Sihui, Wuzhou, and Hong Kong.

We compared seven unique onetime screening strategies against no screening in each studied population. These seven variant-agnostic strategies were:
A₀: EBV BamHI-W DNA plasma PCR [Any Amplification]→Endoscopy
B₀: EBV BamHI-W DNA plasma PCR [Any Amplification]→MRI nasopharynx
C₀: EBV BamHI-W DNA plasma PCR [Any Amplification]→Endoscopy+MRI nasopharynx
D₀: EBV BamHI-W DNA plasma PCR [Any Amplification]→EBV BamHI-W DNA plasma PCR [Any Amplification]→Endoscopy
E₀: EBV BamHI-W DNA plasma PCR [Any Amplification]→EBV BamHI-W DNA plasma PCR [Any Amplification]→MRI nasopharynx
F₀: EBV BamHI-W DNA plasma PCR [Any Amplification]→EBV BamHI-W DNA plasma PCR [Any Amplification]→Endoscopy+MRI nasopharynx
G₀: Serum EBV VCA IgA [>1:5]→EBV BamHI-W DNA nasopharyngeal swab PCR [Mean+2SD]→Endoscopy

Studies were identified via a prior systematic review of the literature.³ Identical screening strategies across studies were pooled and weighted by the number of cases and controls using contingency tables.

In Chan et al. (2013), 1,318 men and women age 40-60 underwent plasma EBV BamHI-W PCR (any amplification) and serologic testing by ELISA for EBV IgA VCA (ratio >1.0).³⁵ All patients with a positive initial test by either method underwent nasoendoscopy and a follow-up plasma EBV DNA PCR. We studied the relative performance of plasma EBV DNA PCR→nasoendoscopy (strategy A) and plasma EBV DNA PCR→PCR→nasoendoscopy (strategy D).

In Chan et al. (2017), 20,174 ethnically Chinese men who were 40-62 years of age residing in Hong Kong underwent screening from 2013-2016.³² Subjects underwent an initial screen for plasma EBV DNA via a quantitative PCR assay amplifying the BamHI-W fragment of the EBV genome.^{2, 37} In subjects with a positive initial screen (any amplification), the PCR was repeated one month later. Those with persistently-positive results (1.5%) underwent endoscopic examination and MRI of the nasopharynx. All screened adults were interviewed annually, and ultimately a total of 35 cases of NPC were detected within one year of screening. Only one of these 35 patients had a negative screen, and presented with stage II NPC within four months of enrollment, corresponding to a sensitivity of 97.1% and specificity of 98.6%. Only 32 unnecessary biopsies were performed among the 20,174 participants (0.2%). We studied scenarios in which all patients were referred for endoscopy and/or MRI after the first positive PCR (strategies A, B, C) and after the second positive PCR (strategies D, E, F). Only 3 of 35 screen-detected patients had negative endoscopic findings with positive MRI findings that prompted a diagnosis of NPC, and therefore we studied triage by endoscopy alone (strategies A, D). MRI alone (strategies B, E), and endoscopy with MRI (strategies C, F). For strategies B and E, patients would need to be referred for endoscopy for examination/biopsy.

Finally, Chen et al. (2015) reported results from a prospective single arm screening study of 22,186 participants.³⁶ In this study, participants first underwent serologic testing for EBV anti-VCA IgA (>1:5). If this was positive, a nasopharyngeal swab was performed and amplified for EBV DNA by PCR (optimal positive cutoff: mean+2SD) (strategy G). This study offered similar sensitivity to other studies but had the highest reported specificity (99.95%).

### BALF2 Variant-Informed Strategies

For subjects who screen positive for plasma or nasopharyngeal EBV DNA, a subset will have low-risk BALF2 haplotypes that may prompt discontinuation of further lifetime screening due to the low proportion of endemic NPC cases harboring low-risk haplotypes. This is based on the assumption that haplotypes are stable over one's lifetime (FIG. 2).

In the base case, a single lifetime screen was evaluated. In sensitivity analyses, repeated interval screening was also evaluated which increase absolute risk reduction. As the cumulative incidence of subjects with at least one positive EBV PCR increases with interval screening, a growing proportion of the population may be genotyped and excluded from further lifetime screening (FIG. 6). This is contingent on the underlying screening strategy (e.g., initial plasma EBV DNA PCR vs. initial anti-VCA IgA ELISA). Accordingly, the impact of variant-informed screening upon referral rates becomes more pronounced with more screening events.

For each of the seven variant-agnostic strategies, we evaluated one additional strategy wherein patients with high-risk BALF2 haplotypes (C-C-C or C-C-T) proceeded to the next step of screening. Subjects testing positive for plasma/nasopharyngeal EBV DNA but with low-risk BALF2 haplotypes were not referred for further screening and underwent no further screening in their lifetime. These seven additional variant-informed strategies were:
A_{BALF2}: EBV BamHI-W DNA plasma PCR [Any Amplification]+BALF2 PCR [CCC/CCT Haplotype]→Endoscopy
B_{BALF2}: EBV BamHI-W DNA plasma PCR [Any Amplification]+BALF2 PCR [CCC/CCT Haplotype]→MRI nasopharynx
C_{BALF2}: EBV BamHI-W DNA plasma PCR [Any Amplification]+BALF2 PCR [CCC/CCT Haplotype]→Endoscopy+MRI nasopharynx
D_{BALF2}: EBV BamHI-W DNA plasma PCR [Any Amplification]+BALF2 PCR [CCC/CCT Haplotype]→EBV BamHI-W DNA plasma PCR [Any Amplification]→Endoscopy
E_{BALF2}: EBV BamHI-W DNA plasma PCR [Any Amplification]+BALF2 PCR [CCC/CCT Haplotype]→EBV BamHI-W DNA plasma PCR [Any Amplification]→MRI nasopharynx
F_{BALF2}: EBV BamHI-W DNA plasma PCR [Any Amplification]+BALF2 PCR [CCC/CCT Haplotype]→EBV BamHI-W DNA plasma PCR [Any Amplification]→Endoscopy+MRI
G_{BALF2}: Serum EBV VCA IgA [>1:5]→EBV BamHI-W DNA nasopharyngeal swab PCR [Mean+2SD]+BALF2 PCR [CCC/CCT Haplotype]→Endoscopy

We considered several other permutations of BALF2 genotyping which were not included in this study. For example, strategies referring only the highest-risk haplotype (C-C-T) rather than both the C-C-T/C-C-C haplotypes increases the number of false negatives and decreases the number of false positives. We did not evaluate this triage strategy further, because effective screening sensitivity decreases to approximately 60% without an appreciable increase in cost-effectiveness. We also considered BALF2 genotyping after the second (rather than first) positive PCR in strategies D-F. This results in identical effective sensitivity and specificity, but increases the number of visits for second phlebotomy while decreasing the number of total genotyping tests required. Both approaches have similar cost-effectiveness, and therefore we studied only triage with BALF2 PCR at the first positive EBV DNA PCR to prioritize decreasing the number of phlebotomy visits while slightly increasing laboratory costs. An added advantage of upfront genotyping occurs with interval testing: rather than 1.5% of individuals subject to genotyping per screening year, 5.5% are genotyped after the first PCR and 40% of these individuals with low-risk haplotypes could defer further screening in their lifetime.

The proportion of NPC cases and non-NPC controls with each haplotype were derived from meta-analysis of the three previously-conducted association studies in predominantly endemic populations.⁴-6 Among the 731 NPC cases in these three studies, 84.7% had the highest-risk haplotype (C-C-T) and 93.0% had either the C-C-C or C-C-T high-risk haplotypes. Among the 826 non-NPC controls in these three studies, 44.3% had the highest-risk haplotype (C-C-T) and 60.5% had either the C-C-C or C-C-T high-risk haplotypes. Relative to variant-agnostic screening, variant-informed screening thereby decreases nasoendoscopy/MRI referral rates by 39.5% with a modest relative decrease in screening sensitivity (7.0%).

We assumed that BALF2 genotyping would be performed in a separate PCR reaction but with the same extracted nucleic acid from plasma or nasopharyngeal EBV DNA PCR. For nasopharyngeal EBV DNA PCR, the cutoff for a positive screen exceeds the BALF2 genotyping qPCR 95% LLOD, and we therefore assumed a 100% genotyping success rate. In contrast, a subset of positive plasma specimens would be unable to be genotyped due to low viral load. We therefore assumed that this subset of specimens with amplification failure would be referred for endoscopy and/or MRI as part of usual variant-agnostic screening. In Lam et al., 5/34 screen-detected NPC cases had viral load below the BALF2 qPCR 95% LLOD (FIG. 1).³⁸ In the base case, the plasma genotyping success rate was therefore set at 85%. A range of 70-100% was studied in sensitivity analyses.

Compliance with Screening

Because screening trials were conducted in differing populations with potential for differing access to healthcare, we used compliance estimates from a single study for the base case (Chan et al. 2017) to avoid introducing selection bias among the screening strategies. In this single-arm prospective trial, compliance with plasma EBV DNA PCR→plasma EBV DNA PCR→nasoendoscopy (strategy D) was 97.1%, which fell to 93.2% with PCR→PCR→nasoendoscopy+MRI (strategy F), as some patients declined or were unable to complete MRI. Therefore, for the base case we assumed all strategies had compliance of 97.1% except for strategy C, which incorporated MRI (93.2%). We assumed that compliance for each screening strategy was uniform across populations given similar or higher HDI relative to China, as screening compliance with other cancer screening programs decreases with decreasing HDI.¹⁶ Because cultural differences and healthcare resources among countries likely have a differential impact upon blood-based NPC screening and cervical cancer screening, we considered a range of country-specific compliance in deterministic and probabilistic sensitivity analysis.

### Currency Conversion, Inflation, Discounting, and Purchasing Power Parity

Costs were analyzed from the payer's perspective and presented in 2021 international dollars, which have the same purchasing power parity (PPP) as 2021 United States Dollars. The price of local materials and services in each economy's currency were converted to international dollars via the PPP exchange rate.^{39·40} The price of commercially-available laboratory reagents and equipment were obtained from vendors in United States Dollars. All costs were inflated to the year 2021, and all costs and utilities were discounted at 3.0% annually.⁴¹ For the purposes of cost estimation in all countries, we collected each country's total population, population age structure, HDI, and PPP-adjusted Gross Domestic Product (GDP) per capita (Table 11).

### Costs of Treatment for Nasopharyngeal Carcinoma

Given that fee schedules for individual medical services (clinical visits, endoscopy, imaging, radiotherapy, labs, etc.) are not publicly available for microcosting in each of the studied populations, the price of medical goods and services in each economy were instead estimated using the WHO-CHOICE methodology.⁴² This was used in combination with the 2021 United States Medicare fee schedules and pharmaceutical average sales prices. These fee schedules set reimbursement for physician services⁴³, practice expenses (equipment, supplies, personnel)⁴³, laboratory testing⁴⁴, and pharmaceuticals⁴⁵ for patients insured under Medicare. The cost of all unit services involved in the diagnosis and treatment of NPC were obtained from these fee schedules and tabulated to estimate the cost in the United States for NPC diagnosis and a course of definitive radiotherapy (Table 7), definitive chemoradiotherapy (Tables 7 and 9), and chemotherapy for recurrent or metastatic disease (Table 9).

To convert the cost of diagnosis and treatment in the United States to estimated costs in each included economy, WHO-CHOICE regression models were used.⁴² WHO-CHOICE is an initiative of the World Health Organization that seeks to assist countries in setting healthcare priorities, with a variety of tools developed for generalized cost-effectiveness analyses applied to epidemiological subregions.⁴⁶ One such tool facilitates estimation of unit costs in the outpatient and inpatient settings for 191 member states based upon a regression model developed from more than 10,000 facility-level observations among these 191 countries.⁴² This model incorporates a given economy's GDP per capita (PPP)⁴⁷, hospital occupancy rate, average length of hospital stay, outpatient volume, provider/patient ratios, and other data to predict the unit cost of outpatient or inpatient care. Given that radiotherapy and chemotherapy are limited resources in many nations, we assumed that all costs of workup and treatment were incurred in a public urban outpatient referral hospital setting.

Using each economy's GDP per capita (PPP), the cost of an average outpatient visit was calculated using the WHO-CHOICE model. Then, the costs of diagnosis and treatment for NPC in each economy were calculated from the costs of Medicare services in the United States using the ratio of outpatient costs relative to the United States. We previously validated modeled estimates of the cost of MRI, radiotherapy, and chemotherapy using published estimates from three countries with lower-middle income (India), upper-middle income (China), and high income (Republic of China).³ Given the variability in these proportions, all costs were subsequently studied in sensitivity analysis.

### Cost of Screening Strategies

Micro-costing was performed to estimate the total cost of screening with BamHI-W plasma EBV DNA PCR, EBNA-1 or BamHI-W nasopharyngeal EBV DNA PCR, single-antigen anti-VCA IgA ELISA, and BALF2 plasma or nasopharyngeal EBV DNA PCR.^{40, 48} The methods for conducting these PCR and ELISA assays have been previously described.^{40, 48-50} We chose to employ micro-costing to account for differences in local personnel and transportation costs despite similar international costs of commercially-available laboratory equipment and reagents.

### Sample Transportation Costs

We estimated the cost of transporting each collected sample to a clinical laboratory using the method described by Goldhaber and Goldie.^{46, 51, 52} This method incorporates each region's population, land area, population density, proportion of the population that could be eligible for screening, laboratory worker density, proportion of paved roads in each region, average driving speed, driver wages, fuel costs, and vehicle costs as of 2021.^{16, 40, 48} Data were extracted from United Nations, World Bank, and WHO-CHOICE databases. For simplicity, we assumed a uniform population density to define the land area that a given clinical laboratory would serve. We assumed that a driver would make daily trips in each lab's area to collect samples and deliver them to the nearby central laboratory for analysis. Fuel costs were calculated as the product of fuel efficiency, distance traveled to collect samples, and the cost of fuel. Vehicle and maintenance costs were estimated from WHO-CHOICE and survey data and distributed over a 10-year lifespan. Due to uncertainty in transportation efficiency and driving length, we studied transportation costs from 80-400% of our base case estimate.

### Sample Collection and Laboratory Resources

We assumed that 10 minutes of one phlebotomist's time would be required for a blood draw.⁴⁰ All blood was drawn into commercially-available standard venipuncture tubes with venipuncture needles. We assumed that 15 minutes of one nurse's time would be required to obtain a nasopharyngeal swab.

We updated prior microcosting models for laboratory assays using the WHO's Laboratory Test Costing Tool (LTCT).⁵³ The LTCT is designed to assist policymakers, health economists, and laboratory directors to estimate the cost of individual assays using the laboratory financial minute (LFM) methodology. The cost to test a single sample with a given assay incorporates the costs of labor (described below), reagents/consumables, and equipment (acquisition, maintenance, time allocation per assay, amortization).

Standard methods for performing the plasma/nasopharyngeal BamHI-W/EBNA-1 EBV DNA PCR and anti-VCA IgA ELISA were based upon original scientific reports, standard operating protocols from an academic clinical laboratory, and clinical trials used to develop and validate these tests in large screened populations.^{1, 2, 32, 37, 54-57} Standard commercially-available reagents and consumables were used for processing samples by laboratory technicians.

We estimated laboratory technician time to perform DNA extraction, PCR, ELISA serum dilution and incubation, and test result documentation based on previously-published data and laboratory standard operating protocols.^{40, 48} This time was uniform across populations. After centrifugation and plasma storage (five minutes per clinical sample), DNA extraction kits were used to extract EBV DNA from plasma at a rate of 12 samples per hour (five minutes per sample or control/calibrator). After DNA extraction, commercially-available PCR master mix, EBV DNA probes, and EBV DNA primers were combined with extracted DNA in 96-well reaction plates (60 minutes per 96 samples or controls/calibrators). Real-time PCR for each plate was then performed over two hours with appropriate internal controls and calibrators (4 controls, 6 calibrators, 86 patient samples). Five minutes of technician time per sample were budgeted for result interpretation/documentation. For one batch of 86 clinical samples and 10 controls/calibrators, 1.520 minutes of technician time was required, equivalent to 17.67 LFM per clinical sample. The time and costs to perform BALF2 PCR were identical to EBNA-1/BamHI-W PCR, except that the costs of phlebotomy, sample processing, and nucleic acid extraction were not duplicated.

For the anti-VCA IgA immunoassay, blood was first centrifugated (five minutes per sample). Serum from blood samples was then incubated with commercially-available purified EBV-VCA antigen in 96-well ELISA kits per manufacturer instructions (3 hours per plate, 91 samples per plate, 5 controls/calibrators per plate). Results were then read with an ELISA microplate reader system. Five minutes of technician time per sample were budgeted for result interpretation/documentation. For one batch of 91 clinical samples and 5 controls/calibrators, 1,090 minutes of technician time was required, equivalent to 11.98 LFM per clinical sample.

The cost of reusable equipment and maintenance were distributed over a 5-10 year lifespan. We accounted for an additional 5% reagent/consumables wastage and 20% overhead (applied to all costs). We budgeted an additional five minutes of an administrative assistant's time per sample, and estimated that a clinical pathologist would require one hour to review results from one batch of PCR or ELISA samples.^{40, 48}

### Personnel Costs

Occupation-specific wage data for each economy were obtained from the International Labour Organization, which archive wage data for various occupational sectors in 170 countries.⁵⁸ The most recent average annual wages in each economy were obtained from the 2020-2021 Global Wage Report, and were assigned as the wage of a laboratory technician based on close agreement with ISCO-08/3 technician wages in occupation-specific ILO and NBER data. Wages were similarly assigned for nurses (ISCO-08/3 associated professional), phlebotomists (ISCO-08/5 service worker), administrative assistants (ISCO-08/4 clerical support worker), vehicle drivers (ISCO-08/9 machine operator), and clinical pathologists (ISCO-08/2 professional) in each of the included economies. We imputed missing data using linear regression models as a function of PPP-adjusted GDP per capita. Wage data were converted to USD by PPP exchange rates, and then inflated to 2021.^{39, 41}

We previously validated modeled cost estimates methodology against published costs in lower-middle income, upper-middle income, and high-income countries in a prior study.³. Using this microcosting framework, the median proportion of observed-to-modeled cost was 81% (range, 71-91%). In sensitivity analyses, we studied a range of 50-200% of total screening costs, 50-200% of individual component costs (phlebotomy, reagents, labor time, labor wages), and 50-400% of transportation costs.

### Screening Nasoendoscopy, Examination, and MRI Costs

The costs of nasoendoscopy and MRI were estimated in each country as a function of PPP-adjusted GDP per capita using the aforementioned WHO-CHOICE models, using United States Medicare Fee Schedules as the reference cost. In addition to the cost of endoscopy itself, we incorporated the cost of a single outpatient visit (as estimated by WHO-CHOICE) to account for a history and physical examination prior to each endoscopy. Complications and incidental findings during endoscopy/MRI were assumed to be negligible.⁵⁹

### Model Analysis

For each population and screening strategy, incremental cost-effectiveness ratios (ICER) were calculated as the incremental cost per incremental quality-adjusted life-years per screened subject over a lifetime horizon. Based on WHO-CHOICE guidelines, a willingness to pay (WTP) threshold was set at double the local PPP-adjusted per capita GDP (GDPₚₚₚ).⁴⁶ To facilitate comparisons across currencies and economies, values were reported as the ICER divided by PPP-adjusted per capita GDP. We also studied lower WTP thresholds of 0.5 and 1.0 QALY/GDPₚₚₚ.

Although a subset of participants at medium or high serologic risk were retested in the Ji et al. randomized trial, most prospective screening trials have reported performance with only onetime screening. While onetime screening reduces mortality for the small number of screen-detected prevalent cases, real-world screening programs would employ uniform or adaptive interval screening. With more frequent screening, per-test mortality reduction decreases while absolute mortality reduction increases. Therefore, to supplement the base case analysis of onetime screening, we also studied the impact of screening interval (every 1-5 years) upon absolute mortality reduction and cost-effectiveness.

To assess the robustness of the base case analysis, we performed one-way deterministic and probabilistic sensitivity analyses by varying model parameters in each populations across the 18 screening strategies. The range and distributions for each of these parameters are listed in Table 6. We studied the impact of varying the costs of reagents, healthcare services, wages, sample transportation, and discount rate. To study uncertainty in laboratory efficiency, the hours required to perform select tasks (phlebotomy, nasopharyngeal swab, PCR, ELISA, etc.) were varied by 50-200% around the base case. To separately study total screening costs independent of these components, we varied costs by 50-200% around the base case.

Healthcare utilities were varied by twice the published standard deviation around the base case.^{17, 60, 61} We sampled from the 1,000 randomly-generated sets of calibrated transition probabilities and the Dirichlet stage distributions of NPC. Given higher incidence rates, we performed a separate subset analysis to study the cost-effectiveness of screening only men.

To ascertain the most cost-effective age to offer screening in each population, we varied the age at first screening from 40-60 in five-year increments. We also studied the impact of variable rates of WHO II/III NPC, compliance with screening regimens, number of radiotherapy fractions, and utilization of palliative radiotherapy. We sampled from the 95% confidence intervals for LRR, DM, and OS models to study uncertainty in recurrence rates. Probabilistic sensitivity analysis was performed with 1,000 iterations in each population to study the impact of parameter uncertainty upon cost-effectiveness. We incorporated correlation in health utility uncertainty distributions based on commonsense preferences for those states using a preference order matrix.⁶² This study was conducted in accordance with CHEERS reporting standards.⁶³

### Statistical Analysis

Positive percent agreement (PPA) and negative percent agreement (NPA) were reported with Clopper-Pearson score 95% binomial confidence intervals using NGS as the reference method. The 95% LLOD was calculated using probit regression for each target. Linear regression was used to fit Cₜ values against nominal concentrations. Odds ratios for high-risk haplotypes (C-C-T and/or C-C-C at positions 162215-162476-163364) were calculated using the common low-risk haplotypes as reference (sum of A-T-C and C-T-C). For EBV-positive NPC cases, the reference group includes all non-NPC controls for each individual study (present cohort, Xu et al., Hui et al., Lam et al.).4-6 Fisher exact tests were used to calculate p-values for SNV and haplotype associations with NPC. For targeted NGS, the p-value threshold for statistical significance was adjusted for the number of evaluated positions using the Bonferroni correction (α=3.68×10⁻⁵). Analyses were conducted using the R statistical software package. This study was reported in accordance STARD and CHEERS guidelines.^{63, 64}

### REFERENCES

1. Abeynayake J, Johnson R, Libiran P, et al. Commutability of the Epstein-Barr virus WHO international standard across two quantitative PCR methods. J Clin Microbiol. 2014; 52 (10): 3802-3804. doi: 10.1128/JCM.01676-14
2. Le Q T, Zhang Q, Cao H, et al. An international collaboration to harmonize the quantitative plasma Epstein-Barr virus DNA assay for future biomarker-guided trials in nasopharyngeal carcinoma. Clin Cancer Res Off J Am Assoc Cancer Res. 2013; 19 (8): 2208-2215. doi: 10.1158/1078-0432.CCR-12-3702
3. Miller J A, Le Q T, Pinsky B A, Wang H. Cost-Effectiveness of Nasopharyngeal Carcinoma Screening With Epstein-Barr Virus Polymerase Chain Reaction or Serology in High-Incidence Populations Worldwide. J Natl Cancer Inst. 2021; 113 (7): 852-862. doi: 10.1093/jnci/djaa198
4. Xu M, Yao Y, Chen H, et al. Genome sequencing analysis identifies Epstein-Barr virus subtypes associated with high risk of nasopharyngeal carcinoma. Nat Genet. 2019; 51 (7): 1131-1136. doi: 10.1038/s41588-019-0436-5
5. Lam W K J, Ji L, Tse O Y O, et al. Sequencing Analysis of Plasma Epstein-Barr Virus DNA Reveals Nasopharyngeal Carcinoma-Associated Single Nucleotide Variant Profiles. Clin Chem. 2020; 66 (4): 598-605. doi: 10.1093/clinchem/hvaa027
6. Hui K F, Chan T F, Yang W, et al. High risk Epstein-Barr virus variants characterized by distinct polymorphisms in the EBER locus are strongly associated with nasopharyngeal carcinoma. Int J Cancer. 2019; 144 (12): 3031-3042. doi: 10.1002/ijc.32049
7. Bray F, Colombet M, Mery L. et al. Cancer Incidence in Five Continents, Vol. XI. Lyon: International Agency for Research on Cancer; 2017. Accessed Jan. 20, 2019. ci5.iarc.fr
8. Forman D, Bray F, Brewstern D, et al. Cancer Incidence in Five Continents, Vol. X. Published 2013. Accessed Jan. 20, 2019. ci5.iarc.fr/CI5-X/Default.aspx
9. Sung H, Ferlay J, Siegel R L, et al. Global Cancer Statistics 2020: GLOBOCAN Estimates of Incidence and Mortality Worldwide for 36 Cancers in 185 Countries. CA Cancer J Clin. 2021; 71 (3): 209-249. doi: 10.3322/caac.21660
10. Taiwan Cancer Registry. Published online 2019. Accessed Jan. 7, 2019. tcr.cph.ntu.edu.tw/main.php?Page=N2
11. Cancer Registry—National Registry Of Diseases Office. Accessed Jan. 22, 2022. nrdo.gov.sg/publications/cancer
12. Hong Kong Cancer Registry: Nasopharyngeal Cancer. Published online 2016. Accessed Jan. 20, 2019. ha.org.hk/cancereg/pdf/factsheet/2016/npc_2016.pdf
13. Directory of RAdiotherapy Centres (DIRAC). Published Apr. 3, 2019. Accessed Jul. 2, 2020. iaea.org/resources/databases/dirac
14. Bray F, Ferlay J, Soerjomataram I, Siegel R L, Torre L A, Jemal A. Global cancer statistics 2018: GLOBOCAN estimates of incidence and mortality worldwide for 36 cancers in 185 countries. CA Cancer J Clin. 2018; 68 (6): 394-424. doi: 10.3322/caac.21492
15. Filipovic-Pierucci A, Zarca K, Wiener M, et al. Heemod: Markov Models for Health Economic Evaluations.; 2017. CRAN.R-project.org/package=heemod
16. The Global Health Observatory 2017 Update. Published online 2017. Accessed Jan. 10, 2019. who.int/gho/en/
17. Pow E H N, Kwong D L W, McMillan A S, et al. Xerostomia and quality of life after intensity-modulated radiotherapy vs. conventional radiotherapy for early-stage nasopharyngeal carcinoma: initial report on a randomized controlled clinical trial. Int J Radiat Oncol Biol Phys. 2006; 66 (4): 981-991. doi: 10.1016/j.ijrobp.2006.06.013
18. Poon D M C, Kam M K M, Johnson D, Mo F, Tong M, Chan ATC. Durability of the parotid-sparing effect of intensity-modulated radiotherapy (IMRT) in early stage nasopharyngeal carcinoma: A 15-year follow-up of a randomized prospective study of IMRT versus two-dimensional radiotherapy. Head Neck. 2021; 43 (6): 1711-1720. doi: 10.1002/hed.26634
19. Chie W C, Hong R L, Lai C C, Ting L L, Hsu M M. Quality of life in patients of nasopharyngeal carcinoma: validation of the Taiwan Chinese version of the EORTC QLQ-C30 and the EORTC QLQ-H&N35. Qual Life Res Int J Qual Life Asp Treat Care Rehabil. 2003; 12 (1): 93-98. doi: 10.1023/a: 1022070220328
20. Harrington K J, Ferris R L, Blumenschein G, et al. Nivolumab versus standard, single-agent therapy of investigator's choice in recurrent or metastatic squamous cell carcinoma of the head and neck (CheckMate 141): health-related quality-of-life results from a randomised, phase 3 trial. Lancet Oncol. 2017; 18 (8): 1104-1115. doi: 10.1016/S1470-2045 (17) 30421-7
21. Noel C W, Stephens R F, Su J S, et al. Mapping the EORTC QLQ-C30 and QLQ-H&N35, onto EQ-5D-5L and HUI-3 indices in patients with head and neck cancer. Head Neck. 2020; 42 (9): 2277-2286. doi: 10.1002/hed.26181
22. Gareen I F, Duan F, Greco E M, et al. Impact of lung cancer screening results on participant health-related quality of life and state anxiety in the National Lung Screening Trial. Cancer. 2014; 120 (21): 3401-3409. doi: 10.1002/cncr.28833
23. Tosteson A N A, Skinner J S, Tosteson T D, et al. The cost effectiveness of surgical versus nonoperative treatment for lumbar disc herniation over two years: evidence from the Spine Patient Outcomes Research Trial (SPORT). Spine. 2008; 33 (19): 2108-2115.
24. Loimu V, Mäkitie A A, Bäck L J, et al. Health-related quality of life of head and neck cancer patients with successful oncological treatment. Eur Arch Oto-Rhino-Laryngol Off J Eur Fed Oto-Rhino-Laryngol Soc EUFOS Affil Ger Soc Oto-Rhino-Laryngol-Head Neck Surg. 2015; 272 (9): 2415-2423. doi: 10.1007/s00405-014-3169-1
25. NCCN Clinical Practice Guidelines in Oncology. Head and Neck Cancers. Version 1.2019. Published online Mar. 6, 2019. Accessed Apr. 1, 2019. nccn.org/professionals/physician_gls/pdf/head-and-neck.pdf
26. Edge S B, Compton C C. The American Joint Committee on Cancer: the 7th edition of the AJCC cancer staging manual and the future of TNM. Ann Surg Oncol. 2010; 17 (6): 1471-1474. doi: 10.1245/s10434-010-0985-4
27. Al-Sarraf M, LeBlanc M, Giri P G, et al. Chemoradiotherapy versus radiotherapy in patients with advanced nasopharyngeal cancer: phase III randomized Intergroup study 0099. J Clin Oncol Off J Am Soc Clin Oncol. 1998; 16 (4): 1310-1317. doi: 10.1200/JCO.1998.16.4.1310
28. Wee J, Tan E H, Tai B C, et al. Randomized trial of radiotherapy versus concurrent chemoradiotherapy followed by adjuvant chemotherapy in patients with American Joint Committee on Cancer/International Union against cancer stage III and IV nasopharyngeal cancer of the endemic variety. J Clin Oncol Off J Am Soc Clin Oncol. 2005; 23 (27): 6730-6738. doi: 10.1200/JCO.2005.16.790
29. Blanchard P, Lee A, Marguet S, et al. Chemotherapy and radiotherapy in nasopharyngeal carcinoma: an update of the MAC-NPC meta-analysis. Lancet Oncol. 2015; 16 (6): 645-655. doi: 10.1016/S1470-2045 (15) 70126-9
30. Zhang Y, Chen L, Hu G Q, et al. Gemcitabine and Cisplatin Induction Chemotherapy in Nasopharyngeal Carcinoma. N Engl J Med. 2019; 381 (12): 1124-1135.
31. Zhang L, Huang Y, Hong S, et al. Gemcitabine plus cisplatin versus fluorouracil plus cisplatin in recurrent or metastatic nasopharyngeal carcinoma: a multicentre, randomised, open-label, phase 3 trial. Lancet Lond Engl. 2016; 388 (10054): 1883-1892. doi: 10.1016/S0140-6736 (16) 31388-5
32. Chan K C A, Woo J K S, King A, et al. Analysis of Plasma Epstein-Barr Virus DNA to Screen for Nasopharyngeal Cancer. N Engl J Med. 2017; 377 (6): 513-522.
33. Ji M F, Sheng W, Cheng W M, et al. Incidence and mortality of nasopharyngeal carcinoma: interim analysis of a cluster randomized controlled screening trial (PRO-NPC-001) in southern China. Ann Oncol. 2019; 30 (10): 1630-1637. doi: 10.1093/annonc/mdz231
34. Liu Z, Ji M F, Huang Q H, et al. Two Epstein-Barr virus-related serologic antibody tests in nasopharyngeal carcinoma screening: results from the initial phase of a cluster randomized controlled trial in Southern China. Am J Epidemiol. 2013; 177 (3): 242-250. doi: 10.1093/aje/kws404
35. Chan K C A, Hung E C W, Woo J K S, et al. Early detection of nasopharyngeal carcinoma by plasma Epstein-Barr virus DNA analysis in a surveillance program. Cancer. 2013; 119 (10): 1838-1844. doi: 10.1002/cncr.28001
36. Chen Y, Zhao W, Lin L, et al. Nasopharyngeal Epstein-Barr Virus Load: An Efficient Supplementary Method for Population-Based Nasopharyngeal Carcinoma Screening. PloS One. 2015; 10 (7): e0132669. doi: 10.1371/journal.pone.0132669
37. Lo Y M, Chan L Y, Lo K W, et al. Quantitative analysis of cell-free Epstein-Barr virus DNA in plasma of patients with nasopharyngeal carcinoma. Cancer Res. 1999; 59 (6): 1188-1191.
38. Lam W K J, Jiang P, Chan K C A, et al. Sequencing-based counting and size profiling of plasma Epstein-Barr virus DNA enhance population screening of nasopharyngeal carcinoma. Proc Natl Acad Sci. 2018; 115 (22): E5115-E5124. doi: 10.1073/pnas. 1804184115
39. OECD. OECD Purchasing power parities (PPP) (indicator). Published 2019. Accessed Jan. 20, 2019.data.oecd.org/conversion/purchasing-power-parities-ppp.htm
40. Goldie S J. Gaffikin L, Goldhaber-Fiebert J D, et al. Cost-effectiveness of cervical-cancer screening in five developing countries. N Engl J Med. 2005; 353 (20): 2158-2168. doi: 10.1056/NEJMsa044278
41. CPI Home: U.S. Bureau of Labor Statistics. Accessed Jan. 20, 2019.bls.gov/cpi/
42. Stenberg K, Lauer J A, Gkountouras G, Fitzpatrick C, Stanciole A. Econometric estimation of WHO-CHOICE country-specific costs for inpatient and outpatient health service delivery. Cost Eff Resour Alloc CE. 2018; 16. doi: 10.1186/s12962-018-0095-x
43. Centers for Medicare and Medicaid Services. 2019 Medicare Physician Fee Schedule. Published 2019. Accessed Jan. 20, 2019.cms.gov/Medicare/Medicare-Fee-for-Service-Payment/PhysicianFeeSched/index.html
44. Centers for Medicare and Medicaid Services. 2019 Medicare Clinical Laboratory Fee Schedule. Published 2019. Accessed Jan. 20, 2019.cms.gov/Medicare/Medicare-Fee-for-Service-Payment/ClinicalLabFeeSched/Clinical-Laboratory-Fee-Schedule-Files-Items/19CLABQ1.html?DLPage=1&DLEntries=10&DLSort=2&DLSortDir=descending
45. Centers for Medicare and Medicaid Services. 2019 Medicare Average Sales Price Drug Pricing Schedule. Published 2019. Accessed Jan. 20, 2019.cms.gov/Medicare/Medicare-Fee-for-Service-Part-B-Drugs/McrPartBDrugAvgSalesPrice/2019ASPFiles.html
46. World Health Organization (Geneva). Choosing interventions that are cost-effective. Published online 2014. Accessed Jan. 20, 2019. who.int/choice/en/
47. International Monetary Fund. International Monetary Fund World Economic Outlook. Published online 2018. Accessed Jan. 20, 2019. imf.org/external/pubs/ft/weo/2018/02/weodata/
48. Goldhaber-Fiebert J D, Goldie S J. Estimating the cost of cervical cancer screening in five developing countries. Cost Eff Resour Alloc C E. 2006; 4:13. doi: 10.1186/1478-7547-4-13
49. Goldie S J, Kuhn L, Denny L, Pollack A, Wright T C. Policy analysis of cervical cancer screening strategies in low-resource settings: clinical benefits and cost-effectiveness. JAMA. 2001; 285 (24): 3107-3115.
50. Dalvie M A, Sinanovic E, London L, Cairncross E, Solomon A, Adam H. Cost analysis of ELISA, solid-phase extraction, and solid-phase microextraction for the monitoring of pesticides in water. Environ Res. 2005; 98 (1): 143-150. doi: 10.1016/j.envres.2004.09.002
51. World Bank. World Bank Open Data. Published online 2018. Accessed Jan. 20, 2019. data.worldbank.org/
52. United Nations Department of Economic and Social Affairs. World Population Prospects: The 2017 Revision. Published 2017. Accessed Jan. 20, 2019. un.org/development/desa/publications/world-population-prospects-the-2017-revision.html
53. Laboratory test costing tool-user manual (2019). Accessed Jan. 22, 2022. euro.who.int/en/health-topics/Health-systems/laboratory-services/publications/laboratory-test-costing-tool-user-manual-2019
54. Chan K H, Gu Y L, Ng F, et al. EBV specific antibody-based and DNA-based assays in serologic diagnosis of nasopharyngeal carcinoma. Int J Cancer. 2003; 105 (5): 706-709. doi: 10.1002/ijc. 11130
55. Fachiroh J, Paramita D K, Hariwiyanto B, et al. Single-assay combination of Epstein-Barr Virus (EBV) EBNA1- and viral capsid antigen-p18-derived synthetic peptides for measuring anti-EBV immunoglobulin G (IgG) and IgA antibody levels in sera from nasopharyngeal carcinoma patients: options for field screening. J Clin Microbiol. 2006; 44 (4): 1459-1467. doi: 10.1128/JCM.44.4.1459-1467.2006
56. Paramita D K, Fachiroh J, Haryana S M, Middeldorp J M. Two-step Epstein-Barr virus immunoglobulin A enzyme-linked immunosorbent assay system for serological screening and confirmation of nasopharyngeal carcinoma. Clin Vaccine Immunol CVI. 2009; 16 (5): 706-711. doi: 10.1128/CVI.00425-08
57. Zong Y S, Sham J S, Ng M H, et al. Immunoglobulin A against viral capsid antigen of Epstein-Barr virus and indirect mirror examination of the nasopharynx in the detection of asymptomatic nasopharyngeal carcinoma. Cancer. 1992; 69 (1): 3-7.
58. International Labour Organization. Global Wage Report 2018/19. Published Jan. 1, 2019. Accessed May 29, 2019. ilo.org/global/research/global-reports/global-wage-report/2018/lang--en/index.htm
59. Lang B H H, Chu K K W, Tsang R K Y, Wong K P, Wong B Y H. Evaluating the Incidence, Clinical Significance and Predictors for Vocal Cord Palsy and Incidental Laryngopharyngeal Conditions before Elective Thyroidectomy: Is There a Case for Routine Laryngoscopic Examination? World J Surg. 2014; 38 (2): 385-391. doi: 10.1007/s00268-013-2259-3
60. Kim S H, Jo M W, Kim H J, Ahn J H. Mapping EORTC QLQ-C30 onto EQ-5D for the assessment of cancer patients. Health Qual Life Outcomes. 2012:10:151. doi: 10.1186/1477-7525-10-151
61. Truong M T, Zhang Q, Rosenthal D I, et al. Quality of Life and Performance Status From a Substudy Conducted Within a Prospective Phase 3 Randomized Trial of Concurrent Accelerated Radiation Plus Cisplatin With or Without Cetuximab for Locally Advanced Head and Neck Carcinoma: NRG Oncology Radiation Therapy Oncology Group 0522. Int J Radiat Oncol Biol Phys. 2017; 97 (4): 687-699. doi: 10.1016/j.ijrobp.2016.08.003
62. Goldhaber-Fiebert J D, Jalal H J. Some Health States Are Better Than Others: Using Health State Rank Order to Improve Probabilistic Analyses. Med Decis Mak Int J Soc Med Decis Mak. 2016; 36 (8): 927-940. doi: 10.1177/0272989X15605091
63. Husereau D. Drummond M, Petrou S, et al. Consolidated Health Economic Evaluation Reporting Standards (CHEERS)—explanation and elaboration: a report of the ISPOR Health Economic Evaluation Publication Guidelines Good Reporting Practices Task Force. Value Health J Int Soc Pharmacoeconomics Outcomes Res. 2013; 16 (2): 231-250. doi: 10.1016/j.jval.2013.02.002
64. Bossuyt P M, Reitsma J B, Bruns D E, et al. STARD 2015: An Updated List of Essential Items for Reporting Diagnostic Accuracy Studies. Radiology. 2015: 277 (3): 826-832. doi: 10.1148/radiol.2015151516

TABLE 1
EBV BALF2 Genotyping qPCR Primer and Probe
Oligonucleotide Sequences and Characteristics

| | | 5′ | 3′ | Tm* | Tm* | Sequence |
|---|---|---|---|---|---|---|
| Oligo- | | Modifi- | Modifi- | Match | Mismatch | Conservation |
| nucleotide | Sequence (5′ → 3′) | cation | cation | (° C.) | (° C.) | (n = 1,050)** |
| V700_FWD | GCGACCTGCCAGA | — | — | 63.4 | — | 1,037 (98.8%) |
| | CCT | | | | | |
| | (SEQ ID NO: 3) | | | | | |
| V700_REV | CACAGCATCAGCAC | — | — | 64.6 | — | 1,042 (99.2%) |
| | CTTGGA | | | | | |
| | (SEQ ID NO: 4) | | | | | |
| V700_WT_ | CAGGGCCGGGTGTAC | FAM | BHQ1ₚₗᵤₛ | 65.2 | 60.7 | 822 (78.3%) |
| FAM | (SEQ ID NO: 12) | | | | | |
| V700L_MT_ | CAGGGCCGGTTGTAC | CAL560 | BHQ1ₚₗᵤₛ | 63.4 | 55.6 | 219 (20.9%) |
| CAL560 | (SEQ ID NO: 5) | | | | | |
| I613_FWD | GGTCAGCAGTGAGCG | — | — | 65.3 | — | 1,036 (98.7%) |
| | GTAAAAC | | | | | |
| | (SEQ ID NO: 6) | | | | | |
| I613_REV | CAAGGACCTGGTCAA | — | — | 64.3 | — | 1,042 (99.2%) |
| | GAGCTG | | | | | |
| | (SEQ ID NO: 7) | | | | | |
| I613V_MT_ | CTGGGTGAAGACGGG | CAL610 | BHQ2ₚₗᵤₛ | 71.3 | 64.3 | 391 (37.2%) |
| CAL610 | GCA | | | | | |
| | (SEQ ID NO: 8) | | | | | |
| V317_FWD | TGTTTGCCGACTGTG | | | 64.4 | — | 1,043 (99.3%) |
| | AGGG | | | | | |
| | (SEQ ID NO: 9) | | | | | |
| V317_REV | GGCTGGCATTATAT | — | — | 65.0 | — | 1,039 (99.0%) |
| | CGGTGTAACG | | | | | |
| | (SEQ ID NO: 10) | | | | | |
| V317M_MT_ | AGAGGCCCGTATGG | Q670 | BHQ2ₚₗᵤₛ | 65.2 | 62.6 | 307 (29.2%) |
| Q670 | CTG | | | | | |
| | (SEQ ID NO: 11) | | | | | |

Values are presented as number (percent). Tm, melt temperature; FWD, forward; REV, reverse; WT, wild-type; MT, mutant.
*Calculated using IDT OligoAnalyzer for primers using qPCR conditions (DNA, 0.2 μM [oligonucleotide], 50 mM [Na⁺], 3 mM [Mg²⁺],0.8 mM [dNTPs]. Calculated using BioSearch RealTimeDesign using BHQplus settings for hydrolysis probes. Mismatch Tm is for wild-type → mutant or mutant → wild-type nucleotide annealing.
**Presence of primer/probe in 1,050 EBV GenBank sequences identified on Nov. 23, 2021 aligning to the EBV BALF2 region of interest (NC_007605.1: 162115-163464) with ≥98% coverage.

TABLE 2
EBV BALF2 Genotyping qPCR dsDNA Control Oligonucleotides

| dsDNA Control | 162215 | 162476 | 163364 | Length | |
|---|---|---|---|---|---|
| Fragment | Allele | Allele | Allele | | Sequence (5′ → 3′) |
| Risk Alleles | C | C | T | 483 | CACCCTGTTCTTGATCTTGATGTTCCTGGGGCACAGCATCAGCAC |
| [wt/mt/mt]* | | | | | CTTGGACATGCGCACAGGCAGCCGCCGGCCGTACACCCGGCCCTG |
| | | | | | CAGGGCCGCGTCCAGGTCTGGCAGGTCGCAGGTGGGCTCCCCATG |
| | | | | | CACCACCTTGGCCTCCTTGGCCGTGAATTCTCATAGCACATACAG |
| | | | | | ATGGGCAGGGAGATGTCCTGCAGGATGGTCAGCAGTGAGCGGTAA |
| | | | | | AACAGCTGGGTGAAGACGGGGCAGGCGGGCTGCGCAAAGGGGTTG |
| | | | | | CACGAGTACTGCATCACGTGGTAGCAGCTCTTGACCAGGTCCTTG |
| | | | | | TAGGTGAGTAGAGGACGGAATTGGTGGCAAAGATCTGCGTGGACA |
| | | | | | CGTGGGGGGCCAGGCTGGCATTATATCGGTGTAACGCAGCCATAC |
| | | | | | GGGCCTCTGGACCCTCACAGTCGGCAAACAGGGGCCACGAGTCGT |
| | | | | | AGTTGAGGCTGGCCGGGGTCTCGTGCGAGGCCT |
| | | | | | (SEQ ID NO: 13) |
| Non-Risk Alleles | A | T | C | 483 | CACCCTGTTCTTGATCTTGATGTTCCTGGGGCACAGCATCAGCAC |
| [mt/wt/wt]* | | | | | CTTGGACATGCGCACAGGCAGCCGCCGGCCGTACAACCGGCCCTG |
| | | | | | CAGGGCCGCGTCCAGGTCTGGCAGGTCGCAGGTGGGCTCCCCATG |
| | | | | | CACCACCTTGGCCTCCTTGGCCGTGAATTCTCATAGCACATACAG |
| | | | | | ATGGGCAGGGAGATGTCCTGCAGGATGGTCAGCAGTGAGCGGTAA |
| | | | | | AACAGCTGGGTGAAGATGGGGCAGGCGGGCTGCGCAAAGGGGTTG |
| | | | | | CACGAGTACTGCATCACGTGGTAGCAGCTCTTGACCAGGTCCTTG |
| | | | | | TAGGTGAGTAGAGGACGGAATTGGTGGCAAAGATCTGCGTGGACA |
| | | | | | CGTGGGGGGCCAGGCTGGCATTATATCGGTGTAACGCAGCCACAC |
| | | | | | GGGCCTCTGGACCCTCACAGTCGGCAAACAGGGGCCACGAGTCGT |
| | | | | | AGTTGAGGCTGGCCGGGGTCTCGTGCGAGGCCT |
| | | | | | (SEQ ID NO: 14) |

wt, wild-type; mt, mutant.
*Denotes wild-type or mutant nucleotide at 162215/162476/163364 positions in EBV reference genome (NCBI RefSeq NC_007605.1).

TABLE 3
EBV BALF2 Genotyping qPCR Reagents and Concentrations

| | Stock | | Volume | PCR Reaction | |
|---|---|---|---|---|---|
| Reagent | Concentration | | (μL) | Concentration | |
| V700_FWD | 3,750 | nM | 2.0* | 300 | nM |
| V700_REV | 3,750 | nM | — | 300 | nM |
| V700_WT_FAM | 625 | nM | — | 50 | nM |
| V700L_MT_CAL560 | 1,875 | nM | — | 150 | nM |
| I613_FWD | 3,750 | nM | — | 300 | nM |
| I613_REV | 3,750 | nM | — | 300 | nM |
| I613V_MT_CAL610 | 625 | nM | — | 50 | nM |
| V317_FWD | 3,750 | nM | — | 300 | nM |
| V317_REV | 3,750 | nM | — | 300 | nM |
| V317M_MT_Q670 | 1,875 | nM | — | 150 | nM |

| | | | |
|---|---|---|---|
| FastStart TaqMan Probe Master Mix** | 2X | 12.5 | 1X |
| Nuclease-free Water | — | 0.5 | — |
| Template*** | — | 10.0 | — |
| Total | — | 25.0 | — |

MT, mutant; WT, wild-type; nM, nanomolar.
*2.0 μL primer/probe mix diluted in Tris-EDTA (10 mM Tris, 1 mM EDTA) at stock concentrations listed above.
**Catalog Numbers 11732-020 and 11732-088.
***IDT gBlock dsDNA control gene fragments (risk or non-risk alleles), wild-type whole-virus control nucleic acids from EBV-infected B95-8 cell line, or extracted nucleic acids from clinical plasma specimens.

TABLE 4
EBV BALF2 Genotyping qPCR Interpretation and Haplotypes

| | | | | | | Interpretation and EBV BALF2 Haplotype | | | | |
|---|---|---|---|---|---|---|---|---|---|---|
| | | | | | | | | | | Haplotype and |
| | Real-Time PCR Result | | | | | V700 | | | | NPC Risk |
| | V700 | V700L | I613V | V317M | Quality | Wild- | V700L | I613V | V317M | (162215-162476- |
| PCR Template | (FAM) | (CAL560) | (CAL610) | (Q670) | Control | Type | Mutation | Mutation | Mutation | 163364) |
| No Template | ndet | ndet | ndet | ndet | NTC QC | Not | Not | Not | Not | No Template Control |
| Control | | | | | Passed | Detected | Detected | Detected | Detected | |
| Wild-Type B95- | Cₜ < 38 | ndet | ndet | ndet | Wild-Type | Detected | Not | Not | Not | C-T-C: Low Risk |
| 8 Control | | | | | QC Passed | | Detected | Detected | Detected | (Wild-Type) |
| High-Risk | Cₜ < 38 | ndet | Cₜ < 38 | Cₜ < 38 | High-Risk | Detected | Not | Detected | Detected | C-C-T: High Risk |
| dsDNA Control | | | | | QC Passed | | Detected | | | Control |
| Low-Risk | ndet | Cₜ < 38 | ndet | ndet | Low-Risk QC | Not | Detected | Not | Not | A-T-C: Low Risk |
| dsDNA Control | | | | | Passed | Detected | | Detected | Detected | Control |
| Patient | Cₜ ≤ 45 | ndet | ndet | ndet | Accept | Detected | Not | Not | Not | C-T-C: Low Risk |
| Specimens | | | | | | | Detected | Detected | Detected | (Wild-Type) |
| | Cₜ ≤ 45 | ndet | Cₜ ≤ 45 | ndet | Accept | Detected | Not | Detected | Not | C-C-C: High Risk |
| | | | | | | | Detected | | Detected | |
| | Cₜ ≤ 45 | ndet | Cₜ ≤ 45 | Cₜ ≤ 45 | Accept | Detected | Not | Detected | Detected | C-C-T: High Risk |
| | | | | | | | Detected | | | |
| | ndet | Cₜ ≤ 45 | ndet | ndet | Accept | Not | Detected | Not | Not | A-T-C: Low Risk |
| | | | | | | Detected | | Detected | Detected | |
| | Cₜ ≤ 45 | ndet | ndet | Cₜ ≤ 45 | Accept | Detected | Not | Not | Detected | C-T-T: Uncommon |
| | | | | | | | Detected | Detected | | Haplotype* |
| | ndet | Cₜ ≤ 45 | Cₜ ≤ 45 | ndet | Accept | Not | Detected | Detected | Not | A-C-C: Uncommon |
| | | | | | | Detected | | | Detected | Haplotype* |
| | ndet | Cₜ ≤ 45 | ndet | Cₜ ≤ 45 | Accept | Not | Detected | Not | Detected | A-T-T: Uncommon |
| | | | | | | Detected | | Detected | | Haplotype* |
| | ndet | Cₜ ≤ 45 | Cₜ ≤ 45 | Cₜ ≤ 45 | Accept | Not | Detected | Detected | Detected | A-C-T: Uncommon |
| | | | | | | Detected | | | | Haplotype* |
| | ndet | ndet | ndet | ndet | Repeat PCR | Not | Not | Not | Not | No Amplification |
| | | | | | | Detected | Detected | Detected | Detected | |
| | Cₜ ≤ 45 | Cₜ ≤ 45 | Any | Any | Review** | Detected | Detected | Any | Any | Cannot Assign |
| | | | | | | | | | | Haplotype** |
| | ndet | ndet | Cₜ ≤ 45 | ndet | Review** | Not | Not | Detected | Not | Cannot Assign |
| | | | | | | Detected | Detected | | Detected | Haplotype** |
| | ndet | ndet | ndet | Cₜ ≤ 45 | Review** | Not | Not | Not | Detected | Cannot Assign |
| | | | | | | Detected | Detected | Detected | | Haplotype** |
| | ndet | ndet | Cₜ ≤ 45 | Cₜ ≤ 45 | Review** | Not | Not | Detected | Detected | Cannot Assign |
| | | | | | | Detected | Detected | | | Haplotype** |

Fixed fluorescence thresholds of 200 relative fluorescence units ([RFU], V700-FAM), 100 RFU (V700L-CAL560), 300 RFU (I613V CAL610), and 300 RFU (V317M-Q670) are used to determine each target's threshold cycle (Cₜ).
Cₜ, cycle threshold; ndet, not detected; NTC, no template control; QC, quality control.
*Uncommon haplotypes: Represent <1% of BALF2 haplotypes in Xu et al. 2019. Haplotype association with NPC risk unknown.
**Cannot assign EBV BALF2 haplotype. May represent mixed infection if both V700 and V700L are detected. May represent amplification failure or non-A/C nucleotide at V700 position if neither V700/V700L are detected.

TABLE 5
Characteristics of Patients with Genotyped Specimens

| | All | | EBV-Positive | | Non-NPC | |
|---|---|---|---|---|---|---|
| Characteristic | Patients | | NPC Cases | | Controls | |
| Patients | 179 | | 24 | | 155 | |
| Age at First Genotyped Specimen (Years) | 42 | [18-64] | 53 | [45-65] | 33 | [18-63] |
| Maximum Viral Load (EBNA-1 IU/mL | 612 | [<100-882,818] | 371 | [<100-582,699] | 614 | [<100-882,818] |
| plasma) | | | | | | |
| Male | 105 | (59%) | 18 | (75%) | 87 | (56%) |
| EBV-Positive NPC | 24 | (13%) | 24 | (100%) | 0 | (0%) |
| Solid Organ Transplantation* | 71 | (40%) | 0 | (0%) | 71 | (46%) |
| Liver | 40 | (22%) | — | | 40 | (26%) |
| Heart | 35 | (20%) | — | | 35 | (23%) |
| Kidney | 30 | (17%) | — | | 30 | (19%) |
| Lung | 23 | (13%) | — | | 23 | (15%) |
| Small Intestine | 5 | (3%) | — | | 5 | (3%) |
| Pancreas | 2 | (1%) | — | | 2 | (1%) |
| Bone Marrow Transplantation | 51 | (28%) | 0 | (0%) | 51 | (33%) |
| Hematologic Neoplasms | 76 | (42%) | 0 | (0%) | 76 | (49%) |
| B-Cell Lymphomas/Leukemias | 34 | (19%) | — | | 34 | (22%) |
| DLBCL | 15 | (8%) | — | | 15 | (10%) |
| B-ALL | 12 | (7%) | — | | 12 | (8%) |
| Hodgkin Lymphoma | 5 | (3%) | — | | 5 | (3%) |
| Burkitt Lymphoma | 1 | (1%) | — | | 1 | (1%) |
| Marginal Zone Lymphoma | 1 | (1%) | — | | 1 | (1%) |
| T-Cell Lymphomas/Leukemias | 22 | (12%) | — | | 22 | (14%) |
| NKTCL | 9 | (5%) | — | | 9 | (6%) |
| Angioimmunoblastic T-Cell NHL | 4 | (2%) | — | | 4 | (3%) |
| T-PLL | 3 | (2%) | — | | 3 | (2%) |
| T-ALL | 3 | (2%) | — | | 3 | (2%) |
| Peripheral T-Cell NHL | 2 | (1%) | — | | 2 | (1%) |
| Cutaneous T-Cell Lymphoma | 1 | (1%) | — | | 1 | (1%) |
| AML | 13 | (7%) | — | | 13 | (8%) |
| MPN/MDS | 6 | (3%) | — | | 6 | (4%) |
| Plasma Cell Neoplasm | 1 | (1%) | — | | 1 | (1%) |
| Other Diagnoses | 19 | (11%) | 0 | (0%) | 19 | (12%) |
| Acute EBV Infection (Mononucleosis) | 10 | (6%) | — | | 10 | (6%) |
| Non-Transplant-Associated EBV | | | — | | | |
| Reactivation | 6 | (3%) | | | 6 | (4%) |
| HLH | 3 | (2%) | — | | 3 | (2%) |
| Pharmacologic Immunosuppression | 111 | (62%) | 0 | (0%) | 111 | (72%) |
| Calcineurin Inhibitors | 109 | (61%) | — | | 109 | (70%) |
| Mycophenolate | 78 | (44%) | — | | 78 | (50%) |
| mTOR Inhibitors | 46 | (26%) | — | | 46 | (30%) |
| Azathioprine | 8 | (4%) | — | | 8 | (5%) |
| Methotrexate | 7 | (4%) | — | | 7 | (5%) |
| Any CMV Viremia | 66 | (37%) | 0 | (0%) | 66 | (43%) |

Values are provided as number (percent) or median (range).
EBV, Epstein-Barr Virus; NPC, nasopharyngeal carcinoma; IU, international units; DLBCL, diffuse large B-cell lymphoma; ALL, acute lymphoblastic leukemia; NKTCL, NK/T-cell lymphoma; NHL, non-Hodgkin lymphoma; PLL, prolymphocytic leukemia; AML, acute myeloid leukemia; MPN/MDS, myeloproliferative neoplasm/myelodysplastic syndrome; HLH, hemophagocytic lymphohistiocytosis; CMV, cytomegalovirus.
*Sum exceeds 71 due to select patients having multi-organ transplantation.

TABLE 6
Modeled Transition Probabilities, Management Assumptions, Health Utilities, and Costs
with Parameter Ranges for Deterministic and Probabilistic Sensitivity Analyses

| Transition | Base | Lower | Upper | |
|---|---|---|---|---|
| Probability | Case | Bound | Bound | Distribution |
| Health to undetected stage I | age/sex/population-specific | | | 1,000 calibrated sets |
| NPC | (incidence databases) | | | |
| Undetected Stage I NPC to | calibrated from screening trials and | | | matrix of 1,000 |
| Undetected Stage II-IVC NPC | SEER detection rates (Miller et al. | | | calibrated sets |
| | 2021) | | | |
| Undetected Stage I-IVC NPC | calibrated from screening trials and | | | matrix of 1,000 |
| to Detected Stage I-IVC NPC | SEER detection rates (Miller et al. | | | calibrated sets |
| | 2021) | | | |
| Screening Test | | | | |
| Sensitivity | strategy-specific (Table 10) | −0.05 | +0.05 | binomial |
| Specificity | strategy-specific (Table 10) | −0.02 | +0.02 | binomial |
| Compliance | strategy-specific (Table 10) | −0.05 | +0.05 | binomial |
| High-Risk BALF2 Haplotype | 93.0% NPC cases, 60.5% non-NPC | 20% | 20% | binomial |
| Prevalence | controls | decrease | increase | |
| Initial Stage Distribution of | | | | |
| Undetected NPC | | | | |
| Stage I | 0.349 | 0.283 | 0.430 | Dirichlet |
| Stage II | 0.381 | 0.317 | 0.449 | Dirichlet |
| Stage III | 0.230 | 0.174 | 0.322 | Dirichlet |
| Stage IVA/B | 0.040 | 0.031 | 0.075 | Dirichlet |
| Stage IVC | 0.000 | 0.000 | 0.000 | |
| Stage I-IVA/B NPC to LRR | stage-specific, time-dependent | Upper | Upper | sampled from |
| | (Supplementary Methods) | 95% CI | 95% CI | modeled 95% CI |
| Stage I-IVA/B NPC to DM | stage-specific, time-dependent | Lower | Upper | sampled from |
| | (Supplementary Methods) | 95% CI | 95% CI | modeled 95% CI |
| Stage I-IVC NPC to Death | stage-specific, time-dependent | Lower | Upper | sampled from |
| | (Supplementary Methods) | 95% CI | 95% CI | modeled 95% CI |
| Proportion with WHO II/III | population-specific (Table 11) | −0.05 | +0.05 | beta |
| Histology | | | | |
| Hazard Ratio for LRR, | 2.080 | 1.820 | 2.370 | normal |
| 2D/3DCRT vs. IMRT | | | | |
| Hazard Ratio for OS, | 1.700 | 1.360 | 2.120 | normal |
| 2D/3DCRT vs. IMRT | | | | |
| Screening Age | 50.000 | 40.000 | 60.000 | |
| Management Assumptions | | | | |
| Radiotherapy modality | IMRT (Table 11) | 3DCRT | IMRT | |
| Radiotherapy Fractions | 35 | 30 | 40 | |
| PET/CT staging | No PET/CT | No | PET/CT | |
| | | PET/CT | | |
| Palliative Radiotherapy | 0.500 | 0.100 | 0.900 | beta |
| Utilization (Recurrent Disease) | | | | |
| Health State Utilities | | | | |
| Perfect health | 1.000 | | | |
| False Positive | 1.000 | 0.900 (1 | 1.000 | beta |
| | | month) | | |
| Recently-diagnosed NPC | 0.910 | 0.860 | 0.960 | beta |
| prior to treatment (true positive) | | | | |
| [months 0-1] | | | | |
| Locoregional recurrence, | 0.620 | 0.570 | 0.670 | beta |
| distant metastasis, or de novo | | | | |
| metastatic disease | | | | |
| Under treatment with | 0.884 | 0.834 | 0.934 | beta |
| intensity-modulated | | | | |
| radiotherapy alone [months 1-3] | | | | |
| Under treatment with | 0.872 | 0.822 | 0.922 | beta |
| 2D/3D radiotherapy alone | | | | |
| [months 1-3] | | | | |
| Under treatment with | 0.833 | 0.783 | 0.883 | beta |
| intensity-modulated | | | | |
| chemoradiotherapy [months 1-3] | | | | |
| Under treatment with | 0.821 | 0.771 | 0.871 | beta |
| 2D/3D chemoradiotherapy | | | | |
| [months 1-3] | | | | |
| Remission after intensity- | 0.895 | 0.845 | 0.945 | beta |
| modulated radiotherapy alone | | | | |
| [months 4-12] | | | | |
| Remission after 2D/3D | 0.893 | 0.843 | 0.943 | beta |
| radiotherapy alone [months 4-12] | | | | |
| Remission after intensity- | 0.847 | 0.797 | 0.897 | beta |
| modulated chemoradiotherapy | | | | |
| [months 4-12] | | | | |
| Remission after 2D/3D | 0.846 | 0.796 | 0.896 | beta |
| chemoradiotherapy [months 4-12] | | | | |
| Remission after intensity- | 0.911 | 0.861 | 0.961 | beta |
| modulated radiotherapy alone | | | | |
| [months 12-24] | | | | |
| Remission after 2D/3D | 0.893 | 0.843 | 0.943 | beta |
| radiotherapy alone [months 12-24] | | | | |
| Remission after intensity- | 0.863 | 0.813 | 0.913 | beta |
| modulated chemoradiotherapy | | | | |
| [months 12-24] | | | | |
| Remission after 2D/3D | 0.845 | 0.795 | 0.895 | beta |
| chemoradiotherapy [months | | | | |
| 12-24] | | | | |
| Remission after intensity- | 0.881 | 0.831 | 0.931 | beta |
| modulated radiotherapy alone | | | | |
| [months 24-96] | | | | |
| Remission after 2D/3D | 0.846 | 0.796 | 0.896 | beta |
| radiotherapy alone [months 24-96] | | | | |
| Remission after intensity- | 0.833 | 0.783 | 0.883 | beta |
| modulated chemoradiotherapy | | | | |
| [months 24-96] | | | | |
| Remission after 2D/3D | 0.798 | 0.748 | 0.848 | beta |
| chemoradiotherapy [months | | | | |
| 24-96] | | | | |
| Remission after intensity- | 0.793 | 0.743 | 0.843 | beta |
| modulated radiotherapy alone | | | | |
| [months 96+] | | | | |
| Remission after 2D/3D | 0.777 | 0.727 | 0.827 | beta |
| radiotherapy alone [months 96+] | | | | |
| Remission after intensity- | 0.745 | 0.695 | 0.795 | beta |
| modulated chemoradiotherapy | | | | |
| [months 96+] | | | | |
| Remission after 2D/3D | 0.729 | 0.679 | 0.779 | beta |
| chemoradiotherapy [months 96+] | | | | |
| Death | 0.000 | | | |
| Costs | | | | |
| Total Screening Costs | country-specific (Table 11) | 0.500 | 2.000 | gamma |
| Radiotherapy (3DCRT or | country-specific (Table 11) | 0.500 | 2.000 | gamma |
| IMRT, Palliative or Definitive) | | | | |
| Chemotherapy | country-specific (Table 11) | 0.500 | 2.000 | gamma |
| (Concurrent, Adjuvant, or | | | | |
| Palliative) | | | | |
| Diagnostic Imaging | country-specific (Table 11) | 0.500 | 2.000 | gamma |
| (Workup, Recurrence, MRI | | | | |
| Screening) | | | | |
| PCR/ELISA Reagents | assay-specific (Table 11) | 0.500 | 2.000 | gamma |
| Sample Transportation | country-specific (Table 11) | 0.500 | 4.000 | gamma |
| Nasoendoscopy | country-specific (Table 11) | 0.500 | 2.000 | gamma |
| Wages and Labor Time | | | | |
| Admin. Assistant | country-specific (Table 11) | 0.500 | 2.000 | gamma |
| Phlebotomist | country-specific (Table 11) | 0.500 | 2.000 | gamma |
| Laboratory | country-specific (Table 11) | 0.500 | 2.000 | gamma |
| Technician | | | | |
| Driver (Sample | country-specific (Table 11) | 0.500 | 2.000 | gamma |
| Transportation) | | | | |
| Clinical Pathologist | country-specific (Table 11) | 0.500 | 2.000 | gamma |
| Nursing | country-specific (Table 11) | 0.500 | 2.000 | gamma |
| (Nasopharyngeal Swab) | | | | |
| Discount Rate | 0.030 | 0.000 | 0.050 | beta |

NPC, nasopharyngeal carcinoma; LRR, locoregional recurrence; 2D/3DCRT, 2D/3D conformal radiotherapy.

TABLE 7
Cost of Diagnosis, Work-up, and Definitive Radiotherapy in the United States

| Services: Diagnosis | | CPT/HCPCS | Unit | Total |
|---|---|---|---|---|
| and Work-up | Units | Code(s) | Cost | Costs |
| MRI nasopharynx with/without contrast | 1 | 70543 | $389.06 | $389.06 |
| CT head/neck with contrast | 1 | 70491 | $204.13 | $204.13 |
| CT chest without contrast | 1 | 71250 | $145.85 | $145.85 |
| Diagnostic nasopharyngolaryngoscopy | 1 | 31575 | $131.20 | $131.20 |
| Needle biopsy | 1 | 38505 | $125.96 | $125.96 |
| Tissue examination by pathologist | 1 | 88305 | $71.53 | $71.53 |
| Total costs | 1 | | | $1,067.73 |

| Services: Definitive Intensity- | | CPT/HCPCS | Unit | Total |
|---|---|---|---|---|
| Modulated Radiotherapy | Units | Code(s) | Cost | Costs |
| Physician consult | 1 | 99205 | $224.36 | $224.36 |
| Clinical treatment Plan | 1 | 77263 | $169.93 | $169.93 |
| Simulation | 1 | 77290 | $501.41 | $501.41 |
| Thermoplastic mask | 1 | 77334 | $128.06 | $128.06 |
| Isodose plan | 1 | 77307 | $128.06 | $128.06 |
| IMRT planning | 1 | 77301 | $1,935.17 | $1,935.17 |
| Multileaf collimator | 1 | 77338 | $480.48 | $480.48 |
| Dosimetry | 1 | 77331 | $65.95 | $65.95 |
| Daily image guidance | 35 | G6002 | $77.11 | $2,698.85 |
| IMRT treatment delivery | 35 | G6015 | $385.57 | $13,494.95 |
| Physics consult | 1 | 77370 | $130.85 | $130.85 |
| Weekly physics quality assurance | 7 | 77336 | $82.70 | $578.90 |
| Weekly treatment management | 7 | 77427 | $191.91 | $1,343.37 |
| Total Costs | | | | $21,880.34 |

CPT, current procedural terminology;
HCPCS, Healthcare Common Procedure Coding System;
MRI, magnetic resonance imaging;
CT, computed tomography;
IMRT, intensity-modulated radiotherapy.
All costs are reported in 2021 United States Dollars.

TABLE 8
Cost of Restaging for Local Recurrence or Distant Metastasis

| | CPT/HCPCS | Unit | Total |
|---|---|---|---|
| Services | Code(s) | Cost | Costs |
| History and physical (three multi-disciplinary | 99205 | $224.36 | $673.08 |
| consults) | | | |
| Diagnostic nasopharyngolaryngoscopy | 31575 | $131.20 | $131.20 |
| CT chest, abdomen, pelvis with IV contrast | 71260, 74177 | $523.74 | $523.74 |
| Needle biopsy | 38505 | $125.96 | $125.96 |
| Tissue examination by pathologist | 88305 | $71.53 | $71.53 |
| Total restaging costs | | | $1,525.51 |

CPT, current procedural terminology; HCPCS, Healthcare Common Procedure Coding System; CT, computed tomography.
Data are from Medicare Physician Fee Schedule 2021.⁶⁵ All costs are reported in 2021 United States Dollars.

TABLE 9
Cost of Chemotherapy and Supportive Care in the United States

| | | | | | | Supportive | | |
|---|---|---|---|---|---|---|---|---|
| | | | | | Agent | Medications | Infusion | Total |
| | CPT/ | | Unit | Cycle | Cost | and | Costs | Costs |
| | HCPCS | Unit | Dosage | Dosage* | Per | Laboratory | Per | Per |
| Chemotherapy Regimen | Code(s) | Cost | (mg) | (mg) | Cycle | Costs | Cycle | Course |
| Concurrent | | | | | | | | |
| Chemotherapy | | | | | | | | |
| Physician consult, once | 99205 | $224.36 | | | | | | $224.36 |
| Follow-up visit, once per | 99213 | $92.47 | | | | | | $277.41 |
| cycle | | | | | | | | |
| Cisplatin 100 mg/m², | J9060, | $1.89 | 10 | 200 | $37.00 | | $113.40 | $453.60 |
| concurrent, every three | 96409 | | | | | | | |
| weeks | | | | | | | | |
| Ondansetron 8 mg PO, | Q0162 | $0.02 | 1 | 224 | | $3.81 | | $11.42 |
| every six hours for one | | | | | | | | |
| week | | | | | | | | |
| Prochlorperazine 10 mg | Q0164 | $0.30 | 5 | 280 | | $17.02 | | $51.07 |
| PO, every six hours for | | | | | | | | |
| one week | | | | | | | | |
| Dexamethasone 8 mg PO, | J8540 | $0.10 | 0.25 | 8 | | $3.04 | | $9.12 |
| once per cycle | | | | | | | | |
| Normal saline 1 L IV, once | J7030, | $2.79 | 1000 | 1000 | | $2.79 | $36.29 | $117.24 |
| per cycle | 96360 | | | | | | | |
| Complete blood count, | 85025 | $7.77 | | | | $7.77 | | $23.31 |
| per cycle | | | | | | | | |
| Complete metabolic | 80053 | $10.56 | | | | $10.56 | | $31.68 |
| panel, per cycle | | | | | | | | |
| Total Costs per Course | | | | | | | | $1,199.22 |
| (3 Cycles) | | | | | | | | |
| Adjuvant Chemotherapy | | | | | | | | |
| Follow-up visit, once per | 99213 | $92.47 | | | | | | $277.41 |
| cycle | | | | | | | | |
| Cisplatin 80 mg/m² d1, | J9060, | $1.89 | 10 | 200 | $37.80 | | $113.40 | $453.60 |
| adjuvant, every four | 96409 | | | | | | | |
| weeks | | | | | | | | |
| 5-Fluorouracil 1000 mg/m² | J9190, | $1.79 | 500 | 8000 | $28.64 | | $147.25 | $527.67 |
| d1-4, adjuvant, every four | 96416 | | | | | | | |
| weeks | | | | | | | | |
| Ondansetron 8 mg PO, | Q0162 | $0.02 | 1 | 224 | | $3.81 | | $11.42 |
| every six hours for one | | | | | | | | |
| week | | | | | | | | |
| Prochlorperazine 10 mg | Q0164 | $0.30 | 5 | 280 | | $17.02 | | $51.07 |
| PO, every six hours for | | | | | | | | |
| one week | | | | | | | | |
| Dexamethasone 8 mg PO, | J8540 | $0.10 | 0.25 | 8 | | $3.20 | | $9.60 |
| once per cycle | | | | | | | | |
| Normal Saline 1 L IV, | J7030, | $2.79 | 1000 | 1000 | | $2.79 | $36.29 | $117.24 |
| once per cycle | 96360 | | | | | | | |
| Complete blood count, | 85025 | $7.77 | | | | $7.77 | | $23.31 |
| per cycle | | | | | | | | |
| Complete metabolic | 80053 | $10.56 | | | | $10.56 | | $31.68 |
| panel, per cycle | | | | | | | | |
| Total Costs per Course | | | | | | | | $1,503.01 |
| (3 Cycles) | | | | | | | | |
| Indefinite | | | | | | | | |
| Chemotherapy for | | | | | | | | |
| Metastatic Disease | | | | | | | | |
| Physician consult, once | 99205 | $224.36 | | | | | | $224.36 |
| Follow-up visit, once per | 99213 | $92.47 | | | | | | $554.82 |
| cycle | | | | | | | | |
| Gemcitabine 1000 mg/m² | J9201, | $4.14 | 200 | 4000 | $82.86 | | $226.80 | $1,857.96 |
| d1, 8, metastatic, every | 96409 | | | | | | | |
| three weeks | | | | | | | | |
| Cisplatin 80 mg/m² d1, | J9060, | | | 200 | $37.80 | | $113.40 | |
| metastatic, every three | 96409 | $1.89 | 10 | | | | | $907.20 |
| weeks | | | | | | | | |
| Ondansetron 8 mg PO, | Q0162 | $0.02 | 1 | 224 | | $3.81 | | $22.85 |
| every six hours for one | | | | | | | | |
| week | | | | | | | | |
| Prochlorperazine 10 mg | Q0164 | $0.30 | 5 | 280 | | $17.02 | | $102.14 |
| PO, every six hours for | | | | | | | | |
| one week | | | | | | | | |
| Dexamethasone 8 mg PO, | J8540 | $0.10 | 0.25 | 8 | | $3.20 | | $19.20 |
| once per cycle | | | | | | | | |
| Normal saline 1 L IV, once | J7030, | $2.79 | 1000 | 1000 | | $2.79 | $36.29 | $234.48 |
| per cycle | 96360 | | | | | | | |
| Complete blood count, | 85025 | $7.77 | | | | $7.77 | | $46.62 |
| per cycle | | | | | | | | |
| Complete metabolic | 80053 | $10.56 | | | | $10.56 | | $63.36 |
| panel, per cycle | | | | | | | | |
| CT chest, abdomen, | 71260, | $523.74 | | | | | | $1,047.48 |
| pelvis with contrast, every | 74177 | | | | | | | |
| three cycles | | | | | | | | |
| Total Costs per Course | | | | | | | | $5,080.47 |
| (6 Cycles) | | | | | | | | |

CPT, current procedural terminology; HCPCS, Healthcare Common Procedure Coding System; PO, oral. Data are from 2021 Medicare Physician Fee Schedule, Laboratory Fee Schedule, and Drug Average Sales Price.^{44, 45, 65} All costs are reported in 2021 United States Dollars.
^{a}Based on 2.0 m² body surface area adult. Palliative chemotherapy is administered for six cycles every three weeks.

TABLE 10
Performance Characteristics and Resource Utilization for Variant-Agnostic and BALF2 Variant-
Informed NPC Screening Strategies. We evaluated seven variant-agnostic and seven corresponding
variant-informed screening strategies, which were each compared with no screening. Variant-informed
screening increased PPV by a median of 46% (range, 26-51%) and decreased absolute screening sensitivity
by 7%. For example, the strategy reported by Chan et al. (strategy F₀, tandem plasma EBV BamHI-
W DNA followed by MRI and endoscopy) had screening sensitivity and PPV of 97.1% and 11.0%,
which changed to 90.4% and 16.0% after triaging the first positive PCR with BALF2 genotyping.
For this identical screened population of 20,174 subjects, this would amount to approximately
2.4 missed NPC cases and 108.5 fewer false-positives.

| | | | | Resource Utilization Per Screened Subject** | | | |
|---|---|---|---|---|---|---|---|
| | | | | Plasma | | | |
| | | Person- | Prevalent | BamHI- | NP | | |
| Strategy ID | Screening Strategy | Years* | Cases* | W PCR | PCR | ELISA | MRI |
| A₀ | Plasma EBV BamHI- | 21,492 | 38 | 1 | 0 | 0 | 0 |
| | W DNA PCR→ | | | | | | |
| | Endoscopy | | | | | | |
| A_{BALF2} | Plasma EBV BamHI- | — | — | 1 | 0 | 0 | 0 |
| | W DNA PCR + BALF2 | | | | | | |
| | PCR→ | | | | | | |
| | Endoscopy | | | | | | |
| B₀ | Plasma EBV BamHI- | 20,174 | 35 | 1 | 0 | 0 | 0.0551 |
| | W DNA PCR→ | | | | | | |
| | MRI nasopharynx | | | | | | |
| B_{BALF2} | Plasma EBV BamHI- | — | — | 1 | 0 | 0 | 0.0339 |
| | W DNA PCR + BALF2 | | | | | | |
| | PCR→ | | | | | | |
| | MRI nasopharynx | | | | | | |
| C₀ | Plasma EBV BamHI- | 20,174 | 35 | 1 | 0 | 0 | 0.0551 |
| | W DNA PCR→ | | | | | | |
| | Endoscopy + MRI | | | | | | |
| | nasopharynx | | | | | | |
| C_{BALF2} | Plasma EBV BamHI- | — | — | 1 | 0 | 0 | 0.0339 |
| | W DNA PCR + BALF2 | | | | | | |
| | PCR→ | | | | | | |
| | Endoscopy + MRI | | | | | | |
| | nasopharynx | | | | | | |
| D₀ | Plasma EBV BamHI- | 21,492 | 38 | 1.0555 | 0 | 0 | 0 |
| | W DNA PCR→ | | | | | | |
| | Plasma EBV BamHI- | | | | | | |
| | W DNA PCR→ | | | | | | |
| | Endoscopy | | | | | | |
| D_{BALF2} | Plasma EBV BamHI- | — | — | 1.0334 | 0 | 0 | 0 |
| | W DNA PCR + BALF2 | | | | | | |
| | PCR→ | | | | | | |
| | Plasma EBV BamHI- | | | | | | |
| | W DNA PCR→ | | | | | | |
| | Endoscopy | | | | | | |
| E₀ | Plasma EBV BamHI- | 20,174 | 35 | 1.0551 | 10 | 0 | 0.0153 |
| | W DNA PCR→ | | | | | | |
| | Plasma EBV BamHI- | | | | | | |
| | W DNA PCR→ | | | | | | |
| | MRI nasopharynx | | | | | | |
| E_{BALF2} | Plasma EBV BamHI- | — | — | 1.0334 | 0 | 0 | 0.0098 |
| | W DNA PCR + BALF2 | | | | | | |
| | PCR→ | | | | | | |
| | Plasma EBV BamHI- | | | | | | |
| | W DNA PCR→ | | | | | | |
| | MRI nasopharynx | | | | | | |
| F₀ | Plasma EBV BamHI- | 20,174 | 35 | 1.0551 | 10 | 0 | 0.0153 |
| | W DNA PCR→ | | | | | | |
| | Plasma EBV BamHI- | | | | | | |
| | W DNA PCR→ | | | | | | |
| | Endoscopy + MRI | | | | | | |
| | nasopharynx | | | | | | |
| F_{BALF2} | Plasma EBV BamHI- | — | — | 1.0334 | 0 | 0 | 0.0098 |
| | W DNA PCR + BALF2 | | | | | | |
| | PCR→ | | | | | | |
| | Plasma EBV BamHI- | | | | | | |
| | W DNA PCR→ | | | | | | |
| | Endoscopy + MRI | | | | | | |
| | nasopharynx | | | | | | |
| G₀ | Serum EBV VCA IgA | 22,186 | 8 | 0 | 0.0482 | 1 | 0 |
| | [>1:5]→ | | | | | | |
| | Nasopharyngeal EBV | | | | | | |
| | BamHI-W DNA PCR | | | | | | |
| | [mean + 2SD]→ | | | | | | |
| | Endoscopy | | | | | | |
| G_{BALF2} | Serum EBV VCA IgA | — | — | 0 | 0.0482 | 1 | 0 |
| | [>1:5]→ | | | | | | |
| | Nasopharyngeal EBV | | | | | | |
| | BamHI-W DNA PCR | | | | | | |
| | [mean + 2SD] + BALF2 | | | | | | |
| | PCR→ | | | | | | |
| | Endoscopy | | | | | | |

| | Resource Utilization Per Screened Subject** | | | | | | |
|---|---|---|---|---|---|---|---|
| | | | Visits | | | | |
| | Exam + | BALF2 | Per | | | | |
| Strategy ID | Endoscopy | PCR | Subject | Sensitivity | Specificity | PPV | Citation(s)* |
| A₀ | 0.0555 | 0 | 1.0555 | 89.47% | 94.67% | 2.89% | ^{32, 35} |
| A_{BALF2} | 0.0338 | 1 | 1.0338 | 82.39% | 96.76% | 4.23% | ^{4-6, 32, 35} |
| B₀ | 0.0206 | 0 | 1.084 | 97.14% | 94.65% | 3.06% | ³² |
| B_{BALF2} | 0.0127 | 1 | 1.0394 | 90.37% | 96.76% | 4.62% | ^{4-6, 32} |
| C₀ | 0.0551 | 0 | 1.110 | 97.14% | 94.65% | 3.06% | ³² |
| C_{BALF2} | 0.0339 | 1 | 1.0678 | 90.37% | 96.76% | 4.62% | ^{4-6, 32} |
| D₀ | 0.0153 | 0 | 1.0708 | 89.47% | 98.63% | 10.43% | ^{32, 35} |
| D_{BALF2} | 0.0097 | 1 | 1.0430 | 82.39% | 99.17% | 14.77% | ^{4-6, 32, 35} |
| E₀ | 0.0057 | 0 | 1.0795 | 97.14% | 98.63% | 11.00% | ³² |
| E_{BALF2} | 0.0037 | 1 | 1.0457 | 90.37% | 99.17% | 15.97% | ^{4-6, 32} |
| F₀ | 0.0153 | 0 | 1.0858 | 97.14% | 98.63% | 11.00% | ³² |
| F_{BALF2} | 0.00982 | 1 | 1.0530 | 90.37% | 99.17% | 15.97% | ^{4-6, 32} |
| G₀ | 0.0009 | 0 | 1.0491 | 87.50% | 99.95% | 41.18% | ³⁶ |
| G_{BALF2} | 0.0007 | 0.0482 | 1.0489 | 81.40% | 99.97% | 51.82% | ^{4-6, 36} |

NPC, nasopharyngeal carcinoma; PCR, polymerase chain reaction; ELISA, enzyme-linked immunosorbent assay; MRI, magnetic resonance imaging; NP, nasopharyngeal; PPV, positive predictive value.
*Screening performance characteristics and resource utilization for variant-agnostic (A₀-I₀) screening strategies are derived from four prospective screening trials. ^{32, 35, 36, 66} For strategies A₀ and D₀, performance and resource utilization was pooled between Chan et al. 2013 and Chan et al. 2017. For BALF2 variant-informed screening strategies, high-risk and low-risk haplotype distributions derived from a meta-analysis of Xu et al. 2019, Hui et al. 2019, and Lam et al. 2020 are used to triage plasma/nasopharyngeal specimens positive for EBV DNA. Person-years refers to the number of screening person-years. Prevalent cases refers to the number of true positives and false negatives within one year of screening. For cited studies above, full details regarding screened population, inclusion criteria, screening years, prevalence estimates, stage distributions, and contingency tables may be found in original publications.
**Average resource utilization per screening subject. For example, 5.5% of individuals screen positive by plasma BamHI-W PCR and undergo a second PCR for strategies D₀, E₀, F₀, resulting in an average of 1.055 plasma PCR per screened subject. Visits per subject refers to the average number of unique in-person visits per screened subject. This is equal to the sum of plasma PCR, NP PCR, ELISA, MRI, and exam + endoscopy. BALF2 qPCR is performed from residual extracted nucleic acids, and does not require an additional visit for
specimen collection.

TABLE 12
Analytical Performance: Multiplex BALF2 Genotyping qPCR Lower Limit of Detection in Replicates of 20

| | Number of Detected Replicates and 95% Lower Limit of Detection | | | | | | | |
|---|---|---|---|---|---|---|---|---|
| dsDNA Control | V700 | | V700L | | I613V | | V317M | |
| Concentration | (FAM) | | (CAL560) | | (CAL610) | | (Q670) | |
| 0.1 copies/μL template | 13/20 | | 12/20 | | 12/20 | | 12/20 | |
| 0.5 copies/μL template | 20/20 | | 20/20 | | 20/20 | | 20/20 | |
| 1.0 copies/μL template | 20/20 | | 20/20 | | 20/20 | | 20/20 | |
| 5.0 copies/μL template | 20/20 | | 20/20 | | 20/20 | | 20/20 | |
| 95% LLOD (95% CI) - copies/μL | 0.19 | (0.13-0.25) | 0.20 | (0.14-0.26) | 0.20 | (0.14-0.26) | 0.20 | (0.14-0.26) |
| template | | | | | | | | |
| 95% LLOD (95% CI) - | 1.9 | (1.3-2.5) | 2.0 | (1.4-2.6) | 2.0 | (1.4-2.6) | 2.0 | (1.4-2.6) |
| copies/reaction* | | | | | | | | |
| 95% LLOD (95% CI) - | 11.4 | (7.8-15.0) | 12.0 | (8.4-15.6) | 12.0 | (8.4-15.6) | 12.0 | (8.4-15.6) |

copies/mL plasma*

LLOD, lower limit of detection; CI, confidence interval.
*10 μL nucleic acid template included per 25 μL reaction. LLOD in plasma calculated by extrapolating from nucleic acid extraction protocol: 1000 μL plasma extracted into 60 μL elution buffer (AVE), with 10 μL template in each 25 μL reaction (6X ratio from copies/reaction to copies/mL plasma).

TABLE 13
Analytical Performance: Multiplex BALF2 Genotyping qPCR Linearity
in Replicates of Three Across Six Orders of Magnitude

| | Replicate Cₜ Values | | | |
|---|---|---|---|---|
| dsDNA Control | V700 | V700L | I613V | V317M |
| Concentration | (FAM) | (CAL560) | (CAL610) | (Q670) |
| 10⁰ copies/μL template | 40.12, 37.56, 38.58 | 36.37, 36.39, 36.91 | 38.11, 36.73, 37.57 | 39.08, 37.70, 39.05 |
| 10¹ copies/μL template | 35.48, 35.23, 35.22 | 35.04, 34.48, 34.09 | 34.57, 34.61, 34.66 | 36.04, 35.78, 35.76 |
| 10² copies/μL template | 32.06, 31.39, 31.43 | 30.56, 30.71, 30.64 | 31.10, 30.73, 30.96 | 32.44, 32.09, 32.20 |
| 10³ copies/μL template | 28.66, 27.99, 28.03 | 27.08, 27.10, 27.08 | 27.51, 27.41, 27.50 | 29.02, 28.75, 28.77 |
| 10⁴ copies/μL template | 24.57, 24.28, 24.12 | 23.12, 23.26, 23.20 | 23.73, 23.66, 23.53 | 25.14, 25.03, 24.95 |
| 10⁵ copies/μL template | 21.58, 21.21, 21.09 | 19.83, 19.98, 19.90 | 21.00, 20.65, 20.56 | 21.98, 22.08, 22.00 |
| R² | 0.9926 | 0.9927 | 0.9963 | 0.9958 |
| 10⁰ copies/μL template | 220.72% | 38.28% | 252.50% | 224.00% |
| 10¹ copies/μL template | 4.20% | 17.09% | 1.52% | 4.93% |
| 10² copies/μL template | 5.18% | 1.10% | 2.72% | 2.67% |
| 10³ copies/μL template | 3.55% | 0.14% | 0.52% | 1.51% |
| 10⁴ copies/μL template | 1.57% | 0.51% | 0.70% | 0.69% |
| 10⁵ copies/μL template | 1.44% | 0.43% | 1.36% | 0.33% |
| R² | — | — | — | |

Cₜ, cycle threshold.
*Based on linear regression standard curves: V700-FAM: copies/μL template = 10\hat{ }(10.910 − 0.281*Cₜ); V700L-CAL560: copies/μL template = 10\hat{ }(10.742 − 0.288*Cₜ); I613V-CAL610: copies/μL template = 10\hat{ }(10.966 − 0.291*Cₜ); V317M-Q670: copies/μL template = 10\hat{ }(11.431 − 0.293*Cₜ).

TABLE 14
Analytical Performance: Multiplex EBV BALF2 Genotyping qPCR
Minor Allele Frequency Detection in Replicates of Three

| Risk Allele | | | | |
|---|---|---|---|---|
| Frequency | Replicate Cₜ Values | | | |
| (V700L, I613V, | V700 | V700L | I613V | V317M |
| V317M)* | (FAM) | (CAL560) | (CAL610) | (Q670) |
| 0% | ndet, ndet, ndet | 30.56, 30.71, 30.64 | ndet, ndet, ndet | ndet, ndet, ndet |
| 1% | ndet, ndet, ndet | 30.48, 30.23, 30.5 | ndet, ndet, ndet | ndet, ndet, ndet |
| 5% | ndet, ndet, 44.11 | 30.19, 30.08, 29.95 | ndet, ndet, ndet | 38.25, 39.51, 38.25 |
| 10% | 40.33, 39.96, 38.88 | 30.1, 30.05, 29.88 | 43.73, 43.75, 44.79 | 37.14, 36.93, 36.29 |
| 20% | 35.06, 34.89, 34.38 | 30.42, 30.44, 30.35 | 34.5, 34.73, 34.76 | 34.5, 34.83, 34.54 |
| 30% | 33.93, 33.82, 33.48 | 30.81, 30.7, 30.61 | 33.43, 33.57, 33.26 | 33.75, 33.93, 33.82 |
| 40% | 32.93, 33.13, 32.64 | 31.01, 31.11, 31.05 | 32.42, 32.58, 32.36 | 33.26, 33.4, 33.04 |
| 50% | 32.44, 32.55, 32.41 | 31.41, 31.65, 31.51 | 32.02, 32.09, 31.87 | 32.97, 32.99, 32.94 |
| 60% | 32.27, 32.15, 32.1 | 31.87, 32.03, 31.8 | 31.64, 31.53, 31.51 | 32.76, 32.64, 32.59 |
| 70% | 32.03, 32.07, 32.01 | 32.98, 32.94, 33.03 | 31.19, 31.33, 31.25 | 32.48, 32.55, 32.54 |
| 80% | 31.36, 31.37, 31.31 | 33.35, 33.49, 33.6 | 30.87, 30.87, 30.81 | 32.23, 32.04, 31.96 |
| 90% | 31.01, 31.02, 31.07 | 37.16, 37.12, 40.85 | 30.34, 30.57, 30.52 | 31.63, 31.73, 31.71 |
| 95% | 31.04, 31.08, 31.05 | ndet, ndet, 40.85 | 30.48, 30.41, 30.41 | 31.73, 31.7, 31.66 |
| 99% | 31.03, 31.1, 31.26 | ndet, ndet, ndet | 30.32, 30.38, 30.49 | 31.66, 31.61, 31.73 |
| 100% | 32.06, 31.39, 31.43 | ndet, ndet, ndet | 31.1, 30.73, 30.96 | 32.44, 32.09, 32.2 |

| Risk Allele | | | | |
|---|---|---|---|---|
| Frequency | Replicate Measured Concentrations (copies/μL template)** | | | |
| (V700L, I613V, | V700 | V700L | I613V | V317M |
| V317M)* | (FAM) | (CAL560) | (CAL610) | (Q670) |
| 0% | 0.00, 0.00, 0.00 | 89.46, 81.11, 84.78 | 0.00, 0.00, 0.00 | 0.00, 0.00, 0.00 |
| 1% | 0.00, 0.00, 0.00 | 94.47, 111.66, 93.28 | 0.00, 0.00, 0.00 | 0.00, 0.00, 0.00 |
| 5% | 0.00, 0.00, 0.03 | 114.74, 122.98, 134.18 | 0.00, 0.00, 0.00 | 1.62, 0.69, 1.62 |
| 10% | 0.37, 0.48, 0.95 | 121.79, 126.02, 140.27 | 0.02, 0.02, 0.01 | 3.42, 3.95, 6.08 |
| 20% | 11.31, 12.69, 17.58 | 98.57, 97.03, 103.32 | 8.76, 7.53, 7.36 | 20.31, 16.27, 19.80 |
| 30% | 23.61, 25.27, 31.47 | 76.16, 81.80, 86.68 | 18.00, 16.39, 20.12 | 33.75, 29.84, 32.16 |
| 40% | 44.94, 39.62, 54.21 | 66.74, 62.26, 64.74 | 35.37, 31.77, 36.66 | 47.12, 42.96, 54.47 |
| 50% | 61.99, 57.69, 63.05 | 51.09, 43.60, 47.76 | 46.14, 44.12, 50.91 | 57.29, 56.39, 58.53 |
| 60% | 69.23, 74.47, 76.99 | 37.66, 33.83, 39.50 | 59.54, 64.08, 64.89 | 65.99, 71.77, 74.09 |
| 70% | 80.61, 78.47, 81.79 | 18.07, 18.52, 17.51 | 80.27, 73.35, 77.17 | 79.60, 75.98, 76.64 |
| 80% | 124.52, 123.62, 128.33 | 14.15, 12.89, 11.99 | 99.20, 99.45, 103.57 | 94.56, 107.20, 113.62 |
| 90% | 155.83, 155.28, 149.77 | 1.14, 1.16, 0.10 | 142.22, 121.60, 125.75 | 141.30, 132.71, 134.40 |
| 95% | 152.77, 148.79, 152.24 | 0.00, 0.00, 0.10 | 129.05, 135.25, 134.99 | 132.68, 134.79, 138.94 |
| 99% | 153.79, 147.16, 133.02 | 0.00, 0.00, 0.00 | 143.72, 138.26, 128.52 | 138.61, 143.36, 132.68 |
| 100% | 79.15, 121.71, 118.49 | 0.00, 0.00, 0.00 | 85.34, 109.20, 93.97 | 81.69, 103.47, 96.21 |

Cₜ, cycle threshold.
*Total template concentration (dsDNA risk control and dsDNA non-risk control) is fixed at 100 copies/μL, and the proportion of each control is varied from 0-100% of the risk allele.
**Based on standard curves: V700-FAM: copies/μL template = 10\hat{ }(10.910 − 0.281*Cₜ); V700L-CAL560: copies/μL template = 10\hat{ }(10.742 − 0.288*Cₜ); I613V-CAL610: copies/μL template = 10\hat{ }(10.966 − 0.291*Cₜ); V317M-Q670: copies/μL template = 10\hat{ }(11.431 − 0.293*Cₜ).

TABLE 15
EBV BALF2 Genotyping qPCR Validation with Targeted Next-Generation Sequencing

| | Targeted BALF2 Next Generation Sequencing | | | | | | | |
|---|---|---|---|---|---|---|---|---|
| | V700 | | V700L | | 1613V | | V317M | |
| BALF2 | | Not | | Not | | Not | | Not |
| qPCR | Detected | Detected | Detected | Detected | Detected | Detected | Detected | Detected |
| Detected | 129 | 0 | 23 | 0 | 23 | 0 | 9 | 0 |
| Not Detected | 0 | 23 | 0 | 129 | 1 | 128 | 0 | 143 |

| | | | | |
|---|---|---|---|---|
| PPA (95% CI)* | 100% (97-100%) | 100% (85-100%) | 100% (85-100%) | 100% (66-100%) |
| NPA (95% CI)* | 100% (85-100%) | 100% (97-100%) | 99% (96-100%) | 100% (97-100%) |

PPA, positive percent agreement; NPA, negative percent agreement; CI, confidence interval.
*BALF2 qPCR relative to next-generation sequencing.

TABLE 16
EBV BALF2 Haplotype Distributions and Association with NPC

| | EBV BALF2 Haplotype | | | | | | | | | | Odds Ratio (95% CI): | | |
|---|---|---|---|---|---|---|---|---|---|---|---|---|---|
| | (162215-162476-166364) | | | | | | | | | | NPC vs. Non-NPC | | |
| | | | | | | | | | | | | | High |
| | Low | | Low | | High | | High | | | | High | High | Risk: |
| | Risk: | | Risk: | | Risk: | | Risk: | | | | Risk: | Risk: | C-C-C or |
| Cohort | A-T-C | | C-T-C | | C-C-C | | C-C-T | | Other | | C-C- C | C-C-T | C-C-T |
| EBV-Positive NPC Cases | 2 | (8.3%) | 4 | (16.7%) | 3 | (12.5%) | 15 | (62.5%) | 0 | (0%) | 7.9 (1.7- | 178.8 (33.1- | 39.0 (12.9- |
| | | | | | | | | | | | 37.1) | 965.3) | 118.2) |
| Non-NPC Controls | 17 | (11.0%) | 126 | (81.3%) | 9 | (5.8%) | 2 | (1.3%) | 1 | (0.6%)* | — | — | — |
| Solid Organ Transplant | 5 | (7.0%) | 63 | (88.7%) | 3 | (4.3%) | 0 | (0.0%) | 0 | (0.0%) | — | — | — |
| Bone Marrow Transplant | 8 | (15.7%) | 37 | (72.5%) | 4 | (7.8%) | 1 | (2.0%) | 1 | (2.0%) | — | — | — |
| Hematologic Neoplasms | 10 | (13.2%) | 60 | (78.9%) | 4 | (5.3%) | 1 | (1.3%) | 1 | (1.3%) | 0.8 (0.2- | 1.0 (0.1- | 0.9 (0.3- |
| | | | | | | | | | | | 3.2) | 17.0) | 3.0) |
| B-Cell | 4 | (11.8%) | 26 | (76.5%) | 3 | (8.8%) | 1 | (2.9%) | 0 | (0.0%) | 1.9 (0.4- | 3.8 (0.2- | 2.2 (0.6- |
| Lymphomas/Leukemia | | | | | | | | | | | 8.0) | 62.0) | 7.8) |
| B-Cell NHL | 1 | (5.9%) | 13 | (76.5%) | 2 | (11.8%) | 1 | (5.9%) | 0 | (0.0%) | 2.6 (0.5- | 9.2 (0.5- | 3.5 (0.8- |
| | | | | | | | | | | | 13.9) | 155.5) | 14.5) |
| B-ALL | 2 | (16.7%) | 9 | (75.0%) | 1 | (8.3%) | 0 | (0.0%) | 0 | (0.0%) | 1.5 (0.2- | — | 1.2 (0.1- |
| | | | | | | | | | | | 13.1) | | 10.3) |
| Hodgkin Lymphoma | 1 | (20%) | 4 | (80%) | 0 | (0%) | 0 | (0.0%) | 0 | (0.0%) | — | — | — |
| T-Cell | 5 | (22.8%) | 15 | (68.2%) | 1 | (4.5%) | 0 | (0.0%) | 1 | (4.5%) | 0.8 (0.1- | — | 0.6 (0.1- |
| Lymphoma/Leukemia | | | | | | | | | | | 6.5) | | 5.1) |
| NKTCL | 4 | (44.4%) | 5 | (55.6%) | 0 | (0.0%) | 0 | (0.0%) | 0 | (0.0%) | — | — | — |
| T-Cell | 1 | (7.7%) | 10 | (76.9%) | 1 | (7.7%) | 0 | (0.0%) | 1 | (7.7%) | 1.5 (0.2- | — | 1.2 (0.1- |
| Lymphomas/Leukemias | | | | | | | | | | | 13.1) | | 10.3) |
| AML | 1 | (7.7%) | 12 | (92.3%) | 0 | (0.0%) | 0 | (0.0%) | 0 | (0.0%) | — | — | — |
| MPN/MDS | 0 | (0.0%) | 6 | (100.0%) | 0 | (0.0%) | 0 | (0.0%) | 0 | (0.0%) | — | — | — |
| Plasma Cell Neoplasm | 0 | (0.0%) | 1 | (100.0%) | 0 | (0.0%) | 0 | (0.0%) | 0 | (0.0%) | — | — | — |
| Acute EBV Infection or | 3 | (15.8%) | 13 | (68.4%) | 2 | (10.5%) | 1 | (5.3%) | 0 | (0.0%) | 2.3 (0.4- | 7.9 (0.5- | 3.0 (0.7- |
| Non-Transplant EBV | | | | | | | | | | | 11.9) | 133.2) | 12.4) |
| Reactivation | | | | | | | | | | | | | |
| Other Published Cohorts | | | | | | | | | | | — | — | — |
| Xu et al. 2019 EBV- | 25 | (3.9%) | 13 | (2.0%) | 57 | (8.9%) | 539 | (84.4%) | 5 | (0.8%) | 3.0 (1.9- | 11.4 (7.9- | 9.0 (6.3- |
| Positive NPC Cases | | | | | | | | | | | 4.8) | 16.6) | 13.0) |
| Xu et al. 2019 Non- | 171 | (26.2%) | 65 | (10.0%) | 118 | (18.1%) | 293 | (44.9%) | 5 | (0.8%) | — | — | — |
| NPC Controls | | | | | | | | | | | | | |
| Hui et al. 2019 | 4 | (6.5%) | 1 | (1.6%) | 2 | (3.2%) | 55 | (88.7%) | 0 | (0.0%) | 2.0 (0.4- | 13.3 (5.0- | 11.1 (4.2- |
| EBV-Positive | | | | | | | | | | | | | |
| NPC Cases | | | | | | | | | | | 11.4) | 35.4) | 29.3) |
| Hui et al. 2019 Non- | 53 | (37.3%) | 17 | (12.0%) | 14 | (9.9%) | 58 | (40.8%) | 0 | (0.0%) | — | — | — |
| NPC Controls | | | | | | | | | | | | | |
| Lam et al. 2020 EBV- | 2 | (6.7%) | 1 | (3.3%) | 2 | (6.7%) | 25 | (83.3%) | 0 | (0.0%) | 5.0 (0.5- | 8.3 (2.1- | 7.9 (2.0- |
| Positive NPC Cases | | | | | | | | | | | 50.8) | 33.6) | 31.6) |
| Lam et al. 2020 Non- | 12 | (37.4%) | 3 | (9.4%) | 2 | (6.3%) | 15 | (46.9%) | 0 | (0.0%) | — | — | — |
| NPC Controls | | | | | | | | | | | | | |
| Xu, Hui, and Lam | 31 | (4.2%) | 15 | (2.1%) | 61 | (8.3%) | 619 | (84.7%) | 5 | (0.7%) | 3.2 (2.1- | 11.8 (8.4- | 9.5 (6.8- |
| Aggregated EBV- | | | | | | | | | | | 4.9) | 16.5) | 13.2) |
| Positive Cases | | | | | | | | | | | | | |
| Xu, Hui, and Lam | 236 | (28.6%) | 85 | (10.3%) | 134 | (16.2%) | 366 | (44.3%) | 5 | (0.6%) | — | — | — |
| Aggregated Non-NPC | | | | | | | | | | | | | |
| Controls | | | | | | | | | | | | | |
| All EBV-Positive | 33 | (4.3%) | 19 | (2.5%) | 64 | (8.5%) | 634 | (84.0%) | 5 | (0.7%) | 4.0 (2.6- | 15.4 (11.2- | 12.2 (9.0- |
| NPC Cases | | | | | | | | | | | 6.0) | 21.0) | 16.6) |
| All Non-NPC Controls | 253 | (25.8%) | 211 | (21.5%) | 143 | (14.6%) | 368 | (37.5%) | 6 | (0.6%) | — | — | — |

Values are presented as number (percent) or odds ratio (95% confidence interval [CI]).
*Odds ratios represents high-risk haplotype (C-C-T and/or C-C-C) relative to sum of common low-risk haplotypes A-T-C and C-T-C (reference). For EBV-Positive NPC cases, the reference group includes all non-NPC controls for each individual study (present cohort, Xu et al., Hui et al., Lam et al.). Odds ratios for select non-NPC hematologic malignancies use all other non-NPC controls as the reference group. EBV, Epstein-Barr Virus; NPC, nasopharyngeal carcinoma; NHL, non-Hodgkin lymphoma, ALL, acute lymphoblastic leukemia; NKTCL, NK/T-cell lymphoma; AML, acute myeloid leukemia; MPN/MDS, myeloproliferative neoplasm/myelodysplastic syndrome.
*One patient with EBV-positive T-ALL had a single specimen positive for V700, V700L, and I613V (EBNA-1 viral load <100 IU/mL; V700, V700L, and I613V Cₜ 40.0, 40.3, 39.1), suggesting multiple infections. Sequencing of this specimen had low coverage, with a single V700L read, two I613 reads, and two V317 reads (haplotype [A/C]-C-C).

TABLE 17
Association Between Observed EBV BALF2 Single Nucleotide
Variants and NPC in Genotyped Specimens.

| Single | Variant | Variant | | | |
|---|---|---|---|---|---|
| Nucleotide | Frequency, | Frequency, | Odds Ratio and 95% CI | | |
| Variant | NPC Cases | Non-NPC Controls | (NPC vs. Non-NPC) | | P-Value |
| 162147G > A | 20% | 20% | 1.00 | (0.02-11.21) | 1.00E+00 |
| 162195A > C | 43% | 7% | 10.71 | (2.30-47.14) | 8.65E−04 |
| 162215C > A (V700L) | 8% | 11% | 0.74 | (0.08-3.48) | 1.00E+00 |
| 162237C > G | 50% | 10% | 8.73 | (2.12-34.76) | 9.21E−04 |
| 162273C > T | 19% | 46% | 0.27 | (0.05-1.06) | 5.63E−02 |
| 162298A > G | 0% | 5% | 0.00 | (0.00-6.07) | 1.00E+00 |
| 162464G > A | 53% | 10% | 10.00 | (2.88-34.21) | 5.87E−05 |
| 162476T > C (I613V) | 75% | 7% | 39.27 | (11.57-141.19) | 1.35E−12* |
| 162507C > T | 26% | 35% | 0.65 | (0.17-2.10) | 6.03E−01 |
| 162577T > C | 0% | 7% | 0.00 | (0.00-6.75) | 1.00E+00 |
| 162675T > C | 100% | 96% | Undefined | | 1.00E+00 |
| 162681G > A | 6% | 3% | 1.65 | (0.03-17.93) | 5.20E−01 |
| 162780A > C | 0% | 3% | 0.00 | (0.00-9.70) | 1.00E+00 |
| 162852G > T | 31% | 42% | 0.64 | (0.16-2.19) | 5.85E−01 |
| 162885G > A | 19% | 46% | 0.27 | (0.05-1.07) | 5.58E−02 |
| 162963A > G | 94% | 95% | 0.82 | (0.08-41.55) | 1.00E+00 |
| 163074C > T | 6% | 5% | 1.38 | (0.03-15.26) | 5.78E−01 |
| 163107A > C | 41% | 11% | 5.74 | (1.47-21.16) | 5.02E−03 |
| 163145G > A | 0% | 6% | 0.00 | (0.00-4.00) | 5.94E−01 |
| 163206C > T | 5% | 5% | 0.95 | (0.02-8.53) | 1.00E+00 |
| 163283C > T | 0% | 4% | 0.00 | (0.00-10.06) | 1.00E+00 |
| 163287G > A | 53% | 7% | 14.20 | (3.63-54.99) | 2.13E−05* |
| 163293G > A | 29% | 28% | 1.05 | (0.27-3.54) | 1.00E+00 |
| 163364C > T (V317M) | 63% | 1% | 127.50 | (22.68-1,182.58) | 6.16E−14* |
| 163377A > G | 78% | 48% | 3.76 | (1.08-16.50) | 2.30E−02 |
| 163404C > A | 50% | 11% | 7.75 | (0.85-65.65) | 3.62E−02 |
| 163422G > T | 60% | 10% | 13.50 | (1.24-175.59) | 1.53E−02 |
| 163458T > C | 80% | 53% | 3.57 | (0.33-180.75) | 3.69E−01 |
| 163464G > A | 60% | 1% | 103.50 | (4.56-5,208.38) | 5.80E−04 |

Includes single nucleotide variants observed in at least 3 patients. NPC, nasopharyngeal carcinoma; CI, confidence interval.
*Exceeds Bonferroni-corrected statistical significance threshold when adjusting for multiple hypothesis testing.

TABLE 18
Association Between Observed EBV BALF2 Single
Nucleotide Variants and BALF2 Haplotypes

| | Variant | Variant | Variant | P-Value | P-Value |
|---|---|---|---|---|---|
| Single | Frequency | Frequency | Frequency | (H-H-L vs. | (H-H-H vs. |
| Nucleotide | H-H-L | H-H-H | Low-Risk | Low-Risk | Low-Risk |
| Variant | Haplotype | Haplotype | Haplotypes | Haplotypes) | Haplotypes) |
| 162147G > A | 0% | 0% | 25% | 1.89E−01 | 1.00E+00 |
| 162195A > C | 36% | 100% | 2% | 1.04E−03 | 2.60E−09* |
| 162237C > G | 82% | 100% | 2% | 1.27E−09* | 2.60E−09* |
| 162273C > T | 9% | 0% | 49% | 1.14E−02 | 3.81E−03 |
| 162298A > G | 18% | 0% | 4% | 1.25E−01 | 1.00E+00 |
| 162464G > A | 85% | 100% | 0% | 2.81E−13* | 4.24E−14* |
| 162507C > T | 15% | 20% | 40% | 1.26E−01 | 3.12E−01 |
| 162577T > C | 0% | 0% | 8% | 1.00E+00 | 1.00E+00 |
| 162675T > C | 100% | 100% | 96% | 1.00E+00 | 1.00E+00 |
| 162681G > A | 42% | 0% | 0% | 8.61E−06* | 1.00E+00 |
| 162780A > C | 17% | 0% | 2% | 6.46E−02 | 1.00E+00 |
| 162852G > T | 42% | 20% | 44% | 1.00E+00 | 1.86E−01 |
| 162885G > A | 25% | 0% | 48% | 2.16E−01 | 4.44E−03 |
| 162963A > G | 100% | 100% | 95% | 1.00E+00 | 1.00E+00 |
| 163074C > T | 36% | 11% | 0% | 1.29E−04 | 1.02E−01 |
| 163107A > C | 46% | 100% | 3% | 1.05E−04 | 1.48E−09* |
| 163145G > A | 15% | 0% | 5% | 2.08E−01 | 1.00E+00 |
| 163206C > T | 38% | 9% | 1% | 7.46E−05 | 2.03E−01 |
| 163283C > T | 31% | 0% | 0% | 1.50E−04 | 1.00E+00 |
| 163287G > A | 54% | 100% | 0% | 7.54E−08* | 4.69E−14* |
| 163293G > A | 23% | 10% | 34% | 5.41E−01 | 1.65E−01 |
| 163377A > G | 85% | 100% | 47% | 1.61E−02 | 6.67E−04 |
| 163404C > A | 67% | 100% | 0% | 7.01E−07* | 1.21E−07* |
| 163422G > T | 56% | 100% | 0% | 1.21E−05* | 1.31E−07* |
| 163458T > C | 78% | 100% | 49% | 1.57E−01 | 5.52E−02 |
| 163464G > A | 0% | 80% | 0% | 1.00E+00 | 7.87E−06* |

Includes single nucleotide variants observed in at least 3 patients, excluding variants that define haplotypes (162215C > A [V700L], 162476T > C [I613V], 163364C > T [V317M].
H-H-L haplotype is defined by V700, I613V, and V317. H-H-H haplotype is defined by V700, I613V, and V317M.
All other haplotypes are considered low-risk for NPC.
NPC, nasopharyngeal carcinoma; CI, confidence interval.
*Exceeds Bonferroni-corrected statistical significance threshold when adjusting for multiple hypothesis testing.

TABLE 19
Base Case Resource Utilization, NPC Mortality Reduction, and Cost-Effectiveness among Men and Women for Variant-
Agnostic and BALF2 Variant-Informed Screening Strategies. For a hypothetical cohort of 50-year-old men and women
who develop NPC in southern China under base case assumptions, 10-year survival improved from 70.4% (95% CI 68.1-
72.5%) in an unscreened cohort to a median of 85.7% (range, 85.4-87.0%) with variant-agnostic screening and 85.2%
(range, 84.3-85.9%) with variant-informed screening). This corresponded to a median 10-year reduction in NPC-specific
death of 51.8% (range, 50.7-56.3%) with variant-agnostic screening and 47.7% (range, 47.1-52.3%) with BALF2 variant-
informed screening. In the highest incidence region, the 7% relative reduction in screening sensitivity after BALF2
triage resulted in approximately 3.4 excess NPC deaths per 100,000 after onetime screening. For strategies A₀-C₀ and G₀,
the corresponding variant-informed strategies reduced screening costs by a median of 25.7% (range, 0.1-49.8%). For
strategies D₀-F₀, the corresponding variant-informed strategies increased costs by a median of 17.2% (range, 5.1-35.8%).
Although per-subject screening costs were slightly increased with strategies D_{BALF2}-F_{BALF2}, total incremental costs
were lower after integrating all costs of screening, work-up, and treatment. Variant-informed screening reduced
referrals for endoscopy and/or MRI by approximately 40% relative to the corresponding variant-agnostic strategy,
which is equal to the population prevalence of low-risk BALF2 haplotypes. This reduction in referrals for the second
and third steps of screening averted a median of 2,969 screening visits per 100,000 subjects (range, 35-5,459). At the
ICER/GDPₚₚₚ ≤ 2.0 willingness-to-pay (WTP) threshold, screening was cost-effective in all populations except Hengdong,
China (due to lower NPC incidence). Variant-informed screening typically offered similar ICERs due to slightly reduced
screening sensitivity (7%), a slight increase in laboratory costs, and a 40% reduction in referrals for endoscopy/MRI.

| | | Resource Utilization Per | | | | | | | |
|---|---|---|---|---|---|---|---|---|---|
| | | 100,000 Screened Subjects* | | | Cost-Effectiveness** | | | | |
| | | | | Total | NPC | | | | |
| Strategy | Screening | | | Screening | Deaths | Incremental | ICER/ | ICER/ | ICER/ |
| ID | Strategy | Endoscopies | MRIs | Visits | Per 100,000 | Cost ($I) | GDPₚₚₚ | GDPₚₚₚ ≤ 2.0 | GDPₚₚₚ ≤ 1.0 |
| | No Screening | — | — | — | 185.22 | — | — | — | — |
| | | | | | (87.39- | | | | |
| | | | | | 452.15) | | | | |
| A₀ | Plasma EBV | 5507 | 0 | 104783 | 172.30 | 34.37 | 1.12 | 11/12 | 5/12 |
| | BamHI-W DNA | (5495- | (0-0) | (104557- | (78.56- | (34.15- | (0.35- | (92%) | (42%) |
| | PCR→ | 5523) | | 105097) | 419.79) | 65.41) | 2.38) | | |
| | Endoscopy | | | | | | | | |
| A_{BALF2} | Plasma EBV | 3311 | 0 | 102587 | 173.67 | 32.63 | 1.15 | 11/12 | 5/12 |
| | BamHI-W DNA | (3304- | (0-0) | (102366- | (79.50- | (32.40- | (0.35- | (92%) | (42%) |
| | PCR + BALF2 | 3321) | | 102895) | 423.23) | 57.11) | 2.60) | | |
| | PCR→ | | | | | | | | |
| | Endoscopy | | | | | | | | |
| B₀ | Plasma EBV | 2044 | 5472 | 106792 | 171.46 | 36.28 | 1.13 | 11/12 | 5/12 |
| | BamHI-W DNA | (2039- | (5460- | (106562- | (77.98- | (35.45- | (0.36- | (92%) | (42%) |
| | PCR→ | 2050) | 5489) | 107112) | 417.67) | 72.11) | 2.33) | | |
| | MRI nasopharynx | | | | | | | | |
| B_{BALF2} | Plasma EBV | 1236 | 3311 | 103823 | 172.83 | 33.53 | 1.12 | 11/12 | 5/12 |
| | BamHI-W DNA | (1234- | (3304- | (103600 | (78.92- | (32.72- | (0.34- | (92%) | (42%) |
| | PCR + BALF2 | 1240) | 3321) | 104135) | 421.12) | 59.89) | 2.47) | | |
| | PCR→ | | | | | | | | |
| | MRI nasopharynx | | | | | | | | |
| C₀ | Plasma EBV | 5472 | 5472 | 110220 | 171.46 | 40.02 | 1.29 | 11/12 | 5/12 |
| | BamHI-W DNA | (5460- | (5460- | (109983- | (77.98- | (39.20- | (0.43- | (92%) | (42%) |
| | PCR→ | 5489) | 5489) | 110551) | 417.67) | 88.34) | 2.57) | | |
| | Endoscopy + MRI | | | | | | | | |
| | nasopharynx | | | | | | | | |
| C_{BALF2} | Plasma EBV | 3311 | 3311 | 105897 | 172.83 | 35.80 | 1.25 | 11/12 | 5/12 |
| | BamHI-W DNA | (3304- | (3304- | (105670- | (78.92- | (34.99- | (0.39- | (92%) | (42%) |
| | PCR + BALF2 | 3321) | 3321) | 106215) | 421.12) | 68.69) | 2.63) | | |
| | PCR→ | | | | | | | | |
| | Endoscopy + MRI | | | | | | | | |
| | nasopharynx | | | | | | | | |
| D₀ | Plasma EBV | 1520 | 0 | 106305 | 172.30 | 31.06 | 0.89 | 11/12 | 6/12 |
| | BamHI-W DNA | (1516- | (0-0) | (106076- | (78.56- | (30.84- | (0.26- | (92%) | (50%) |
| | PCR→ | 1524) | | 106624) | 419.79) | 48.82) | 2.15) | | |
| | Plasma EBV | | | | | | | | |
| | BamHI-W DNA | | | | | | | | |
| | PCR→ | | | | | | | | |
| | Endoscopy | | | | | | | | |
| D_{BALF2} | Plasma EBV | 920 | 0 | 103507 | 173.67 | 30.44 | 0.98 | 11/12 | 6/12 |
| | BamHI-W DNA | (918- | (0-0) | (103284- | (79.50- | (30.21- | (0.28- | (92%) | (50%) |
| | PCR + BALF2 | 923) | | 103817) | 423.23) | 46.46) | 2.42) | | |
| | PCR→ | | | | | | | | |
| | Plasma EBV | | | | | | | | |
| | BamHI-W DNA | | | | | | | | |
| | PCR→ | | | | | | | | |
| | Endoscopy | | | | | | | | |
| E₀ | Plasma EBV | 568 | 1521 | 106839 | 171.46 | 31.15 | 0.84 | 11/12 | 7/12 |
| | BamHI-W DNA | (567- | (1517- | (106610- | (77.98- | (30.33- | (0.24- | (92%) | (58%) |
| | PCR→ | 570) | 1525) | 107160) | 417.67) | 48.59) | 2.00) | | |
| | Plasma EBV | | | | | | | | |
| | BamHI-W DNA | | | | | | | | |
| | PCR→ | | | | | | | | |
| | MRI nasopharynx | | | | | | | | |
| E_{BALF2} | Plasma EBV | 344 | 920 | 103850 | 172.83 | 30.25 | 0.90 | 11/12 | 6/12 |
| | BamHI-W DNA | (343- | (918- | (103627- | (78.92- | (29.44- | (0.25- | (92%) | (50%) |
| | PCR + BALF2 | 345) | 923) | 104162) | 421.12) | 46.11) | 2.22) | | |
| | PCR→ | | | | | | | | |
| | Plasma EBV | | | | | | | | |
| | BamHI-W DNA | | | | | | | | |
| | PCR→ | | | | | | | | |
| | MRI nasopharynx | | | | | | | | |
| F₀ | Plasma EBV | 1521 | 1521 | 107792 | 171.46 | 32.19 | 0.89 | 11/12 | 7/12 |
| | BamHI-W DNA | (1517- | (1517- | (107560- | (77.98- | (31.37- | (0.26- | (92%) | (58%) |
| | PCR→ | 1525) | 1525) | 108116) | 417.67) | 52.49) | 2.07) | | |
| | Plasma EBV | | | | | | | | |
| | BamHI-W DNA | | | | | | | | |
| | PCR→ | | | | | | | | |
| | Endoscopy + MRI | | | | | | | | |
| | nasopharynx | | | | | | | | |
| F_{BALF2} | Plasma EBV | 920 | 920 | 104427 | 172.83 | 30.88 | 0.94 | 11/12 | 6/12 |
| | BamHI-W DNA | (918- | (918- | (104202- | (78.92- | (30.07- | (0.27- | (92%) | (50%) |
| | PCR + BALF2 | 923) | 923) | 104740) | 421.12) | 47.89) | 2.27) | | |
| | PCR→ | | | | | | | | |
| | Plasma EBV | | | | | | | | |
| | BamHI-W DNA | | | | | | | | |
| | PCR→ | | | | | | | | |
| | Endoscopy + MRI | | | | | | | | |
| | nasopharynx | | | | | | | | |
| G₀ | Serum EBV VCA | 90 | 0 | 104154 | 172.69 | 14.97 | 0.44 | 12/12 | 11/12 |
| | IgA [>1:5]→ | (90- | (0-0) | (103930- | (78.83- | (14.72- | (0.14- | (100%) | (92%) |
| | Nasopharyngeal | 90) | | 104467) | 420.76) | 26.14) | 1.06) | | |
| | EBV BamHI-W | | | | | | | | |
| | DNA PCR | | | | | | | | |
| | [mean + 2SD]→ | | | | | | | | |
| | Endoscopy | | | | | | | | |
| G_{BALF2} | Serum EBV VCA | 54 | 0 | 104118 | 173.83 | 14.93 | 0.49 | 12/12 | 11/12 |
| | IgA [>1:5]→ | (54- | (0-0) | (103894- | (79.61- | (14.68- | (0.16- | (100%) | (92%) |
| | Nasopharyngeal | 55) | | 104431) | 423.64) | 26.01) | 1.20) | | |
| | EBV BamHI-W | | | | | | | | |
| | DNA PCR | | | | | | | | |
| | [mean + 2SD] + BAL | | | | | | | | |
| | F2 PCR→ | | | | | | | | |
| | Endoscopy | | | | | | | | |

Values are reported as median (range) or counts (percent) across the 12 high-risk populations in southern China, Hong Kong SAR, Macao SAR, Singapore, and the Republic of China.
NPC, nasopharyngeal carcinoma; PCR, polymerase chain reaction; ELISA, enzyme-linked immunosorbent assay; MRI, magnetic resonance imaging; $I, 2021 international dollars; ICER/GDPₚₚₚ, incremental cost-effectiveness ratio divided by the purchasing power parity-adjusted gross domestic product per capita in the population of interest. Willingness to pay thresholds of 2.0 and 1.0 were evaluated.
*Median (range) resource utilization per 100,000 screened subjects. For example, 5.5% of individuals screen positive by plasma BamHI-W PCR and undergo immediate nasoendoscopy for strategy A₀, yielding 5,507 endoscopies per 100,000 screened subjects. Total screening visits refers to all unique in-person screening visits. This is equal to the sum of plasma PCR, NP PCR, ELISA, MRI, and exam + endoscopy. BALF2 qPCR is performed from residual extracted nucleic acids, and does not require an additional visit for specimen collection.
**Measures of cost-effectiveness for each onetime screening strategy across the 12 included populations, including median (range) incremental cost per screened subject, median (range) NPC deaths over lifetime horizon, median (range) ICER/GDPₚₚₚ, and the number (percent) of screened populations which were cost-effective for each strategy at two willingness to pay thresholds (1.0 and 2.0 ICER/GDPₚₚₚ).

TABLE 20
Base Case Resource Utilization, NPC Mortality Reduction, and Cost-Effectiveness among
Only Men for Variant-Agnostic and BALF2 Variant-Informed Screening Strategies. Screening
was more cost-effective when limited only to men due to higher incidence.

| | | Resource Utilization Per | | | | | | | |
|---|---|---|---|---|---|---|---|---|---|
| | | 100,000 Screened Subjects* | | | Cost-Effectiveness** | | | | |
| | | | | Total | NPC | | | | |
| Strategy | Screening | | | Screening | Deaths | Incremental | ICER/GD | ICER/GD | ICER/GD |
| ID | Strategy | Endoscopies | MRIs | Visits | Per 100,000 | Cost ($I) | Pₚₚₚ | Pₚₚₚ ≤2.0 | Pₚₚₚ ≤1.0 |
| | No Screening | — | — | — | 274.90 | — | — | — | — |
| | | | | | (118.56- | | | | |
| | | | | | 674.12) | | | | |
| A₀ | Plasma EBV | 5490 | 0 | 104475 | 256.06 | 34.56 | 0.85 | 12/12 | 10/12 |
| | BamHI-W | (5473- | (0-0) | (104149- | (105.64- | (34.14- | (0.23- | (100%) | (83%) |
| | DNA PCR→ | 5517) | | 104985) | 625.02) | 65.92) | 1.74) | | |
| | Endoscopy | | | | | | | | |
| A_{BALF2} | Plasma EBV | 3301 | 0 | 102286 | 258.06 | 32.82 | 0.89 | 12/12 | 8/12 |
| | BamHI-W | (3291- | (0-0) | (101966- | (107.02- | (32.40- | (0.23- | (100%) | (67%) |
| | DNA | 3317) | | 102785) | 630.27) | 57.65) | 1.91) | | |
| | PCR + BALF2 | | | | | | | | |
| | PCR→ | | | | | | | | |
| | Endoscopy | | | | | | | | |
| B₀ | Plasma EBV | 2038 | 5456 | 106479 | 241.98 | 36.20 | 0.86 | 12/12 | 9/12 |
| | BamHI-W | (2031- | (5439- | (106146- | (104.78- | (34.85- | (0.22- | (100%) | (75%) |
| | DNA PCR→ | 2047) | 5483) | 106998) | 621.76) | 71.16) | 1.69) | | |
| | MRI | | | | | | | | |
| | nasopharynx | | | | | | | | |
| B_{BALF2} | Plasma EBV | 1233 | 3301 | 103519 | 256.83 | 33.46 | 0.85 | 12/12 | 10/12 |
| | BamHI-W | (1229- | (3291- | (103195- | (106.18- | (32.13- | (0.21- | (100%) | (83%) |
| | DNA | 1239) | 3317) | 104024) | 627.06) | 59.05) | 1.80) | | |
| | PCR + BALF2 | | | | | | | | |
| | PCR→ | | | | | | | | |
| | MRI | | | | | | | | |
| | nasopharynx | | | | | | | | |
| C₀ | Plasma EBV | 5456 | 5456 | 109897 | 241.98 | 39.93 | 0.95 | 12/12 | 7/12 |
| | BamHI-W | (5439- | (5439- | (109554- | (104.78- | (38.58- | (0.27- | (100%) | (58%) |
| | DNA PCR→ | 5483) | 5483) | 110434) | 621.76) | 87.38) | 1.87) | | |
| | Endoscopy + | | | | | | | | |
| | MRI | | | | | | | | |
| | nasopharynx | | | | | | | | |
| C_{BALF2} | Plasma EBV | 3301 | 3301 | 105587 | 256.83 | 35.72 | 0.94 | 12/12 | 8/12 |
| | BamHI-W | (3291- | (3291- | (105257- | (106.18- | (34.38- | (0.25- | (100%) | (67%) |
| | DNA | 3317) | 3317) | 106102) | 627.06) | 67.84) | 1.92) | | |
| | PCR + BALF2 | | | | | | | | |
| | PCR→ | | | | | | | | |
| | Endoscopy + | | | | | | | | |
| | MRI | | | | | | | | |
| | nasopharynx | | | | | | | | |
| D₀ | Plasma EBV | 1515 | 0 | 105993 | 256.06 | 31.26 | 0.68 | 12/12 | 10/12 |
| | BamHI-W | (1511- | (0-0) | (105662- | (105.64- | (30.84- | (0.17- | (100%) | (83%) |
| | DNA PCR→ | 1523) | | 106511) | 625.02) | 49.37) | 1.57) | | |
| | Plasma EBV | | | | | | | | |
| | BamHI-W | | | | | | | | |
| | DNA PCR→ | | | | | | | | |
| | Endoscopy | | | | | | | | |
| D_{BALF2} | Plasma EBV | 917 | 0 | 103203 | 258.06 | 30.64 | 0.74 | 12/12 | 10/12 |
| | BamHI-W | (914- | (0-0) | (102880- | (107.02- | (30.21- | (0.19- | (100%) | (83%) |
| | DNA | 922) | | 103707) | 630.27) | 46.92) | 1.78) | | |
| | PCR + BALF2 | | | | | | | | |
| | PCR→ | | | | | | | | |
| | Plasma EBV | | | | | | | | |
| | BamHI-W | | | | | | | | |
| | DNA PCR→ | | | | | | | | |
| | Endoscopy | | | | | | | | |
| E₀ | Plasma EBV | 566 | 1516 | 106526 | 241.98 | 31.08 | 0.63 | 12/12 | 10/12 |
| | BamHI-W | (564- | (1511- | (106193- | (104.78- | (29.74- | (0.15- | (100%) | (83%) |
| | DNA PCR→ | 569) | 1524) | 107046) | 621.76) | 47.91) | 1.46) | | |
| | Plasma EBV | | | | | | | | |
| | BamHI-W | | | | | | | | |
| | DNA PCR→ | | | | | | | | |
| | MRI | | | | | | | | |
| | nasopharynx | | | | | | | | |
| E_{BALF2} | Plasma EBV | 343 | 917 | 103546 | 256.83 | 30.18 | 0.68 | 12/12 | 10/12 |
| | BamHI-W | (341- | (914- | (103222- | (106.18- | (28.85- | (0.16- | (100%) | (83%) |
| | DNA | 344) | 922) | 104051) | 627.06) | 45.44) | 1.62) | | |
| | PCR + BALF2 | | | | | | | | |
| | PCR→ | | | | | | | | |
| | Plasma EBV | | | | | | | | |
| | BamHI-W | | | | | | | | |
| | DNA PCR→ | | | | | | | | |
| | MRI | | | | | | | | |
| | nasopharynx | | | | | | | | |
| F₀ | Plasma EBV | 1516 | 1516 | 107476 | 241.98 | 32.12 | 0.67 | 12/12 | 10/12 |
| | BamHI-W | (1511- | (1511- | (107140- | (104.78- | (30.78- | (0.16- | (100%) | (83%) |
| | DNA PCR→ | 1524) | 1524) | 108001) | 621.76) | 51.64) | 1.50) | | |
| | Plasma EBV | | | | | | | | |
| | BamHI-W | | | | | | | | |
| | DNA PCR→ | | | | | | | | |
| | Endoscopy + | | | | | | | | |
| | MRI | | | | | | | | |
| | nasopharynx | | | | | | | | |
| F_{BALF2} | Plasma EBV | 917 | 917 | 104120 | 256.83 | 30.81 | 0.71 | 12/12 | 10/12 |
| | BamHI-W | (914- | (914- | (103795- | (106.18- | (29.48- | (0.17- | (100%) | (83%) |
| | DNA | 922) | 922) | 104629) | 627.06) | 47.22) | 1.66) | | |
| | PCR + BALF2 | | | | | | | | |
| | PCR→ | | | | | | | | |
| | Plasma EBV | | | | | | | | |
| | BamHI-W | | | | | | | | |
| | DNA PCR→ | | | | | | | | |
| | Endoscopy + | | | | | | | | |
| | MRI | | | | | | | | |
| | nasopharynx | | | | | | | | |
| G₀ | Serum EBV | 90 | 0 | 103849 | 256.62 | 15.22 | 0.34 | 12/12 | 12/12 |
| | VCA IgA | (89- | (0-0) | (103524- | (106.03- | (14.76- | (0.09- | (100%) | (100%) |
| | [>1:5]> | 90) | | 104355) | 626.51) | 26.36) | 0.78) | | |
| | Nasopharyngeal | | | | | | | | |
| | EBV BamHI-W | | | | | | | | |
| | DNA PCR | | | | | | | | |
| | [mean + 2SD] → | | | | | | | | |
| | Endoscopy | | | | | | | | |
| G_{BALF2} | Serum EBV | 54 | 0 | 103813 | 258.30 | 15.18 | 0.38 | 12/12 | 12/12 |
| | VCA IgA | (54- | (0-0) | (103489- | (107.20- | (14.72- | (0.10- | (100%) | (100%) |
| | [>1:5]→ | 55) | | 104320) | 630.93) | 26.24) | 0.88) | | |
| | Nasopharyngeal | | | | | | | | |
| | EBV BamHI-W | | | | | | | | |
| | DNA PCR | | | | | | | | |
| | [mean + 2SD] + | | | | | | | | |
| | BALF2 PCR→ | | | | | | | | |
| | Endoscopy | | | | | | | | |

Values are reported as median (range) or counts (percent) across the 12 high-risk populations in southern China, Hong Kong SAR, Macao SAR, Singapore, and the Republic of China.
NPC, nasopharyngeal carcinoma; PCR, polymerase chain reaction; ELISA, enzyme-linked immunosorbent assay; MRI, magnetic resonance imaging; $I, 2021 international dollars; ICER/GDPₚₚₚ, incremental cost-effectiveness ratio divided by the purchasing power parity-adjusted gross domestic product per capita in the population of interest. Willingness to pay thresholds of 2.0 and 1.0 were evaluated.
*Median (range) resource utilization per 100,000 screened subjects. For example, 5.5% of individuals screen positive by plasma BamHI-W PCR and undergo immediate nasoendoscopy for strategy A₀, yielding 5,507 endoscopies per 100,000 screened subjects. Total screening visits refers to all unique in-person screening visits. This is equal to the sum of plasma PCR, NP PCR, ELISA, MRI, and exam + endoscopy. BALF2 qPCR is performed from residual extracted nucleic acids, and does not require an additional visit for specimen collection.
**Measures of cost-effectiveness for each onetime screening strategy across the 12 included populations, including median (range) incremental cost per screened subject, median (range) NPC deaths over lifetime horizon, median (range) ICER/GDPₚₚₚ, and the number (percent) of screened populations which were cost-effective for each strategy at two willingness to pay thresholds (1.0 and 2.0 ICER/GDPₚₚₚ).

TABLE 21
Cost-Effectiveness for Variant-Agnostic and BALF2 Variant-Informed Screening Strategies with Variable Screening
Age and Interval. Initial screening age was varied from 40-60 in five-year increments, and screening interval
was varied from every 1-5 years in addition to once-lifetime screening. All screening ended after age
70. Across the 12 populations and 18 screening strategies, an initial screening age of 40 or 45 tended
to be most cost-effective irrespective of screening interval. Interval screening was never as cost-effective
as once-lifetime screening, commensurate with the prevalence of undiagnosed preclinical NPC. However,
screening intervals as short as every two years could be cost-effective in the majority of populations
and screening strategies. As screening frequency increased, the absolute number of NPC deaths averted
increased while per-screen mortality reduction decreased. Variant-informed and variant-agnostic screening
had similar ICERs with once-lifetime screening, whereas variant-informed screening became more cost-effective
as the number of lifetime screens increased. This was due to the increasing proportion of individuals
known to have low-risk BALF2 haplotypes that were never subsequently screened.

| Screening | Median (Range) ICER/GDPₚₚₚ | | | | | Proportion of Scenarios Cost-Effective* | | | | |
|---|---|---|---|---|---|---|---|---|---|---|
| Frequency | Initial Screening Age | | | | | Initial Screening Age | | | | |
| Men and Women | 40 | 45 | 50 | 55 | 60 | 40 | 45 | 50 | 55 | 60 |
| Variant-Agnostic | | | | | | | | | | |
| Screening | | | | | | | | | | |
| Once Lifetime | 0.84 | 0.72 | 0.97 | 1.08 | 1.77 | 92.6% | 92.6% | 90.7% | 81.5% | 58.3% |
| | (0.11- | (0.12- | (0.14- | (0.21- | (0.22- | | | | | |
| | 2.83) | 3.27) | 2.88) | 3.81) | 11.48) | | | | | |
| Every Five Years | 1.41 | 1.45 | 1.59 | 1.80 | 2.28 | 66.7% | 67.6% | 61.1% | 55.6% | 41.7% |
| | (0.21- | (0.22- | (0.24- | (0.27- | (0.27- | | | | | |
| | 4.58) | 4.42) | 5.16) | 6.00) | 10.01) | | | | | |
| Every Four Years | 1.60 | 1.73 | 1.80 | 2.18 | 3.01 | 63.0% | 58.3% | 55.6% | 48.1% | 33.3% |
| | (0.24- | (0.26- | (0.26- | (0.31- | (0.34- | | | | | |
| | 5.16) | 5.18) | 5.81) | 7.23) | 11.96) | | | | | |
| Every Three Years | 1.62 | 1.80 | 1.98 | 2.27 | 3.35 | 61.1% | 55.6% | 50.9% | 44.4% | 29.6% |
| | (0.24- | (0.27- | (0.28- | (0.31- | (0.39- | | | | | |
| | 4.82) | 5.36) | 6.36) | 7.68) | 12.59) | | | | | |
| Every Two Years | 1.81 | 1.97 | 2.14 | 2.68 | 3.39 | 53.7% | 51.9% | 46.3% | 35.2% | 28.7% |
| | (0.27- | (0.29- | (0.31- | (0.35- | (0.37- | | | | | |
| | 5.52) | 5.85) | 6.85) | 8.85) | 12.35) | | | | | |
| Every One Year | 2.41 | 2.58 | 2.85 | 3.46 | 4.42 | 39.8% | 38.0% | 32.4% | 25.0% | 15.7% |
| | (0.36- | (0.38- | (0.41- | (0.43- | (0.48- | | | | | |
| | 7.34) | 7.78) | 9.27) | 12.00) | 17.74) | | | | | |
| BALF2 | | | | | | | | | | |
| Variant-Informed | | | | | | | | | | |
| Screening | | | | | | | | | | |
| Once Lifetime | 0.87 | 0.72 | 1.00 | 1.09 | 1.84 | 92.6% | 92.6% | 92.6% | 80.6% | 53.7% |
| | (0.12- | (0.13- | (0.16- | (0.23- | (0.25- | | | | | |
| | 2.78) | 3.22) | 2.85) | 3.81) | 11.61) | | | | | |
| Every Five Years | 1.45 | 1.36 | 1.55 | 1.80 | 2.51 | 67.6% | 67.6% | 57.4% | 54.6% | 38.9% |
| | (0.23- | (0.24- | (0.27- | (0.30- | (0.30- | | | | | |
| | 4.23) | 4.13) | 4.92) | 5.84) | 10.01) | | | | | |
| Every Four Years | 1.58 | 1.60 | 1.69 | 2.13 | 3.19 | 63.0% | 57.4% | 55.6% | 48.1% | 30.6% |
| | (0.26- | (0.28- | (0.29- | (0.35- | (0.39- | | | | | |
| | 4.68) | 4.75) | 5.47) | 6.95) | 11.90) | | | | | |
| Every Three Years | 1.57 | 1.62 | 1.84 | 2.21 | 3.52 | 63.9% | 56.5% | 51.9% | 45.4% | 27.8% |
| | (0.26- | (0.30- | (0.31- | (0.34- | (0.43- | | | | | |
| | 4.28) | 4.84) | 5.85) | 7.28) | 12.43) | | | | | |
| Every Two Years | 1.66 | 1.74 | 1.92 | 2.52 | 3.54 | 59.3% | 55.6% | 51.9% | 38.9% | 27.8% |
| | (0.29- | (0.32- | (0.34- | (0.38- | (0.41- | | | | | |
| | 4.76) | 5.13) | 6.15) | 8.16) | 12.03) | | | | | |
| Every One Year | 2.02 | 2.11 | 2.35 | 3.06 | 4.38 | 49.1% | 47.2% | 38.0% | 26.9% | 18.5% |
| | (0.38- | (0.41- | (0.45- | (0.47- | (0.53- | | | | | |
| | 5.93) | 6.43) | 7.88) | 10.67) | 16.66) | | | | | |
| Men Only | 40 | 45 | 50 | 55 | 60 | 40 | 45 | 50 | 55 | 60 |
| Variant-Agnostic | | | | | | | | | | |
| Screening | | | | | | | | | | |
| Once Lifetime | 0.56 | 0.50 | 0.73 | 0.78 | 1.29 | 100.0% | 97.2% | 99.1% | 92.6% | 76.9% |
| | (0.08- | (0.08- | (0.09- | (0.15- | (0.23- | | | | | |
| | 1.86) | 2.40) | 2.10) | 3.19) | 10.12) | | | | | |
| Every Five Years | 1.06 | 1.02 | 1.14 | 1.34 | 1.70 | 82.4% | 81.5% | 82.4% | 75.9% | 62.0% |
| | (0.15- | (0.16- | (0.18- | (0.23- | (0.28- | | | | | |
| | 3.07) | 3.28) | 4.01) | 5.04) | 10.14) | | | | | |
| Every Four Years | 1.17 | 1.21 | 1.25 | 1.55 | 2.16 | 79.6% | 76.9% | 75.9% | 65.7% | 46.3% |
| | (0.17- | (0.19- | (0.20- | (0.26- | (0.32- | | | | | |
| | 3.49) | 3.99) | 4.51) | 5.96) | 14.00) | | | | | |
| Every Three Years | 1.18 | 1.24 | 1.38 | 1.63 | 2.42 | 79.6% | 76.9% | 71.3% | 63.9% | 41.7% |
| | (0.17- | (0.20- | (0.23- | (0.26- | (0.33- | | | | | |
| | 3.52) | 4.10) | 4.98) | 6.49) | 16.36) | | | | | |
| Every Two Years | 1.32 | 1.36 | 1.45 | 1.88 | 2.43 | 75.0% | 69.4% | 65.7% | 52.8% | 42.6% |
| | (0.19- | (0.22- | (0.25- | (0.29- | (0.32- | | | | | |
| | 3.87) | 4.62) | 5.33) | 7.71) | 13.91) | | | | | |
| Every One Year | 1.75 | 1.77 | 1.94 | 2.39 | 3.19 | 57.4% | 55.6% | 50.9% | 44.4% | 33.3% |
| | (0.26- | (0.28- | (0.34- | (0.37- | (0.40- | | | | | |
| | 5.22) | 6.00) | 7.26) | 11.15) | 16.55) | | | | | |
| BALF2 | | | | | | | | | | |
| Variant-Informed | | | | | | | | | | |
| Screening | | | | | | | | | | |
| Once Lifetime | 0.58 | 0.51 | 0.73 | 0.77 | 1.42 | 100.0% | 94.4% | 99.1% | 92.6% | 72.2% |
| | (0.09- | (0.09- | (0.10- | (0.17- | (0.26- | | | | | |
| | 1.83) | 2.37) | 2.08) | 3.20) | 10.33) | | | | | |
| Every Five Years | 0.97 | 0.93 | 1.07 | 1.26 | 1.80 | 83.3% | 83.3% | 79.6% | 70.4% | 54.6% |
| | (0.17- | (0.18- | (0.21- | (0.26- | (0.32- | | | | | |
| | 2.84) | 3.08) | 3.84) | 4.93) | 10.29) | | | | | |
| Every Four Years | 1.04 | 1.08 | 1.16 | 1.49 | 2.31 | 80.6% | 77.8% | 76.9% | 60.2% | 44.4% |
| | (0.19- | (0.21- | (0.23- | (0.29- | (0.36- | | | | | |
| | 3.18) | 3.69) | 4.26) | 5.78) | 14.34) | | | | | |
| Every Three Years | 1.05 | 1.09 | 1.26 | 1.56 | 2.53 | 81.5% | 76.9% | 71.3% | 60.2% | 41.7% |
| | (0.19- | (0.23- | (0.25- | (0.29- | (0.37- | | | | | |
| | 3.15) | 3.73) | 4.63) | 6.24) | 16.79) | | | | | |
| Every Two Years | 1.09 | 1.17 | 1.31 | 1.77 | 2.51 | 78.7% | 75.9% | 67.6% | 53.7% | 44.4% |
| | (0.21- | (0.24- | (0.28- | (0.32- | (0.36- | | | | | |
| | 3.35) | 4.08) | 4.82) | 7.27) | 13.72) | | | | | |
| Every One Year | 1.34 | 1.41 | 1.59 | 2.07 | 3.10 | 68.5% | 64.8% | 54.6% | 48.1% | 36.1% |
| | (0.28- | (0.30- | (0.37- | (0.40- | (0.44- | | | | | |
| | 4.23) | 4.99) | 6.23) | 10.09) | 15.58) | | | | | |

Values are reported as median (range) or percentages across the 18 screening strategies (9 variant-agnostic, 9 variant-informed) and 12 high-risk populations in southern China, Hong Kong SAR, Macao SAR, Singapore, and the Republic of China.
ICER/GDPₚₚₚ, incremental cost-effectiveness ratio divided by the purchasing power parity-adjusted gross domestic product per capita in the population of interest.
*Cost-effective defined as willingness to pay threshold of 2.0 ICER/GDPₚₚₚ.

TABLE 22
Base Case Cost-Effectiveness and Probabilistic Sensitivity Analysis for Example Population of 50-Year Old Men and Women Screened
Once in Guangzhou, China. Probabilistic sensitivity analysis (PSA) assessed uncertainty around base case estimates. All base
case estimates for NPC mortality reduction, incremental costs, and ICERs were within the PSA 95% confidence intervals. Median ICERs
from PSA were slightly lower than base case estimates. In Guangzhou, nearly 100% of simulations were below the ICER/GDPₚₚₚ ≤
2.0 WTP threshold, whereas 38-100% of simulations were below a WTP threshold of 1.0, depending on screening strategy.

| | | | | | Probabilistic Sensitivity Analysis | | | | | |
|---|---|---|---|---|---|---|---|---|---|---|
| | | Base Case | | | | | | Simulations | Simulations | Simulations |
| | | NPC Deaths | | | NPC Deaths | | | ICER/ | ICER/ | ICER/ |
| Strategy | Screening | Averted | Incremental | ICER/ | Averted | Incremental | ICER/ | GDPₚₚₚ ≤ | GDPₚₚₚ ≤ | GDPₚₚₚ ≤ |
| ID | Strategy | per 100,000 | Cost | GDPₚₚₚ | per 100,000 | Cost | GDPₚₚₚ | 2.0 | 1.0 | 0.5 |
| A₀ | Plasma EBV | 16.40 | 34.22 | 1.28 | 20.65 | 33.05 | 0.90 | 100% | 63% | 2% |
| | BamHI-W | | | | (14.66- | (21.64- | (0.56- | | | |
| | DNA PCR→ | | | | 26.17) | 46.27) | 1.66) | | | |
| | Endoscopy | | | | | | | | | |
| A_{BALF2} | Plasma EBV | 14.66 | 32.47 | 1.40 | 18.39 | 32.26 | 0.97 | 99% | 52% | 0% |
| | BamHI-W | | | | (13.31- | (21.78- | (0.64- | | | |
| | DNA | | | | 23.55) | 36.51) | 1.71) | | | |
| | PCR + BALF2 | | | | | | | | | |
| | PCR→ | | | | | | | | | |
| | Endoscopy | | | | | | | | | |
| B₀ | Plasma EBV | 17.48 | 36.08 | 1.24 | 21.90 | 33.84 | 0.88 | 100% | 64% | 4% |
| | BamHI-W | | | | (15.44- | (23.85- | (0.52- | | | |
| | DNA PCR→ | | | | 27.63) | 48.43) | 1.59) | | | |
| | MRI | | | | | | | | | |
| | nasopharynx | | | | | | | | | |
| B_{BALF2} | Plasma EBV | 15.73 | 33.33 | 1.32 | 19.79 | 32.95 | 0.93 | 100% | 59% | 0% |
| | BamHI-W | | | | (14.08- | (23.43- | (0.60- | | | |
| | DNA | | | | 25.16) | 37.53) | 1.64) | | | |
| | PCR + BALF2 | | | | | | | | | |
| | PCR→ | | | | | | | | | |
| | MRI | | | | | | | | | |
| | nasopharynx | | | | | | | | | |
| C₀ | Plasma EBV | 17.48 | 39.82 | 1.37 | 21.90 | 37.87 | 0.97 | 100% | 56% | 1% |
| | BamHI-W | | | | (15.44- | (25.61- | (0.58- | | | |
| | DNA PCR→ | | | | 27.63) | 52.46) | 1.76) | | | |
| | Endoscopy + | | | | | | | | | |
| | MRI | | | | | | | | | |
| | nasopharynx | | | | | | | | | |
| C_{BALF2} | Plasma EBV | 15.73 | 35.60 | 1.41 | 19.79 | 35.06 | 1.00 | 99% | 49% | 0% |
| | BamHI-W | | | | (14.08- | (25.28- | (0.65- | | | |
| | DNA | | | | 25.16) | 39.73) | 1.73) | | | |
| | PCR + BALF2 | | | | | | | | | |
| | PCR→ | | | | | | | | | |
| | Endoscopy + | | | | | | | | | |
| | MRI | | | | | | | | | |
| | nasopharynx | | | | | | | | | |
| D₀ | Plasma EBV | 16.40 | 30.91 | 1.16 | 20.65 | 27.02 | 0.76 | 96% | 68% | 27. |
| | BamHI-W | | | | (14.66- | (6.70- | (0.15- | | | |
| | DNA PCR→ | | | | 26.17) | 57.22) | 2.04) | | | |
| | Plasma EBV | | | | | | | | | |
| | BamHI-W | | | | | | | | | |
| | DNA PCR→ | | | | | | | | | |
| | Endoscopy | | | | | | | | | |
| D_{BALF2} | Plasma EBV | 14.66 | 30.28 | 1.31 | 18.39 | 30.01 | 0.89 | 100% | 58% | 0% |
| | BamHI-W | | | | (13.31- | (19.89- | (0.61- | | | |
| | DNA | | | | 23.55) | 35.02) | 1.63) | | | |
| | PCR + BALF2 | | | | | | | | | |
| | PCR→ | | | | | | | | | |
| | Plasma EBV | | | | | | | | | |
| | BamHI-W | | | | | | | | | |
| | DNA PCR→ | | | | | | | | | |
| | Endoscopy | | | | | | | | | |
| E₀ | Plasma EBV | 17.48 | 30.95 | 1.07 | 21.90 | 27.19 | 0.72 | 98% | 71% | 30% |
| | BamHI-W | | | | (15.44- | (6.39- | (0.13- | | | |
| | DNA PCR→ | | | | 27.63) | 58.03) | 1.88) | | | |
| | Plasma EBV | | | | | | | | | |
| | BamHI-W | | | | | | | | | |
| | DNA PCR→ | | | | | | | | | |
| | MRI | | | | | | | | | |
| | nasopharynx | | | | | | | | | |
| E_{BALF2} | Plasma EBV | 15.73 | 30.05 | 1.19 | 19.79 | 29.91 | 0.82 | 100% | 73% | 3% |
| | BamHI-W | | | | (14.08- | (19.63- | (0.53- | | | |
| | DNA | | | | 25.16) | 34.72) | 1.50) | | | |
| | PCR + BALF2 | | | | | | | | | |
| | PCR→ | | | | | | | | | |
| | Plasma EBV | | | | | | | | | |
| | BamHI-W | | | | | | | | | |
| | DNA PCR→ | | | | | | | | | |
| | MRI | | | | | | | | | |
| | nasopharynx | | | | | | | | | |
| F₀ | Plasma EBV | 17.48 | 31.99 | 1.10 | 21.90 | 27.85 | 0.74 | 98% | 70% | 29% |
| | BamHI-W | | | | (15.44- | (6.70- | (0.13- | | | |
| | DNA PCR→ | | | | 27.63) | 59.51) | 1.95) | | | |
| | Plasma EBV | | | | | | | | | |
| | BamHI-W | | | | | | | | | |
| | DNA PCR→ | | | | | | | | | |
| | Endoscopy + | | | | | | | | | |
| | MRI | | | | | | | | | |
| | nasopharynx | | | | | | | | | |
| F_{BALF2} | Plasma EBV | 15.73 | 30.68 | 1.21 | 19.79 | 30.37 | 0.84 | 100% | 71% | 2% |
| | BamHI-W | | | | (14.08- | (20.36- | (0.54- | | | |
| | DNA | | | | 25.16) | 35.23) | 1.53) | | | |
| | PCR + BALF2 | | | | | | | | | |
| | PCR→ | | | | | | | | | |
| | Plasma EBV | | | | | | | | | |
| | BamHI-W | | | | | | | | | |
| | DNA PCR→ | | | | | | | | | |
| | Endoscopy + | | | | | | | | | |
| | MRI | | | | | | | | | |
| | nasopharynx | | | | | | | | | |
| G₀ | Serum EBV | 15.91 | 14.80 | 0.58 | 19.96 | 14.66 | 0.40 | 100% | 100% | 64% |
| | VCA IgA | | | | (14.25- | (9.70- | (0.25- | | | |
| | [>1:5]→ | | | | 25.44) | 18.80) | 0.78) | | | |
| | Nasopharyngeal | | | | | | | | | |
| | EBV BamHI-W | | | | | | | | | |
| | DNA PCR | | | | | | | | | |
| | [mean + 2SD] → | | | | | | | | | |
| | Endoscopy | | | | | | | | | |
| G_{BALF2} | Serum EBV | 14.45 | 14.76 | 0.65 | 18.08 | 14.62 | 0.44 | 100% | 100% | 59% |
| | VCA IgA | | | | (13.13- | (9.67- | (0.29- | | | |
| | [>1:5]→ | | | | 23.18) | 18.73) | 0.85) | | | |
| | Nasopharyngeal | | | | | | | | | |
| | EBV BamHI-W | | | | | | | | | |
| | DNA PCR | | | | | | | | | |
| | [mean + | | | | | | | | | |
| | 2SD] + | | | | | | | | | |
| | BALF2 | | | | | | | | | |
| | PCR→ | | | | | | | | | |
| | Endoscopy | | | | | | | | | |

Base case results are reported for population of 50 year-old men and women screened once in Guangzhou, China. Corresponding results from probabilistic sensitivity analysis are reported as median (95% confidence interval) and percentages.
NPC, nasopharyngeal carcinoma; PCR, polymerase chain reaction; ELISA, enzyme-linked immunosorbent assay; MRI, magnetic resonance imaging; ICER/GDPₚₚₚ, incremental cost-effectiveness ratio divided by the purchasing power parity-adjusted gross domestic product per capita in the population of interest. Willingness to pay thresholds of 2.0, 1.0, and 0.5 were evaluated.

What is claimed is:

1. A method for genotyping one or more polymorphisms in a BALF2 gene of Epstein-Barr virus (EBV) using a nucleic acid amplification assay, the method comprising:
(a) obtaining a biological sample suspected of containing EBV nucleic acids from a subject;
(b) amplifying the EBV nucleic acids, if present, with a set of primers comprising:
(i) a forward primer comprising or consisting of the nucleotide sequence of SEQ ID NO: 3 and a reverse primer comprising or consisting of the nucleotide sequence of SEQ ID NO: 4,
(ii) a forward primer comprising or consisting of the nucleotide sequence of SEQ ID NO: 6 and a reverse primer comprising or consisting of the nucleotide sequence of SEQ ID NO: 7;
(iii) a forward primer comprising or consisting of the nucleotide sequence of SEQ ID NO: 9 and a reverse primer comprising or consisting of the nucleotide sequence of SEQ ID NO: 10;
(iv) a forward primer and a reverse primer comprising at least one nucleotide sequence that differs from the corresponding nucleotide sequence of the forward primer and the reverse primer of a set selected from the group consisting of (i)-(iii) in that the forward primer or the reverse primer has up to three nucleotide changes compared to the corresponding nucleotide sequence, wherein the forward primer and the reverse primer are capable of hybridizing to and amplifying the EBV nucleic acids in the nucleic acid amplification assay;
(v) a forward primer and a reverse primer that are complements of the corresponding nucleotide sequences of the forward primer and the reverse primer of a set selected from the group consisting of (i)-(iv); or
(vi) any combination of (i)-(v); and
(c) genotyping the BALF2 gene by detecting the presence of one or more alleles at the one or more polymorphisms of the BALF2 gene in the amplified nucleic acids using one or more detectably labeled allele-specific probes selected from:
(i) a detectably labeled allele-specific probe comprising or consisting of the nucleotide sequence of SEQ ID NO:5,
(ii) a detectably labeled allele-specific probe comprising or consisting of the nucleotide sequence of SEQ ID NO:8,
(iii) a detectably labeled allele-specific probe comprising or consisting of the nucleotide sequence of SEQ ID NO:11,
(iv) a detectably labeled allele-specific probe comprising or consisting of the nucleotide sequence of SEQ ID NO:12,
(v) a detectably labeled allele-specific probe comprising a nucleotide sequence having up to three nucleotide changes in a nucleotide sequence selected from the group consisting of SEQ ID NO:5, SEQ ID NO:8, SEQ ID NO:11, and SEQ ID NO:12, wherein the probe retains allele specificity of a probe selected from (i)-(iv),
(vi) a detectably labeled allele-specific probe having a nucleotide sequence that is complementary to the corresponding nucleotide sequence of a detectably labeled allele-specific probe selected from the group consisting of (i)-(v); or
(vii) any combination of (i)-(vi); and
wherein detection of binding of the detectably labeled allele-specific probe comprising or consisting of the nucleotide sequence of SEQ ID NO:5 to the EBV nucleic acids or an amplicon thereof produced by the forward primer comprising or consisting of the nucleotide sequence of SEQ ID NO: 3 and the reverse primer comprising or consisting of the nucleotide sequence of SEQ ID NO:4, if present, indicates the BALF2 gene has an adenine (A) at nucleotide position 162215,
wherein detection of binding of the detectably labeled allele-specific probe comprising or consisting of the nucleotide sequence of SEQ ID NO:8 to the EBV nucleic acids or an amplicon thereof produced by the forward primer comprising or consisting of the nucleotide sequence of SEQ ID NO: 6 and the reverse primer comprising or consisting of the nucleotide sequence of SEQ ID NO:7, if present, indicates the BALF2 gene has a cytosine (C) at nucleotide position 162476,
wherein detection of binding of the detectably labeled allele-specific probe comprising or consisting of the nucleotide sequence of SEQ ID NO:11 to the EBV nucleic acids or an amplicon thereof produced by the forward primer comprising or consisting of the nucleotide sequence of SEQ ID NO: 9 and the reverse primer comprising or consisting of the nucleotide sequence of SEQ ID NO: 10, if present, indicates the BALF2 gene has a thymine (T) at nucleotide position 163364,
wherein detection of binding of the detectably labeled allele-specific probe comprising or consisting of the nucleotide sequence of SEQ ID NO:12 to the EBV nucleic acids or an amplicon thereof produced by the forward primer comprising or consisting of the nucleotide sequence of SEQ ID NO: 3 and the reverse primer comprising or consisting of the nucleotide sequence of SEQ ID NO:4, if present, indicates the BALF2 gene has a cytosine (C) at position 162215, and
wherein the nucleotide positions are numbered relative to the reference nucleotide sequence of SEQ ID NO:2.

2. The method of claim 1, wherein the set of primers comprises the forward primer comprising or consisting of the nucleotide sequence of SEQ ID NO:3 and the reverse primer comprising or consisting of the nucleotide sequence of SEQ ID NO:4; and the one or more detectably labeled allele-specific probes comprise the detectably labeled allele-specific probe comprising or consisting of the nucleotide sequence of SEQ ID NO:5.

3. The method of claim 2, wherein the one or more detectably labeled allele-specific probes further comprise the detectably labeled allele-specific probe comprising or consisting of the nucleotide sequence of SEQ ID NO:12.

4. The method of any one of claims 1-3, wherein the set of primers comprises the forward primer comprising or consisting of the nucleotide sequence of SEQ ID NO:6 and the reverse primer comprising or consisting of the nucleotide sequence of SEQ ID NO:7; and the one or more detectably labeled allele-specific probes comprise the detectably labeled allele-specific probe comprising or consisting of the nucleotide sequence of SEQ ID NO:8.

5. The method of any one of claims 1-4, wherein the set of primers comprises the forward primer comprising or consisting of the nucleotide sequence of SEQ ID NO:9 and the reverse primer comprising or consisting of the nucleotide sequence of SEQ ID NO:10; and the one or more detectably labeled allele-specific probes comprise the detectably labeled allele-specific probe comprising or consisting of the nucleotide sequence of SEQ ID NO:11.

6. The method of claim 1, wherein the set of primers comprises: the forward primer comprising or consisting of the nucleotide sequence of SEQ ID NO:3 and the reverse primer comprising or consisting of the nucleotide sequence of SEQ ID NO:4, the forward primer comprising or consisting of the nucleotide sequence of SEQ ID NO:6 and the reverse primer comprising or consisting of the nucleotide sequence of SEQ ID NO:7; and the forward primer comprising or consisting of the nucleotide sequence of SEQ ID NO:9 and the reverse primer comprising or consisting of the nucleotide sequence of SEQ ID NO:10; and the one or more detectably labeled allele-specific probes comprise: the detectably labeled allele-specific probe comprising or consisting of the nucleotide sequence of SEQ ID NO:5, the detectably labeled allele-specific probe comprising or consisting of the nucleotide sequence of SEQ ID NO:8, the detectably labeled allele-specific probe comprising or consisting of the nucleotide sequence of SEQ ID NO:11, and the detectably labeled allele-specific probe comprising or consisting of the nucleotide sequence of SEQ ID NO:12.

7. The method of any one of claims 1-6, further comprising using a nucleic acid comprising or consisting of the nucleotide sequence of SEQ ID NO:13 as a risk allele control.

8. The method of any one of claims 1-7, further comprising using a nucleic acid comprising or consisting of the nucleotide sequence of SEQ ID NO:14 as a non-risk allele control.

9. The method of any one of claims 1-8, wherein each allele-specific probe is detectably labeled with a different fluorophore.

10. The method of any one of claims 1-9, wherein each allele-specific probe is detectably labeled with a 5′-fluorophore and a 3′-quencher.

11. The method of claim 10, wherein the 3′-quencher is a black hole quencher (BHQ) or tetramethyl rhodamine (TAMRA).

12. The method of any one of claims 1-11, wherein said amplifying comprises performing polymerase chain reaction (PCR) or isothermal amplification.

13. The method of claims 1-12, wherein the PCR is quantitative PCR.

14. The method of any one of claims 1-13, wherein the biological sample comprises blood, plasma, B cells, or epithelial cells.

15. The method of any one of claims 1-14, further comprising measuring EBV viral load in the biological sample.

16. The method of one of claims 1-15, further comprising determining whether the subject has a BALF2 haplotype associated with nasopharyngeal carcinoma (NPC), wherein detection of a cytosine (C) at nucleotide position 162215, a cytosine (C) at nucleotide position 162476, and a thymine (T) or a cytosine (C) at nucleotide position 163364 indicates the subject has a BALF2 haplotype associated with nasopharyngeal carcinoma (NPC) and is at risk of developing nasopharyngeal carcinoma.

17. The method of claim 16, further comprising performing further screening of the subject for nasopharyngeal carcinoma if the subject is identified as having a BALF2 haplotype associated with nasopharyngeal carcinoma (NPC).

18. The method of claim 17, wherein said performing further screening comprises performing an endoscopy or magnetic resonance imaging (MRI).

19. The method of claim 17 or 18, further comprising treating the subject for nasopharyngeal carcinoma if the subject is identified as having nasopharyngeal carcinoma based on said genotyping and further screening.

20. A method for genotyping one or more polymorphisms in a BALF2 gene of Epstein-Barr virus (EBV) using a nucleic acid amplification assay, the method comprising:
(a) obtaining a biological sample suspected of containing EBV nucleic acids from a subject;
(b) amplifying the EBV nucleic acids, if present, with a set of primers comprising:
(i) a forward primer comprising or consisting of the nucleotide sequence of SEQ ID NO: 3 and a reverse primer comprising or consisting of the nucleotide sequence of SEQ ID NO: 4,
(ii) a forward primer comprising or consisting of the nucleotide sequence of SEQ ID NO: 6 and a reverse primer comprising or consisting of the nucleotide sequence of SEQ ID NO: 7; and
(iii) a forward primer comprising or consisting of the nucleotide sequence of SEQ ID NO: 9 and a reverse primer comprising or consisting of the nucleotide sequence of SEQ ID NO: 10; and
(c) genotyping the BALF2 gene by detecting the presence of one or more alleles at the one or more polymorphisms of the BALF2 gene in the amplified nucleic acids using a set of detectably labeled allele-specific probes comprising:
(i) a detectably labeled allele-specific probe comprising or consisting of the nucleotide sequence of SEQ ID NO:5,
(ii) a detectably labeled allele-specific probe comprising or consisting of the nucleotide sequence of SEQ ID NO:8,
(iii) a detectably labeled allele-specific probe comprising or consisting of the nucleotide sequence of SEQ ID NO:11, and
(iv) a detectably labeled allele-specific probe comprising or consisting of the nucleotide sequence of SEQ ID NO:12;
wherein detection of binding of the detectably labeled allele-specific probe comprising or consisting of the nucleotide sequence of SEQ ID NO:5 to the EBV nucleic acids or an amplicon thereof produced by the forward primer comprising or consisting of the nucleotide sequence of SEQ ID NO: 3 and the reverse primer comprising or consisting of the nucleotide sequence of SEQ ID NO:4, if present, indicates the BALF2 gene has an adenine (A) at nucleotide position 162215,
wherein detection of binding of the detectably labeled allele-specific probe comprising or consisting of the nucleotide sequence of SEQ ID NO:8 to the EBV nucleic acids or an amplicon thereof produced by the forward primer comprising or consisting of the nucleotide sequence of SEQ ID NO: 6 and the reverse primer comprising or consisting of the nucleotide sequence of SEQ ID NO:7, if present, indicates the BALF2 gene has a cytosine (C) at nucleotide position 162476,
wherein detection of binding of the detectably labeled allele-specific probe comprising or consisting of the nucleotide sequence of SEQ ID NO:11 to the EBV nucleic acids or an amplicon thereof produced by the forward primer comprising or consisting of the nucleotide sequence of SEQ ID NO: 9 and the reverse primer comprising or consisting of the nucleotide sequence of SEQ ID NO: 10, if present, indicates the BALF2 gene has a thymine (T) at nucleotide position 163364,
wherein detection of binding of the detectably labeled allele-specific probe comprising or consisting of the nucleotide sequence of SEQ ID NO:12 to the EBV nucleic acids or an amplicon thereof produced by the forward primer comprising or consisting of the nucleotide sequence of SEQ ID NO: 3 and the reverse primer comprising or consisting of the nucleotide sequence of SEQ ID NO:4, if present, indicates the BALF2 gene has a cytosine (C) at position 162215, and
wherein the nucleotide positions are numbered relative to the reference nucleotide sequence of SEQ ID NO:2.

21. The method of any one of claims 1-20, wherein the allele-specific probes comprise one or more propynyl-modified bases.

22. A composition for genotyping one or more polymorphisms in a BALF2 gene of Epstein-Barr virus (EBV) in a biological sample using a nucleic acid amplification assay, the composition comprising a set of primers and allele-specific probes comprising:
(a) a forward primer comprising or consisting of the nucleotide sequence of SEQ ID NO:3, a reverse primer comprising or consisting of the nucleotide sequence of SEQ ID NO:4, an allele-specific probe comprising or consisting of the nucleotide sequence of SEQ ID NO:5, and an allele-specific probe comprising or consisting of the nucleotide sequence of SEQ ID NO:12;
(b) a forward primer comprising or consisting of the nucleotide sequence of SEQ ID NO:6, a reverse primer comprising or consisting of the nucleotide sequence of SEQ ID NO:7, and an allele-specific probe comprising or consisting of the nucleotide sequence of SEQ ID NO:8;
(c) a forward primer comprising or consisting of the nucleotide sequence of SEQ ID NO:9, a reverse primer comprising or consisting of the nucleotide sequence of SEQ ID NO:10, and an allele-specific probe comprising or consisting of the nucleotide sequence of SEQ ID NO:11;
(d) a forward primer, a reverse primer, and an allele-specific probe comprising at least one nucleotide sequence that differs from the corresponding nucleotide sequence of the forward primer, the reverse primer, and the allele-specific probe of a set selected from the group consisting of (a)-(c) in that the forward primer, the reverse primer, or the allele-specific probe has up to three nucleotide changes compared to the corresponding nucleotide sequence, wherein the forward primer and the reverse primer are capable of hybridizing to and amplifying the EBV nucleic acids in the nucleic acid amplification assay, and wherein the allele-specific probe retains allele-specificity;
(e) a forward primer, a reverse primer, and an allele-specific probe comprising nucleotide sequences that are complements of the corresponding nucleotide sequences of the forward primer, reverse primer, and the allele-specific probe of a set selected from the group consisting of (a)-(i); or
(f) any combination of (a)-(e).

23. The composition of claim 22, wherein the set of primers and allele-specific probes comprises:
(a) a forward primer comprising or consisting of the nucleotide sequence of SEQ ID NO:3, a reverse primer comprising or consisting of the nucleotide sequence of SEQ ID NO:4, an allele-specific probe comprising or consisting of the nucleotide sequence of SEQ ID NO:5, and an allele-specific probe comprising or consisting of the nucleotide sequence of SEQ ID NO:12;
(b) a forward primer comprising or consisting of the nucleotide sequence of SEQ ID NO:6, a reverse primer comprising or consisting of the nucleotide sequence of SEQ ID NO:7, and an allele-specific probe comprising or consisting of the nucleotide sequence of SEQ ID NO:8; and
(c) a forward primer comprising or consisting of the nucleotide sequence of SEQ ID NO:9, a reverse primer comprising or consisting of the nucleotide sequence of SEQ ID NO:10, and an allele-specific probe comprising or consisting of the nucleotide sequence of SEQ ID NO:11.

24. The composition of claim 22 or 23, wherein the allele-specific probes are detectably labeled.

25. The composition of claim 24, wherein each allele-specific probe is labeled with a different fluorophore.

26. The composition of claim 25, wherein each allele-specific probe is detectably labeled with a 5′-fluorophore and a 3′-quencher.

27. The composition of claim 26, wherein the 3′-quencher is a black hole quencher (BHQ) or tetramethyl rhodamine (TAMRA).

28. The composition of any one of claims 22-27, wherein the allele-specific probes comprise one or more propynyl-modified bases.

29. A kit comprising the composition of any one of claims 12-28 and instructions for genotyping one or more polymorphisms in a BALF2 gene of Epstein-Barr virus (EBV) in a biological sample.

30. The kit of claim 28, further comprising Taq polymerase and deoxyribonucleotide triphosphates.
